(19) United States
(12) Patent Application Publication
(10) Pub. No.: US 2024/0368282 A1
(43) Pub. Date: Nov. 7, 2024

(54) METHODS AND COMPOSITIONS FOR STIMULATING IMMUNE ACTIVITY

(71) Applicant: BIOSCEPTRE (AUST) PTY LTD, North Ryde (AU)

(72) Inventors: Patrick Schlegel, North Ryde (AU); Alexander Joechner, North Ryde (AU)

(21) Appl. No.: 18/688,086

(22) PCT Filed: Sep. 1, 2022
(86) PCT No.: PCT/AU22/51070

§ 371 Date: Feb. 29, 2024

(30) Foreign Application Priority Data
Sep. 1, 2021 (AU) 2021902832

(51) Int. Cl. C07K 16/28; A61K 35/17; A61K 39/00; A61P 35/00

(52) U.S. Cl. CPC C07K 16/2809; A61K 35/17; A61K 39/4611; A61K 39/4631; A61P 35/00; A61K 2239/11; C07K 2317/31; C07K 2317/565; C07K 2317/567

(57) ABSTRACT

The present invention relates to compositions and methods or uses of those compositions for amplifying immune activity to treat a variety of diseases or conditions, particularly cancer. A method for treating a condition comprising administering to a subject an antigen binding protein comprising (i) a first antigen binding domain that binds to a tumour-specific antigen; and (ii) a second antigen binding domain that binds to a cell surface molecule on an immune cell, thereby treating the condition. Preferably, further comprising a bridging molecule comprising (i) a targeting moiety that binds to a cell surface molecule on a target cell; and (ii) a tumour-specific antigen epitope moiety that is bound by the first antigen binding domain of the antigen binding protein.

### FIELD OF THE INVENTION

The present invention relates to compositions and methods or uses of those compositions for stimulating immune activity to treat a variety of diseases or conditions, particularly cancer.

### RELATED APPLICATION

This application claims priority from Australian provisional application AU 2021902832, the entire contents of which are hereby incorporated by reference.

### BACKGROUND OF THE INVENTION

One of the most promising advances is a new therapeutic class called active cellular immunotherapy (ACI). Cancer immunotherapies can be either passive or active. Passive therapy is based on the adoptive transfer of immunomodulators including cytokines, tumour specific antibodies or immune cells. These substances or cells are then administered to the patient to initiate an anti-tumour action. In general these therapies do not generate immunologic memory and therefore require chronic infusion based treatment. Active immunotherapies, on the other hand, stimulate the patient's immune system with the intent of promoting an antigen specific anti-tumour effect using the body's own immune cells. In addition active immunotherapies seek to create durable anti-tumour response that can protect against minimal residual disease and tumour recurrences.

Accordingly, despite advances in cancer therapy and infectious disease immunotherapy/vaccine technology, there remains an urgent need for new or improved effective immunotherapy approaches to the treatment of such diseases.

Reference to any prior art in the specification is not an acknowledgment or suggestion that this prior art forms part of the common general knowledge in any jurisdiction or that this prior art could reasonably be expected to be understood, regarded as relevant, and/or combined with other pieces of prior art by a skilled person in the art.

### SUMMARY OF THE INVENTION

In one aspect, the present invention provides a method for treating a condition comprising administering to a subject:
an antigen binding protein comprising:
   (i) a first antigen binding domain that binds to a tumour-specific antigen; and
   (ii) a second antigen binding domain that binds to a cell surface molecule on an immune cell,
thereby treating the condition.

In another aspect, the present invention provides a composition comprising:
an antigen binding protein comprising:
(i) a first antigen binding domain that binds to a tumour-specific antigen; and
(ii) a second antigen binding domain that binds to a cell surface molecule on an immune cell,
   preferably the composition further comprises a pharmaceutically acceptable carrier, diluent or excipient.

In another aspect, the present invention provides a kit comprising:
an antigen binding protein comprising:
(i) a first antigen binding domain that binds to a tumour-specific antigen; and
(ii) a second antigen binding domain that binds to a cell surface molecule on an immune cell,
   preferably the kit further comprises written instructions to use the antigen binding protein a method as described herein.

In one aspect, the present invention provides a method for treating a condition comprising administering to a subject:
(a) an antigen binding protein comprising:
   (i) a first antigen binding domain that binds to a tumour-specific antigen; and
   (ii) a second antigen binding domain that binds to a cell surface molecule on an immune cell; and
(b) a bridging molecule comprising:
   (i) a targeting moiety that binds to a cell surface molecule on a target cell; and
   (ii) a tumour-specific antigen epitope moiety that is bound by the first antigen binding domain of the antigen binding protein, thereby treating a condition in the subject.

In one aspect, the present invention provides a two component therapeutic comprising:
(a) an antigen binding protein comprising:
   (i) a first antigen binding domain that binds to a tumour-specific antigen; and
   (ii) a second antigen binding domain that binds to a cell surface molecule on an immune cell; and
(b) a bridging molecule comprising:
   (i) a targeting moiety that binds to a cell surface molecule on a target cell; and
   (ii) a tumour-specific antigen epitope moiety that is bound by the first antigen binding domain of the antigen binding protein.

In another aspect, the present invention provides a composition comprising:
(a) an antigen binding protein comprising:
   (i) a first antigen binding domain that binds to a tumour-specific antigen; and
   (ii) a second antigen binding domain that binds to a cell surface molecule on an immune cell; and
(b) a bridging molecule comprising:
   (i) a targeting moiety that binds to a cell surface molecule on a target cell; and
   (ii) a tumour-specific antigen epitope moiety that is bound by the first antigen binding domain of the antigen binding protein, preferably the composition further comprises a pharmaceutically acceptable carrier, diluent or excipient.

In another aspect, the present invention provides a kit comprising:
(a) an antigen binding protein comprising:
   (i) a first antigen binding domain that binds to a tumour-specific antigen; and
   (ii) a second antigen binding domain that binds to a cell surface molecule on an immune cell; and
(b) a bridging molecule comprising:
   (i) a targeting moiety that binds to a cell surface molecule on a target cell; and
   (ii) a tumour-specific antigen epitope moiety that is bound by the first antigen binding domain of the antigen binding protein,
      preferably the kit further comprises written instructions to use the antigen binding protein a method as described herein.

In another aspect, the present invention provides an antigen binding protein comprising:
(i) a first antigen binding domain that binds to a tumour-specific antigen; and
(ii) a second antigen binding domain that binds to a cell surface molecule on an immune cell.

In any aspect, the tumour-specific antigen is an antigen expressed on a solid or liquid tumour. In one embodiment, the tumour-specific antigen is any one of dysfunctional P2X₇ receptor, EGFRvIII or CLDN6. In any aspect, the first antigen binding domain binds to, or specifically binds to, a dysfunctional P2X₇, EGFRvIII or CLDN6. Preferably the tumour-specific antigen is dysfunctional P2X₇.

Accordingly, in a preferred aspect, there is provided an antigen binding protein comprising:
(i) a first antigen binding domain that binds to dysfunctional P2X₇ receptor; and
(ii) a second antigen binding domain that binds to a cell surface molecule on an immune cell.

In any embodiment, the antigen binding protein comprising the first and second antigen binding domains (further defined herein as an “orchestration molecule”) may be at least a bivalent molecule, or may be a multivalent molecule such as a tetravalent molecule. For example, the antigen binding protein may comprise a single binding domain for binding to the tumour-specific antigen (preferably dysfunctional P2X₇) and a single binding domain for binding to a cell surface molecule on an immune cell, making the molecule a bivalent molecule. Non-limiting examples of such a molecule may be a fusion protein comprising an scFv for binding to each of the first and second antigens, or a fusion protein comprising a monomeric IgG and an scFv. Alternatively, tetravalent molecules may comprise an antigen binding protein in the form of a dimeric IgG molecule for binding to a cell surface molecule on an immune cell, to which is fused (eg via the C terminus of each heavy chain) scFvs for binding to a tumour-specific antigen (such as dysfunctional P2X₇ receptor). Further still are contemplated IgG-derived binding proteins with or without CH2 and/or CH3 domains. Still further, the Fc-binding domains of the CH2 and CH3 domain (if included) may be modified to either attenuate or increase FcRN binding. Non-limiting examples of suitable architectures for various OR molecules are provided herein in the Examples and also in the Figures. It will be within the purview of the skilled person to be able to design and obtain a suitable OR molecule based on those exemplary architectures.

In another aspect, the present invention provides a nucleic acid comprising a nucleotide sequence encoding an antigen binding protein as described herein. Preferably, the nucleic acid comprises a first nucleotide sequence encoding a first antigen binding domain and a second nucleotide sequence encoding a second antigen binding domain. In any aspect, the nucleic acid may be DNA or RNA.

In another aspect, the present invention provides a nucleic acid comprising a nucleotide sequence encoding an antigen binding protein as described herein and a nucleotide sequence encoding a bridging molecule as described herein. Preferably, the nucleic acid comprises a first nucleotide sequence encoding a first antigen binding domain and a second nucleotide sequence encoding a second antigen binding domain.

In any aspect, the present invention further comprises an immune cell or progenitor thereof, expressing a receptor comprising an antigen-recognition domain and a signalling domain. Preferably, the antigen-recognition domain binds to a tumour-specific antigen expressed on a cell surface. For example, in any method of the invention, the method further comprises administering an immune cell or progenitor thereof, expressing a receptor comprising an antigen-recognition domain and a signalling domain. Preferably, the antigen-recognition domain binds to a tumour-specific antigen expressed on a cell surface. Also, in another aspect, the present invention provides a three-component therapeutic that includes the two-component therapeutic as described herein and further comprising an immune cell or progenitor thereof, expressing a receptor comprising an antigen-recognition domain and a signalling domain. Preferably, the antigen-recognition domain binds to a tumour-specific antigen expressed on a cell surface. In any embodiment, the immune cell is a T cell expressing a chimeric antigen receptor (CAR), i.e. a CAR-T cell.

In any embodiment, the first antigen binding domain binds to an epitope associated with an adenosine triphosphate (ATP)-binding site of the dysfunctional P2X₇ receptor. In some embodiments, the dysfunctional P2X₇ receptor has a reduced capacity to bind ATP at the ATP-binding site compared to an ATP-binding capacity of a functional P2X₇ receptor (e.g., a receptor having wild-type sequence and having a conformation or fold of an ATP-binding receptor). In some embodiments the dysfunctional P2X₇ receptor cannot bind ATP at the ATP-binding site.

In any embodiment, the dysfunctional P2X₇ receptor has a conformational change that renders the receptor dysfunctional. In some embodiments, the conformational change is a change of an amino acid from the trans-conformation to the cis-conformation. In some embodiments, the amino acid that has changed from a trans-conformation to a cis-conformation is proline at amino acid position 210 of the dysfunctional P2X₇ receptor.

In any embodiment, the first antigen binding domain binds to an epitope that includes the proline at amino acid position 210 of the dysfunctional P2X₇ receptor. In some embodiments, the first antigen binding site binds to an epitope that includes one or more amino acid residues spanning from glycine at amino acid position 200 to cysteine at amino acid position 216, inclusive, of the dysfunctional P2X₇ receptor.

The first antigen binding domain present can be any suitable molecule that can interact with and specifically binds to a dysfunctional P2X₇ receptor. However, in some embodiments, the first antigen binding domain includes amino acid sequence homology to the amino acid sequence of an antibody, or a fragment thereof, which binds to the dysfunctional P2X₇ receptor. In some embodiments, the first antigen binding domain includes amino acid sequence homology to the amino acid sequence of a fragment-antigen binding (Fab) portion of an antibody that binds to a dysfunctional P2X₇ receptor. In some embodiments, the antibody is a humanised antibody.

In any embodiment, the first antigen binding domain includes amino acid sequence homology to the amino acid sequence of a single-chain variable fragment (scFv) or a multivalent scFv that binds to a dysfunctional P2X₇ receptor. In some embodiments, the multivalent scFv is a divalent or trivalent scFv.

In any embodiment, the first antigen binding domain includes amino acid sequence homology to a single-antibody domain (sdAb) that binds to a dysfunctional P2X₇ receptor.

In any embodiment, the first antigen binding domain includes a binding polypeptide that includes amino acid sequence homology to one or more complementarity determining regions (CDRs) of an antibody that binds to a dysfunctional P2X₇ receptor. In any embodiment, the binding polypeptide includes amino acid sequence homology to the CDR1, 2 and 3 domains of the V_{H} and/or V_{L} chain of an antibody that binds to a dysfunctional P2X₇ receptor. In preferred embodiments, the binding polypeptide comprises the amino acid sequence of the CDRs of the V_{H} and/or V_{L} chain of an antibody, or the amino acid sequence of the V_{H} and/or V_{L} chains of an antibody, or the amino acid sequence of an antibody or fragment thereof, wherein the antibody or fragment thereof comprises the amino acid sequences of any antibody described in PCT/AU2002/000061 or PCT/AU2002/001204 (or in any one of the corresponding U.S. Pat. Nos. 7,326,415, 7,888,473, 7,531,171, 8,080,635, 8,399,617, 8,709,425, 9,663,584, or U.S. Pat. No. 10,450,380), PCT/AU2007/001540 (or in corresponding U.S. Pat. No. 8,067,550), PCT/AU2007/001541 (or in corresponding US publication US 2010-0036101), PCT/AU2008/001364 (or in any one of the corresponding U.S. Pat. Nos. 8,440,186, 9,181,320, 9,944,701 or U.S. Pat. No. 10,597,451), PCT/AU2008/001365 (or in any one of the corresponding U.S. Pat. No. 8,293,491 or U.S. Pat. No. 8,658,385), PCT/AU2009/000869 (or in any one of the corresponding U.S. Pat. No. 8,597,643, 9,328,155 or 10,238,716), PCT/AU2010/001070 (or in any one of the corresponding publications WO/2011/020155, U.S. Pat. Nos. 9,127,059, 9,688,771, or U.S. Pat. No. 10,053,508), and PCT/AU2010/001741 (or in any one of the corresponding publications WO 2011/075789 or U.S. Pat. No. 8,835,609) the entire contents of which are hereby incorporated by reference. Preferably the antibody comprises the CDR amino acid sequences of 2-2-1 described in PCT/AU2010/001070 (or in any one of the corresponding U.S. Pat. Nos. 9,127,059, 9,688,771, or U.S. Pat. No. 10,053,508) or BPM09 described in PCT/AU2007/001541 (or in corresponding US publication US 2010-0036101) and produced by the hybridoma AB253 deposited with the European Collection of Cultures (ECACC) under Accession no. 06080101.

In any aspect, the cell surface molecule on an immune cell is present on the surface of a lymphoid or myeloid lineage cell. The lymphocyte may be a subtype of innate lymphoid cell, invariant NK cell, NK cell or a T lymphocyte (e.g. cytotoxic T cell, immunomodulatory T cell, γδ T cell, or NKT cell) or a subset of B lymphocyte. The myeloid lineage cell may be a monocyte, a macrophage, dendritic cell or subtype of granulocyte. The cell surface molecule on an immune cell may be any molecule that is present on an immune cell that can be bound by or detected by an antigen binding domain. Preferably, the cell surface molecule is only present on an immune cell and not present on a non-immune cell. Preferably the cell surface molecule is a receptor that directly or indirectly causes activation of the immune cell. Typically, activation of the immune cell results in an increased ability to reduce the viability of a cancer cell.

In any aspect, the second antigen binding domain binds to, or specifically binds to, a cell surface molecule on an immune cell as described herein. In one embodiment, the second antigen binding domain binds to or specifically binds to a cell surface molecule on a T cell; optionally wherein the cell surface molecule is a T cell receptor or a molecule associated with a T cell receptor, such as a TCR-alpha or beta chain, or a chain of the CD3 T cell receptor complex, e.g. epsilon chain. In another embodiment, the cell surface molecule is a costimulatory receptor, such as CD27, CD28, CD30, CD40, DAP10, OX40, 4-1 BB (CD137) and ICOS. In another embodiment, the cell surface molecule may be an Fc receptor, or portion thereof, such as FcγRI (CD64), FcγRIIa (CD32), FcγRIIb (CD32), FcγRIIIa (CD16a), FcγRIIIb (CD16b). In other examples, the second antigen binding domain binds to or specifically binds to a cell surface molecule on an innate immune effector cell, preferably an innate immune effector cell. Examples of cell surface molecules expressed on innate immune effector cells include CD16 (also known as FcγRIIIa), NKp46, NKG2D, NKp44 and DNAM-1 and others.

The second antigen binding domain may be any molecule that binds to a cell surface molecule on an immune cell. For example, the second antigen binding domain may comprise, or be part of, an antibody or antigen binding fragment thereof. Alternatively, the second antigen binding domain may be an Fc region or part thereof capable of binding to an Fc receptor such as FcγRI (CD64), FcγRIIa (CD32), FcγRIIb (CD32), FcγRIIIa (CD16a), FcγRIIIb (CD16b). In any aspect or embodiment, the second antigen binding domain may be an Fc region of an antibody or a polypeptide comprising an Fc receptor binding domain.

In any aspect, the Fc region of the antibody is an Fc region of an IgG, more preferably IgG1, more preferably a human IgG1. In some embodiments, the Fc region of an IgG is a mouse IG1.

In any aspect, the one or more of the 2 or more polypeptides, or one or both of the receptor binding domains in the dimer of the chimeric or fusion protein, may be fused at the C-terminus to the Fc region. Alternatively, one or more of the 2 or more polypeptides, or one or both of the receptor binding domains in the dimer of the chimeric or fusion protein may be fused via a linker at the C-terminus to the Fc region.

Preferably, the Fc region comprises two heavy chain fragments, more preferably the CH2 and CH3 domains of said heavy chain. In one embodiment, the heavy chain fragments are linked via disulphide linkages. Alternatively, neither the heavy chain fragments nor the Fc region is disulphide linked, or linked in any way.

In any embodiment, the bridging molecule may be a polypeptide, or a polypeptide conjugated to a molecule with the function of a bridging molecule, e.g. a DNA aptamer. The polypeptide may be expressed by the immune cell or progenitor thereof. Alternatively, the therapeutic, composition or kit may comprise the polypeptide, or a nucleic acid encoding said polypeptide.

The bridging molecule may be a polypeptide, for example a fusion or chimeric protein. In alternative embodiments, the bridging molecule may comprise polypeptides or peptides that are linked via linking molecules.

In any embodiment, the cell surface molecule to which the targeting moiety binds, or specifically binds, may comprise an antigen, preferably an antigen as described herein.

The cell surface molecule may be selected from a protein, a lipid moiety, a glycoprotein, a glycolipid, a carbohydrate, a polysaccharide, a nucleic acid, an MHC-bound peptide, or a combination thereof.

The cell surface molecule may comprise parts (e.g., coats, capsules, cell walls, flagella, fimbrae, and toxins) of bacteria, viruses, and other microorganisms. The cell surface molecule may be expressed by the target cell.

The cell surface molecule may not be expressed by the target cell. By way of non-limiting example, the cell surface molecule may be a ligand expressed by a cell that is not the target cell and that is bound to the target cell or a cell surface molecule of the target cell. Also, by non-limiting example, the cell surface molecule may be a toxin, exogenous molecule or viral protein that is bound to a cell surface or cell surface receptor of the target cell.

The target cell may be a cancer cell, or a cell capable of presenting a peptide from an infectious agent on an MHC class receptor. The target cell may or may not express a tumour-specific antigen, for example a dysfunctional P2X₇ receptor.

In any aspect, the target cell may be any cell expressing a dysfunctional P2X₇ receptor, for example a cancer cell.

In any embodiment, 2 or more bridging molecules may be administered to a subject, each bridging molecule comprising a targeting moiety that binds to a different cell surface molecule on a target cell. For example, in the context of a method of treating cancer, each bridging molecule administered may comprise different targeting moieties and may therefore bind to a different tumour associated antigen present on the cancer cells. Such embodiments facilitate redirection of a single class of CAR T cell to multiple antigens present on tumour antigens (including at the same time) and therefore provide a multi-pronged approach for killing of cancer cells.

Accordingly, in any embodiment, the method of treating cancer comprises administering 2 or more bridging molecules, wherein each bridging molecule comprises targeting moieties for binding to different cell surface antigens on a target cell.

In further embodiments, the bridging molecules may bind to different epitopes on the same cell surface antigen expressed by the cancer cell. Accordingly, in further embodiments, the methods of the invention comprise treating cancer comprises administering 2 or more bridging molecules, wherein each bridging molecule comprises targeting moieties for binding to different epitopes on the same cell surface antigen on a target cell.

Further still, the invention provides for methods wherein bridging molecules for redirecting an immune cell to different cancer antigens, can be administered synchronously to a subject in need thereof. This allows for fine-tuning of the therapeutic approach, such that an immune cell may be directed to binding cancer cells via different antigens, at different times during the course of the patient's therapeutic regimen.

In further embodiments, a single bridging molecule may comprise more than one targeting moiety, such that a single molecule comprises targeting moieties for more than one cell surface molecule on a target cell.

Further still, a single bridging molecule may comprise more than one targeting moiety, such that a single molecule comprises targeting moieties for the same cell surface molecule on a target cell, but wherein the targeting moieties bind to different epitopes on the cell surface molecule.

In any aspect, the targeting moiety that binds to a cell surface molecule on a target cell comprises or consists of a peptide or antibody or antibody fragment. Alternatively, the targeting moiety may comprise a ligand or binding partner for a protein or receptor present on the target cell surface.

The targeting moiety may further comprise a soluble T cell receptor (TcR) or a single chain T cell receptor binding motif or a T cell receptor-like mAb. In such embodiments, the targeting moiety is particularly suitable for the binding of peptides derived from intracellularly processed proteins from infectious agents that are presented on a cell surface via MHC (HLA) I and II molecules. The targeting moiety may also be suitable for binding of peptides presented by MHC molecules, wherein the peptides comprise mutations associated with cancers, such as the cancer testis antigens (WT1, NY-ESO-1, PRAME family (e.g. PRA100, PRA142, PRA300, PRA425 and others), MAGE family (e.g., MAGE-A1, MAGE-A3, MAGE-A4, MAGE-A12 and others), CT83, SSX2, GAGE, BAGE, PAGE) or other cancer specific mutations.

In any aspect or embodiment, the targeting moiety of the bridging molecule does not bind to the same antigen or epitope as the antigen-recognition of the receptor. For example, the targeting moiety of the bridging molecule does not bind to a dysfunctional P2X₇ receptor, the E200, E300, or E200/E300 composite epitope, or any other epitope present on a dysfunctional P2X₇ receptor as described herein.

The targeting moiety may be a targeting antibody or antibody fragment. The targeting antibody or antibody fragment may be an immunoglobulin (Ig). The immunoglobulin may be selected from an IgG, an IgA, an IgD, an IgE, an IgM, a fragment thereof or a modification thereof. The immunoglobulin may be IgG. The IgG may be IgG1. The IgG may be any IgG subclass.

In any embodiment, a bridging molecule of the invention may comprise more than one targeting moiety. For example, in certain non-limiting embodiments, the bridging molecule may comprise two different antibodies, or fragment thereof. The antibodies may bind different epitopes of the same cell surface molecule on the target cell. Alternatively, the antibodies may bind epitopes of different cell surface molecules on the target cell.

In any embodiment of aspects directed to methods of treatment herein, the antigen binding protein and/or bridging molecule may be delivered via infusion to the subject or may be expressed by the immune cell (for example one expressing a chimeric antigen receptor). The antigen binding protein and/or bridging molecule may be a polypeptide, which is encoded in an inducible or a constitutive expression construct contained in the immune cell.

In any aspect, tumour-specific antigen epitope moiety comprises or consists of an epitope from a tumour specific antigen. Typically, the tumour specific antigen is any one of dysfunctional P2X₇, EGFRvIII or CLDN6. In any aspect, the tumour-specific antigen epitope moiety is capable of being bound by the first antigen binding domain of an antigen binding protein as described herein. The tumour-specific antigen epitope moiety may be any one described herein.

In any aspect or embodiment, the tumour-specific antigen epitope moiety may be a dysfunctional P2X₇ receptor epitope moiety. The dysfunctional P2X₇ receptor epitope moiety may be provided in the form of a P2X₇ receptor, or a fragment of a P2X₇ receptor that has at least one of the three ATP binding sites that are formed at the interface between adjacent correctly packed monomers that are unable to bind ATP. Such receptors are unable to extend the opening of the non-selective calcium channels to apoptotic pores.

In any aspect, the dysfunctional P2X₇ receptor epitope moiety comprises or consists of a fragment of a dysfunctional P2X₇ receptor. Exemplary fragments include GHNYTTRNILPGLNITC (SEQ ID NO: 2; also referred to herein as the “E200 epitope”) and variants thereof (exemplary variants are provided in SEQ ID NOs: 3 to 10 and 15 to 30,168,361-396, 437 and 438); KYYKENNVEKRTLIKVF (SEQ ID NO: 12 and 13; also referred to herein as the “E300” epitope); or GHNYTTRNILPGAGAKYYKENNVEK (SEQ ID NO: 14; also referred to herein as the “E200/E300” or “composite” epitope).

In any aspect, the dysfunctional P2X₇ receptor epitope moiety is bound by an antibody that binds to dysfunctional P2X₇ receptors, but is not bound by antibodies that bind to functional P2X₇ receptors.

In any aspect, a bridging molecule may comprise 2 or more dysfunctional P2X₇ receptor epitope moieties. The 2 or more dysfunctional P2X₇ receptor epitope moieties may comprise or consist of the same sequence, or of different sequences. For example, in any aspect, a bridging molecule may comprise a dysfunctional P2X₇ receptor epitope moiety in the form of the E200 epitope and a further dysfunctional P2X₇ receptor epitope moiety in the form of the E300 epitope. Alternatively, in any aspect, a bridging molecule may comprise a dysfunctional P2X₇ receptor epitope moiety in the form of the E200 epitope and a further dysfunctional P2X₇ receptor epitope moiety in the form of the composite epitope. Still further, in any aspect, a bridging molecule may comprise a first dysfunctional P2X₇ receptor epitope moiety in the form of the E200 epitope and a further dysfunctional P2X₇ receptor epitope moiety in the form of the E200 epitope.

In further aspects of the invention, there is provided an antigen binding protein comprising an antigen binding domain that binds to a P2X₇ receptor that has an impaired response to ATP such that it is unable to form an apoptotic pore under physiological conditions (i.e., a dysfunctional or non-functional P2X₇ receptor as herein defined). Preferably, the antigen binding protein does not bind to P2X₇ receptors that function normally in response to ATP.

Preferably, the antigen binding protein comprising an antigen binding domain that comprises:

FR1-CDR1-FR2-CDR2-FR3-CDR3-FR4,
wherein:
FR1, FR2, FR3 and FR4 are each framework regions;
CDR1, CDR2 and CDR3 are each complementarity determining regions;
wherein the sequence of any of complementarity determining regions are as described herein in Table 2.

Preferably, the framework regions have an amino acid sequence also as described in Table 2, including amino acid variation at particular residues which can be determined by aligning the various framework regions derived from each antibody.

Further, the present invention provides an antigen binding protein that binds to or specifically binds to a dysfunctional P2X₇ receptor, wherein the antigen binding protein comprises an antigen binding domain comprising:

a variable heavy (VH) chain comprising FR1-CDR1-FR2-CDR2-FR3-CDR3-FR4,
wherein:
FR1, FR2, FR3 and FR4 are each framework regions;
CDR1, CDR2 and CDR3 are each complementarity determining regions;
and wherein the antigen binding domain comprises the CDRs1-3 of any of the VH chains as defined in any one of SEQ ID NOs: 400 or 402. Preferably, the CDRs of the antigen binding proteins may be determined using the Kabat Chothia or IMGT domain gap numbering system, or Martin systems, more preferably, using the Kabat system.

In one aspect, the invention provides an antigen binding protein, comprising, consisting or consisting essentially of an amino acid sequence as set forth in any one of SEQ ID NOs: 400, 402 or 411.

In another aspect, the present invention provides an antigen binding protein comprising an antigen binding domain of an antibody, wherein the antigen binding domain binds to or specifically binds to a dysfunctional P2X₇ receptor, wherein the antigen binding domain comprises at least one of:
(i) a VH comprising a complementarity determining region (CDR) 1 comprising a sequence at least about 80%, at least 85%, at least 90%, at least 92%, at least 95%, at least 97%, at least 99% identical to a sequence set forth in SEQ ID NO: 397, a CDR2 comprising a sequence at least about 80%, at least 85%, at least 90%, at least 92%, at least 95%, at least 97%, at least 99% identical to a sequence set forth in SEQ ID NO: 398 and a CDR3 comprising a sequence at least about 80%, at least 85%, at least 90%, at least 92%, at least 95%, at least 97%, at least 99% identical to a sequence set forth in any one of SEQ ID NOs: 399 or 401;
(ii) a VH comprising a sequence at least about 95% or 96% or 97% or 98% or 99% identical to a sequence set forth in any one of SEQ ID NOs: 400 or 402;
(iii) a VH comprising a CDR1 comprising a sequence set forth SEQ ID NO: 397, a CDR2 comprising a sequence set forth in any one of SEQ ID NO: 398, and a CDR3 comprising a sequence set forth in any one of SEQ ID NOs: 399 or 401;
(iv) a VH comprising a sequence set forth in any one of SEQ ID NOs: 400 or 402.

In any embodiment, the antigen binding domain further comprises at least one of:
(i) a VH comprising a framework region (FR) 1 comprising a sequence at least about 80%, at least 85%, at least 90%, at least 92%, at least 95%, at least 97%, at least 99% identical to a sequence set forth in any one of SEQ ID NOs: 403 or 404, a FR2 comprising a sequence at least about 80%, at least 85%, at least 90%, at least 92%, at least 95%, at least 97%, at least 99% identical to a sequence set forth in SEQ ID NO: 405, a FR3 comprising a sequence at least about 80%, at least 85%, at least 90%, at least 92%, at least 95%, at least 97%, at least 99% identical to a sequence set forth in SEQ ID NO: 406, and a FR4 comprising a sequence at least about 80%, at least 85%, at least 90%, at least 92%, at least 95%, at least 97%, at least 99% identical to a sequence set forth in SEQ ID NO: 407; or
(ii) a VH comprising a FR1 comprising a sequence set forth in any one of SEQ ID NOs: 403 or 404, a FR2 comprising a sequence set forth in SEQ ID NO: 405, a FR3 comprising a sequence set forth in SEQ ID NO: 406, and a FR4 comprising a sequence set forth in SEQ ID NO: 407.

The present invention also provides an antigen binding protein that binds to or specifically binds to a dysfunctional P2X₇ receptor and wherein the antigen binding protein competitively inhibits binding of an antigen binding protein comprising, consisting or consisting essentially of an amino acid sequence as set forth in any one of SEQ ID NOs: 400 or 402.

In another aspect, the present invention also provides an antigen binding protein comprising an antigen binding domain of an antibody, wherein the antigen binding domain binds to or specifically binds to a dysfunctional P2X₇ receptor, wherein the antigen binding domain comprises at least one of:
(i) a VH comprising a complementarity determining region (CDR) 1 comprising a sequence at least about 80%, at least 85%, at least 90%, at least 92%, at least 95%, at least 97%, at least 99% identical to a sequence set forth in SEQ ID NO: 408, 416, 423, or 430, a CDR2 comprising a sequence at least about 80%, at least 85%, at least 90%, at least 92%, at least 95%, at least 97%, at least 99% identical to a sequence set forth in SEQ ID NO: 409, 417, 424 or 431 and a CDR3 comprising a sequence at least about 80%, at least 85%, at least 90%, at least 92%, at least 95%, at least 97%, at least 99% identical to a sequence set forth in any one of SEQ ID NOs: 410, 418, 424 or 432;
(ii) a VH comprising a sequence at least about 95% or 96% or 97% or 98% or 99% identical to a sequence set forth in SEQ ID NO: 411;
(iii) a VH comprising a CDR1 comprising a sequence set forth SEQ ID NO: 408, 416, 423, or 430, a CDR2 comprising a sequence set forth in any one of SEQ ID NO: 409, 417, 424 or 431, and a CDR3 comprising a sequence set forth in any one of SEQ ID NOs: 410, 418, 424 or 432;
(iv) a VH comprising a sequence set forth in SEQ ID NO: 411.

The antigen binding domain may further comprises at least one of:
(i) a VH comprising a framework region (FR) 1 comprising a sequence at least about 80%, at least 85%, at least 90%, at least 92%, at least 95%, at least 97%, at least 99% identical to a sequence set forth in any one of SEQ ID NOs: 412, 419, 426 or 433, a FR2 comprising a sequence at least about 80%, at least 85%, at least 90%, at least 92%, at least 95%, at least 97%, at least 99% identical to a sequence set forth in SEQ ID NO: 413, 420, 427 or 434, a FR3 comprising a sequence at least about 80%, at least 85%, at least 90%, at least 92%, at least 95%, at least 97%, at least 99% identical to a sequence set forth in SEQ ID NO: 414, 421, 428 or 435, and a FR4 comprising a sequence at least about 80%, at least 85%, at least 90%, at least 92%, at least 95%, at least 97%, at least 99% identical to a sequence set forth in SEQ ID NO: 415, 422, 429 or 436; or
(ii) a VH comprising a FR1 comprising a sequence set forth in any one of SEQ ID NOs: 412, 419, 426 or 433, a FR2 comprising a sequence set forth in SEQ ID NO: 413, 420, 427 or 434, a FR3 comprising a sequence set forth in SEQ ID NO: 414, 421, 428 or 435, and a FR4 comprising a sequence set forth in SEQ ID NO: 415, 422, 429 or 436.

In any embodiment of the above two aspects, the antigen binding protein may additionally comprise a FR1a-CDR1a-FR2a-CDR2a-FR3a-CDR3a-FR4a, wherein FR1a, FR2a, FR3a and FR4a are each framework regions; and CDR1a, CDR2a and CDR3a are each complementarity determining regions. In certain embodiments, FR1a-CDR1a-FR2a-CDR2a-FR3a-CDR3a-FR4a corresponds to the sequence of a variable light (VL) chain.

In certain embodiments, the antigen binding protein comprises FR1-CDR1-FR2-CDR2-FR3-CDR3-FR4-linker-FR1a-CDR1a-FR2a-CDR2a-FR3a-CDR3a-FR4a; or FR1a-CDR1a-FR2a-CDR2a-FR3a-CDR3a-FR4a-FR1-CDR1-FR2-CDR2-FR3-CDR3-FR4.

As defined herein, the linker may be a chemical, one or more amino acids, or a disulphide bond formed between two cysteine residues. In preferred embodiments, the linker is comprised of one or more amino acid residues.

In any embodiment, the antigen binding protein comprises a variable light chain (VL) comprising a CDR of any of the sequences defined in any one of SEQ ID NOs: 309, 310, 311, 312, 319, 330, 331, 332 or 333. In any embodiment, the antigen binding protein comprises a variable light chain (VL) comprising a CDR1, CDR2 and CDR3 of any of the sequences defined in any one of SEQ ID NOs: 309, 310, 311, 312, 319, 330, 331, 332 or 333. Preferably, the CDRs may be determined using the Kabat Chothia, or IMGT domain gap numbering system, or Martin systems, more preferably, using the Kabat system.

In any embodiment, the antigen binding protein comprises a variable light chain (VL) comprising a the sequence as defined in any one of SEQ ID NOs: 309, 310, 311, 312, 319, 330, 331, 332 or 333, or sequences at least about 80%, 81%, 82%, 83%, 84%, 85%, 86%, 87%, 88%, 89%, 90%, 91%, 92%, 93%, 94%, 95%, 96%, 97%, 98% or at least 99% identical thereto.

In certain preferred embodiments, the invention provides an antigen binding protein comprising, consisting essentially of or consisting of the amino acid sequence of (in order of N to C terminus or C to N terminus) i) any of SEQ ID NOs: 400, 402 or 411; and ii) any of SEQ ID NOs: 309, 310, 311, 312, 319, 330, 331, 332 or 333.

As described herein, the antigen binding protein may be in the form of:
(i) a single chain Fv fragment (scFv);
(ii) a dimeric scFv (di-scFv); or
(iii) one of (i) or (ii) linked to a constant region of an antibody, Fc or a heavy chain constant domain (CH) 2 and/or CH3.

Further, as described herein, the antigen binding protein may be in the form of:
(i) a diabody;
(ii) a triabody;
(iii) a tetrabody;
(iv) a Fab;
(v) a F(ab′)2;
(vi) a Fv;
(vii) a bispecific antibody or other form of multispecific antibody (including a BiTE); or
(viii) one of (i) to (vii) linked to a constant region of an antibody, Fc or a heavy chain constant domain (CH) 2 and/or CH3.

In certain embodiments, the antigen binding protein of the present invention is a protein that does not comprise a constant region from an immunoglobulin. For example, the antigen binding protein may be an scFv, a dimeric scFv, an Fv fragment, a single domain antibody (dAb), a diabody, or fusion protein or conjugate comprising the same.

In certain embodiments, the antigen binding protein is in the form of a fusion protein as described herein in the context of an “orchestration molecule” (eg, comprising one antigen binding domain for binding to nfP2X₇ receptor and being joined or fused to a second antigen binding domain for binding to an antigen on an immune cell, preferably an immune effector cell including an innate immune effector cell).

The foregoing antigen binding proteins can also be referred to as antigen binding domains of antibodies.

In certain embodiments, the complementarity determining region sequences (CDRs) of an antigen binding protein of the invention may be defined according to the IMGT numbering system, Kabat, Martin or Chothia systems.

Reference herein to a protein or antibody that “binds to” dysfunctional P2X₇ receptor (nfP2X₇ receptor) provides literal support for a protein or antibody that “binds specifically to” or “specifically binds to” nfP2X₇ receptor.

Preferably, an antigen binding protein as described herein is an antibody or antigen binding fragment thereof. Typically, the antigen binding protein is an antibody, for example, a monoclonal antibody. The antigen binding protein may be in the form of a recombinant or modified antibody (e.g., chimeric antibody, humanised antibody, human antibody, CDR-grafted antibody, primatised antibody, de-immunised antibody, synhumanised antibody, half-antibody, bispecific antibody, trispecific antibody or multispecific antibody). The antibody may further comprise a chemical modification, such as conjugation to an active agent or radiolabel, or an agent for improving solubility or other modification described herein.

As used herein the antigen binding protein may be a variable domain.

In any aspect of the invention and in any antigen binding protein described herein, there further includes an Fc region that is engineered to have reduced capacity to induce antibody-dependent cell-mediated cytotoxicity (ADCC). Preferably, the reduced capacity to induce ADCC is conferred by mutation, deletion or modification of amino acids in the Fc region which interact with an Fc receptor.

The invention provides an antigen binding protein as described herein wherein an amino acid sequence forming one or more of FR1, CDR1, FR2, CDR2, FR3, CDR3 and FR4 is a human sequence.

The invention provides an anti-nfP2X₇ antigen binding protein, immunoglobulin variable domain, antibody, dab, scFv, Fab, Fab′, F(ab′)2, Fv fragment, diabody, triabody, linear antibody, single-chain antibody molecule, or multispecific antibody comprising an antigen binding protein having a sequence as described herein, or including a CDR and/or FR sequence as described herein.

An antigen binding protein as described herein may comprise a human constant region, e.g., an IgG constant region, such as an IgG1, IgG2, IgG3 or IgG4 constant region or mixtures thereof. In the case of an antibody or protein comprising a VH and a VL, the VH can be linked to a heavy chain constant region and the VL can be linked to a light chain constant region.

In one example, an antigen binding protein as described herein comprises a constant region of an IgG4 antibody or a stabilised constant region of an IgG4 antibody. In one example, the protein or antibody comprises an IgG4 constant region with a proline at position 241 (according to the numbering system of Kabat (Kabat et al., Sequences of Proteins of Immunological Interest Washington DC United States Department of Health and Human Services, 1987 and/or 1991)).

In one example, an antigen binding protein as described herein or a composition of an antigen binding protein as described herein, comprises a heavy chain constant region, comprising a stabilised heavy chain constant region, comprising a mixture of sequences fully or partially with or without the C-terminal lysine residue.

In one example, an antigen binding protein comprises a VH disclosed herein linked or fused to an IgG4 constant region or stabilised IgG4 constant region (e.g., as discussed above) and the VL is linked to or fused to a kappa light chain constant region.

In any aspect of the present invention, the antibody is a naked antibody. Specifically, the antibody is in a non-conjugated form and is not adapted to form a conjugate.

The invention also provides a conjugate in the form of an antigen binding protein, immunoglobulin variable domain, antibody, dab, scFv, Fab, Fab′, F(ab′)2, Fv fragment, diabody, triabody, linear antibody, single-chain antibody molecule, or multispecific antibody or fusion protein as described herein conjugated to a label or a cytotoxic agent.

In aspects of the invention directed to multiple polypeptide chains that form an antigen binding protein, an expression construct comprises a nucleic acid encoding a polypeptide comprising, e.g., a VH operably linked to a promoter and a nucleic acid encoding a polypeptide comprising, e.g., a VL operably linked to a promoter.

In another example, the expression construct is a bicistronic expression construct, e.g., comprising the following operably linked components in 5′ to 3′ order:
(i) a promoter
(ii) a nucleic acid encoding a first polypeptide;
(iii) an internal ribosome entry site; and
(iv) a nucleic acid encoding a second polypeptide,
wherein the first polypeptide comprises a VH and the second polypeptide comprises a VL, or vice versa.

The present invention also contemplates separate expression constructs one of which encodes a first polypeptide comprising a VH and another of which encodes a second polypeptide comprising a VL. For example, the present invention also provides a composition comprising:
(i) a first expression construct comprising a nucleic acid encoding a polypeptide comprising a VH operably linked to a promoter; and
(ii) a second expression construct comprising a nucleic acid encoding a polypeptide comprising a VL operably linked to a promoter.

The invention provides a cell comprising a vector or nucleic acid described herein. Preferably, the cell is isolated, substantially purified or recombinant. In one example, the cell comprises the expression construct of the invention or:
(i) a first expression construct comprising a nucleic acid encoding a polypeptide comprising a VH operably linked to a promoter; and
(ii) a second expression construct comprising a nucleic acid encoding a polypeptide comprising a VL operably linked to a promoter,
wherein the first and second polypeptides associate to form an antigen binding protein of the present invention.

Examples of cells of the present invention include bacterial cells, yeast cells, insect cells or mammalian cells.

The invention provides a nucleic acid encoding an antigen binding protein, immunoglobulin variable domain, antibody, dab, scFv, Fab, Fab′, F(ab′)2, Fv fragment, diabody, triabody, linear antibody, single-chain antibody molecule, or multispecific antibody, fusion protein or conjugate as described herein.

The invention provides a vector comprising a nucleic acid described herein.

The invention provides a cell comprising a vector or nucleic acid described herein.

The invention provides a pharmaceutical composition comprising an antigen binding protein, or including a CDR and/or FR sequence as described herein, or an immunoglobulin variable domain, antibody, dab, scFv, Fab, Fab′, F(ab′)2, Fv fragment, diabody, triabody, linear antibody, single-chain antibody molecule, or multispecific antibody, fusion protein, or conjugate as described herein and a pharmaceutically acceptable carrier, diluent or excipient.

The invention provides a diagnostic composition comprising an antigen binding protein, or including a CDR and/or FR sequence as described herein, or antigen binding protein, immunoglobulin variable domain, antibody, dab, scFv, Fab, Fab′, F(ab′)2, Fv fragment, diabody, triabody, linear antibody, single-chain antibody molecule, or multispecific antibody, fusion protein or conjugate as described herein, a diluent and optionally a label.

The invention provides a kit or article of manufacture comprising an antigen binding protein, or including a CDR and/or FR sequence as described herein or an immunoglobulin variable domain, antibody, dab, scFv, Fab, Fab′, F(ab′)2, Fv fragment, diabody, triabody, linear antibody, single-chain antibody molecule, or multispecific antibody, fusion protein or conjugate as described herein.

The invention provides use of a sequence according to one or more of CDR1, CDR2, FR1, FR2, FR3 and FR4 as described herein to produce an antigen binding protein for binding to a nfP2X₇ receptor.

The invention provides use of an antigen binding protein or a CDR and/or FR sequence as described herein to produce an anti nfP2X₇ receptor antigen binding protein having increased affinity for nfP2X₇ receptor.

The invention provides a library of nucleic acid molecules produced from the mutation of an antigen binding protein or a CDR and/or FR sequence as described herein, wherein at least one nucleic acid molecule in said library encodes an antigen binding protein for binding to an nfP2X₇ receptor.

The invention provides a method for producing an antigen binding protein for binding to a nfP2X₇ receptor as described herein comprising expressing a nucleic acid as described herein in a cell or animal as described herein.

The functional characteristics of an antigen binding protein of the invention will be taken to apply mutatis mutandis to an antibody of the invention.

An antigen binding protein as described herein may be purified, substantially purified, isolated and/or recombinant.

An antigen binding protein of the invention may be part of a supernatant taken from media in which a hybridoma expressing an antigen binding protein of the invention has been grown.

The invention provides a method for the prevention or treatment a condition or disease associated with expression of nfP2X₇ in an individual comprising the step of providing an antigen binding protein, immunoglobulin variable domain, antibody, dab, scFv, Fab, Fab′, F(ab′)2, Fv fragment, diabody, triabody, linear antibody, single-chain antibody molecule, or multispecific antibody, fusion protein, conjugate or pharmaceutical composition as described herein to an individual requiring treatment for said condition or disease. The disease or condition associated with expression of nfP2X₇ is preferably a cancer.

In another aspect, the present invention also provides for a method of treating or preventing a cancer in a subject, the method comprising administering an antigen binding protein of the invention to the subject, thereby treating or preventing a cancer in the subject. As used herein, methods of treating cancer include methods of inhibiting, preventing or minimising spread or progression of a cancer, including inhibiting or preventing metastasis of cancer.

In another aspect, the present invention also provides for the use of an antigen binding protein of the invention, in the manufacture of a medicament for the treatment or prevention of cancer in a subject.

In another aspect, the invention provides for an antigen binding protein or a pharmaceutical composition comprising an antigen binding protein of the invention, for use in the treatment or prevention of cancer in a subject.

As used herein, except where the context requires otherwise, the term “comprise” and variations of the term, such as “comprising”, “comprises” and “comprised”, are not intended to exclude further additives, components, integers or steps.

Further aspects of the present invention and further embodiments of the aspects described in the preceding paragraphs will become apparent from the following description, given by way of example and with reference to the accompanying drawings.

### BRIEF DESCRIPTION OF THE DRAWINGS

FIG. 1: Three different scenarios are illustrated as the potential mode of action using an example of an antigen binding protein (also referred to herein as an Orchestration molecule (OR)) and a bridging molecule (also referred to herein as a BRiDGE molecule). Scenario I. shows the direct recruitment of immune effector cells to cancer cells via OR molecules. Scenario II. shows the indirect recruitment of immune effector cells to cancerous cells via BRiDGE molecules. Due to the fact that the BRiDGE molecules co-express the targeted nfP2X₇ E200 derived epitope on the BRiDGE molecules, the OR molecules may detect the cancer cells despite the fact that they may express nfP2X₇ only at very low levels or may not express nfP2X₇ at all. Scenario III. illustrates the most likely combinatorial mode of action in which both the direct as well as the indirect targeting may occur.

FIG. 2: Exemplary OR molecules with a first antigen binding domain targeting dysfunctional P2X₇ receptor and a second antigen binding domain targeting CD3. OR19_6 is a CD19/CD3 control based on blinatumomab. (a) OR1, OR2-3, OR13-3, OR14, OR15, OR17, and CD33 bridging molecule. (b) Table of additional exemplary OR molecule formats.

FIG. 3: Binding of orchestration molecule variants on MOLM-13 (AML) wildtype cell line. Indirect staining of artificial proteins on MOLM-13 cells via anti-HIS staining. As a control fusion protein the CD33 BRiDGE (B031) was used. There is a clear binding to MOLM-13 cells compared to the isotype control which was anti-HIS antibody only. Histogram overlay of HIS-FITC positivity on MOLM-13. Grey: Isotype control (left), Red: binding of HIS ab (right-shifted). HIS-ab was used according to manufacturer with 1 μl per test, target cells were incubated with 50 μl of supernatant from Lenti-X™ 293T cells for 10 min at room temperature. Supernatant harvested 48 h after transfection. Transfection with CD33 Fab as positive control. (a) OR1, (b) OR2-3, (c) OR13-3, (d) OR14, (e) OR15, (f) OR17, and (g) CD33 bridging molecule.

FIG. 4: Flow cytometric detection of binding capacity of OR molecules on T cells. Histogram overlay of HIS-FITC positivity on T-cells. Grey: Isotype control (left), Red: binding of HIS antibody (right shifted). HIS-antibody was used according to manufacturer with 1 μl per test, target cells were incubated with 50 μl of supernatant from Lenti-X™ 293T cells for 10 min at room temperature. Supernatant harvested 48 h after transfection. T-Cells were isolated by and cultured in TexMacs medium with IL-7/IL-15 for 7 days. (a) OR13-3, (b) OR14, (c) OR16, (d) OR17, and (e) OR19_6.

FIG. 5: Bridging molecules in Fab format with a single E200 epitope either directly linked to the V_{H} ((a) and (b)) or via a linker ((c) and (d)) binds to CD19 on JeKo-1 (mantle cell lymphoma) cell line and the E200 epitope is available for binding to an antibody (BIL03_2-2-1—AF647). HIS tag is detected by FITC antibody. (a) and (c) show anti-HIS antibody binding, (b) and (d) show binding of antibody to dysfunctional P2X₇ receptor epitope.

FIG. 6: Bridging molecules in scFv format with a single E200 epitope either directly linked to the V_{H} ((a) and (b)) or via a linker ((C) and (d)) binds to CD19 on JeKo-1 (mantle cell lymphoma) cell line and the E200 epitope is available for binding to an antibody. (a) and (c) show anti-HIS antibody binding, (b) and (d) show binding of antibody to dysfunctional P2X₇ receptor epitope.

FIG. 7: Bridging molecules in Fab format with a single E200 epitope either directly linked to the V_{L} ((a) and (b)) or via a linker ((c) and (d)) binds to CD19 on JeKo-1 (mantle cell lymphoma) cell line and the E200 epitope is available for binding to an antibody. (a) and (c) show anti-HIS antibody binding, (b) and (d) show binding of antibody to dysfunctional P2X₇ receptor epitope.

FIG. 8: Bridging molecules in scFv format with a single E200 epitope either directly linked to the V_{L} ((a) and (b)) or via a linker ((c) and (d)) binds to CD19 on JeKo-1 (mantle cell lymphoma) cell line and the E200 epitope is available for binding to an antibody. (a) and (c) show anti-HIS antibody binding, (b) and (d) show binding of antibody to dysfunctional P2X₇ receptor epitope.

FIG. 9: Binding of bridging molecules to various antigens CD37, CD79B, ROR1, CD33, CD38, CD123, CD135, BCMA, EGFR, PDL1, CD22, CD70 and CD20. (a), (c), (e), (g), (i), (k), (m), (o), (q), (s), (u), (w) and (y) show anti-HIS antibody binding, (b), (d), (f), (h), (j), (1), (n), (p), (r), (t), (v), (x) and (z) show binding of antibody to dysfunctional P2X₇ receptor epitope.

FIG. 10: “painting” of JeKo-1 cells with CD19 targeted Fab bridging molecules in the illustrated format as detected by flow cytometry. Cells were incubated at indicated concentrations with Fab bridging molecules. CD33 targeted Fab bridging molecules served as negative control in JeKo-1 at 10 ng/mL and 1000 ng/mL. CD19 targeted Fab bridging molecules were used at 1 ng/mL, 10 ng/mL, 100 ng/mL and 1000 ng/mL.

FIG. 11: “painting” of MOLM-13 cells with CD33 targeted Fab bridging molecules in the illustrated format as detected by flow cytometry. Cells were incubated at indicated concentrations with Fab bridging molecules. CD19 targeted Fab bridging molecules served as negative control in JeKo-1 at 10 ng/mL and 1000 ng/mL. CD33 targeted Fab bridging molecules were used at 1 ng/mL, 10 ng/mL, 100 ng/mL and 1000 ng/mL.

FIG. 12: The gating strategy is illustrated starting with a cell gate, a singlet gate to exclude doublets, a live-dead discrimination gate using only cells alive. T cells were discriminated from MOLM-13 via CD3 APC and MOLM-13 were generated to constitutively express eGFP. Only the T cell population was analysed for CD25 APCVio770 and CD69 VioGreen expression.

FIG. 13: T cells were incubated with MOLM-13 cells under different conditions. In the left panel neither OR molecules nor BRiDGE molecules were added and the basic T cell activation referred to as CD25 and CD69 double positive cells was 3.65%. In the middle panel OR17 was added and the specific activation is increased to 8.05% whereas the combination of OR17 and B031 increased the specific activation to 31%.

FIG. 14: Luciferase-based cytotoxicity assay T cells versus MOLM-13. Cytolysis was measured by the bioluminescence activity of MOLM-13 wildtype cell line transduced to constitutively express firefly luciferase and eGFP. OR molecules alone lead to a significant reduction of cancer cells, especially after 24 h, however this was further improved by the presence of a bridging molecule. (a) % viability of MOLM-13 cells after 20 hour incubation with T-cells in the presence of OR molecules, without, or with bridging molecules are various concentrations. (b) Summary of OR15, with and without CD33 bridging molecule (B031). (c) Summary of OR17, with and without CD33 bridging molecule (B031). (d) Summary of various OR molecules, with and without CD33 bridging molecule (B031).

FIG. 15: Kinetics of luciferase-based cytotoxicity assay T cells from healthy donor versus JeKo-1 cells without bridging molecules. Cytolysis was measured by the bioluminescence activity of Jeko-1 cell line transduced to constitutively express firefly luciferase and eGFP. OR molecules alone lead to a significant reduction of cancer cells, especially after 23 h. Relevantly, OR17 was as potent as OR19_6 (which corresponds to a very similar protein like Blinatumomab the CD3×CD19 bispecific T cell engager [BiTE]) and OR13+3 that was significantly more potent than any other OR molecule tested. N=1 healthy donor and 6 technical replicates.

FIG. 16: Bridging molecule B19_8_Fab significantly increased the potency of many OR molecules as compared to the data in FIG. 15.

FIG. 17: Bridging molecule B19_8_IgG1 significantly increased the potency of many OR molecules as compared to the data in FIG. 15.

FIG. 18: Bridging molecule variants.

FIG. 19: Viability of JeKo-1 cells without and with OR molecules after 24 hour incubation and an effector cells to target ratio of 10:1. No bridging molecules present. T cells from 2 healthy volunteer donors and 6 technical replicates.

FIG. 20: Viability of JeKo-1 cells without and with OR molecules after 24 hour incubation and an effector cells to target ratio of 10:1. B19_8_Fab bridging molecules present at 100 ng/mL. CD19 targeted BRiDGE derived from tafasitamab. T cells from 2 healthy volunteer donors and 6 technical replicates.

FIG. 21: Viability of JeKo-1 cells without and with OR molecules after 24 hour incubation and an effector cells to target ratio of 10:1. B19_8_IgG1 bridging molecules present at 100 ng/mL. CD19 targeted BRiDGE derived from tafasitamab. T cells from 2 healthy volunteer donors and 6 technical replicates.

FIG. 22: Additional bridging molecule variants with linkers or extended E200 epitope moieties.

FIG. 23: Viability of JeKo-1 cells without and with OR17 after 24 hour incubation and an effector cells to target ratio of 10:1. B19_10_Fab bridging molecules present various concentrations. CD19 targeted BRiDGE derived from tafasitamab. T cells from 1 healthy volunteer donors and 6 technical replicates.

FIG. 24: Viability of JeKo-1 cells without and with OR17 after 24 hour incubation and an effector cells to target ratio of 10:1. B19_10_IgG1 bridging molecules present various concentrations. CD19 targeted BRiDGE derived from tafasitamab. T cells from 1 healthy volunteer donors and 6 technical replicates.

FIG. 25: Viability of JeKo-1 cells without and with OR17 after 24 hour incubation and an effector cells to target ratio of 10:1. B19_11_Fab bridging molecules present various concentrations. CD19 targeted BRiDGE derived from tafasitamab. T cells from 1 healthy volunteer donors and 6 technical replicates.

FIG. 26: Viability of JeKo-1 cells without and with OR17 after 24 hour incubation and an effector cells to target ratio of 10:1. B19_11_IgG1 bridging molecules present various concentrations. CD19 targeted BRiDGE derived from tafasitamab. T cells from 1 healthy volunteer donors and 6 technical replicates.

FIG. 27: Comparison of different BRiDGE variants at the same concentration 100 ng/mL in Fab format.

FIG. 28: Comparison of different BRiDGE variants at the same concentration 100 ng/mL in IgG1 format.

FIG. 29: Viability of MOLM-13 cells without and with OR molecules after 30 hour incubation and an effector cells to target ratio of 10:1. B027 bridging molecules targeting CD38 present. T cells from a healthy volunteer donor and 5 technical replicates.

FIG. 30: Different representation of the data from FIG. 29.

FIG. 31: Demonstrates the activation of T cells measured by CD25+CD69+ cells with the variables+/−OR17 and the different bridging molecules in Fab and IgG1 format and tag variants OR19_7 (control without E200), B19_8, B19_10, B19_11. 2 healthy donors. Effector/target ratio 10:1. Bridging molecule in Fab/IgG1 format, final concentration 100 ng/mL, incubation time 24 h.

FIG. 32: Cytokine secretion of GMCSF under different conditions T cells targeting JeKo-1 with the variables+/−OR17 and different BRiDGE molecule variants in the Fab format with B19_7 as the control BRiDGE and the B19_8, B19_10 and B19_11. Effector/target ratio 10:1, 24 hour incubation.

FIG. 33: Cytokine secretion of IL2 under different conditions T cells targeting JeKo-1 with the variables+/−OR17 and different BRiDGE molecule variants in the Fab format with B19_7 as the control BRiDGE and the B19_8, B19_10 and B19_11. Effector/target ratio 10:1, 24 hour incubation.

FIG. 34: Cytokine secretion of TNFa under different conditions T cells targeting JeKo-1 with the variables+/−OR17 and different BRiDGE molecule variants in the Fab format with B19_7 as the control BRiDGE and the B19_8, B19_10 and B19_11. Effector/target ratio 10:1, 24 hour incubation.

FIG. 35: Cytokine secretion of IFNgamma under different conditions T cells targeting JeKo-1 with the variables+/−OR17 and different BRiDGE molecule variants in the Fab format with B19_7 as the control BRiDGE and the B19_8, B19_10 and B19_11. Effector/target ratio 10:1, 24 hour incubation.

FIG. 36: Architecture of bispecific and tetravalent molecules (Orchestration molecules) for engaging innate immune cells and cancer cells via nfP2X₇ receptor.

FIG. 37: Binding of innate immune cell engaging orchestration molecules to NK cells.

FIG. 38: Cell killing of JeKo cancer cells by PBMCs in presence of orchestration molecules+/−BRiDGE molecules.

FIG. 39: Cell killing of MOLM-13 cells by PBMCs in the presence of orchestration molecules+/−BRiDGE molecules.

TABLE 1
Sequence information

| | | |
|---|---|---|
| | | SEQ |
| | | ID |
| Description | Sequence | NO: |

Exemplary dysfunctional P2X₇ receptor
epitope moiety sequences

| | | |
|---|---|---|
| Human P2X₇ receptor | MPACCSCSDVFQYETNKVTRIQSMNYGTIKWFFHVIIFSYVCFA | 1 |
| | LVSDKLYQRKEPVISSVHTKVKGIAEVKEEIVENGVKKLVHSVF | |
| | DTADYTFPLQGNSFFVMTNFLKTEGQEQRLCPEYPTRRTLCS | |
| | SDRGCKKGWMDPQSKGIQTGRCVVYEGNQKTCEVSAWCPIE | |
| | AVEEAPRPALLNSAENFTVLIKNNIDFPGHNYTTRNILPGLNITC | |
| | TFHKTQNPQCPIFRLGDIFRETGDNFSDVAIQGGIMGIEIYWDC | |
| | NLDRWFHHCRPKYSFRRLDDKTTNVSLYPGYNFRYAKYYKEN | |
| | NVEKRTLIKVFGIRFDILVFGTGGKFDIIQLVVYIGSTLSYFGLAA | |
| | VFIDFLIDTYSSNCCRSHIYPWCKCCQPCVVNEYYYRKKCESIV | |
| | EPKPTLKYVSFVDESHIRMVNQQLLGRSLQDVKGQEVPRPAM | |
| | DFTDLSRLPLALHDTPPIPGQPEEIQLLRKEATPRSRDSPVWC | |
| | QCGSCLPSQLPESHRCLEELCCRKKPGACITTSELFRKLVLSR | |
| | HVLQFLLLYQEPLLALDVDSTNSRLRHCAYRCYATWRFGSQD | |
| | MADFAILPSCCRWRIRKEFPKSEGQYSGFKSPY | |
| Exemplary E200 | GHNYTTRNILPGLNITC | 2 |
| epitope | | |
| Variant E200 epitope | GHNYTTRNILPGLNIT | 3 |
| peptide | | |
| (E200′) | | |
| E200 epitope Cys to | GHNYTTRNILPGLNITS | 4 |
| Ser modification | | |
| Extended E200 Cys to | GHNYTTRNILPGLNITSTFHK | 5 |
| Ser modification | | |
| Extended E200′ | GHNYTTRNILPGLNITSTFHKT | 6 |
| Cys to Ser modification | | |
| (22 aa) | | |
| Extended E200″ | GHNYTTRNILPGLNITSTFHKTC | 7 |
| Cys to Ser modification | | |
| Pep16 | DFPGHNYTTRNILPGC | 8 |
| Pep17 | GHNYTTRNILPGLNITSTFHKTS | 9 |
| Extended Pep17 (27 | GHNYTTRNILPGLNITSTFHKTSGSGK | 10 |
| aa) | | |
| Minimum sequence | NYTTRNILPGL | 11 |
| E200 peptide (target | | |
| epitope) | | |
| Exemplary E300 | KYYKENNVEKRTLIK | 12 |
| epitope | | |
| Variant E300 epitope | KYYKENNVEKRTLIKVF | 13 |
| peptide | | |
| (E30′) | | |
| Exemplary E200/E300 | GHNYTTRNILPGAGAKYYKENNVEK | 14 |
| or composite epitope | | |
| E200_G4S | GHNYTTRNILPGLNITSGGGGS | 15 |
| E200_2xG4S | GHNYTTRNILPGLNITSGGGGSGGGGS | 16 |
| E200_3xG4S | GHNYTTRNILPGLNITSGGGGSGGGGSGGGGS | 168 |
| E200_extended | GHNYTTRNILPGLNITSTFHKTGS | 17 |
| peptide 17v3 (24 aa) | | |
| E200_extended | GHNYTTRNILPGLNITSTFHGS | 18 |
| peptide 17v4 (22 aa) | | |
| E200_extended | GHNYTTRNILPGLNITSGS | 19 |
| peptide 17v5 (19 aa) | | |
| E200_extended | DFPGHNYTTRNILPGLNITSGS | 20 |
| peptide 17v6 (22 aa) | | |
| E200_extended | DFPGHNYTTRNILPGLNITSGGGGS | 21 |
| peptide 17v7 (25 aa) | | |
| E200_extended | DFPGHNYTTRNILPGLNITSGGGGSGGGGS | 22 |
| peptide 17v8 (30 aa) | | |
| E200_extended | DFPGHNYTTRNILPGLNITSGGGGGGGGSGGGGS | 23 |
| peptide 17v9 (35 aa) | | |
| E200_extended | DFPGHNYTTRNILPGLNITSTFHKTSGSGK | 24 |
| peptide 17v10 (30 aa) | | |
| E200_extended | DFPGHNYTTRNILPGLNITSTFHKTSGSGKGS | 25 |
| peptide 17v11 (32 aa) | | |
| E200_extended | DFPGHNYTTRNILPGLNITSTFHKTSGSGKGGGGS | 26 |
| peptide 17v12 (35 aa) | | |
| E200_extended | DFPGHNYTTRNILPGLNITSTFHGGGGS | 27 |
| peptide 17v13 (25 aa) | | |
| E200_extended | GHNYTTRNILPGLNITSTFHGGGGS | 28 |
| peptide 17v14 (22 aa) | | |
| E200_extended | DFPGHNYTTRNILPGLNITSTFHKTGGGGS | 29 |
| peptide 17v15 (30 aa) | | |
| E200_extended | GHNYTTRNILPGLNITSTFHKTGGGGS | 30 |
| peptide 17v16 (27 aa) | | |

Exemplary targeting moiety sequences and exemplary bridging molecules
Constructs based on FMC63 (for binding CD19)

| | | |
|---|---|---|
| CD19 binder heavy | EVKLQESGPGLVAPSQSLSVTCTVSGVSLPDYGVSWIRQPPR | 31 |
| chain | KGLEWLGVIWGSETTYYNSALKSRLTIIKDNSKSQVFLKMNSL | |
| CD19, FMC63, | QTDDTAIYYCAKHYYYGGSYAMDYWGQGTSVTVSSASTKGP | |
| B001_Heavy Chain | SVFPLAPSSKSTSGGTAALGCLVKDYFPEPVTVSWNSGALTS | |
| | GVHTFPAVLQSSGLYSLSSVVTVPSSSLGTQTYICNVNHKPSN | |
| | TKVDKKVEPKSCHHHHHH | |
| CD19 binder light chain | DIQMTQTTSSLSASLGDRVTISCRASQDISKYLNWYQQKPDGT | 32 |
| CD19, FMC63, | VKLLIYHTSRLHSGVPSRFSGSGSGTDYSLTISNLEQEDIATYF | |
| B001_Light Chain | CQQGNTLPYTFGGGTKLEITKARTVAAPSVFIFPPSDEQLKSG | |
| | TASVVCLLNNFYPREAKVQWKVDNALQSGNSQESVTEQDSK | |
| | DSTYSLSSTLTLSKADYEKHKVYACEVTHQGLSSPVTKSFNRG | |
| | EC | |
| E200 + CD19 binder | GHNYTTRNILPGLNITSEVKLQESGPGLVAPSQSLSVTCTVSG | 33 |
| (nfP2X₇ epitope | VSLPDYGVSWIRQPPRKGLEWLGVIWGSETTYYNSALKSRLTII | |
| underlined) | KDNSKSQVFLKMNSLQTDDTAIYYCAKHYYYGGSYAMDYWG | |
| CD19, FMC63, B002- | QGTSVTVSSASTKGPSVFPLAPSSKSTSGGTAALGCLVKDYFP | |
| 1_Heavy Chain | EPVTVSWNSGALTSGVHTFPAVLQSSGLYSLSSVVTVPSSSLG | |
| | TQTYICNVNHKPSNTKVDKKVEPKSCHHHHHH | |
| E200 + CD19 binder | GHNYTTRNILPGLNITSGGGGSEVKLQESGPGLVAPSQSLSVT | 34 |
| (nfP2X₇ epitope | CTVSGVSLPDYGVSWIRQPPRKGLEWLGVIWGSETTYYNSAL | |
| underlined) | KSRLTIIKDNSKSQVFLKMNSLQTDDTAIYYCAKHYYYGGSYA | |
| CD19, FMC63, B002- | MDYWGQGTSVTVSSASTKGPSVFPLAPSSKSTSGGTAALGCL | |
| 2_Heavy Chain | VKDYFPEPVTVSWNSGALTSGVHTFPAVLQSSGLYSLSSVVTV | |
| | PSSSLGTQTYICNVNHKPSNTKVDKKVEPKSCHHHHHH | |
| E200 + CD19 binder | GHNYTTRNILPGLNITSDIQMTQTTSSLSASLGDRVTISCRASQ | 35 |
| (nfP2X₇ epitope | DISKYLNWYQQKPDGTVKLLIYHTSRLHSGVPSRFSGSGSGTD | |
| underlined) | YSLTISNLEQEDIATYFCQQGNTLPYTFGGGTKLEITKARTVAA | |
| CD19, FMC63, Light | PSVFIFPPSDEQLKSGTASVVCLLNNFYPREAKVQWKVDNALQ | |
| chain B003-1 | SGNSQESVTEQDSKDSTYSLSSTLTLSKADYEKHKVYACEVTH | |
| | QGLSSPVTKSFNRGEC | |
| E200 + CD19 binder | GHNYTTRNILPGLNITSGGGGSDIQMTQTTSSLSASLGDRVTIS | 36 |
| (nfP2X₇ epitope | CRASQDISKYLNWYQQKPDGTVKLLIYHTSRLHSGVPSRFSGS | |
| underlined) | GSGTDYSLTISNLEQEDIATYFCQQGNTLPYTFGGGTKLEITKA | |
| CD19, FMC63, Light | RTVAAPSVFIFPPSDEQLKSGTASVVCLLNNFYPREAKVQWKV | |
| chain B003-2 | DNALQSGNSQESVTEQDSKDSTYSLSSTLTLSKADYEKHKVY | |
| | ACEVTHQGLSSPVTKSFNRGEC | |
| E200 + CD19 binder | EVKLQESGPGLVAPSQSLSVTCTVSGVSLPDYGVSWIRQPPR | 37 |
| (nfP2X₇ epitope | KGLEWLGVIWGSETTYYNSALKSRLTIIKDNSKSQVFLKMNSL | |
| underlined) | QTDDTAIYYCAKHYYYGGSYAMDYWGQGTSVTVSSASTKGP | |
| CD19, FMC63, | SVFPLAPSSKSTSGGTAALGCLVKDYFPEPVTVSWNSGALTS | |
| B005_Heavy Chain | GVHTFPAVLQSSGLYSLSSVVTVPSSSLGTQTYICNVNHKPSN | |
| | TKVDKKVEPKSCGHNYTTRNILPGLNITSHHHHHH | |
| CD19, FMC63, | DIQMTQTTSSLSASLGDRVTISCRASQDISKYLNWYQQKPDGT | 38 |
| B005_Light Chain | VKLLIYHTSRLHSGVPSRFSGSGSGTDYSLTISNLEQEDIATYF | |
| (His tagged version of | CQQGNTLPYTFGGGTKLEITKARTVAAPSVFIFPPSDEQLKSG | |
| SEQ ID NO: 32) | TASVVCLLNNFYPREAKVQWKVDNALQSGNSQESVTEQDSK | |
| | DSTYSLSSTLTLSKADYEKHKVYACEVTHQGLSSPVTKSFNRG | |
| | ECHHHHHH | |
| E200 + CD19 binder | DIQMTQTTSSLSASLGDRVTISCRASQDISKYLNWYQQKPDGT | 39 |
| (nfP2X₇ epitope | VKLLIYHTSRLHSGVPSRFSGSGSGTDYSLTISNLEQEDIATYF | |
| underlined) | CQQGNTLPYTFGGGTKLEITKARTVAAPSVFIFPPSDEQLKSG | |
| CD19, FMC63, | TASVVCLLNNFYPREAKVQWKVDNALQSGNSQESVTEQDSK | |
| B006_Light Chain | DSTYSLSSTLTLSKADYEKHKVYACEVTHQGLSSPVTKSFNRG | |
| | ECGHNYTTRNILPGLNITS | |
| CD19 binder scFv | DIQMTQTTSSLSASLGDRVTISCRASQDISKYLNWYQQKPDGT | 40 |
| format | VKLLIYHTSRLHSGVPSRFSGSGSGTDYSLTISNLEQEDIATYF | |
| CD19, FMC63, | CQQGNTLPYTFGGGTKLEITGGGGSGGGGSGGGGSEVKLQE | |
| B011_scFv_Light/ | SGPGLVAPSQSLSVTCTVSGVSLPDYGVSWIRQPPRKGLEWL | |
| Heavy | GVIWGSETTYYNSALKSRLTIIKDNSKSQVFLKMNSLQTDDTAI | |
| | YYCAKHYYYGGSYAMDYWGQGTSVTVSSHHHHHH | |
| CD19 binder scFv | EVKLQESGPGLVAPSQSLSVTCTVSGVSLPDYGVSWIRQPPR | 41 |
| format | KGLEWLGVIWGSETTYYNSALKSRLTIIKDNSKSQVFLKMNSL | |
| CD19, FMC63, | QTDDTAIYYCAKHYYYGGSYAMDYWGQGTSVTVSSGGGGSG | |
| B012_scFv_Heavy/ | GGGSGGGGSDIQMTQTTSSLSASLGDRVTISCRASQDISKYLN | |
| Light | WYQQKPDGTVKLLIYHTSRLHSGVPSRFSGSGSGTDYSLTISN | |
| | LEQEDIATYFCQQGNTLPYTFGGGTKLEITHHHHHH | |
| E200 + CD19 binder in | GHNYTTRNILPGLNITSDIQMTQTTSSLSASLGDRVTISCRASQ | 42 |
| scFv format (nfP2X₇ | DISKYLNWYQQKPDGTVKLLIYHTSRLHSGVPSRFSGSGSGTD | |
| epitope underlined) | YSLTISNLEQEDIATYFCQQGNTLPYTFGGGTKLEITGGGGSG | |
| CD19, FMC63, B013- | GGGSGGGGSEVKLQESGPGLVAPSQSLSVTCTVSGVSLPDY | |
| 1_scFv_LH | GVSWIRQPPRKGLEWLGVIWGSETTYYNSALKSRLTIIKDNSK | |
| | SQVFLKMNSLQTDDTAIYYCAKHYYYGGSYAMDYWGQGTSV | |
| | TVSSHHHHHH | |
| E200 + CD19 binder in | GHNYTTRNILPGLNITSGGGGSDIQMTQTTSSLSASLGDRVTIS | 43 |
| scFv format (nfP2X₇ | CRASQDISKYLNWYQQKPDGTVKLLIYHTSRLHSGVPSRFSGS | |
| epitope underlined) | GSGTDYSLTISNLEQEDIATYFCQQGNTLPYTFGGGTKLEITGG | |
| CD19, FMC63, B013- | GGSGGGGSGGGGSEVKLQESGPGLVAPSQSLSVTCTVSGVS | |
| 2_scFv_LH | LPDYGVSWIRQPPRKGLEWLGVIWGSETTYYNSALKSRLTIIKD | |
| | NSKSQVFLKMNSLQTDDTAIYYCAKHYYYGGSYAMDYWGQG | |
| | TSVTVSSHHHHHH | |
| E200 + CD19 binder in | GHNYTTRNILPGLNITSEVKLQESGPGLVAPSQSLSVTCTVSG | 44 |
| scFv format (nfP2X₇ | VSLPDYGVSWIRQPPRKGLEWLGVIWGSETTYYNSALKSRLTII | |
| epitope underlined) | KDNSKSQVFLKMNSLQTDDTAIYYCAKHYYYGGSYAMDYWG | |
| CD19, FMC63, B014- | QGTSVTVSSGGGGSGGGGSGGGGSDIQMTQTTSSLSASLGD | |
| 1_scFv_HL | RVTISCRASQDISKYLNWYQQKPDGTVKLLIYHTSRLHSGVPS | |
| | RFSGSGSGTDYSLTISNLEQEDIATYFCQQGNTLPYTFGGGTK | |
| | LEITHHHHHH | |
| E200 + CD19 binder in | GHNYTTRNILPGLNITSGGGGSEVKLQESGPGLVAPSQSLSVT | 45 |
| scFv format (nfP2X₇ | CTVSGVSLPDYGVSWIRQPPRKGLEWLGVIWGSETTYYNSAL | |
| epitope underlined) | KSRLTIIKDNSKSQVFLKMNSLQTDDTAIYYCAKHYYYGGSYA | |
| CD19, FMC63, B014- | MDYWGQGTSVTVSSGGGGSGGGGSGGGGSDIQMTQTTSSL | |
| 2_scFv_HL | SASLGDRVTISCRASQDISKYLNWYQQKPDGTVKLLIYHTSRL | |
| | HSGVPSRFSGSGSGTDYSLTISNLEQEDIATYFCQQGNTLPYT | |
| | FGGGTKLEITHHHHHH | |
| E200 + CD19 binder in | EVKLQESGPGLVAPSQSLSVTCTVSGVSLPDYGVSWIRQPPR | 46 |
| scFv format (nfP2X₇ | KGLEWLGVIWGSETTYYNSALKSRLTIIKDNSKSQVFLKMNSL | |
| epitope underlined) | QTDDTAIYYCAKHYYYGGSYAMDYWGQGTSVTVSSGGGGSG | |
| CD19, FMC63, | GGGSGGGGSDIQMTQTTSSLSASLGDRVTISCRASQDISKYLN | |
| B015_scFv_HL | WYQQKPDGTVKLLIYHTSRLHSGVPSRFSGSGSGTDYSLTISN | |
| | LEQEDIATYFCQQGNTLPYTFGGGTKLEITGHNYTTRNILPGLN | |
| | ITSHHHHHH | |
| E200 + CD19 binder in | DIQMTQTTSSLSASLGDRVTISCRASQDISKYLNWYQQKPDGT | 360 |
| scFv format (nfP2X₇ | VKLLIYHTSRLHSGVPSRFSGSGSGTDYSLTISNLEQEDIATYF | |
| epitope underlined) | CQQGNTLPYTFGGGTKLEITGGGGSGGGGSGGGGSEVKLQE | |
| CD19, FMC63, | SGPGLVAPSQSLSVTCTVSGVSLPDYGVSWIRQPPRKGLEWL | |
| B016_scFv_LH | GVIWGSETTYYNSALKSRLTIIKDNSKSQVFLKMNSLQTDDTAI | |
| | YYCAKHYYYGGSYAMDYWGQGTSVTVSS | |
| | GHNYTTRNILPGLNITSHHHHHH | |
| E200 + CD19 binder in | GHNYTTRNILPGLNITSDIQMTQTTSSLSASLGDRVTISCRASQ | 47 |
| scFv format (nfP2X₇ | DISKYLNWYQQKPDGTVKLLIYHTSRLHSGVPSRFSGSGSGTD | |
| epitope underlined) | YSLTISNLEQEDIATYFCQQGNTLPYTFGGGTKLEITGGGGSG | |
| CD19, FMC63, B017- | GGGSGGGGSEVKLQESGPGLVAPSQSLSVTCTVSGVSLPDY | |
| 1_scFv_LH | GVSWIRQPPRKGLEWLGVIWGSETTYYNSALKSRLTIIKDNSK | |
| | SQVFLKMNSLQTDDTAIYYCAKHYYYGGSYAMDYWGQGTSV | |
| | TVSSGHNYTTRNILPGLNITSHHHHHH | |
| E200 + CD19 binder in | GHNYTTRNILPGLNITSGGGGSDIQMTQTTSSLSASLGDRVTIS | 48 |
| scFv format (nfP2X₇ | CRASQDISKYLNWYQQKPDGTVKLLIYHTSRLHSGVPSRFSGS | |
| epitope underlined) | GSGTDYSLTISNLEQEDIATYFCQQGNTLPYTFGGGTKLEITGG | |
| CD19, FMC63, B017- | GGSGGGGSGGGGSEVKLQESGPGLVAPSQSLSVTCTVSGVS | |
| 2_scFv_LH | LPDYGVSWIRQPPRKGLEWLGVIWGSETTYYNSALKSRLTIIKD | |
| | NSKSQVFLKMNSLQTDDTAIYYCAKHYYYGGSYAMDYWGQG | |
| | TSVTVSSGHNYTTRNILPGLNITSHHHHHH | |
| E200 + CD19 binder in | GHNYTTRNILPGLNITSEVKLQESGPGLVAPSQSLSVTCTVSG | 49 |
| scFv format (nfP2X₇ | VSLPDYGVSWIRQPPRKGLEWLGVIWGSETTYYNSALKSRLTII | |
| epitope underlined) | KDNSKSQVFLKMNSLQTDDTAIYYCAKHYYYGGSYAMDYWG | |
| CD19, FMC63, B018- | QGTSVTVSSGGGGSGGGGSGGGGSDIQMTQTTSSLSASLGD | |
| 1_scFv_HL | RVTISCRASQDISKYLNWYQQKPDGTVKLLIYHTSRLHSGVPS | |
| | RFSGSGSGTDYSLTISNLEQEDIATYFCQQGNTLPYTFGGGTK | |
| | LEITGHNYTTRNILPGLNITSHHHHHH | |
| E200 + CD19 binder in | GHNYTTRNILPGLNITSGGGGSEVKLQESGPGLVAPSQSLSVT | 50 |
| scFv format (nfP2X₇ | CTVSGVSLPDYGVSWIRQPPRKGLEWLGVIWGSETTYYNSAL | |
| epitope underlined) | KSRLTIIKDNSKSQVFLKMNSLQTDDTAIYYCAKHYYYGGSYA | |
| CD19, FMC63, B018- | MDYWGQGTSVTVSSGGGGSGGGGSGGGGSDIQMTQTTSSL | |
| 2_scFv_HL | SASLGDRVTISCRASQDISKYLNWYQQKPDGTVKLLIYHTSRL | |
| | HSGVPSRFSGSGSGTDYSLTISNLEQEDIATYFCQQGNTLPYT | |
| | FGGGTKLEITGHNYTTRNILPGLNITSHHHHHH | |

Constructs based on Tafasitamab (for binding CD19)

| | | |
|---|---|---|
| E200 + CD19 binder | GHNYTTRNILPGLNITSDIVMTQSPATLSLSPGERATLSCRSSK | 51 |
| (nfP2X₇ epitope | SLQNVNGNTYLYWFQQKPGQSPQLLIYRMSNLNSGVPDRFSG | |
| underlined) | SGSGTEFTLTISSLEPEDFAVYYCMQHLEYPITFGAGTKLEIKR | |
| CD19, Tafasitamab, | TVAAPSVFIFPPSDEQLKSGTASVVCLLNNFYPREAKVQWKVD | |
| B020-1_Light Chain | NALQSGNSQESVTEQDSKDSTYSLSSTLTLSKADYEKHKVYA | |
| | CEVTHQGLSSPVTKSFNRGECHHHHHH | |
| CD19, Tafasitamab, | EVQLVESGGGLVKPGGSLKLSCAASGYTFTSYVMHWVRQAP | 52 |
| B020-2_Heavy Chain | GKGLEWIGYINPYNDGTKYNEKFQGRVTISSDKSISTAYMELSS | |
| | LRSEDTAMYYCARGTYYYGTRVFDYWGQGTLVTVSSASTKG | |
| | PSVFPLAPSSKSTSGGTAALGCLVKDYFPEPVTVSWNSGALTS | |
| | GVHTFPAVLQSSGLYSLSSVVTVPSSSLGTQTYICNVNHKPSN | |
| | TKVDKKVEPKSCHHHHHH | |

Constructs for binding CD20

| | | |
|---|---|---|
| E200 + CD20 binder | GHNYTTRNILPGLNITSDIQMTQSPSSLSASVGDRVTITCRASS | 53 |
| (nfP2X₇ epitope | SVSYMHWYQQKPGKAPKPLIYAPSNLASGVPSRFSGSGSGTD | |
| underlined) | FTLTISSLOPEDFATYYCQQWSFNPPTFGQGTKVEIKRTVAAP | |
| CD20, Ocrelizumab, | SVFIFPPSDEQLKSGTASVVCLLNNFYPREAKVQWKVDNALQS | |
| B021-1_Light chain | GNSQESVTEQDSKDSTYSLSSTLTLSKADYEKHKVYACEVTH | |
| | QGLSSPVTKSFNRGECHHHHHH | |
| CD20, Ocrelizumab, | EVQLVESGGGLVQPGGSLRLSCAASGYTFTSYNMHWVRQAP | 54 |
| B021-1_Heavy Chain | GKGLEWVGAIYPGNGDTSYNQKFKGRFTISVDKSKNTLYLQM | |
| | NSLRAEDTAVYYCARVVYYSNSYWYFDVWGQGTLVTVSSAST | |
| | KGPSVFPLAPSSKSTSGGTAALGCLVKDYFPEPVTVSWNSGA | |
| | LTSGVHTFPAVLQSSGLYSLSSVVTVPSSSLGTQTYICNVNHK | |
| | PSNTKVDKKVEPKSCHHHHHH | |
| E200 + CD20 binder | GHNYTTRNILPGLNITSEIVLTQSPATLSLSPGERATLSCRASQ | 55 |
| (nfP2X₇ epitope | SVSSYLAWYQQKPGQAPRLLIYDASNRATGIPARFSGSGSGT | |
| underlined) | DFTLTISSLEPEDFAVYYCQQRSNWPITFGQGTRLEIKRTVAAP | |
| CD20, Ofatumumab, | SVFIFPPSDEQLKSGTASVVCLLNNFYPREAKVQWKVDNALQS | |
| B022-1_Light Chain | GNSQESVTEQDSKDSTYSLSSTLTLSKADYEKHKVYACEVTH | |
| | QGLSSPVTKSFNRGECHHHHHH | |
| CD20, Ofatumumab, | EVQLVESGGGLVQPGRSLRLSCAASGFTFNDYAMHWVRQAP | 56 |
| B022-2_Heavy Chain | GKGLEWVSTISWNSGSIGYADSVKGRFTISRDNAKKSLYLQMN | |
| | SLRAEDTALYYCAKDIQYGNYYYGMDVWGQGTTVTVSSASTK | |
| | GPSVFPLAPSSKSTSGGTAALGCLVKDYFPEPVTVSWNSGAL | |
| | TSGVHTFPAVLQSSGLYSLSSVVTVPSSSLGTQTYICNVNHKP | |
| | SNTKVDKKVEPKSCHHHHHH | |

Constructs for binding CD22

| | | |
|---|---|---|
| E200 + CD22 binder | GHNYTTRNILPGLNITSDIQMIQSPSSLSASVGDRVTITCRASQT | 57 |
| (nfP2X₇ epitope | IWSYLNWYRQRPGEAPNLLIYAASSLQSGVPSRFSGRGSGTD | |
| underlined) | FTLTISSLQAEDFATYYCQQSYSIPQTFGQGTKLEIKRTVAAPS | |
| CD22, m971-L7, B023- | VFIFPPSDEQLKSGTASVVCLLNNFYPREAKVQWKVDNALQS | |
| 1 LC | GNSQESVTEQDSKDSTYSLSSTLTLSKADYEKHKVYACEVTH | |
| | QGLSSPVTKSFNRGECHHHHHH | |
| CD22, m971-L7, B023- | QVQLQQSGPGMVKPSQTLSLTCAISGDSVSSNSVAWNWIRQS | 58 |
| 2_Heavy Chain | PSRGLEWLGRTYYRSTWYNDYAVSMKSRITINPDTNKNQFSL | |
| | QLNSVTPEDTAVYYCAREVTGDLEDAFDIWGQGTMVTVSSAS | |
| | TKGPSVFPLAPSSKSTSGGTAALGCLVKDYFPEPVTVSWNSG | |
| | ALTSGVHTFPAVLQSSGLYSLSSVVTVPSSSLGTQTYICNVNH | |
| | KPSNTKVDKKVEPKSCHHHHHH | |
| E200 + CD22 binder | GHNYTTRNILPGLNITSDVQVTQSPSSLSASVGDRVTITCRSSQ | 59 |
| (nfP2X₇ epitope | SLANSYGNTFLSWYLHKPGKAPQLLIYGISNRFSGVPDRFSGS | |
| underlined) | GSGTDFTLTISSLQPEDFATYYCLQGTHQPYTFGQGTKVEIKR | |
| CD22, Inotuzumab, | TVAAPSVFIFPPSDEQLKSGTASVVCLLNNFYPREAKVQWKVD | |
| B024-1_LC | NALQSGNSQESVTEQDSKDSTYSLSSTLTLSKADYEKHKVYA | |
| | CEVTHQGLSSPVTKSFNRGECHHHHHH | |
| CD22, Inotuzumab, | EVQLVQSGAEVKKPGASVKVSCKASGYRFTNYWIHWVRQAP | 60 |
| B024-2_HC | GQGLEWIGGINPGNNYATYRRKFQGRVTMTADTSTSTVYMEL | |
| | SSLRSEDTAVYYCTREGYGNYGAWFAYWGQGTLVTVSSAST | |
| | KGPSVFPLAPSSKSTSGGTAALGCLVKDYFPEPVTVSWNSGA | |
| | LTSGVHTFPAVLQSSGLYSLSSVVTVPSSSLGTQTYICNVNHK | |
| | PSNTKVDKKVEPKSCHHHHHH | |

Constructs for binding CD79B

| | | |
|---|---|---|
| E200 + CD79B binder | GHNYTTRNILPGLNITSDIQLTQSPSSLSASVGDRVTITCKASQ | 61 |
| (nfP2X₇ epitope | SVDYEGDSFLNWYQQKPGKAPKLLIYAASNLESGVPSRFSGS | |
| underlined) | GSGTDFTLTISSLOPEDFATYYCQQSNEDPLTFGQGTKVEIKR | |
| CD79B, Polatuzumab, | TVAAPSVFIFPPSDEQLKSGTASVVCLLNNFYPREAKVQWKVD | |
| B025-1 LC | NALQSGNSQESVTEQDSKDSTYSLSSTLTLSKADYEKHKVYA | |
| | CEVTHQGLSSPVTKSFNRGECHHHHHH | |
| CD79B, Polatuzumab, | EVQLVESGGGLVQPGGSLRLSCAASGYTFSSYWIEWVRQAP | 62 |
| B025-2_HC | GKGLEWIGEILPGGGDTNYNEIFKGRATFSADTSKNTAYLQMN | |
| | SLRAEDTAVYYCTRRVPIRLDYWGQGTLVTVSSASTKGPSVFP | |
| | LAPSSKSTSGGTAALGCLVKDYFPEPVTVSWNSGALTSGVHT | |
| | FPAVLQSSGLYSLSSVVTVPSSSLGTQTYICNVNHKPSNTKVD | |
| | KKVEPKSCHHHHHH | |

Constructs for binding CD37

| | | |
|---|---|---|
| E200 + CD37 binder | GHNYTTRNILPGLNITSEIVLTQSPATLSLSPGERATLSCRASEN | 63 |
| (nfP2X₇ epitope | VYSYLAWYQQKPGQAPRLLIYFAKTLAEGIPARFSGSGSGTDF | |
| underlined) | TLTISSLEPEDFAVYYCQHHSDNPWTFGQGTKVEIKRTVAAPS | |
| CD37, Otlertuzumab, | VFIFPPSDEQLKSGTASVVCLLNNFYPREAKVQWKVDNALQS | |
| B026-1_LC | GNSQESVTEQDSKDSTYSLSSTLTLSKADYEKHKVYACEVTH | |
| | QGLSSPVTKSFNRGECHHHHHH | |
| CD37, Otlertuzumab, | EVQLVQSGAEVKKPGESLKISCKGSGYSFTGYNMNWVRQMP | 64 |
| B026-2_HC | GKGLEWMGNIDPYYGGTTYNRKFKGQVTISADKSISTAYLQW | |
| | SSLKASDTAMYYCARSVGPFDSWGQGTLVTVSSASTKGPSVF | |
| | PLAPSSKSTSGGTAALGCLVKDYFPEPVTVSWNSGALTSGVH | |
| | TFPAVLQSSGLYSLSSVVTVPSSSLGTQTYICNVNHKPSNTKV | |
| | DKKVEPKSCHHHHHH | |

Constructs for binding CD38

| | | |
|---|---|---|
| E200 + CD38 binder | GHNYTTRNILPGLNITSEIVLTQSPATLSLSPGERATLSCRASQ | 65 |
| (nfP2X₇ epitope | SVSSYLAWYQQKPGQAPRLLIYDASNRATGIPARFSGSGSGT | |
| underlined) | DFTLTISSLEPEDFAVYYCQQRSNWPPTFGQGTKVEIKRTVAA | |
| CD38, Daratumumab, | PSVFIFPPSDEQLKSGTASVVCLLNNFYPREAKVQWKVDNALQ | |
| B027-1_LC | SGNSQESVTEQDSKDSTYSLSSTLTLSKADYEKHKVYACEVTH | |
| | QGLSSPVTKSFNRGECHHHHHH | |
| CD38, Daratumumab, | EVQLLESGGGLVQPGGSLRLSCAVSGFTFNSFAMSWVRQAP | 66 |
| B027-2_HC | GKGLEWVSAISGSGGGTYYADSVKGRFTISRDNSKNTLYLQM | |
| | NSLRAEDTAVYFCAKDKILWFGEPVFDYWGQGTLVTVSSAST | |
| | KGPSVFPLAPSSKSTSGGTAALGCLVKDYFPEPVTVSWNSGA | |
| | LTSGVHTFPAVLQSSGLYSLSSVVTVPSSSLGTQTYICNVNHK | |
| | PSNTKVDKKVEPKSCHHHHHH | |

Constructs for binding CD70

| | | |
|---|---|---|
| E200 + CD70 binder | GHNYTTRNILPGLNITSQAVVTQEPSLTVSPGGTVTLTCGLKSG | 67 |
| (nfP2X₇ epitope | SVTSDNFPTWYQQTPGQAPRLLIYNTNTRHSGVPDRFSGSILG | |
| underlined) | NKAALTITGAQADDEAEYFCALFISNPSVEFGGGTQLTVLKRTV | |
| CD70, Cusatuzumab, | AAPSVFIFPPSDEQLKSGTASVVCLLNNFYPREAKVQWKVDNA | |
| B028-1_LC | LQSGNSQESVTEQDSKDSTYSLSSTLTLSKADYEKHKVYACEV | |
| | THQGLSSPVTKSFNRGECHHHHHH | |
| CD70, Cusatuzumab, | EVQLVESGGGLVQPGGSLRLSCAASGFTFSVYYMNWVRQAP | 68 |
| B028-2_HC | GKGLEWVSDINNEGGTTYYADSVKGRFTISRDNSKNSLYLQM | |
| | NSLRAEDTAVYYCARDAGYSNHVPIFDSWGQGTLVTVSSAST | |
| | KGPSVFPLAPSSKSTSGGTAALGCLVKDYFPEPVTVSWNSGA | |
| | LTSGVHTFPAVLQSSGLYSLSSVVTVPSSSLGTQTYICNVNHK | |
| | PSNTKVDKKVEPKSCHHHHHH | |

Constructs for binding CD30

| | | |
|---|---|---|
| E200 + CD30 binder | GHNYTTRNILPGLNITSDIVLTQSPASLAVSLGQRATISCKASQS | 69 |
| (nfP2X₇ epitope | VDFDGDSYMNWYQQKPGQPPKVLIYAASNLESGIPARFSGSG | |
| underlined) | SGTDFTLNIHPVEEEDAATYYCQQSNEDPWTFGGGTKLEIKRT | |
| CD30, Brentuximab, | VAAPSVFIFPPSDEQLKSGTASVVCLLNNFYPREAKVQWKVDN | |
| B029-1_LC | ALQSGNSQESVTEQDSKDSTYSLSSTLTLSKADYEKHKVYACE | |
| | VTHQGLSSPVTKSFNRGECHHHHHH | |
| CD30, Brentuximab, | QIQLQQSGPEVVKPGASVKISCKASGYTFTDYYITWVKQKPGQ | 70 |
| B029-2_HC | GLEWIGWIYPGSGNTKYNEKFKGKATLTVDTSSSTAFMQLSSL | |
| | TSEDTAVYFCANYGNYWFAYWGQGTQVTVSAASTKGPSVFP | |
| | LAPSSKSTSGGTAALGCLVKDYFPEPVTVSWNSGALTSGVHT | |
| | FPAVLQSSGLYSLSSVVTVPSSSLGTQTYICNVNHKPSNTKVD | |
| | KKVEPKSCHHHHHH | |

Constructs for binding CD33

| | | |
|---|---|---|
| E200 + CD33 binder | GHNYTTRNILPGLNITSDIQLTQSPSTLSASVGDRVTITCRASES | 71 |
| (nfP2X₇ epitope | LDNYGIRFLTWFQQKPGKAPKLLMYAASNQGSGVPSRFSGSG | |
| underlined) | SGTEFTLTISSLQPDDFATYYCQQTKEVPWSFGQGTKVEVKRT | |
| CD33, Gemtuzumab, | VAAPSVFIFPPSDEQLKSGTASVVCLLNNFYPREAKVQWKVDN | |
| B030-1_LC | ALQSGNSQESVTEQDSKDSTYSLSSTLTLSKADYEKHKVYACE | |
| | VTHQGLSSPVTKSFNRGECHHHHHH | |
| CD33, Gemtuzumab, | EVQLVQSGAEVKKPGSSVKVSCKASGYTITDSNIHWVRQAPG | 72 |
| B030-2_Heavy Chain | QSLEWIGYIYPYNGGTDYNQKFKNRATLTVDNPTNTAYMELSS | |
| | LRSEDTAFYYCVNGNPWLAYWGQGTLVTVSSASTKGPSVFPL | |
| | APSSKSTSGGTAALGCLVKDYFPEPVTVSWNSGALTSGVHTF | |
| | PAVLQSSGLYSLSSVVTVPSSSLGTQTYICNVNHKPSNTKVDK | |
| | KVEPKSCHHHHHH | |
| E200 + CD33 binder | GHNYTTRNILPGLNITSDIQMTQSPSSLSASVGDRVTITCRASE | 73 |
| (nfP2X₇ epitope | SVDNYGISFMNWFQQKPGKAPKLLIYAASNQGSGVPSRFSGS | |
| underlined) | GSGTDFTLTISSLQPDDFATYYCQQSKEVPWTFGQGTKVEIKR | |
| CD33, Lintuzumab, | TVAAPSVFIFPPSDEQLKSGTASVVCLLNNFYPREAKVQWKVD | |
| B031-1_LC | NALQSGNSQESVTEQDSKDSTYSLSSTLTLSKADYEKHKVYA | |
| | CEVTHQGLSSPVTKSFNRGECHHHHHH | |
| CD33, Lintuzumab, | QVQLVQSGAEVKKPGSSVKVSCKASGYTFTDYNMHWVRQAP | 74 |
| B031-2_HC | GQGLEWIGYIYPYNGGTGYNQKFKSKATITADESTNTAYMELS | |
| | SLRSEDTAVYYCARGRPAMDYWGQGTLVTVSSASTKGPSVF | |
| | PLAPSSKSTSGGTAALGCLVKDYFPEPVTVSWNSGALTSGVH | |
| | TFPAVLQSSGLYSLSSVVTVPSSSLGTQTYICNVNHKPSNTKV | |
| | DKKVEPKSCHHHHHH | |

Constructs for binding Her2

| | | |
|---|---|---|
| E200 + Her2 binder | GHNYTTRNILPGLNITSDIQMTQSPSSLSASVGDRVTITCKASQ | 75 |
| (nfP2X₇ epitope | DVSIGVAWYQQKPGKAPKLLIYSASYRYTGVPSRFSGSGSGT | |
| underlined) | DFTLTISSLQPEDFATYYCQQYYIYPYTFGQGTKVEIKRTVAAP | |
| Her2, Pertuzumab, | SVFIFPPSDEQLKSGTASVVCLLNNFYPREAKVQWKVDNALQS | |
| B032-1_LC | GNSQESVTEQDSKDSTYSLSSTLTLSKADYEKHKVYACEVTH | |
| | QGLSSPVTKSFNRGECHHHHHH | |
| Her2, Pertuzumab, | EVQLVESGGGLVQPGGSLRLSCAASGFTFTDYTMDWVRQAP | 76 |
| B032-2_HC | GKGLEWVADVNPNSGGSIYNQRFKGRFTLSVDRSKNTLYLQM | |
| | NSLRAEDTAVYYCARNLGPSFYFDYWGQGTLVTVSSASTKGP | |
| | SVFPLAPSSKSTSGGTAALGCLVKDYFPEPVTVSWNSGALTS | |
| | GVHTFPAVLQSSGLYSLSSVVTVPSSSLGTQTYICNVNHKPSN | |
| | TKVDKKVEPKSCHHHHHH | |
| E200 + Her2 binder | GHNYTTRNILPGLNITSDIQMTQSPSSLSASVGDRVTITCRASQ | 77 |
| (nfP2X₇ epitope | DVNTAVAWYQQKPGKAPKLLIYSASFLYSGVPSRFSGSRSGT | |
| underlined) | DFTLTISSLQPEDFATYYCQQHYTTPPTFGQGTKVEIKRTVAAP | |
| Her2, Trastuzumab, | SVFIFPPSDEQLKSGTASVVCLLNNFYPREAKVQWKVDNALQS | |
| B033-1_LC | GNSQESVTEQDSKDSTYSLSSTLTLSKADYEKHKVYACEVTH | |
| | QGLSSPVTKSFNRGECHHHHHH | |
| Her2, Trastuzumab, | EVQLVESGGGLVQPGGSLRLSCAASGFNIKDTYIHWVRQAPG | 78 |
| B033-2_HC | KGLEWVARIYPTNGYTRYADSVKGRFTISADTSKNTAYLQMNS | |
| | LRAEDTAVYYCSRWGGDGFYAMDYWGQGTLVTVSSASTKGP | |
| | SVFPLAPSSKSTSGGTAALGCLVKDYFPEPVTVSWNSGALTS | |
| | GVHTFPAVLQSSGLYSLSSVVTVPSSSLGTQTYICNVNHKPSN | |
| | TKVDKKVEPKSCHHHHHH | |

Constructs for binding EGFR

| | | |
|---|---|---|
| E200 + EGFR binder | GHNYTTRNILPGLNITSEIVMTQSPATLSLSPGERATLSCRASQ | 79 |
| (nfP2X₇ epitope | SVSSYLAWYQQKPGQAPRLLIYDASNRATGIPARFSGSGSGT | |
| underlined) | DFTLTISSLEPEDFAVYYCHQYGSTPLTFGGGTKAEIKRTVAAP | |
| EGFR, Necitumumab, | SVFIFPPSDEQLKSGTASVVCLLNNFYPREAKVQWKVDNALQS | |
| B034-1 LC | GNSQESVTEQDSKDSTYSLSSTLTLSKADYEKHKVYACEVTH | |
| | QGLSSPVTKSFNRGECHHHHHH | |
| EGFR, Necitumumab, | QVQLQESGPGLVKPSQTLSLTCTVSGGSISSGDYYWSWIRQP | 80 |
| B034-2_HC | PGKGLEWIGYIYYSGSTDYNPSLKSRVTMSVDTSKNQFSLKVN | |
| | SVTAADTAVYYCARVSIFGVGTFDYWGQGTLVTVSSASTKGP | |
| | SVFPLAPSSKSTSGGTAALGCLVKDYFPEPVTVSWNSGALTS | |
| | GVHTFPAVLQSSGLYSLSSVVTVPSSSLGTQTYICNVNHKPSN | |
| | TKVDKKVEPKSCHHHHHH | |
| E200 + EGFR binder | GHNYTTRNILPGLNITSDIQMTQSPSSLSASVGDRVTITCSASS | 81 |
| (nfP2X₇ epitope | SVTYMYWYQQKPGKAPKLLIYDTSNLASGVPSRFSGSGSGTD | |
| underlined) | YTFTISSLQPEDIATYYCQQWSSHIFTFGQGTKVEIKRTVAAPS | |
| EGFR, Matuzumab, | VFIFPPSDEQLKSGTASVVCLLNNFYPREAKVQWKVDNALQS | |
| B035-1_LC | GNSQESVTEQDSKDSTYSLSSTLTLSKADYEKHKVYACEVTH | |
| | QGLSSPVTKSFNRGECHHHHHH | |
| EGFR, Matuzumab, | QVQLVQSGAEVKKPGASVKVSCKASGYTFTSHWMHWVRQAP | 82 |
| B035-2_HC | GQGLEWIGEFNPSNGRTNYNEKFKSKATMTVDTSTNTAYMEL | |
| | SSLRSEDTAVYYCASRDYDYDGRYFDYWGQGTLVTVSSASTK | |
| | GPSVFPLAPSSKSTSGGTAALGCLVKDYFPEPVTVSWNSGAL | |
| | TSGVHTFPAVLQSSGLYSLSSVVTVPSSSLGTQTYICNVNHKP | |
| | SNTKVDKKVEPKSCHHHHHH | |
| E200 + EGFR binder | GHNYTTRNILPGLNITSDIQMTQSPSSLSASVGDRVTITCQASQ | 83 |
| (nfP2X₇ epitope | DISNYLNWYQQKPGKAPKLLIYDASNLETGVPSRFSGSGSGTD | |
| underlined) | FTFTISSLQPEDIATYFCQHFDHLPLAFGGGTKVEIKRTVAAPS | |
| EGFR, Panitumumab, | VFIFPPSDEQLKSGTASVVCLLNNFYPREAKVQWKVDNALQS | |
| B036-1_LC | GNSQESVTEQDSKDSTYSLSSTLTLSKADYEKHKVYACEVTH | |
| | QGLSSPVTKSFNRGECHHHHHH | |
| EGFR, Panitumumab, | QVQLQESGPGLVKPSETLSLTCTVSGGSVSSGDYYWTWIRQS | 84 |
| B036-2_HC | PGKGLEWIGHIYYSGNTNYNPSLKSRLTISIDTSKTQFSLKLSSV | |
| | TAADTAIYYCVRDRVTGAFDIWGQGTMVTVSSASTKGPSVFPL | |
| | APSSKSTSGGTAALGCLVKDYFPEPVTVSWNSGALTSGVHTF | |
| | PAVLQSSGLYSLSSVVTVPSSSLGTQTYICNVNHKPSNTKVDK | |
| | KVEPKSCHHHHHH | |

Constructs for binding CD276

| | | |
|---|---|---|
| E200 + CD276 binder | GHNYTTRNILPGLNITSEIVMTQSPATLSVSPGERVTLSCRASQ | 85 |
| (nfP2X₇ epitope | SISDYLYWYQQKSHESPRLLIKYASQSISGIPARFSGSGSGSEF | |
| underlined) | TLTINSVEPEDVGVYYCQNGHSFPLTFGQGTKLELKRTVAAPS | |
| CD276, hu8H9-6m, | VFIFPPSDEQLKSGTASVVCLLNNFYPREAKVQWKVDNALQS | |
| B037-1_LC | GNSQESVTEQDSKDSTYSLSSTLTLSKADYEKHKVYACEVTH | |
| | QGLSSPVTKSFNRGECHHHHHH | |
| CD276, hu8H9-6m, | QVQLVQSGAEVVKPGASVKLSCKTSGYTFTNYDINWVRQRPG | 86 |
| B037-2_HC | QGLEWIGWIFPGDDSTQYNEKFKGKATLTTDTSTSTAYMELSS | |
| | LRSEDTAVYFCARQTTGTWFAYWGQGTLVTVSSASTKGPSVF | |
| | PLAPSSKSTSGGTAALGCLVKDYFPEPVTVSWNSGALTSGVH | |
| | TFPAVLQSSGLYSLSSVVTVPSSSLGTQTYICNVNHKPSNTKV | |
| | DKKVEPKSCHHHHHH | |

Constructs for binding GD2

| | | |
|---|---|---|
| E200 + GD2 binder | GHNYTTRNILPGLNITSKIVMTQTPATLSVSAGERVTITCKASQS | 87 |
| (nfP2X₇ epitope | VSNHVTWYQQKPGQAPRLLIYSASNRYSGVPARFSGSGYGTE | |
| underlined) | FTFTISSVQSEDFAVYFCQQDYSSFGQGTKLEIKRTVAAPSVFI | |
| GD2, Naxitamab, | FPPSDEQLKSGTASVVCLLNNFYPREAKVQWKVDNALQSGNS | |
| B038-1_LC | QESVTEQDSKDSTYSLSSTLTLSKADYEKHKVYACEVTHQGLS | |
| | SPVTKSFNRGECHHHHHH | |
| GD2, Naxitamab, | QVQLVESGPGVVQPGRSLRLSCAVSGFSVTNYGVHWVRQPP | 88 |
| B038-2_HC | GKGLEWLGVIWAGGITNYNSSVKGRLTISKDNSKNTVYLQMNS | |
| | LRAEDTAVYYCASRGGHYGYALDYWGQGTLVTVSSASTKGP | |
| | SVFPLAPSSKSTSGGTAALGCLVKDYFPEPVTVSWNSGALTS | |
| | GVHTFPAVLQSSGLYSLSSVVTVPSSSLGTQTYICNVNHKPSN | |
| | TKVDKKVEPKSCHHHHHH | |

Constructs for binding BCMA

| | | |
|---|---|---|
| E200 + BCMA binder | GHNYTTRNILPGLNITSDIQMTQSPSSLSASVGDRVTITCSASQ | 89 |
| (nfP2X₇ epitope | DISNYLNWYQQKPGKAPKLLIYYTSNLHSGVPSRFSGSGSGTD | |
| underlined) | FTLTISSLOPEDFATYYCQQYRKLPWTFGQGTKLEIKRTVAAPS | |
| BCMA, clone CA8 | VFIFPPSDEQLKSGTASVVCLLNNFYPREAKVQWKVDNALQS | |
| J9M0, B039-1_LC | GNSQESVTEQDSKDSTYSLSSTLTLSKADYEKHKVYACEVTH | |
| | QGLSSPVTKSFNRGECHHHHHH | |
| BCMA, clone CA8 | QVQLVQSGAEVKKPGSSVKVSCKGSGYTFTNYWMHWVRQA | 90 |
| J9MO, B039-2_HC | PGQGLEWIGATYRGHSDTYYNQKFKGRATLTADTSTSTAYME | |
| | LSSLRSEDTAVYYCTRGAIYDGYDVLDNWGQGTLVTVSSAST | |
| | KGPSVFPLAPSSKSTSGGTAALGCLVKDYFPEPVTVSWNSGA | |
| | LTSGVHTFPAVLQSSGLYSLSSVVTVPSSSLGTQTYICNVNHK | |
| | PSNTKVDKKVEPKSCHHHHHH | |

Constructs for binding CD371

| | | |
|---|---|---|
| E200 + CD371 binder | GHNYTTRNILPGLNITSDIVMTQSPSSVSASVGDRVTITCRASQ | 91 |
| (nfP2X₇ epitope | DISSWLAWYQQKPGKAPKLLIYAASSLQSGVPSRFNGSGSGT | |
| underlined) | DFTLTISSLQPEDFATYYCQQSYSTPLTFGGGTKVEIKRTVAAP | |
| US10568947_CAR9, | SVFIFPPSDEQLKSGTASVVCLLNNFYPREAKVQWKVDNALQS | |
| B040-1_LC | GNSQESVTEQDSKDSTYSLSSTLTLSKADYEKHKVYACEVTH | |
| | QGLSSPVTKSFNRGECHHHHHH | |
| CD371, | QVQLVQSGAEVKEPGASVKVSCKAPANTFSDHVMHWVRQAP | 92 |
| US10568947_CAR9, | GQRFEWMGYIHAANGGTHYSQKFQDRVTITRDTSANTVYMDL | |
| B040-2_HC | SSLRSEDTAVYYCARGGYNSDAFDIWGQGTMVTVSSASTKGP | |
| | SVFPLAPSSKSTSGGTAALGCLVKDYFPEPVTVSWNSGALTS | |
| | GVHTFPAVLQSSGLYSLSSVVTVPSSSLGTQTYICNVNHKPSN | |
| | TKVDKKVEPKSCHHHHHH | |

Constructs for binding CD135

| | | |
|---|---|---|
| E200 + CD135 binder | GHNYTTRNILPGLNITSDIVLTQSPATLSVTPGDSVSLSCRASQ | 93 |
| (nfP2X₇ epitope | SISNNLHWYQQKSHESPRLLIKYASQSISGIPSRFSGSGSGTDF | |
| underlined) | TLSINSVETEDFGVYFCQQSNTWPYTFGGGTKLEIKRTVAAPS | |
| CD135, clone 4G8, | VFIFPPSDEQLKSGTASVVCLLNNFYPREAKVQWKVDNALQS | |
| B041-1_LC | GNSQESVTEQDSKDSTYSLSSTLTLSKADYEKHKVYACEVTH | |
| | QGLSSPVTKSFNRGECHHHHHH | |
| CD135, clone 4G8, | QVQLQQPGAELVKPGASLKLSCKSSGYTFTSYWMHWVRQRP | 94 |
| B041-2_HC | GHGLEWIGEIDPSDSYKDYNQKFKDKATLTVDRSSNTAYMHLS | |
| | SLTSDDSAVYYCARAITTTPFDFWGQGTTLTVSSASTKGPSVF | |
| | PLAPSSKSTSGGTAALGCLVKDYFPEPVTVSWNSGALTSGVH | |
| | TFPAVLQSSGLYSLSSVVTVPSSSLGTQTYICNVNHKPSNTKV | |
| | DKKVEPKSCHHHHHH | |

Constructs for binding CD123

| | | |
|---|---|---|
| E200 + CD123 binder | GHNYTTRNILPGLNITSDIVLTQSPASLAVSLGQRATISCRASES | 95 |
| (nfP2X₇ epitope | VDNYGNTFMHWYQQKPGQPPKLLIYRASNLESGIPARFSGSG | |
| underlined) | SRTDFTLTINPVEADDVATYYCQQSNEDPPTFGAGTKLELKRT | |
| CD123, clone 32716, | VAAPSVFIFPPSDEQLKSGTASVVCLLNNFYPREAKVQWKVDN | |
| B042-1_LC | ALQSGNSQESVTEQDSKDSTYSLSSTLTLSKADYEKHKVYACE | |
| | VTHQGLSSPVTKSFNRGECHHHHHH | |
| CD123, clone 32716, | QIQLVQSGPELKKPGETVKISCKASGYIFTNYGMNWVKQAPGK | 96 |
| B042-2_HC | SFKWMGWINTYTGESTYSADFKGRFAFSLETSASTAYLHINDL | |
| | KNEDTATYFCARSGGYDPMDYWGQGTSVTVSSASTKGPSVF | |
| | PLAPSSKSTSGGTAALGCLVKDYFPEPVTVSWNSGALTSGVH | |
| | TFPAVLQSSGLYSLSSVVTVPSSSLGTQTYICNVNHKPSNTKV | |
| | DKKVEPKSCHHHHHH | |

Constructs for binding CD105

| | | |
|---|---|---|
| E200 + CD105 binder | GHNYTTRNILPGLNITSQIVLSQSPAILSASPGEKVTMTCRASS | 97 |
| (nfP2X₇ epitope | SVSYMHWYQQKPGSSPKPWIYATSNLASGVPVRFSGSGSGT | |
| underlined) | SYSLTISRVEAEDAATYYCQQWSSNPLTFGAGTKLELKRTVAA | |
| CD105, Carotuximab, | PSVFIFPPSDEQLKSGTASVVCLLNNFYPREAKVQWKVDNALQ | |
| B043-1_LC | SGNSQESVTEQDSKDSTYSLSSTLTLSKADYEKHKVYACEVTH | |
| | QGLSSPVTKSFNRGECHHHHHH | |
| CD105, Carotuximab, | EVKLEESGGGLVQPGGSMKLSCAASGFTFSDAWMDWVRQS | 98 |
| B043-2_HC | PEKGLEWVAEIRSKASNHATYYAESVKGRFTISRDDSKSSVYL | |
| | QMNSLRAEDTGIYYCTRWRRFFDSWGQGTTLTVSSASTKGPS | |
| | VFPLAPSSKSTSGGTAALGCLVKDYFPEPVTVSWNSGALTSG | |
| | VHTFPAVLQSSGLYSLSSVVTVPSSSLGTQTYICNVNHKPSNT | |
| | KVDKKVEPKSCHHHHHH | |

Constructs for binding ROR-1

| | | |
|---|---|---|
| E200 + ROR-1 binder | GHNYTTRNILPGLNITSEIVLSQSPAITAASLGQKVTITCSASSN | 99 |
| (nfP2X₇ epitope | VSYIHWYQQRSGTSPRPWIYEISKLASGVPVRFSGSGSGTSYS | |
| underlined) ROR-1, | LTISSMEAEDAAIYYCQQWNYPLITFGSGTKLEIQRTVAAPSVFI | |
| clone D10v3, B044- | FPPSDEQLKSGTASVVCLLNNFYPREAKVQWKVDNALQSGNS | |
| 1 LC | QESVTEQDSKDSTYSLSSTLTLSKADYEKHKVYACEVTHQGLS | |
| | SPVTKSFNRGECHHHHHH | |
| ROR-1, clone D10v3, | QVQLKESGPGLVAPSQTLSITCTVSGFSLTSYGVHWVRQPPG | 100 |
| B044-2_HC | KGLEWLGVIWAGGFTNYNSALKSRLSISKDNSKSQVLLKMTSL | |
| | QTDDTAMYYCARRGSSYSMDYWGQGTSVTVSSASTKGPSVF | |
| | PLAPSSKSTSGGTAALGCLVKDYFPEPVTVSWNSGALTSGVH | |
| | TFPAVLQSSGLYSLSSVVTVPSSSLGTQTYICNVNHKPSNTKV | |
| | DKKVEPKSCHHHHHH | |

Constructs for binding PD-L1

| | | |
|---|---|---|
| E200 + PD-L1 binder | GHNYTTRNILPGLNITSDIQMTQSPSSLSASVGDRVTITCRASQ | 101 |
| (nfP2X₇ epitope | DVSTAVAWYQQKPGKAPKLLIYSASFLYSGVPSRFSGSGSGT | |
| underlined) PD-L1, | DFTLTISSLQPEDFATYYCQQYLYHPATFGQGTKVEIKRTVAAP | |
| Atezolizumab, B045- | SVFIFPPSDEQLKSGTASVVCLLNNFYPREAKVQWKVDNALQS | |
| 1 LC | GNSQESVTEQDSKDSTYSLSSTLTLSKADYEKHKVYACEVTH | |
| | QGLSSPVTKSFNRGECHHHHHH | |
| PD-L1, Atezolizumab, | EVQLVESGGGLVQPGGSLRLSCAASGFTFSDSWIHWVRQAP | 102 |
| B045-2_HC | GKGLEWVAWISPYGGSTYYADSVKGRFTISADTSKNTAYLQM | |
| | NSLRAEDTAVYYCARRHWPGGFDYWGQGTLVTVSSASTKGP | |
| | SVFPLAPSSKSTSGGTAALGCLVKDYFPEPVTVSWNSGALTS | |
| | GVHTFPAVLQSSGLYSLSSVVTVPSSSLGTQTYICNVNHKPSN | |
| | TKVDKKVEPKSCHHHHHH | |

Constructs for binding MET-R

| | | |
|---|---|---|
| E200 + MET-R binder | GHNYTTRNILPGLNITSDIQMTQSPSSLSASVGDRVTITCKSSQ | 103 |
| (nfP2X₇ epitope | SLLYTSSQKNYLAWYQQKPGKAPKLLIYWASTRESGVPSRFS | |
| underlined) MET-R, | GSGSGTDFTLTISSLOPEDFATYYCQQYYAYPWTFGQGTKVEI | |
| Onartuzumab, B046- | KRTVAAPSVFIFPPSDEQLKSGTASVVCLLNNFYPREAKVQWK | |
| 1 LC | VDNALQSGNSQESVTEQDSKDSTYSLSSTLTLSKADYEKHKV | |
| | YACEVTHQGLSSPVTKSFNRGECHHHHHH | |
| MET-R, Onartuzumab, | EVQLVESGGGLVQPGGSLRLSCAASGYTFTSYWLHWVRQAP | 104 |
| B046-2_HC | GKGLEWVGMIDPSNSDTRFNPNFKDRFTISADTSKNTAYLQM | |
| | NSLRAEDTAVYYCATYRSYVTPLDYWGQGTLVTVSSASTKGP | |
| | SVFPLAPSSKSTSGGTAALGCLVKDYFPEPVTVSWNSGALTS | |
| | GVHTFPAVLQSSGLYSLSSVVTVPSSSLGTQTYICNVNHKPSN | |
| | TKVDKKVEPKSCHHHHHH | |

Constructs for binding PDGFRalpha

| | | |
|---|---|---|
| E200 + PDGFR binder | GHNYTTRNILPGLNITSDIQMTQSPSSLSASVGDRVSITCRPSQ | 105 |
| (nfP2X₇ epitope | SFSRYINWYQQKPGKAPKLLIHAASSLVGGVPSRFSGSGSGT | |
| underlined) | DFTLTISSLQPEDFATYYCQQTYSNPPITFGQGTRLEMKRTVA | |
| PDGFRalpha, | APSVFIFPPSDEQLKSGTASVVCLLNNFYPREAKVQWKVDNAL | |
| Tovetumab, B047- | QSGNSQESVTEQDSKDSTYSLSSTLTLSKADYEKHKVYACEV | |
| 1 LC | THQGLSSPVTKSFNRGECHHHHHH | |
| PDGFRalpha, | QVQLVESGGGLVKPGGSLRLSCAASGFTFSDYYMNWIRQAP | 106 |
| Tovetumab, B047- | GKGLEWVSYISSSGSIIYYADSVKGRFTISRDNAKNSLYLQMNS | |
| 2_HC | LRAEDTAVYYCAREGRIAARGMDVWGQGTTVTVSSASTKGPS | |
| | VFPLAPSSKSTSGGTAALGCLVKDYFPEPVTVSWNSGALTSG | |
| | VHTFPAVLQSSGLYSLSSVVTVPSSSLGTQTYICNVNHKPSNT | |
| | KVDKKVEPKSCHHHHHH | |
| E200 + PDGFR binder | GHNYTTRNILPGLNITSEIVLTQSPATLSLSPGERATLSCRASQ | 107 |
| (nfP2X₇ epitope | SVSSYLAWYQQKPGQAPRLLIYDASNRATGIPARFSGSGSGT | |
| underlined) | DFTLTISSLEPEDFAVYYCQQRSNWPPAFGQGTKVEIKRTVAA | |
| PDGFRalpha, | PSVFIFPPSDEQLKSGTASVVCLLNNFYPREAKVQWKVDNALQ | |
| Olaratumab, B048- | SGNSQESVTEQDSKDSTYSLSSTLTLSKADYEKHKVYACEVTH | |
| 1 LC | QGLSSPVTKSFNRGECHHHHHH | |
| PDGFRalpha, | QLQLQESGPGLVKPSETLSLTCTVSGGSINSSSYYWGWLRQS | 108 |
| Olaratumab, B048- | PGKGLEWIGSFFYTGSTYYNPSLRSRLTISVDTSKNQFSLMLS | |
| 2_HC | SVTAADTAVYYCARQSTYYYGSGNYYGWFDRWDQGTLVTVS | |
| | SASTKGPSVFPLAPSSKSTSGGTAALGCLVKDYFPEPVTVSW | |
| | NSGALTSGVHTFPAVLQSSGLYSLSSVVTVPSSSLGTQTYICN | |
| | VNHKPSNTKVDKKVEPKSCHHHHHH | |

Constructs for binding Her3

| | | |
|---|---|---|
| E200 + Her3 binder | GHNYTTRNILPGLNITSQSALTQPASVSGSPGQSITISCTGTSS | 109 |
| (nfP2X₇ epitope | DVGSYNVVSWYQQHPGKAPKLIIYEVSQRPSGVSNRFSGSKS | |
| underlined) Her3, | GNTASLTISGLQTEDEADYYCCSYAGSSIFVIFGGGTKVTVLRT | |
| Seribantumab, B049- | VAAPSVFIFPPSDEQLKSGTASVVCLLNNFYPREAKVQWKVDN | |
| 1 LC | ALQSGNSQESVTEQDSKDSTYSLSSTLTLSKADYEKHKVYACE | |
| | VTHQGLSSPVTKSFNRGECHHHHHH | |
| Her3, Seribantumab, | EVQLLESGGGLVQPGGSLRLSCAASGFTFSHYVMAWVRQAP | 110 |
| B049-2_HC | GKGLEWVSSISSSGGWTLYADSVKGRFTISRDNSKNTLYLQM | |
| | NSLRAEDTAVYYCTRGLKMATIFDYWGQGTLVTVSSASTKGP | |
| | SVFPLAPSSKSTSGGTAALGCLVKDYFPEPVTVSWNSGALTS | |
| | GVHTFPAVLQSSGLYSLSSVVTVPSSSLGTQTYICNVNHKPSN | |
| | TKVDKKVEPKSCHHHHHH | |

Constructs for binding FRalpha

| | | |
|---|---|---|
| E200 + FRa binder | GHNYTTRNILPGLNITSDIQLTQSPSSLSASVGDRVTITCSVSSS | 111 |
| (nfP2X₇ epitope | ISSNNLHWYQQKPGKAPKPWIYGTSNLASGVPSRFSGSGSGT | |
| underlined) FRalpha, | DYTFTISSLQPEDIATYYCQQWSSYPYMYTFGQGTKVEIKRTV | |
| Farletuzumab, B050- | AAPSVFIFPPSDEQLKSGTASVVCLLNNFYPREAKVQWKVDNA | |
| 1 LC | LQSGNSQESVTEQDSKDSTYSLSSTLTLSKADYEKHKVYACEV | |
| | THQGLSSPVTKSFNRGECHHHHHH | |
| FRalpha, | EVQLVESGGGVVQPGRSLRLSCSASGFTFSGYGLSWVRQAP | 112 |
| Farletuzumab, B050- | GKGLEWVAMISSGGSYTYYADSVKGRFAISRDNAKNTLFLQM | |
| 2_HC | DSLRPEDTGVYFCARHGDDPAWFAYWGQGTPVTVSSASTKG | |
| | PSVFPLAPSSKSTSGGTAALGCLVKDYFPEPVTVSWNSGALTS | |
| | GVHTFPAVLQSSGLYSLSSVVTVPSSSLGTQTYICNVNHKPSN | |
| | TKVDKKVEPKSCHHHHHH | |

Constructs for binding GPC3

| | | |
|---|---|---|
| E200 + GPC3 binder | GHNYTTRNILPGLNITSDVVMTQSPLSLPVTPGEPASISCRSSQ | 113 |
| (nfP2X₇ epitope | SLVHSNRNTYLHWYLQKPGQSPQLLIYKVSNRFSGVPDRFSG | |
| underlined) GPC3, | SGSGTDFTLKISRVEAEDVGVYYCSQNTHVPPTFGQGTKLEIK | |
| Codrituzumab, B051- | RTVAAPSVFIFPPSDEQLKSGTASVVCLLNNFYPREAKVQWKV | |
| 1_LC | DNALQSGNSQESVTEQDSKDSTYSLSSTLTLSKADYEKHKVY | |
| | ACEVTHQGLSSPVTKSFNRGECHHHHHH | |
| GPC3, Codrituzumab, | QVQLVQSGAEVKKPGASVKVSCKASGYTFTDYEMHWVRQAP | 114 |
| B051-2_HC | GQGLEWMGALDPKTGDTAYSQKFKGRVTLTADKSTSTAYMEL | |
| | SSLTSEDTAVYYCTRFYSYTYWGQGTLVTVSSASTKGPSVFPL | |
| | APSSKSTSGGTAALGCLVKDYFPEPVTVSWNSGALTSGVHTF | |
| | PAVLQSSGLYSLSSVVTVPSSSLGTQTYICNVNHKPSNTKVDK | |
| | KVEPKSCHHHHHH | |

Constructs for binding SLAMF7

| | | |
|---|---|---|
| E200 + SLAMF7 binder | GHNYTTRNILPGLNITSDIQMTQSPSSLSASVGDRVTITCKASQ | 115 |
| (nfP2X₇ epitope | DVGIAVAWYQQKPGKVPKLLIYWASTRHTGVPDRFSGSGSGT | |
| underlined) SLAMF7, | DFTLTISSLQPEDVATYYCQQYSSYPYTFGQGTKVEIKRTVAAP | |
| Elotuzumab, B052- | SVFIFPPSDEQLKSGTASVVCLLNNFYPREAKVQWKVDNALQS | |
| 1 LC | GNSQESVTEQDSKDSTYSLSSTLTLSKADYEKHKVYACEVTH | |
| | QGLSSPVTKSFNRGECHHHHHH | |
| SLAMF7, Elotuzumab, | EVQLVESGGGLVQPGGSLRLSCAASGFDFSRYWMSWVRQAP | 116 |
| B052-2_HC | GKGLEWIGEINPDSSTINYAPSLKDKFIISRDNAKNSLYLQMNSL | |
| | RAEDTAVYYCARPDGNYWYFDVWGQGTLVTVSSASTKGPSV | |
| | FPLAPSSKSTSGGTAALGCLVKDYFPEPVTVSWNSGALTSGV | |
| | HTFPAVLQSSGLYSLSSVVTVPSSSLGTQTYICNVNHKPSNTK | |
| | VDKKVEPKSCHHHHHH | |

Constructs for binding TNFRSF10B

| | | |
|---|---|---|
| E200 + TNFRSF10B | GHNYTTRNILPGLNITSDIQMTQSPSSLSASVGDRVTITCKASQ | 117 |
| binder (nfP2X₇ epitope | DVGTAVAWYQQKPGKAPKLLIYWASTRHTGVPSRFSGSGSGT | |
| underlined), | DFTLTISSLOPEDFATYYCQQYSSYRTFGQGTKVEIKRTVAAPS | |
| Tigatuzumab, B053- | VFIFPPSDEQLKSGTASVVCLLNNFYPREAKVQWKVDNALQS | |
| 1 LC | GNSQESVTEQDSKDSTYSLSSTLTLSKADYEKHKVYACEVTH | |
| | QGLSSPVTKSFNRGECHHHHHH | |
| TNFRSF10B, | EVQLVESGGGLVQPGGSLRLSCAASGFTFSSYVMSWVRQAP | 118 |
| Tigatuzumab, B053- | GKGLEWVATISSGGSYTYYPDSVKGRFTISRDNAKNTLYLQMN | |
| 2_HC | SLRAEDTAVYYCARRGDSMITTDYWGQGTLVTVSSASTKGPS | |
| | VFPLAPSSKSTSGGTAALGCLVKDYFPEPVTVSWNSGALTSG | |
| | VHTFPAVLQSSGLYSLSSVVTVPSSSLGTQTYICNVNHKPSNT | |
| | KVDKKVEPKSCHHHHHH | |

Constructs for binding GPNMB

| | | |
|---|---|---|
| E200 + GPNMB binder | GHNYTTRNILPGLNITSEIVMTQSPATLSVSPGERATLSCRASQ | 119 |
| (nfP2X₇ epitope | SVDNNLVWYQQKPGQAPRLLIYGASTRATGIPARFSGSGSGT | |
| underlined), GPNMB, | EFTLTISSLQSEDFAVYYCQQYNNWPPWTFGQGTKVEIKRTVA | |
| Glembatumumab, | APSVFIFPPSDEQLKSGTASVVCLLNNFYPREAKVQWKVDNAL | |
| B054-1 LC | QSGNSQESVTEQDSKDSTYSLSSTLTLSKADYEKHKVYACEV | |
| | THQGLSSPVTKSFNRGECHHHHHH | |
| GPNMB, | QVQLQESGPGLVKPSQTLSLTCTVSGGSISSFNYYWSWIRHH | 120 |
| Glembatumumab, | PGKGLEWIGYIYYSGSTYSNPSLKSRVTISVDTSKNQFSLTLSS | |
| B054-2_HC | VTAADTAVYYCARGYNWNYFDYWGQGTLVTVSSASTKGPSV | |
| | FPLAPSSKSTSGGTAALGCLVKDYFPEPVTVSWNSGALTSGV | |
| | HTFPAVLQSSGLYSLSSVVTVPSSSLGTQTYICNVNHKPSNTK | |
| | VDKKVEPKSCHHHHHH | |

Constructs for binding VEGFR2

| | | |
|---|---|---|
| E200 + VEGFR2 binder | GHNYTTRNILPGLNITSDIQMTQSPSSVSASIGDRVTITCRASQ | 121 |
| (nfP2X₇ epitope | GIDNWLGWYQQKPGKAPKLLIYDASNLDTGVPSRFSGSGSGT | |
| underlined), | YFTLTISSLQAEDFAVYFCQQAKAFPPTFGGGTKVDIKRTVAAP | |
| Ramucirumab, B055- | SVFIFPPSDEQLKSGTASVVCLLNNFYPREAKVQWKVDNALQS | |
| 1 LC | GNSQESVTEQDSKDSTYSLSSTLTLSKADYEKHKVYACEVTH | |
| | QGLSSPVTKSFNRGECHHHHHH | |
| VEGFR2, | EVQLVQSGGGLVKPGGSLRLSCAASGFTFSSYSMNWVRQAP | 122 |
| Ramucirumab, B055- | GKGLEWVSSISSSSSYIYYADSVKGRFTISRDNAKNSLYLQMN | |
| 2_HC | SLRAEDTAVYYCARVTDAFDIWGQGTMVTVSSASTKGPSVFP | |
| | LAPSSKSTSGGTAALGCLVKDYFPEPVTVSWNSGALTSGVHT | |
| | FPAVLQSSGLYSLSSVVTVPSSSLGTQTYICNVNHKPSNTKVD | |
| | KKVEPKSCHHHHHH | |

Constructs for binding a4B7 &/or α4β7

| | | |
|---|---|---|
| E200 + α4β7 and | GHNYTTRNILPGLNITSDIQMTQSPSSLSASVGDRVTITCRASE | 123 |
| α4β7binder (nfP2X₇ | SVDDLLHWYQQKPGKAPKLLIKYASQSISGVPSRFSGSGSGTD | |
| epitope underlined), | FTLTISSLOPEDFATYYCQQGNSLPNTFGQGTKVEIKRTVAAPS | |
| Etrolizumab, B056- | VFIFPPSDEQLKSGTASVVCLLNNFYPREAKVQWKVDNALQS | |
| 1 LC | GNSQESVTEQDSKDSTYSLSSTLTLSKADYEKHKVYACEVTH | |
| | QGLSSPVTKSFNRGECHHHHHH | |
| a4β7 & α4β7, | EVQLVESGGGLVQPGGSLRLSCAASGFFITNNYWGWVRQAP | 124 |
| Etrolizumab, B056- | GKGLEWVGYISYSGSTSYNPSLKSRFTISRDTSKNTFYLQMNS | |
| 2_HC | LRAEDTAVYYCARTGSSGYFDFWGQGTLVTVSSASTKGPSVF | |
| | PLAPSSKSTSGGTAALGCLVKDYFPEPVTVSWNSGALTSGVH | |
| | TFPAVLQSSGLYSLSSVVTVPSSSLGTQTYICNVNHKPSNTKV | |
| | DKKVEPKSCHHHHHH | |
| E200 + a4β7 binder | GHNYTTRNILPGLNITSDIQMTQSPSSVSASVGDRVTITCRASQ | 125 |
| (nfP2X₇ epitope | GISSWLAWYQQKPGKAPKLLIYGASNLESGVPSRFSGSGSGT | |
| underlined), a4β7, | DFTLTISSLQPEDFANYYCQQANSFPWTFGQGTKVEIKRTVAA | |
| Abrilumab, B057-1_LC | PSVFIFPPSDEQLKSGTASVVCLLNNFYPREAKVQWKVDNALQ | |
| | SGNSQESVTEQDSKDSTYSLSSTLTLSKADYEKHKVYACEVTH | |
| | QGLSSPVTKSFNRGECHHHHHH | |
| a4β7, Abrilumab, | QVQLVQSGAEVKKPGASVKVSCKVSGYTLSDLSIHWVRQAPG | 126 |
| B057-2_HC | KGLEWMGGFDPQDGETIYAQKFQGRVTMTEDTSTDTAYMEL | |
| | SSLKSEDTAVYYCATGSSSSWFDPWGQGTLVTVSSASTKGPS | |
| | VFPLAPSSKSTSGGTAALGCLVKDYFPEPVTVSWNSGALTSG | |
| | VHTFPAVLQSSGLYSLSSVVTVPSSSLGTQTYICNVNHKPSNT | |
| | KVDKKVEPKSCHHHHHH | |

Constructs for binding CSPG4

| | | |
|---|---|---|
| E200 + CSPG4 binder | GHNYTTRNILPGLNITSRSTQSALTQPASVSGSPGQSITISCTG | 127 |
| (nfP2X₇ epitope | TSSDVGGYNYVSWYQQHPGKAPKLMIYDVSNRPSGVSKRFS | |
| underlined), CSPG4, | GSKSGNTASLTISGLQAEDEADYYCSSYTSSSTRHVFGTGTQL | |
| D2A-1h10-UC12, | TVLGRTVAAPSVFIFPPSDEQLKSGTASVVCLLNNFYPREAKV | |
| B058-1_LC | QWKVDNALQSGNSQESVTEQDSKDSTYSLSSTLTLSKADYEK | |
| | HKVYACEVTHQGLSSPVTKSFNRGECHHHHHH | |
| CSPG4, D2A-1h10- | EVQLVESGAEVKKPGDSLKISCKGSGYSFTSYWIGWVRQMPG | 128 |
| UC12, B058-2_HC | KGLEWMGIIYPGDSVTTYSPAFQGDVTISVDKSISTAYLQWNSL | |
| | KASDTGIYYCARRRGNYYMDVWGNGTLVTVSSASTKGPSVFP | |
| | LAPSSKSTSGGTAALGCLVKDYFPEPVTVSWNSGALTSGVHT | |
| | FPAVLQSSGLYSLSSVVTVPSSSLGTQTYICNVNHKPSNTKVD | |
| | KKVEPKSCHHHHHH | |

Constructs for binding CD80

| | | |
|---|---|---|
| E200 + CD80 binder | GHNYTTRNILPGLNITSESALTQPPSVSGAPGQKVTISCTGSTS | 129 |
| (nfP2X₇ epitope | NIGGYDLHWYQQLPGTAPKLLIYDINKRPSGISDRFSGSKSGTA | |
| underlined), | ASLAITGLQTEDEADYYCQSYDSSLNAQVFGGGTRLTVLRTVA | |
| Galiximab, B059-1_LC | APSVFIFPPSDEQLKSGTASVVCLLNNFYPREAKVQWKVDNAL | |
| | QSGNSQESVTEQDSKDSTYSLSSTLTLSKADYEKHKVYACEV | |
| | THQGLSSPVTKSFNRGECHHHHHH | |
| CD80, Galiximab, | QVQLQESGPGLVKPSETLSLTCAVSGGSISGGYGWGWIRQPP | 130 |
| B059-2_HC | GKGLEWIGSFYSSSGNTYYNPSLKSQVTISTDTSKNQFSLKLN | |
| | SMTAADTAVYYCVRDRLFSVVGMVYNNWFDVWGPGVLVTVS | |
| | SASTKGPSVFPLAPSSKSTSGGTAALGCLVKDYFPEPVTVSW | |
| | NSGALTSGVHTFPAVLQSSGLYSLSSVVTVPSSSLGTQTYICN | |
| | VNHKPSNTKVDKKVEPKSCHHHHHH | |

Constructs for binding CCR4

| | | |
|---|---|---|
| E200 + CCR4 binder | GHNYTTRNILPGLNITSDVLMTQSPLSLPVTPGEPASISCRSSR | 131 |
| (nfP2X₇ epitope | NIVHINGDTYLEWYLQKPGQSPQLLIYKVSNRFSGVPDRFSGS | |
| underlined), | GSGTDFTLKISRVEAEDVGVYYCFQGSLLPWTFGQGTKVEIKR | |
| Mogamulizumab, | TVAAPSVFIFPPSDEQLKSGTASVVCLLNNFYPREAKVQWKVD | |
| B060-1_LC | NALQSGNSQESVTEQDSKDSTYSLSSTLTLSKADYEKHKVYA | |
| | CEVTHQGLSSPVTKSFNRGECHHHHHH | |
| CCR4, | EVQLVESGGDLVQPGRSLRLSCAASGFIFSNYGMSWVRQAP | 132 |
| Mogamulizumab, | GKGLEWVATISSASTYSYYPDSVKGRFTISRDNAKNSLYLQMN | |
| B060-2_HC | SLRVEDTALYYCGRHSDGNFAFGYWGQGTLVTVSSASTKGPS | |
| | VFPLAPSSKSTSGGTAALGCLVKDYFPEPVTVSWNSGALTSG | |
| | VHTFPAVLQSSGLYSLSSVVTVPSSSLGTQTYICNVNHKPSNT | |
| | KVDKKVEPKSCHHHHHH | |

Constructs for binding CD115

| | | |
|---|---|---|
| E200 + CD115 binder | GHNYTTRNILPGLNITSDIQMTQSPSSLSASVGDRVTITCRASE | 133 |
| (nfP2X₇ epitope | DVNTYVSWYQQKPGKAPKLLIYAASNRYTGVPSRFSGSGSGT | |
| underlined), CD115- | DFTLTISSLQPEDFATYYCQQSFSYPTFGQGTKLEIKRTVAAPS | |
| CSF-1R, | VFIFPPSDEQLKSGTASVVCLLNNFYPREAKVQWKVDNALQS | |
| Emactuzumab, B061- | GNSQESVTEQDSKDSTYSLSSTLTLSKADYEKHKVYACEVTH | |
| 1_LC | QGLSSPVTKSFNRGECHHHHHH | |
| CD115-CSF-1R, | QVQLVQSGAEVKKPGASVKVSCKASGYTFTSYDISWVRQAPG | 134 |
| Emactuzumab, B061- | QGLEWMGVIWTDGGTNYAQKLQGRVTMTTDTSTSTAYMELR | |
| 2 HC | SLRSDDTAVYYCARDQRLYFDVWGQGTTVTVSSASTKGPSVF | |
| | PLAPSSKSTSGGTAALGCLVKDYFPEPVTVSWNSGALTSGVH | |
| | TFPAVLQSSGLYSLSSVVTVPSSSLGTQTYICNVNHKPSNTKV | |
| | DKKVEPKSCHHHHHH | |

Constructs for binding ENOX-2

| | | |
|---|---|---|
| E200 + ENOX-2 binder | GHNYTTRNILPGLNITSENVLTQSPAIMSASPGERVTMTCSASS | 135 |
| (nfP2X₇ epitope | SIRYIYWYQQKPGSSPRLLIYDTSNVAPGVPFRFSGSGSGTSY | |
| underlined), ENOX-2, | SLTINRMEAEDAATYYCQEWSGYPYTFGGGTKLELKRTVAAP | |
| US9459256, B062- | SVFIFPPSDEQLKSGTASVVCLLNNFYPREAKVQWKVDNALQS | |
| 1 LC | GNSQESVTEQDSKDSTYSLSSTLTLSKADYEKHKVYACEVTH | |
| | QGLSSPVTKSFNRGECHHHHHH | |
| ENOX-2, US9459256, | EVKLQESGTEVVKPGASVKLSCKASGYIFTSYDIDWVRQTPEQ | 136 |
| B062-2_HC | GLEWIGWIFPGEGSTEYNEKFKGRATLSVDKSSSTAYMELTRL | |
| | TSEDSAVYFCARGDYYRRYFDLWGQGTTVTVSSASTKGPSVF | |
| | PLAPSSKSTSGGTAALGCLVKDYFPEPVTVSWNSGALTSGVH | |
| | TFPAVLQSSGLYSLSSVVTVPSSSLGTQTYICNVNHKPSNTKV | |
| | DKKVEPKSCHHHHHH | |

Constructs for binding CD56

| | | |
|---|---|---|
| E200 + CD56 binder | GHNYTTRNILPGLNITSDVVMTQSPLSLPVTLGQPASISCRSSQ | 137 |
| (nfP2X₇ epitope | IIIHSDGNTYLEWFQQRPGQSPRRLIYKVSNRFSGVPDRFSGS | |
| underlined), CD56, | GSGTDFTLKISRVEAEDVGVYYCFQGSHVPHTFGQGTKVEIKR | |
| Lorvotuzumab, B063- | TVAAPSVFIFPPSDEQLKSGTASVVCLLNNFYPREAKVQWKVD | |
| 1_LC | NALQSGNSQESVTEQDSKDSTYSLSSTLTLSKADYEKHKVYA | |
| | CEVTHQGLSSPVTKSFNRGECHHHHHH | |
| CD56, ILorvotuzumab, | QVQLVESGGGVVQPGRSLRLSCAASGFTFSSFGMHWVRQAP | 138 |
| B063-2_HC | GKGLEWVAYISSGSFTIYYADSVKGRFTISRDNSKNTLYLQMN | |
| | SLRAEDTAVYYCARMRKGYAMDYWGQGTLVTVSSASTKGPS | |
| | VFPLAPSSKSTSGGTAALGCLVKDYFPEPVTVSWNSGALTSG | |
| | VHTFPAVLQSSGLYSLSSVVTVPSSSLGTQTYICNVNHKPSNT | |
| | KVDKKVEPKSCHHHHHH | |

Constructs for binding huVH1-69

| | | |
|---|---|---|
| E200 + huVH1-69 | GHNYTTRNILPGLNITSDIQLTQSPSSLSASVGDRVTITCRASQ | 139 |
| binder (nfP2X₇ epitope | GISSNIVWLQQKPGKAPKGLIYHGTNLESGVPSRFSGSGSGTD | |
| underlined), huVH1- | YTLTISSLEPEDFATYYCVQYSQFPPTFGQGTKLEIKRTVAAPS | |
| 69, B075-1_LC | VFIFPPSDEQLKSGTASVVCLLNNFYPREAKVQWKVDNALQS | |
| | GNSQESVTEQDSKDSTYSLSSTLTLSKADYEKHKVYACEVTH | |
| | QGLSSPVTKSFNRGECHHHHHH | |
| huVH1-69, B075-2_HC | QVQLVQSGAEVVKPGASVKVSCKASGYTFTSYWMHWVKQAP | 140 |
| | GQGLEWIGAVSPGNSDTSYNEKFKGKATLTVDTSASTAYMEL | |
| | SSLRSEDTAVYYCTRSRYGNNALDYWGQGTLVTVSASTKGPS | |
| | VFPLAPSSKSTSGGTAALGCLVKDYFPEPVTVSWNSGALTSG | |
| | VHTFPAVLQSSGLYSLSSVVTVPSSSLGTQTYICNVNHKPSNT | |
| | KVDKKVEPKSCHHHHHH | |

Constructs for binding CD19 (IgG1 format)

| | | |
|---|---|---|
| CD19, Tafasitamab, | EVQLVESGGGLVKPGGSLKLSCAASGYTFTSYVMHWVRQAP | 141 |
| OR19_1, wt-IgG1, HC | GKGLEWIGYINPYNDGTKYNEKFQGRVTISSDKSISTAYMELSS | |
| | LRSEDTAMYYCARGTYYYGTRVFDYWGQGTLVTVSSASTKG | |
| | PSVFPLAPSSKSTSGGTAALGCLVKDYFPEPVTVSWNSGALTS | |
| | GVHTFPAVLQSSGLYSLSSVVTVPSSSLGTQTYICNVNHKPSN | |
| | TKVDKKVEPKSCDKTHTCPPCPAPELLGGPSVFLFPPKPKDTL | |
| | MISRTPEVTCVVVDVSHEDPEVKFNWYVDGVEVHNAKTKPRE | |
| | EQYNSTYRVVSVLTVLHQDWLNGKEYKCKVSNKALPAPIEKTI | |
| | SKAKGQPREPQVYTLPPSRDELTKNQVSLTCLVKGFYPSDIAV | |
| | EWESNGQPENNYKTTPPVLDSDGSFFLYSKLTVDKSRWQQG | |
| | NVFSCSVMHEALHNHYTQKSLSLSPGKHHHHHH | |
| CD19, Tafasitamab, | EVQLVESGGGLVKPGGSLKLSCAASGYTFTSYVMHWVRQAP | 142 |
| OR19_2, IgG1-SDIE, | GKGLEWIGYINPYNDGTKYNEKFQGRVTISSDKSISTAYMELSS | |
| HC | LRSEDTAMYYCARGTYYYGTRVFDYWGQGTLVTVSSASTKG | |
| | PSVFPLAPSSKSTSGGTAALGCLVKDYFPEPVTVSWNSGALTS | |
| | GVHTFPAVLQSSGLYSLSSVVTVPSSSLGTQTYICNVNHKPSN | |
| | TKVDKKVEPKSCDKTHTCPPCPAPELLGGPDVFLFPPKPKDTL | |
| | MISRTPEVTCVVVDVSHEDPEVKFNWYVDGVEVHNAKTKPRE | |
| | EQYNSTYRVVSVLTVLHQDWLNGKEYKCKVSNKALPAPEEKTI | |
| | SKAKGQPREPQVYTLPPSRDELTKNQVSLTCLVKGFYPSDIAV | |
| | EWESNGQPENNYKTTPPVLDSDGSFFLYSKLTVDKSRWQQG | |
| | NVFSCSVMHEALHNHYTQKSLSLSPGKHHHHHH | |

Constructs for binding CD19 (Fab format)

| | | |
|---|---|---|
| CD19, Tafasitamab, | EVQLVESGGGLVKPGGSLKLSCAASGYTFTSYVMHWVRQAP | 143 |
| B020-2_Heavy chain | GKGLEWIGYINPYNDGTKYNEKFQGRVTISSDKSISTAYMELSS | |
| | LRSEDTAMYYCARGTYYYGTRVFDYWGQGTLVTVSSASTKG | |
| | PSVFPLAPSSKSTSGGTAALGCLVKDYFPEPVTVSWNSGALTS | |
| | GVHTFPAVLQSSGLYSLSSVVTVPSSSLGTQTYICNVNHKPSN | |
| | TKVDKKVEPKSCHHHHHH | |
| CD19, Tafasitamab, | DIVMTQSPATLSLSPGERATLSCRSSKSLQNVNGNTYLYWFQ | 144 |
| OR19_7 | QKPGQSPQLLIYRMSNLNSGVPDRFSGSGSGTEFTLTISSLEP | |
| Light chain | EDFAVYYCMQHLEYPITFGAGTKLEIKRTVAAPSVFIFPPSDEQ | |
| | LKSGTASVVCLLNNFYPREAKVQWKVDNALQSGNSQESVTEQ | |
| | DSKDSTYSLSSTLTLSKADYEKHKVYACEVTHQGLSSPVTKSF | |
| | NRGEC | |
| E200 + CD19 binder | GHNYTTRNILPGLNITSDIVMTQSPATLSLSPGERATLSCRSSK | 145 |
| (nfP2X₇ epitope | SLQNVNGNTYLYWFQQKPGQSPQLLIYRMSNLNSGVPDRFSG | |
| underlined), CD19, | SGSGTEFTLTISSLEPEDFAVYYCMQHLEYPITFGAGTKLEIKR | |
| Tafasitamab, Light | TVAAPSVFIFPPSDEQLKSGTASVVCLLNNFYPREAKVQWKVD | |
| chain | NALQSGNSQESVTEQDSKDSTYSLSSTLTLSKADYEKHKVYA | |
| OR19_8 | CEVTHQGLSSPVTKSFNRGEC | |
| E200 + CD19 binder | GHNYTTRNILPGLNITSGGGGSDIVMTQSPATLSLSPGERATLS | 146 |
| (nfP2X₇ epitope | CRSSKSLQNVNGNTYLYWFQQKPGQSPQLLIYRMSNLNSGVP | |
| underlined), CD19, | DRFSGSGSGTEFTLTISSLEPEDFAVYYCMQHLEYPITFGAGT | |
| Tafasitamab, Light | KLEIKRTVAAPSVFIFPPSDEQLKSGTASVVCLLNNFYPREAKV | |
| chain | QWKVDNALQSGNSQESVTEQDSKDSTYSLSSTLTLSKADYEK | |
| OR19_9 | HKVYACEVTHQGLSSPVTKSFNRGEC | |
| E200 + CD19 binder | GHNYTTRNILPGLNITSGGGGSGGGGSGGGGSDIVMTQSPAT | 147 |
| (nfP2X₇ epitope | LSLSPGERATLSCRSSKSLQNVNGNTYLYWFQQKPGQSPQLL | |
| underlined), CD19, | IYRMSNLNSGVPDRFSGSGSGTEFTLTISSLEPEDFAVYYCMQ | |
| Tafasitamab, Light | HLEYPITFGAGTKLEIKRTVAAPSVFIFPPSDEQLKSGTASVVCL | |
| chain | LNNFYPREAKVQWKVDNALQSGNSQESVTEQDSKDSTYSLSS | |
| OR19_10 | TLTLSKADYEKHKVYACEVTHQGLSSPVTKSFNRGEC | |
| E200 + CD19 binder | GHNYTTRNILPGLNITSTFHKTSGSGKDIVMTQSPATLSLSPGE | 148 |
| (nfP2X₇ epitope | RATLSCRSSKSLQNVNGNTYLYWFQQKPGQSPQLLIYRMSNL | |
| underlined), CD19, | NSGVPDRFSGSGSGTEFTLTISSLEPEDFAVYYCMQHLEYPIT | |
| Tafasitamab, Light | FGAGTKLEIKRTVAAPSVFIFPPSDEQLKSGTASVVCLLNNFYP | |
| chain | REAKVQWKVDNALQSGNSQESVTEQDSKDSTYSLSSTLTLSK | |
| OR19 11 | ADYEKHKVYACEVTHQGLSSPVTKSFNRGEC | |
| E200 + CD19 binder | GHNYTTRNILPGLNITSTFHKTDIVMTQSPATLSLSPGERATLS | 149 |
| (nfP2X₇ epitope | CRSSKSLQNVNGNTYLYWFQQKPGQSPQLLIYRMSNLNSGVP | |
| underlined), CD19, | DRFSGSGSGTEFTLTISSLEPEDFAVYYCMQHLEYPITFGAGT | |
| tafasitamab, Light | KLEIKRTVAAPSVFIFPPSDEQLKSGTASVVCLLNNFYPREAKV | |
| chain | QWKVDNALQSGNSQESVTEQDSKDSTYSLSSTLTLSKADYEK | |
| OR19_12 | HKVYACEVTHQGLSSPVTKSFNRGEC | |
| E200 + CD19 binder | GHNYTTRNILPGLNITSTFHKTGSDIVMTQSPATLSLSPGERAT | 150 |
| (nfP2X₇ epitope | LSCRSSKSLQNVNGNTYLYWFQQKPGQSPQLLIYRMSNLNSG | |
| underlined), CD19, | VPDRFSGSGSGTEFTLTISSLEPEDFAVYYCMQHLEYPITFGA | |
| Tafasitamab, Light | GTKLEIKRTVAAPSVFIFPPSDEQLKSGTASVVCLLNNFYPREA | |
| chain | KVQWKVDNALQSGNSQESVTEQDSKDSTYSLSSTLTLSKADY | |
| OR19_13 | EKHKVYACEVTHQGLSSPVTKSFNRGEC | |
| E200 + CD19 binder | GHNYTTRNILPGLNITSTFHGSDIVMTQSPATLSLSPGERATLS | 151 |
| (nfP2X₇ epitope | CRSSKSLQNVNGNTYLYWFQQKPGQSPQLLIYRMSNLNSGVP | |
| underlined), CD19, | DRFSGSGSGTEFTLTISSLEPEDFAVYYCMQHLEYPITFGAGT | |
| Tafasitamab, Light | KLEIKRTVAAPSVFIFPPSDEQLKSGTASVVCLLNNFYPREAKV | |
| chain OR19_14 | QWKVDNALQSGNSQESVTEQDSKDSTYSLSSTLTLSKADYEK | |
| | HKVYACEVTHQGLSSPVTKSFNRGEC | |
| E200 + CD19 binder | GHNYTTRNILPGLNITSGSDIVMTQSPATLSLSPGERATLSCRS | 152 |
| (nfP2X₇ epitope | SKSLQNVNGNTYLYWFQQKPGQSPQLLIYRMSNLNSGVPDRF | |
| underlined), CD19, | SGSGSGTEFTLTISSLEPEDFAVYYCMQHLEYPITFGAGTKLEI | |
| Tafasitamab, Light | KRTVAAPSVFIFPPSDEQLKSGTASVVCLLNNFYPREAKVQWK | |
| chain OR19_15 | VDNALQSGNSQESVTEQDSKDSTYSLSSTLTLSKADYEKHKV | |
| | YACEVTHQGLSSPVTKSFNRGEC | |
| E200 + CD19 binder | DFPGHNYTTRNILPGLNITSGSDIVMTQSPATLSLSPGERATLS | 153 |
| (nfP2X₇ epitope | CRSSKSLQNVNGNTYLYWFQQKPGQSPQLLIYRMSNLNSGVP | |
| underlined), CD19, | DRFSGSGSGTEFTLTISSLEPEDFAVYYCMQHLEYPITFGAGT | |
| Tafasitamab, Light | KLEIKRTVAAPSVFIFPPSDEQLKSGTASVVCLLNNFYPREAKV | |
| chain OR19_NEW_001 | QWKVDNALQSGNSQESVTEQDSKDSTYSLSSTLTLSKADYEK | |
| | HKVYACEVTHQGLSSPVTKSFNRGEC | |
| E200 + CD19 binder | DFPGHNYTTRNILPGLNITSGGGGSDIVMTQSPATLSLSPGER | 154 |
| (nfP2X₇ epitope | ATLSCRSSKSLQNVNGNTYLYWFQQKPGQSPQLLIYRMSNLN | |
| underlined), CD19, | SGVPDRFSGSGSGTEFTLTISSLEPEDFAVYYCMQHLEYPITF | |
| Tafasitamab, Light | GAGTKLEIKRTVAAPSVFIFPPSDEQLKSGTASVVCLLNNFYPR | |
| chain OR19_NEW_002 | EAKVQWKVDNALQSGNSQESVTEQDSKDSTYSLSSTLTLSKA | |
| | DYEKHKVYACEVTHQGLSSPVTKSFNRGEC | |
| E200 + CD19 binder | DFPGHNYTTRNILPGLNITSGGGGSGGGGSDIVMTQSPATLSL | 155 |
| (nfP2X₇ epitope | SPGERATLSCRSSKSLQNVNGNTYLYWFQQKPGQSPQLLIYR | |
| underlined), CD19, | MSNLNSGVPDRFSGSGSGTEFTLTISSLEPEDFAVYYCMQHLE | |
| Tafasitamab, Light | YPITFGAGTKLEIKRTVAAPSVFIFPPSDEQLKSGTASVVCLLNN | |
| chain OR19_NEW_003 | FYPREAKVQWKVDNALQSGNSQESVTEQDSKDSTYSLSSTLT | |
| | LSKADYEKHKVYACEVTHQGLSSPVTKSFNRGEC | |
| E200 + CD19 binder | DFPGHNYTTRNILPGLNITSGGGGSGGGGSGGGGSDIVMTQS | 156 |
| (nfP2X₇ epitope | PATLSLSPGERATLSCRSSKSLQNVNGNTYLYWFQQKPGQSP | |
| underlined), CD19, | QLLIYRMSNLNSGVPDRFSGSGSGTEFTLTISSLEPEDFAVYYC | |
| Tafasitamab, Light | MQHLEYPITFGAGTKLEIKRTVAAPSVFIFPPSDEQLKSGTASV | |
| chain OR19_NEW_004 | VCLLNNFYPREAKVQWKVDNALQSGNSQESVTEQDSKDSTY | |
| | SLSSTLTLSKADYEKHKVYACEVTHQGLSSPVTKSFNRGEC | |
| E200 + CD19 binder | DFPGHNYTTRNILPGLNITSTFHKTSGSGKDIVMTQSPATLSLS | 157 |
| (nfP2X₇ epitope | PGERATLSCRSSKSLQNVNGNTYLYWFQQKPGQSPQLLIYRM | |
| underlined), CD19, | SNLNSGVPDRFSGSGSGTEFTLTISSLEPEDFAVYYCMQHLEY | |
| Tafasitamab, Light | PITFGAGTKLEIKRTVAAPSVFIFPPSDEQLKSGTASVVCLLNNF | |
| chain OR19_NEW_005 | YPREAKVQWKVDNALQSGNSQESVTEQDSKDSTYSLSSTLTL | |
| | SKADYEKHKVYACEVTHQGLSSPVTKSFNRGEC | |
| E200 + CD19 binder | DFPGHNYTTRNILPGLNITSTFHKTSGSGKGSDIVMTQSPATLS | 158 |
| (nfP2X₇ epitope | LSPGERATLSCRSSKSLQNVNGNTYLYWFQQKPGQSPQLLIY | |
| underlined), CD19, | RMSNLNSGVPDRFSGSGSGTEFTLTISSLEPEDFAVYYCMQH | |
| Tafasitamab, Light | LEYPITFGAGTKLEIKRTVAAPSVFIFPPSDEQLKSGTASVVCLL | |
| chainOR19_NEW_006 | NNFYPREAKVQWKVDNALQSGNSQESVTEQDSKDSTYSLSS | |
| | TLTLSKADYEKHKVYACEVTHQGLSSPVTKSFNRGEC | |
| E200 + CD19 binder | DFPGHNYTTRNILPGLNITSTFHKTSGSGKGGGGSDIVMTQSP | 159 |
| (nfP2X₇ epitope | ATLSLSPGERATLSCRSSKSLQNVNGNTYLYWFQQKPGQSPQ | |
| underlined), CD19, | LLIYRMSNLNSGVPDRFSGSGSGTEFTLTISSLEPEDFAVYYC | |
| Tafasitamab, Light | MQHLEYPITFGAGTKLEIKRTVAAPSVFIFPPSDEQLKSGTASV | |
| chain OR19_NEW_007 | VCLLNNFYPREAKVQWKVDNALQSGNSQESVTEQDSKDSTY | |
| | SLSSTLTLSKADYEKHKVYACEVTHQGLSSPVTKSFNRGEC | |
| E200 + CD19 binder | DFPGHNYTTRNILPGLNITSTFHGGGGSDIVMTQSPATLSLSPG | 160 |
| (nfP2X₇ epitope | ERATLSCRSSKSLQNVNGNTYLYWFQQKPGQSPQLLIYRMSN | |
| underlined), CD19, | LNSGVPDRFSGSGSGTEFTLTISSLEPEDFAVYYCMQHLEYPIT | |
| Tafasitamab, Light | FGAGTKLEIKRTVAAPSVFIFPPSDEQLKSGTASVVCLLNNFYP | |
| chain OR19_NEW_008 | REAKVQWKVDNALQSGNSQESVTEQDSKDSTYSLSSTLTLSK | |
| | ADYEKHKVYACEVTHQGLSSPVTKSFNRGEC | |
| E200 + CD19 binder | GHNYTTRNILPGLNITSTFHGGGGSDIVMTQSPATLSLSPGER | 161 |
| (nfP2X₇ epitope | ATLSCRSSKSLQNVNGNTYLYWFQQKPGQSPQLLIYRMSNLN | |
| underlined), CD19, | SGVPDRFSGSGSGTEFTLTISSLEPEDFAVYYCMQHLEYPITF | |
| Tafasitamab, Light | GAGTKLEIKRTVAAPSVFIFPPSDEQLKSGTASVVCLLNNFYPR | |
| chain OR19_NEW_009 | EAKVQWKVDNALQSGNSQESVTEQDSKDSTYSLSSTLTLSKA | |
| | DYEKHKVYACEVTHQGLSSPVTKSFNRGEC | |
| E200 + CD19 binder | DFPGHNYTTRNILPGLNITSTFHKTGGGGSDIVMTQSPATLSLS | 162 |
| (nfP2X₇ epitope | PGERATLSCRSSKSLQNVNGNTYLYWFQQKPGQSPQLLIYRM | |
| underlined), CD19, | SNLNSGVPDRFSGSGSGTEFTLTISSLEPEDFAVYYCMQHLEY | |
| Tafasitamab, Light | PITFGAGTKLEIKRTVAAPSVFIFPPSDEQLKSGTASVVCLLNNF | |
| chain OR19_NEW_010 | YPREAKVQWKVDNALQSGNSQESVTEQDSKDSTYSLSSTLTL | |
| | SKADYEKHKVYACEVTHQGLSSPVTKSFNRGEC | |
| E200 + CD19 binder | GHNYTTRNILPGLNITSTFHKTGGGGSDIVMTQSPATLSLSPGE | 163 |
| (nfP2X₇ epitope | RATLSCRSSKSLQNVNGNTYLYWFQQKPGQSPQLLIYRMSNL | |
| underlined), CD19, | NSGVPDRFSGSGSGTEFTLTISSLEPEDFAVYYCMQHLEYPIT | |
| Tafasitamab, Light | FGAGTKLEIKRTVAAPSVFIFPPSDEQLKSGTASVVCLLNNFYP | |
| chain OR19_NEW_011 | REAKVQWKVDNALQSGNSQESVTEQDSKDSTYSLSSTLTLSK | |
| | ADYEKHKVYACEVTHQGLSSPVTKSFNRGEC | |
| E200 + CD19 binder | GHNYTTRNILPGLNITSGGGGSGGGGSDIVMTQSPATLSLSPG | 164 |
| (nfP2X₇ epitope | ERATLSCRSSKSLQNVNGNTYLYWFQQKPGQSPQLLIYRMSN | |
| underlined), CD19, | LNSGVPDRFSGSGSGTEFTLTISSLEPEDFAVYYCMQHLEYPIT | |
| Tafasitamab, Light | FGAGTKLEIKRTVAAPSVFIFPPSDEQLKSGTASVVCLLNNFYP | |
| chain OR19_NEW_012 | REAKVQWKVDNALQSGNSQESVTEQDSKDSTYSLSSTLTLSK | |
| | ADYEKHKVYACEVTHQGLSSPVTKSFNRGEC | |

Exemplary CAR constructs

| | | |
|---|---|---|
| CAR7 | MALPVTALLLPLALLLHAARPEVQLLESGGGLVQPGGSLRLSC | 165 |
| | AASGFTFRNHDMGWVRQAPGKGLEWVSAISGSGGSTYYANS | |
| | VKGRFTISRDNSKNTLYLQMNSLRAEDTAVYYCAEPKPMDTEF | |
| | DYRSPGTLVTVSSRAAAIEVMYPPPYLDNEKSNGTIIHVKGKHL | |
| | CPSPLFPGPSKPFWVLVVVGGVLACYSLLVTVAFIIFWVRSKR | |
| | SRLLHSDYMNMTPRRPGPTRKHYQPYAPPRDFAAYRSKRGR | |
| | KKLLYIFKQPFMRPVQTTQEEDGCSCRFPEEEEGGCELRVKF | |
| | SRSADAPAYKQGQNQLYNELNLGRREEYDVLDKRRGRDPEM | |
| | GGKPRRKNPQEGLYNELQKDKMAEAYSEIGMKGERRRGKGH | |
| | DGLYQGLSTATKDTYDALHMQALPPRLEGGGEGRGSLLTCGD | |
| | VEENPGPRMLLLVTSLLLCELPHPAFLLIPRKVONGIGIGEFKDS | |
| | LSINATNIKHFKNCTSISGDLHILPVAFRGDSFTHTPPLDPQELDI | |
| | LKTVKEITGFLLIQAWPENRTDLHAFENLEIIRGRTKQHGQFSLA | |
| | VVSLNITSLGLRSLKEISDGDVIISGNKNLCYANTINWKKLFGTS | |
| | GQKTKIISNRGENSCKATGQVCHALCSPEGCWGPEPRDCVSC | |
| | RNVSRGRECVDKCNLLEGEPREFVENSECIQCHPECLPQAMN | |
| | ITCTGRGPDNCIQCAHYIDGPHCVKTCPAGVMGENNTLVWKY | |
| | ADAGHVCHLCHPNCTYGCTGPGLEGCPTNGPKIPSIATGMVG | |
| | ALLLLLVVALGIGLFM | |
| CAR10 | MALPVTALLLPLALLLHAARPEVQLLESGGGLVQPGGSLRLSC | 166 |
| | AASGFTFRNHDMGWVRQAPGKGLEWVSAISGSGGSTYYANS | |
| | VKGRFTISRDNSKNTLYLQMNSLRAEDTAVYYCAEPKPMDTEF | |
| | DYRSPGTLVTVSSTTTPAPRPPTPAPTIASQPLSLRPEACRPAA | |
| | GGAVHTRGLDFACDFWVLVVVGGVLACYSLLVTVAFIIFWVRS | |
| | KRSRLLHSDYMNMTPRRPGPTRKHYQPYAPPRDFAAYRSKR | |
| | GRKKLLYIFKQPFMRPVQTTQEEDGCSCRFPEEEEGGCELRV | |
| | KFSRSADAPAYKQGQNQLYNELNLGRREEYDVLDKRRGRDP | |
| | EMGGKPRRKNPQEGLYNELQKDKMAEAYSEIGMKGERRRGK | |
| | GHDGLYQGLSTATKDTYDALHMQALPPRLEGGGEGRGSLLTC | |
| | GDVEENPGPRMLLLVTSLLLCELPHPAFLLIPRKVONGIGIGEF | |
| | KDSLSINATNIKHFKNCTSISGDLHILPVAFRGDSFTHTPPLDPQ | |
| | ELDILKTVKEITGFLLIQAWPENRTDLHAFENLEIIRGRTKQHGQ | |
| | FSLAVVSLNITSLGLRSLKEISDGDVIISGNKNLCYANTINWKKL | |
| | FGTSGQKTKIISNRGENSCKATGQVCHALCSPEGCWGPEPRD | |
| | CVSCRNVSRGRECVDKCNLLEGEPREFVENSECIQCHPECLP | |
| | QAMNITCTGRGPDNCIQCAHYIDGPHCVKTCPAGVMGENNTL | |
| | VWKYADAGHVCHLCHPNCTYGCTGPGLEGCPTNGPKIPSIAT | |
| | GMVGALLLLLVVALGIGLFM | |
| CAR16 | MALPVTALLLPLALLLHAARPEVQLLESGGGLVQPGGSLRLSC | 167 |
| | AASGFTFRNHDMGWVRQAPGKGLEWVSAISGSGGSTYYANS | |
| | VKGRFTISRDNSKNTLYLQMNSLRAEDTAVYYCAEPKPMDTEF | |
| | DYRSPGTLVTVSSESKYGPPCPPCPFWVLVVVGGVLACYSLL | |
| | VTVAFIIFWVRSKRSRLLHSDYMNMTPRRPGPTRKHYQPYAPP | |
| | RDFAAYRSKRGRKKLLYIFKQPFMRPVQTTQEEDGCSCRFPE | |
| | EEEGGCELRVKFSRSADAPAYKQGQNQLYNELNLGRREEYD | |
| | VLDKRRGRDPEMGGKPRRKNPQEGLYNELQKDKMAEAYSEI | |
| | GMKGERRRGKGHDGLYQGLSTATKDTYDALHMQALPPRLEG | |
| | GGEGRGSLLTCGDVEENPGPRMLLLVTSLLLCELPHPAFLLIP | |
| | RKVCNGIGIGEFKDSLSINATNIKHFKNCTSISGDLHILPVAFRG | |
| | DSFTHTPPLDPQELDILKTVKEITGFLLIQAWPENRTDLHAFENL | |
| | EIIRGRTKQHGQFSLAVVSLNITSLGLRSLKEISDGDVIISGNKNL | |
| | CYANTINWKKLFGTSGQKTKIISNRGENSCKATGQVCHALCSP | |
| | EGCWGPEPRDCVSCRNVSRGRECVDKCNLLEGEPREFVENS | |
| | ECIQCHPECLPQAMNITCTGRGPDNCIQCAHYIDGPHCVKTCP | |
| | AGVMGENNTLVWKYADAGHVCHLCHPNCTYGCTGPGLEGCP | |
| | TNGPKIPSIATGMVGALLLLLVVALGIGLFM | |

Constructs for binding to CD117

| | | |
|---|---|---|
| E200 + CD117 binder | GHNYTTRNILPGLNITSDIQMTQSPSSLSASVGDRVTITCRASQ | 169 |
| (nfP2X₇ epitope | SINSYLNWYQQKPGKAPKLLIYAASSLQSGVPSRFSGSGSGTD | |
| underlined), | FTLTISSLOPEDFATYYCQQGVSDITFGGGTKVEIKRTVAAPSV | |
| WO2019084067, mAb- | FIFPPSDEQLKSGTASVVCLLNNFYPREAKVQWKVDNALQSG | |
| 55, B076-1_LC | NSQESVTEQDSKDSTYSLSSTLTLSKADYEKHKVYACEVTHQ | |
| | GLSSPVTKSFNRGECHHHHHH | |
| CD117, | QVQLVQSGAEVKKPGSSVKVSCKASGGTFRIYAISWVRQAPG | 170 |
| WO2019084067, mAb- | QGLEWMGGIIPDFGVANYAQKFQGRVTITADESTSTAYMELSS | |
| 55, B076-2_HC | LRSEDTAVYYCARGGLDTDEFDLWGRGTLVTVSSASTKGPSV | |
| | FPLAPSSKSTSGGTAALGCLVKDYFPEPVTVSWNSGALTSGV | |
| | HTFPAVLQSSGLYSLSSVVTVPSSSLGTQTYICNVNHKPSNTK | |
| | VDKKVEPKSCHHHHHH | |

Constructs for binding to CD133

| | | |
|---|---|---|
| E200 + CD133 binder | GHNYTTRNILPGLNITSDIQMTQSPSSLSASVGDRVTITCRASQ | 171 |
| (nfP2X₇ epitope | GSSYVAWYQQKPGKAPKLLIYSASYLYSGVPSRFSGSRSGTD | |
| underlined), | FTLTISSLOPEDFATYYCQQGVWSLITFGQGTKVEIKRTVAAPS | |
| CA2962157, RW03, | VFIFPPSDEQLKSGTASVVCLLNNFYPREAKVQWKVDNALQS | |
| B077-1_LC | GNSQESVTEQDSKDSTYSLSSTLTLSKADYEKHKVYACEVTH | |
| | QGLSSPVTKSFNRGECHHHHHH | |
| CD133, CA2962157, | EVQLVESGGGLVQPGGSLRLSCAASGFNLSSSSIHWVRQAPG | 172 |
| RW03, B077-2_HC | KGLEWVAYIYPYYSYTYYADSVKGRFTISADTSKNTAYLQMNS | |
| | LRAEDTAVYYCAREGSVAGEDYWGQGTLVTVSSASTKGPSVF | |
| | PLAPSSKSTSGGTAALGCLVKDYFPEPVTVSWNSGALTSGVH | |
| | TFPAVLQSSGLYSLSSVVTVPSSSLGTQTYICNVNHKPSNTKV | |
| | DKKVEPKSCHHHHHH | |

Constructs for binding to MUC1

| | | |
|---|---|---|
| E200 + MUC1 binder | GHNYTTRNILPGLNITSDIVMTQSPDSLAVSLGERATINCKSSQ | 173 |
| (nfP2X₇ epitope | SLLNSGDQKNYLTWYQQKPGQPPKLLIYWASTRESGVPDRFS | |
| underlined), | GSGSGTDFTLTISSLQAEDVAVYYCONDYSYPLTFGQGTKVEI | |
| WO2016130726A1, | KRTVAAPSVFIFPPSDEQLKSGTASVVCLLNNFYPREAKVQWK | |
| B078-1_LC | VDNALQSGNSQESVTEQDSKDSTYSLSSTLTLSKADYEKHKV | |
| | YACEVTHQGLSSPVTKSFNRGECHHHHHH | |
| MUC1, | QVQLVQSGAEVKKTGSSVKVSCKASGYTFTDHAIHWVRQAPG | 174 |
| WO2016130726A1, | QALEWMGHFSPGNTDIKYNDKFKGRVTLTVDRSMSTAYMELS | |
| B078-2_HC | SLRSEDTAMYYCKTSTFFFDYWGQGTMVTVSSASTKGPSVFP | |
| | LAPSSKSTSGGTAALGCLVKDYFPEPVTVSWNSGALTSGVHT | |
| | FPAVLQSSGLYSLSSVVTVPSSSLGTQTYICNVNHKPSNTKVD | |
| | KKVEPKSCHHHHHH | |

Constructs for binding to Mesothelin (MSLN)

| | | |
|---|---|---|
| E200 + MSLN binder | GHNYTTRNILPGLNITSDIQMTQSPSSLSASVGDRVTITCRASQ | 175 |
| (nfP2X₇ epitope | SISSYLNWYQQKPGKAPKLLIYAASSLQSGVPSGFSGSGSGTD | |
| underlined), | FTLTISSLOPEDFATYYCQQSYSTPLTFGGGTKVEIKRTVAAPS | |
| WO2009120769, | VFIFPPSDEQLKSGTASVVCLLNNFYPREAKVQWKVDNALQS | |
| B079-1_LC | GNSQESVTEQDSKDSTYSLSSTLTLSKADYEKHKVYACEVTH | |
| | QGLSSPVTKSFNRGECHHHHHH | |
| MSLN, | QVQLQESGPGLVKPSETLSLTCTVSGGSVSSGSYYWSWIRQP | 176 |
| WO2009120769, | PGKGLEWIGYIYYSGSTNYNPSLKSRVTISVDTSKNQFSLKLSS | |
| B079-2_HC | VTAADTAVYYCAREGKNGAFDIWGQGTMVTVSSASTKGPSVF | |
| | PLAPSSKSTSGGTAALGCLVKDYFPEPVTVSWNSGALTSGVH | |
| | TFPAVLQSSGLYSLSSVVTVPSSSLGTQTYICNVNHKPSNTKV | |
| | DKKVEPKSCHHHHHH | |

Constructs for binding to ROR2

| | | |
|---|---|---|
| E200 + ROR2 binder | GHNYTTRNILPGLNITSQSALTQPASVSGSPGQSITISCTGTSG | 177 |
| (nfP2X₇ epitope | DVGGYNYVSWYQHHPGKAPKLIIYDVNKRPSGFSDRFSGSKS | |
| underlined), | GNTASLTISGLQAEDEADYYCSSYTSTSTVFGGGTKLTVLGKR | |
| WO2016142768A1, | TVAAPSVFIFPPSDEQLKSGTASVVCLLNNFYPREAKVQWKVD | |
| B080-1_LC | NALQSGNSQESVTEQDSKDSTYSLSSTLTLSKADYEKHKVYA | |
| | CEVTHQGLSSPVTKSFNRGECHHHHHH | |
| ROR2, | QITLKESGPELVKPTQTLTLTCTFSGFSLSTSGMSVSWIRQPP | 178 |
| WO2016142768A1, | GKALEWLARIDWDDDKYYSTSLKTRLTISKDTSKNQVVLTMTN | |
| B080-2_HC | TDPVDTATYYCARGFYLAYGSYDSWGQGTLVTVSSASTKGPS | |
| | VFPLAPSSKSTSGGTAALGCLVKDYFPEPVTVSWNSGALTSG | |
| | VHTFPAVLQSSGLYSLSSVVTVPSSSLGTQTYICNVNHKPSNT | |
| | KVDKKVEPKSCHHHHHH | |

Constructs for binding to IL13Ra2

| | | |
|---|---|---|
| E200 + IL13Ra2 binder | GHNYTTRNILPGLNITSDIQMTQSPSSLSASVGDRVTITCTASL | 179 |
| (nfP2X₇ epitope | SVSSTYLHWYQQKPGSSPKLWIYSTSNLASGVPSRFSGSGSG | |
| underlined), | TSFTLTISSLOPEDFATYYCHQYHRSPLTFGGGTKVEIKRTVAA | |
| WO2014072888A1, | PSVFIFPPSDEQLKSGTASVVCLLNNFYPREAKVQWKVDNALQ | |
| B081-1_LC | SGNSQESVTEQDSKDSTYSLSSTLTLSKADYEKHKVYACEVTH | |
| | QGLSSPVTKSFNRGECHHHHHH | |
| IL13Ra2, | EVQLVESGGGLVQPGGSLRLSCAASGFSLTKYGVHWVRQAP | 180 |
| WO2014072888A1, | GKGLEWVGVKWAGGSTDYNSALMSRFTISKDNAKNSLYLQM | |
| B081-2_HC | NSLRAEDTAVYYCARDHRDAMDYWGQGTLVTVSSASTKGPS | |
| | VFPLAPSSKSTSGGTAALGCLVKDYFPEPVTVSWNSGALTSG | |
| | VHTFPAVLQSSGLYSLSSVVTVPSSSLGTQTYICNVNHKPSNT | |
| | KVDKKVEPKSCHHHHHH | |

Constructs for binding to IL13Ra2

| | | |
|---|---|---|
| IL13Ra2, ligand, | HHHHHHGPVPPSTALRYLIEELVNITQNQKAPLCNGSMVWSIN | 181 |
| US20180265844, B082 | LTAGMYCAALESLINVSGCSAIEKTQRMLSGFCPHKVSAGQFS | |
| | SLHVRDTKIEVAQFVKDLLLHLKKLFREGRFNGGGGS | |
| | GHNYTTRNILPGLNITS | |

Constructs for binding to EPHA2

| | | |
|---|---|---|
| E200 + EPHA2 binder | GHNYTTRNILPGLNITSDIQLTQSPSSLSASVGDRVTITCKASQ | 182 |
| (nfP2X₇ epitope | DINNYLSWYQQKPGQAPRLLIYRANRLVDGVPDRFSGSGYGT | |
| underlined), | DFTLTINNIESEDAAYYFCLKYDVFPYTFGQGTKVEIKRTVAAP | |
| WO2007073499, | SVFIFPPSDEQLKSGTASVVCLLNNFYPREAKVQWKVDNALQS | |
| B083-1_LC | GNSQESVTEQDSKDSTYSLSSTLTLSKADYEKHKVYACEVTH | |
| | QGLSSPVTKSFNRGECHHHHHH | |
| EPHA2, | QVQLLESGGGLVQPGGSLRLSCAASGFTFSSYTMSWVRQAP | 183 |
| WO2007073499, | GQALEWMGTISSGGTYTYYPDSVKGRFTISRDNAKNSLYLQM | |
| B083-2_HC | NSLRAEDTAVYYCAREAIFTYWGRGTLVTSSASTKGPSVFPLA | |
| | PSSKSTSGGTAALGCLVKDYFPEPVTVSWNSGALTSGVHTFP | |
| | AVLQSSGLYSLSSVVTVPSSSLGTQTYICNVNHKPSNTKVDKK | |
| | VEPKSCHHHHHH | |

Constructs for binding to EGFRvIII

| | | |
|---|---|---|
| E200 + EGFRvIII binder | GHNYTTRNILPGLNITSDIQMTQSPSSLSASVGDRVTITCRASQ | 184 |
| (nfP2X₇ epitope | GIRNNLAWYQQKPGKAPKRLIYAASNLQSGVPSRFTGSGSGT | |
| underlined), | EFTLIVSSLOPEDFATYYCLQHHSYPLTSGGGTKVEIKRTVAAP | |
| WO2013185010, | SVFIFPPSDEQLKSGTASVVCLLNNFYPREAKVQWKVDNALQS | |
| B084-1_LC | GNSQESVTEQDSKDSTYSLSSTLTLSKADYEKHKVYACEVTH | |
| | QGLSSPVTKSFNRGECHHHHHH | |
| EGFRvIII, | EVQVLESGGGLVQPGGSLRLSCAASGFTFSSYAMSWVRQAP | 185 |
| WO2013185010, | GKGLEWVSAISGSGGSTNYADSVKGRFTISRDNSKNTLYLQM | |
| B084-2_HC | NSLRAEDTAVYYCAGSSGWSEYWGQGTLVTVSSASTKGPSV | |
| | FPLAPSSKSTSGGTAALGCLVKDYFPEPVTVSWNSGALTSGV | |
| | HTFPAVLQSSGLYSLSSVVTVPSSSLGTQTYICNVNHKPSNTK | |
| | VDKKVEPKSCHHHHHH | |

Constructs for binding to PSMA

| | | |
|---|---|---|
| E200 + PSMA binder | GHNYTTRNILPGLNITSDIVMTQSPSSLSASVGDRVTITCKASQ | 186 |
| (nfP2X₇ epitope | DVGTAVDWYQQKPGKAPKLLIYWASTRHTGVPDRFTGSGSG | |
| underlined), | TDFTLTISSLOPEDFADYFCQQYNSYPLTFGGGTKLEIKRTVAA | |
| US20190300622, | PSVFIFPPSDEQLKSGTASVVCLLNNFYPREAKVQWKVDNALQ | |
| B085-1_LC | SGNSQESVTEQDSKDSTYSLSSTLTLSKADYEKHKVYACEVTH | |
| | QGLSSPVTKSFNRGECHHHHHH | |
| PSMA, | EVQLVQSGAEVKKPGASVKISCKTSGYTFTEYTIHWVKQASGK | 187 |
| US20190300622, | GLEWIGNINPNNGGTTYNQKFEDRATLTVDKSTSTAYMELSSL | |
| B085-2_HC | RSEDTAVYYCAAGWNFDYWGQGTTVTVSSASTKGPSVFPLA | |
| | PSSKSTSGGTAALGCLVKDYFPEPVTVSWNSGALTSGVHTFP | |
| | AVLQSSGLYSLSSVVTVPSSSLGTQTYICNVNHKPSNTKVDKK | |
| | VEPKSCHHHHHH | |

Constructs for binding to CEA

| | | |
|---|---|---|
| E200 + CEA binder | GHNYTTRNILPGLNITSDIVLTQSPASLTVSLGLRATISCRASKS | 188 |
| (nfP2X₇ epitope | VSASGYSYMHWYQQRPGQPPKLLIYLASNLQSGVPARFSGSG | |
| underlined), | SGTDFTLNIHPVEEEDAATYYCQHSRELPTFGGGTKLEIKRTVA | |
| WO1999043817, | APSVFIFPPSDEQLKSGTASVVCLLNNFYPREAKVQWKVDNAL | |
| B086-1_LC | QSGNSQESVTEQDSKDSTYSLSSTLTLSKADYEKHKVYACEV | |
| | THQGLSSPVTKSFNRGECHHHHHH | |
| CEA, WO1999043817, | EVQLQQSGAELVRSGASVKMSCTASGFNIKDYYMHWVKQRP | 189 |
| B086-2_HC | EQGLEWIGWIDPENGDTEYAPKFQGKATMTTDYSSNTAYLQL | |
| | SSLTSEDTAVYYCNTRGLSTMITTRWFFDVWGAGTTVAVSSA | |
| | STKGPSVFPLAPSSKSTSGGTAALGCLVKDYFPEPVTVSWNS | |
| | GALTSGVHTFPAVLQSSGLYSLSSVVTVPSSSLGTQTYICNVN | |
| | HKPSNTKVDKKVEPKSCHHHHHH | |

Constructs for binding to PSCA

| | | |
|---|---|---|
| E200 + PSCA binder | GHNYTTRNILPGLNITSDIQLTQSPSSLSASVGDRVTITCSASSS | 190 |
| (nfP2X₇ epitope | VRFIHWYQQKPGKAPKRLIYDTSKLASGVPSRFSGSGSGTDFT | |
| underlined), | LTISSLQPEDFATYYCQQWSSSPFTFGQGTKVEIKRTVAAPSV | |
| US20120077962, | FIFPPSDEQLKSGTASVVCLLNNFYPREAKVQWKVDNALQSG | |
| B087-1_LC | NSQESVTEQDSKDSTYSLSSTLTLSKADYEKHKVYACEVTHQ | |
| | GLSSPVTKSFNRGECHHHHHH | |
| PSCA, | EVQLVESGGGLVQPGGSLRLSCAASGFNIKDYYIHWVRQAPG | 191 |
| US20120077962, | KGLEWVAWIDPENGDTEFADSVKGRFTISADTSKNTAYLQMN | |
| B087-2_HC | SLRAEDTAVYYCKTGGFWGQGTLVTVSSASTKGPSVFPLAPS | |
| | SKSTSGGTAALGCLVKDYFPEPVTVSWNSGALTSGVHTFPAV | |
| | LQSSGLYSLSSVVTVPSSSLGTQTYICNVNHKPSNTKVDKKVE | |
| | PKSCHHHHHH | |

Constructs for binding to Lewis Y

| | | |
|---|---|---|
| E200 + Lewis Y binder | GHNYTTRNILPGLNITSDIQMTQSPSSLSASVGDRVTITCRSSQ | 192 |
| (nfP2X₇ epitope | RIVHSNGNTYLEWYQQTPGKAPKLLIYKVSNRFSGVPSRFSGS | |
| underlined), | GSGTDFTFTISSLQPEDIATYYCFQGSHVPFTFGQGTKLQITKR | |
| EP0749482, B088- | TVAAPSVFIFPPSDEQLKSGTASVVCLLNNFYPREAKVQWKVD | |
| 1_LC | NALQSGNSQESVTEQDSKDSTYSLSSTLTLSKADYEKHKVYA | |
| | CEVTHQGLSSPVTKSFNRGECHHHHHH | |
| Lewis Y, EP0749482, | EVQLVESGGGVVQPGRSLRLSCSSSGFTFSDYYMYWVRQAP | 193 |
| B088-2_HC | GKGLEWVAYMSNVGAITDYPDTVKGRFTISRDNSKNTLFLQM | |
| | DSLRPEDTGVYFCARGTRDGSWFAYWGQGTPVTVSSASTKG | |
| | PSVFPLAPSSKSTSGGTAALGCLVKDYFPEPVTVSWNSGALTS | |
| | GVHTFPAVLQSSGLYSLSSVVTVPSSSLGTQTYICNVNHKPSN | |
| | TKVDKKVEPKSCHHHHHH | |

Constructs for binding to CD171 L1CAM

| | | |
|---|---|---|
| E200 + CD171_L1CAM | GHNYTTRNILPGLNITSDIQMTQSPSSLSASVGDRVTITCRASQ | 194 |
| binder (nfP2X₇ epitope | DISNYLNWYQQKPGKAPKLLIYYTSRLHSGVPSRFSGSGSGTD | |
| underlined), | YTLTISSLQPEDFATYFCQQGNTLPWTFGGGTKLEIKRTVAAP | |
| WO2008151819, | SVFIFPPSDEQLKSGTASVVCLLNNFYPREAKVQWKVDNALQS | |
| B089-1_LC | GNSQESVTEQDSKDSTYSLSSTLTLSKADYEKHKVYACEVTH | |
| | QGLSSPVTKSFNRGECHHHHHH | |
| CD171_L1CAM, | EVQLVQSGGGLVQSGGSLRLSCRASGYTFTRYWMLWVRQRP | 195 |
| WO2008151819, | GHGLEWVGEINPRNDRTNYNEKFKTRFTISVDRSKSTAYLQM | |
| B089-2_HC | DSLRAEDTAVYFCALGGGYAMDYWGQGTLVTVSSASTKGPS | |
| | VFPLAPSSKSTSGGTAALGCLVKDYFPEPVTVSWNSGALTSG | |
| | VHTFPAVLQSSGLYSLSSVVTVPSSSLGTQTYICNVNHKPSNT | |
| | KVDKKVEPKSCHHHHHH | |

Constructs for binding to EpCAM

| | | |
|---|---|---|
| E200 + EpCAM binder | GHNYTTRNILPGLNITSEIVMTQSPATLSVSPGERATLSCRASQ | 196 |
| (nfP2X₇ epitope | SVSSNLAWYQQKPGQAPRLIIYGASTTASGIPARFSASGSGTD | |
| underlined), | FTLTISSLQSEDFAVYYCQQYNNWPPAYTFGQGTKLEIKRTVA | |
| WO2010142990A1, | APSVFIFPPSDEQLKSGTASVVCLLNNFYPREAKVQWKVDNAL | |
| B090-1_LC | QSGNSQESVTEQDSKDSTYSLSSTLTLSKADYEKHKVYACEV | |
| | THQGLSSPVTKSFNRGECHHHHHH | |
| EpCAM, | QVQLVQSGAEVKKPGSSVKVSCKASGGTFSSYAISWVRQAPG | 197 |
| WO2010142990A1, | QGLEWMGGIIPIFGTANYAQKFQGRVTITADESTSTAYMELSSL | |
| B090-2_HC | RSEDTAVYYCARGLLWNYWGQGTLVTVSSASTKGPSVFPLAP | |
| | SSKSTSGGTAALGCLVKDYFPEPVTVSWNSGALTSGVHTFPA | |
| | VLQSSGLYSLSSVVTVPSSSLGTQTYICNVNHKPSNTKVDKKV | |
| | EPKSCHHHHHH | |

Constructs for binding to ALK

| | | |
|---|---|---|
| E200 + ALK binder | GHNYTTRNILPGLNITSEIVLTQSPATLSLSPGERATLSCRASES | 198 |
| (nfP2X₇ epitope | VDNYGISFAWYQQKPGQAPRLLIYRASRATGIPARFSGSGSGT | |
| underlined), | DFTLTISSLEPEDFAVYYCQQNNKDPPTFFGQGTKLEIKRTVAA | |
| WO2015069922A2, | PSVFIFPPSDEQLKSGTASVVCLLNNFYPREAKVQWKVDNALQ | |
| B091-1 LC | SGNSQESVTEQDSKDSTYSLSSTLTLSKADYEKHKVYACEVTH | |
| | QGLSSPVTKSFNRGECHHHHHH | |
| ALK, | QVQLQQSGAEVKKPGSSVKVSCKASGYAFSSYISWVRQAPG | 199 |
| WO2015069922A2, | QGLEWMGGQIYPGDGDTNYAQKFQGRVTITADESTSTAYMEL | |
| B091-2_HC | SSLRSEDTAVYYCVRYYYGSSGYFDYWWGQGTMVTVSSAST | |
| | KGPSVFPLAPSSKSTSGGTAALGCLVKDYFPEPVTVSWNSGA | |
| | LTSGVHTFPAVLQSSGLYSLSSVVTVPSSSLGTQTYICNVNHK | |
| | PSNTKVDKKVEPKSCHHHHHH | |

Constructs for binding to IGF-1R CD221

| | | |
|---|---|---|
| E200 + IGF-1R binder | GHNYTTRNILPGLNITSDVVMTQTPLSLPVSLGDPASISCRSSQ | 200 |
| (nfP2X₇ epitope | SIVHSNVNTYLEWYLQKPGQSPRLLIYKVSNRFSGVPDRFSGS | |
| underlined), | GAGTDFTLRISRVEAEDLGIYYCFQGSHVPPTFGGGTKLEIKRT | |
| US7985842B2, B092- | VAAPSVFIFPPSDEQLKSGTASVVCLLNNFYPREAKVQWKVDN | |
| 1 LC | ALQSGNSQESVTEQDSKDSTYSLSSTLTLSKADYEKHKVYACE | |
| | VTHQGLSSPVTKSFNRGECHHHHHH | |
| IGF-1R, | QVQLVQSGAEVVKPGASVKLSCKASGYTFTSYWMHWVKQRP | 201 |
| US7985842B2, B092- | GQGLEWIGEINPSNGRTNYNQKFQGKATLTVDKSSSTAYMQL | |
| 2 HC | SSLTSEDSAVYYFARGRPDYYGSSKWYFDVWGQGTTVTVSS | |
| | ASTKGPSVFPLAPSSKSTSGGTAALGCLVKDYFPEPVTVSWN | |
| | SGALTSGVHTFPAVLQSSGLYSLSSVVTVPSSSLGTQTYICNV | |
| | NHKPSNTKVDKKVEPKSCHHHHHH | |

Constructs for binding to Nectin 4

| | | |
|---|---|---|
| E200 + Nectin 4 binder | GHNYTTRNILPGLNITSSIVMTQTPKFLLVSAGDRVTITCKASQS | 202 |
| (nfP2X₇ epitope | VSNDVAWYQQKPGQSPKLLIYYASNRYTGVPDRFTGSGYGTD | |
| underlined), | FTFTISAVQAEDLAVYFCQQDYSSPYTFGGGTKLEIKRTVAAPS | |
| US20210130459, | VFIFPPSDEQLKSGTASVVCLLNNFYPREAKVQWKVDNALQS | |
| B093-1_LC | GNSQESVTEQDSKDSTYSLSSTLTLSKADYEKHKVYACEVTH | |
| | QGLSSPVTKSFNRGECHHHHHH | |
| Nectin 4, | QVQLQQSGPELVKPGASVRISCKASGYTFTTYYIHWVKQRPG | 203 |
| US20210130459, | QGLEWIGWIYPGNVNTKNNEKFKVKATLTADKSSSTAYMQLS | |
| B093-2_HC | SLTSEDSAVYFCARSNPYVMDYWGQGTSVTVSSASTKGPSVF | |
| | PLAPSSKSTSGGTAALGCLVKDYFPEPVTVSWNSGALTSGVH | |
| | TFPAVLQSSGLYSLSSVVTVPSSSLGTQTYICNVNHKPSNTKV | |
| | DKKVEPKSCHHHHHH | |

Constructs for binding to FAP

| | | |
|---|---|---|
| E200 + FAP binder | GHNYTTRNILPGLNITSEIVLTQSPGTLSLSPGERATLSCRASQ | 204 |
| (nfP2X₇ epitope | SVTSSYLAWYQQKPGQAPRLLINVGSRRATGIPDRFSGSGSG | |
| underlined), | TDFTLTISRLEPEDFAVYYCQQGIMLPPTFGQGTKVEIKRTVAA | |
| EP2603530, B094- | PSVFIFPPSDEQLKSGTASVVCLLNNFYPREAKVQWKVDNALQ | |
| 1_LC | SGNSQESVTEQDSKDSTYSLSSTLTLSKADYEKHKVYACEVTH | |
| | QGLSSPVTKSFNRGECHHHHHH | |
| FAP, EP2603530, | EVQLLESGGGLVQPGGSLRLSCAASGFTFSSYAMSWVRQAP | 205 |
| B094-2_HC | GKGLEWVSAIIGSGSITYYADSVKGRFTISRDNSKNTLYLQMNS | |
| | LRAEDTAVYYCAKGWFGGFNYWGQGTLVTVSSASTKGPSVF | |
| | PLAPSSKSTSGGTAALGCLVKDYFPEPVTVSWNSGALTSGVH | |
| | TFPAVLQSSGLYSLSSVVTVPSSSLGTQTYICNVNHKPSNTKV | |
| | DKKVEPKSCHHHHHH | |

Constructs for binding to AXL

| | | |
|---|---|---|
| E200 + AXL binder | GHNYTTRNILPGLNITSDVVMTQSPLSLPVTLGQPASISCRSSQ | 206 |
| (nfP2X₇ epitope | NIVHTNGNTYLEWYQQKPGKAPELLIYKVSNRFSGVPDRFSGS | |
| underlined), | GSGTDFTLKISRVEAEDVGVYYCFQGSHLLEPFTFGQGTKLEI | |
| EP2431393, B095- | KRTVAAPSVFIFPPSDEQLKSGTASVVCLLNNFYPREAKVQWK | |
| 1_LC | VDNALQSGNSQESVTEQDSKDSTYSLSSTLTLSKADYEKHKV | |
| | YACEVTHQGLSSPVTKSFNRGECHHHHHH | |
| AXL, EP2431393, | QVTLKESGPVLVKPTETLTLTCTVSGFSLSSFGVDWVRQAPGK | 207 |
| B095-2_HC | GLEWMGVIWGGGSTNYNSALKSRLTISKDNSKSQVVLTMTNM | |
| | DPVDTATYYCAGEGSKYGAWFAYWGQGTLVTVSSASTKGPS | |
| | VFPLAPSSKSTSGGTAALGCLVKDYFPEPVTVSWNSGALTSG | |
| | VHTFPAVLQSSGLYSLSSVVTVPSSSLGTQTYICNVNHKPSNT | |
| | KVDKKVEPKSCHHHHHH | |

Constructs for binding to CD138

| | | |
|---|---|---|
| E200 + CD138 binder | GHNYTTRNILPGLNITSDIQMTQSTSSLSASLGDRVTISCSASQ | 208 |
| (nfP2X₇ epitope | GINNYLNWYQQKPDGTVELLIYYTSTLQSGVPSRFSGSGSGTD | |
| underlined), | YSLTISNLEPEDIGTYYCQQYSKLPRTFGGGTKLEIKRTVAAPS | |
| US20090175863, | VFIFPPSDEQLKSGTASVVCLLNNFYPREAKVQWKVDNALQS | |
| B096-1_LC | GNSQESVTEQDSKDSTYSLSSTLTLSKADYEKHKVYACEVTH | |
| | QGLSSPVTKSFNRGECHHHHHH | |
| CD138, | QVQLQQSGSELMMPGASVKISCKATGYTFSNYWIEWVKQRP | 209 |
| US20090175863, | GHGLEWIGEILPGTGRTIYNEKFKGKATFTADISSNTVQMQLSS | |
| B096-2_HC | LTSEDSAVYYCARRDYYGNFYYAMDYWGQGTSVTVSSASTK | |
| | GPSVFPLAPSSKSTSGGTAALGCLVKDYFPEPVTVSWNSGAL | |
| | TSGVHTFPAVLQSSGLYSLSSVVTVPSSSLGTQTYICNVNHKP | |
| | SNTKVDKKVEPKSCHHHHHH | |

Constructs for binding to CLDN6

| | | |
|---|---|---|
| E200 + CLDN6 binder | GHNYTTRNILPGLNITSDIQMTQSPSSLSASVGDRVTITCRISEN | 210 |
| (nfP2X₇ epitope | IYSYLAWYQQKPGKAPKLLIYNAKILVEGVPSRFSGSGSGTDFT | |
| underlined), | LTISSLQPEDFATYYCQHHYTVPWTFGQGTKLEIKRTVAAPSV | |
| WO2019056023, | FIFPPSDEQLKSGTASVVCLLNNFYPREAKVQWKVDNALQSG | |
| B097-1_LC | NSQESVTEQDSKDSTYSLSSTLTLSKADYEKHKVYACEVTHQ | |
| | GLSSPVTKSFNRGECHHHHHH | |
| CLDN6, | EVQLLESGGGLVQPGGSMRLSCAASGFTFSNYWMNWVRQA | 211 |
| WO2019056023, | PGKGLEWVAQIRLKSDNYATHYADSVKGRFTISRDDSKNTVYL | |
| B097-2_HC | QMNSLRAEDTGVYYCNDGPPSGYWGQGTLLTVSSASTKGPS | |
| | VFPLAPSSKSTSGGTAALGCLVKDYFPEPVTVSWNSGALTSG | |
| | VHTFPAVLQSSGLYSLSSVVTVPSSSLGTQTYICNVNHKPSNT | |
| | KVDKKVEPKSCHHHHHH | |

Constructs for binding to Her4

| | | |
|---|---|---|
| E200 + Her4 binder | GHNYTTRNILPGLNITSQSVLTQPASVSGSPGQSITISCAGTSS | 212 |
| (nfP2X₇ epitope | DVGGSSYVSWYQQHPGKAPKLMIYYDSYRPSGVSNRFSGSK | |
| underlined), | SGNTASLTISGLQAEDEADYYCSSNTYYSTRVFGGGTKLAVLG | |
| WO2021116119, | KRTVAAPSVFIFPPSDEQLKSGTASVVCLLNNFYPREAKVQWK | |
| B098-1_LC | VDNALQSGNSQESVTEQDSKDSTYSLSSTLTLSKADYEKHKV | |
| | YACEVTHQGLSSPVTKSFNRGECHHHHHH | |
| Her4, WO2021116119, | EVQLVESGGSLVKPGGSLRLSCAASGFTFSNYYMNWVRQAP | 213 |
| B098-2_HC | GKGLEWISSIDGSSRYIDYADFVKGRFTISRDNATNSLYLQMNS | |
| | LRAEDTAVYYCVRSSSDYFGGGMDVWGRGTLVTVSSASTKG | |
| | PSVFPLAPSSKSTSGGTAALGCLVKDYFPEPVTVSWNSGALTS | |
| | GVHTFPAVLQSSGLYSLSSVVTVPSSSLGTQTYICNVNHKPSN | |
| | TKVDKKVEPKSCHHHHHH | |

Constructs for binding to Claudin 18.2

| | | |
|---|---|---|
| E200 + Claudin 18.2 | GHNYTTRNILPGLNITSDIVMTQSPSSLTVTAGEKVTMSCKSSQ | 214 |
| binder (nfP2X₇ epitope | SLLNSGNQKNYLTWYQQKPGQPPKLLIYWASTRESGVPDRFT | |
| underlined), | GSGSGTDFTLTISSVQAEDLAVYYCONDYSYPFTFGSGTKLEI | |
| SG10201609510U, | KRTVAAPSVFIFPPSDEQLKSGTASVVCLLNNFYPREAKVQWK | |
| B099-1_LC | VDNALQSGNSQESVTEQDSKDSTYSLSSTLTLSKADYEKHKV | |
| | YACEVTHQGLSSPVTKSFNRGECHHHHHH | |
| Claudin 18.2, | QVQLQQPGAELVRPGASVKLSCKASGYTFTSYWINWVKQRP | 215 |
| SG10201609510U, | GQGLEWIGNIYPSDSYTNYNQKFKDKATLTVDKSSSTAYMQLS | |
| B099-2_HC | SPTSEDSAVYYCTRSWRGNSFDYWGQGTTLTVSSASTKGPS | |
| | VFPLAPSSKSTSGGTAALGCLVKDYFPEPVTVSWNSGALTSG | |
| | VHTFPAVLQSSGLYSLSSVVTVPSSSLGTQTYICNVNHKPSNT | |
| | KVDKKVEPKSCHHHHHH | |

Constructs for binding to O-acetylated GD2

| | | |
|---|---|---|
| E200 + O-acetylated | GHNYTTRNILPGLNITSDVVMTQSPLSLPVTLGQPASISCRSSQ | 216 |
| GD2 binder (nfP2X₇ | SLLKNNGNTFLHWYQQRPGQSPRLLIYKVSNRLSGVPDRFSG | |
| epitope underlined), | SGSGTDFTLKISRVEAEDVGVYFCSQSTHIPYTFGGGTKVEIKR | |
| EP3269739A1, B100- | TVAAPSVFIFPPSDEQLKSGTASVVCLLNNFYPREAKVQWKVD | |
| 1_LC | NALQSGNSQESVTEQDSKDSTYSLSSTLTLSKADYEKHKVYA | |
| | CEVTHQGLSSPVTKSFNRGECHHHHHH | |
| O-acetylated GD2, | EVQLVESGGGLVQPGRSLRLSCTTSEFTFTDYYMTWVRQAPG | 217 |
| EP3269739A1, B100- | KGLEWLGFIRNRANGYTTEYNPSVKGRFTISRDNSKSILYLQM | |
| 2_HC | NSLKTEDTAVYYCARVSNWAFDYWGQGTLVTVSSASTKGPSV | |
| | FPLAPSSKSTSGGTAALGCLVKDYFPEPVTVSWNSGALTSGV | |
| | HTFPAVLQSSGLYSLSSVVTVPSSSLGTQTYICNVNHKPSNTK | |
| | VDKKVEPKSCHHHHHH | |

Constructs for binding to GD3

| | | |
|---|---|---|
| E200 + GD3 binder | GHNYTTRNILPGLNITSDIQMTQSPSSLSASVGDRVTITCSASQ | 218 |
| (nfP2X₇ epitope | DISNYLNWYQQKPDKAVKLLIFYSSNLHSGVPSRFSGGGSGTD | |
| underlined), | YTLTISSLQPEDIATYFCHQYSKLPWTFGQGTKVEIKRTVAAPS | |
| US7253263, B101- | VFIFPPSDEQLKSGTASVVCLLNNFYPREAKVQWKVDNALQS | |
| 1_LC | GNSQESVTEQDSKDSTYSLSSTLTLSKADYEKHKVYACEVTH | |
| | QGLSSPVTKSFNRGECHHHHHH | |
| GD3, US7253263, | EVQLVESGGDFVQPGGSLRVSCAASGFAFSHYAMSWVRQAP | 219 |
| B101-2_HC | GKGLEWVAYISSGGSGTYYSDSVKGRFTISRDNSKNTLYLQM | |
| | RSLRAEDSAVYFCTRVKLGTYYFDSWGQGTLLTVSSASTKGP | |
| | SVFPLAPSSKSTSGGTAALGCLVKDYFPEPVTVSWNSGALTS | |
| | GVHTFPAVLQSSGLYSLSSVVTVPSSSLGTQTYICNVNHKPSN | |
| | TKVDKKVEPKSCHHHHHH | |

Constructs for binding to GM2

| | | |
|---|---|---|
| E200 + GM2 binder | GHNYTTRNILPGLNITSDIQLTQSPSSLSASPGDRVTITCSASSS | 220 |
| (nfP2X₇ epitope | VSYMHWFQQKPGKAPKLWIYSTSNLASGVPSRFSGSGSGTS | |
| underlined), | YSLTISRLQPEDIATYYCQQRSSYPYTFGGGTKVEIKRTVAAPS | |
| US5939532, B102- | VFIFPPSDEQLKSGTASVVCLLNNFYPREAKVQWKVDNALQS | |
| 1 LC | GNSQESVTEQDSKDSTYSLSSTLTLSKADYEKHKVYACEVTH | |
| | QGLSSPVTKSFNRGECHHHHHH | |
| GM2, US5939532, | EVQLVQSGAEVKKPGASVKVSCKASGYTFTDYNMDWVRQAP | 221 |
| B102-2_HC | GQGLEWMGYIYPNNGGTGYNQKFKSKVTITVDTSTSTAYMEL | |
| | HSLRSEDTAVYYCATYGHYYGYMFAYWGQGTLVTVSSASTK | |
| | GPSVFPLAPSSKSTSGGTAALGCLVKDYFPEPVTVSWNSGAL | |
| | TSGVHTFPAVLQSSGLYSLSSVVTVPSSSLGTQTYICNVNHKP | |
| | SNTKVDKKVEPKSCHHHHHH | |

Constructs for binding to TM4SF1

| | | |
|---|---|---|
| E200 + TM4SF1 binder | GHNYTTRNILPGLNITSAVVMTQTPLSLPVSLGDQASISCRSSQ | 222 |
| (nfP2X₇ epitope | SLVHSNGNTYLHWYMQKPGQSPKVLIYKVSNRFSGVPDRFSG | |
| underlined), | SGSGTDFTLKISRVEADDLGIYFCSQSTHIPLAFGAGTKLELKR | |
| WO2015054427A1, | TVAAPSVFIFPPSDEQLKSGTASVVCLLNNFYPREAKVQWKVD | |
| B103-1_LC | NALQSGNSQESVTEQDSKDSTYSLSSTLTLSKADYEKHKVYA | |
| | CEVTHQGLSSPVTKSFNRGECHHHHHH | |
| TM4SF1, | EVILVESGGGLVKPGGSLKLSCAASGFTFSSFAMSWVRQTPE | 223 |
| WO2015054427A1, | KRLEWVATISSGSIYIYYTDGVKGRFTISRDNAKNTVHLQMSSL | |
| B103-2_HC | RSEDTAMYYCARRGIYYGYDGYAMDYWGQGTSVTVSSASTK | |
| | GPSVFPLAPSSKSTSGGTAALGCLVKDYFPEPVTVSWNSGAL | |
| | TSGVHTFPAVLQSSGLYSLSSVVTVPSSSLGTQTYICNVNHKP | |
| | SNTKVDKKVEPKSCHHHHHH | |

Constructs for binding to CD147

| | | |
|---|---|---|
| E200 + CD147 binder | GHNYTTRNILPGLNITSDIQMTQSPSTLSASVGDRVTLSCKASE | 224 |
| (nfP2X₇ epitope | NVGTYVSWYQQKPGKAPKLLIYGASNRYTGVPSRFTGSGSGT | |
| underlined), | DFTLTISSLQPEDFATYYCGQSYSYPFTFGSGTKLEIKRTVAAP | |
| WO2017186182, | SVFIFPPSDEQLKSGTASVVCLLNNFYPREAKVQWKVDNALQS | |
| B104-1_LC | GNSQESVTEQDSKDSTYSLSSTLTLSKADYEKHKVYACEVTH | |
| | QGLSSPVTKSFNRGECHHHHHH | |
| CD147, | EVQLVESGGGLVQPGGSLRLSCAASGFTFSNFWMNWVRQAP | 225 |
| WO2017186182, | GKGLEWVSEIRLKSNNYATHYAESVKGRFTISRDDSKNTLYLQ | |
| B104-2_HC | MNSLKTEDTAVYYCTSYDYEYWGQGTLVTVSAASTKGPSVFP | |
| | LAPSSKSTSGGTAALGCLVKDYFPEPVTVSWNSGALTSGVHT | |
| | FPAVLQSSGLYSLSSVVTVPSSSLGTQTYICNVNHKPSNTKVD | |
| | KKVEPKSCHHHHHH | |

Constructs for binding to CEACAM5

| | | |
|---|---|---|
| E200 + CEACAM5 | GHNYTTRNILPGLNITSDIQLTQSPSSLSASVGDRVTITCKASQ | 226 |
| binder (nfP2X₇ epitope | DVGTSVAWYQQKPGKAPKLLIYWTSTRHTGVPSRFSGSGSGT | |
| underlined), | DFTFTISSLQPEDIATYYCQQYSLYRSFGQGTKVEIKRTVAAPS | |
| WO2015069430, | VFIFPPSDEQLKSGTASVVCLLNNFYPREAKVQWKVDNALQS | |
| B105-1 LC | GNSQESVTEQDSKDSTYSLSSTLTLSKADYEKHKVYACEVTH | |
| | QGLSSPVTKSFNRGECHHHHHH | |
| CEACAM5, | EVQLVESGGGVVQPGRSLRLSCSASGFDFTTYWMSWVRQAP | 227 |
| WO2015069430, | GKGLEWIGEIHPDSSTINYAPSLKDRFTISRDNAKNTLFLQMDS | |
| B105-2_HC | LRPEDTGVYFCASLYFGFPWFAYWGQGTPVTVSSASTKGPSV | |
| | FPLAPSSKSTSGGTAALGCLVKDYFPEPVTVSWNSGALTSGV | |
| | HTFPAVLQSSGLYSLSSVVTVPSSSLGTQTYICNVNHKPSNTK | |
| | VDKKVEPKSCHHHHHH | |

Constructs for binding to VEGFR-1

| | | |
|---|---|---|
| E200 + VEGFR-1 | GHNYTTRNILPGLNITSDIVMTQSPDSLAVSLGERATINCSASS | 228 |
| binder (nfP2X₇ epitope | SVSYMHWYQQKPGQPPKLLIYRTSNLASGVPDRFSGSGSGTD | |
| underlined), | FTLTISSLQAEDVAVYYCHQWSMYTFGQGTKVEIKRTVAAPSV | |
| US7615214, B106- | FIFPPSDEQLKSGTASVVCLLNNFYPREAKVQWKVDNALQSG | |
| 1_LC | NSQESVTEQDSKDSTYSLSSTLTLSKADYEKHKVYACEVTHQ | |
| | GLSSPVTKSFNRGECHHHHHH | |
| VEGFR-1, US7615214, | QVQLVQSGAEVKKPGASVKVSCKASGYTFINYNMHWVRQAP | 229 |
| B106-2_HC | GQGLEWMGAIFPGNGFTSYNQKFKGRVTITVDKSTSTAYMEL | |
| | SSLRSEDTAVYYCARDGDYYFDYWGQGTLVTVSSASTKGPSV | |
| | FPLAPSSKSTSGGTAALGCLVKDYFPEPVTVSWNSGALTSGV | |
| | HTFPAVLQSSGLYSLSSVVTVPSSSLGTQTYICNVNHKPSNTK | |
| | VDKKVEPKSCHHHHHH | |

Constructs for binding to Podoplanin (PDPN)

| | | |
|---|---|---|
| E200 + PDPN binder | GHNYTTRNILPGLNITSDVLMTQTPLSLPVSLGDQASISCRSSR | 230 |
| (nfP2X₇ epitope | NIVQSTGNTYLEWYLQKPGQSPKLLIFKVSNRFSGVPDRFSGS | |
| underlined), | GSGTDFTLKISRVEAEDLGVYYCFQGSHVPPWTFGGGTKLEIK | |
| US20160347834A1, | RTVAAPSVFIFPPSDEQLKSGTASVVCLLNNFYPREAKVQWKV | |
| B107-1 LC | DNALQSGNSQESVTEQDSKDSTYSLSSTLTLSKADYEKHKVY | |
| | ACEVTHQGLSSPVTKSFNRGECHHHHHH | |
| PDPN, | DVQLVESGGGLVQPGGSRKLSCAASGFTFSGFGMHWVRQAP | 231 |
| US20160347834A1, | EKGLEWVAYISSVSSRIYYADTVKGRFTISRDNPKNTLFLQMTS | |
| B107-2_HC | LRSEDTAMYYCAREQTGPAWFAYWGQGTLVTVSAASTKGPS | |
| | VFPLAPSSKSTSGGTAALGCLVKDYFPEPVTVSWNSGALTSG | |
| | VHTFPAVLQSSGLYSLSSVVTVPSSSLGTQTYICNVNHKPSNT | |
| | KVDKKVEPKSCHHHHHH | |

Constructs for binding to WT1

| | | |
|---|---|---|
| E200 + WT1 binder | GHNYTTRNILPGLNITSQTWTQPPSASGTPGQRVTISCSGSSS | 232 |
| (nfP2X₇ epitope | NIGSNYVYWYQQLPGTAPKLVLLIYRSNQRPSGVPDRFSGSKS | |
| underlined), | GTSASLAISGPRSVDEADYYCAAWDDSLNGVVFGGGTKLTVL | |
| EP2694553A2, B108- | GKRTVAAPSVFIFPPSDEQLKSGTASVVCLLNNFYPREAKVQW | |
| 1 LC | KVDNALQSGNSQESVTEQDSKDSTYSLSSTLTLSKADYEKHK | |
| | VYACEVTHQGLSSPVTKSFNRGECHHHHHH | |
| WT1, EP2694553A2, | QVQLVQSGAEVKKPGSSVKVSCKASGGTFSSYAISWVRQAPG | 233 |
| B108-2_HC | QGLEVHWMGGIIPIFGTANYAQKFQGRVTITADESTSTAYMEL | |
| | SSLRSEDTAVYYCARRIPPYYGMDVWGQGTTVTVSSASTKGP | |
| | SVFPLAPSSKSTSGGTAALGCLVKDYFPEPVTVSWNSGALTS | |
| | GVHTFPAVLQSSGLYSLSSVVTVPSSSLGTQTYICNVNHKPSN | |
| | TKVDKKVEPKSCHHHHHH | |

Constructs for binding to GPC2

| | | |
|---|---|---|
| E200 + GPC2 binder | GHNYTTRNILPGLNITSENVLTQSPAIMSASLGEKVTMSCRASS | 234 |
| (nfP2X₇ epitope | SVNYIYWYQQKSDASPKLWIYYTSNLAPGVPARFSGSGSGNS | |
| underlined), | YSLTISSMEGEDAATYYCQQFSSSPSTFGTGTKLELKRTVAAP | |
| WO2020033430, | SVFIFPPSDEQLKSGTASVVCLLNNFYPREAKVQWKVDNALQS | |
| B109-1_LC | GNSQESVTEQDSKDSTYSLSSTLTLSKADYEKHKVYACEVTH | |
| | QGLSSPVTKSFNRGECHHHHHH | |
| GPC2, | EVQLQQSGPELVKPGASVKMSCKASRFTFTDYNIHWVKQSPG | 235 |
| WO2020033430, | KTLEWIGYINPNNGDIFYKQKFNGKATLTINKSSNTAYMELRSL | |
| B109-2_HC | TSEDSAVYYCVRSSNIRYTFDRFFDVWGTGTTVTVSSASTKGP | |
| | SVFPLAPSSKSTSGGTAALGCLVKDYFPEPVTVSWNSGALTS | |
| | GVHTFPAVLQSSGLYSLSSVVTVPSSSLGTQTYICNVNHKPSN | |
| | TKVDKKVEPKSCHHHHHH | |

Constructs for binding to FGFR4

| | | |
|---|---|---|
| E200 + FGFR4 binder | GHNYTTRNILPGLNITSDIQMTQSPSSLSASVGDRVTITCRASE | 236 |
| (nfP2X₇ epitope | SVSTLMHWYQQKPGKAPKLLIYGTSNLESGVPSRFSGSGSGT | |
| underlined), | DFTLTISSLQPEDFATYYCQQSWNDPPTFGGGTKVEIKRTVAA | |
| WO2019034427A1, | PSVFIFPPSDEQLKSGTASVVCLLNNFYPREAKVQWKVDNALQ | |
| B110-1_LC | SGNSQESVTEQDSKDSTYSLSSTLTLSKADYEKHKVYACEVTH | |
| | QGLSSPVTKSFNRGECHHHHHH | |
| FGFR4, | EVQLVESGGGLVQPGGSLRLSCAASGFTFSNYYMSWVRQAP | 237 |
| WO2019034427A1, | GKGLEWVATINPSGTRTYYPDSVKGRFTISRDNAKNSLYLQMN | |
| B110-2_HC | SLRAEDTAVYYCARLYNNYAFDYWGQGTLVTVSSASTKGPSV | |
| | FPLAPSSKSTSGGTAALGCLVKDYFPEPVTVSWNSGALTSGV | |
| | HTFPAVLQSSGLYSLSSVVTVPSSSLGTQTYICNVNHKPSNTK | |
| | VDKKVEPKSCHHHHHH | |

Constructs for binding to EphB4

| | | |
|---|---|---|
| E200 + EphB4 binder | GHNYTTRNILPGLNITSDIQMTQSPSSLSASVGDRVTITCRASQ | 238 |
| (nfP2X₇ epitope | DVSTAVAWYQQKPGKAPKLLIYSASFLYSGVPSRFSGSGSGT | |
| underlined), | DFTLTISSLQPEDFATYYCQQTAQTPETFGQGTKVEIKRTVAAP | |
| EP1973950, B111- | SVFIFPPSDEQLKSGTASVVCLLNNFYPREAKVQWKVDNALQS | |
| 1_LC | GNSQESVTEQDSKDSTYSLSSTLTLSKADYEKHKVYACEVTH | |
| | QGLSSPVTKSFNRGECHHHHHH | |
| EphB4, EP1973950, | EVQLVESGGGLVQPGGSLRLSCAASGFTISGYYIHWVRQAPG | 239 |
| B111-2_HC | KGLEWVGGIYLYSGSTDYADSVKGYADSVKGRFTISADTSKNT | |
| | AYLQMNSLRAEDTAVYYCARGSGLRLGGLDYAMDYWGQGTL | |
| | VTASTKGPSVFPLAPSSKSTSGGTAALGCLVKDYFPEPVTVSW | |
| | NSGALTSGVHTFPAVLQSSGLYSLSSVVTVPSSSLGTQTYICN | |
| | VNHKPSNTKVDKKVEPKSCHHHHHH | |

Constructs for binding to STEAP-1

| | | |
|---|---|---|
| E200 + STEAP-1 binder | GHNYTTRNILPGLNITSDIQMTQSPSSLSASVGDRVTITCKSSQ | 240 |
| (nfP2X₇ epitope | SLLYRSNQKNYLAWYQQKPGKAPKLLIYWASTRESGVPSRFS | |
| underlined), | GSGSGTDFTLTISSLQPEDFATYYCQQYYNYPRTFGQGTKVEI | |
| WO2015112909, | KRTVAAPSVFIFPPSDEQLKSGTASVVCLLNNFYPREAKVQWK | |
| B112-1_LC | VDNALQSGNSQESVTEQDSKDSTYSLSSTLTLSKADYEKHKV | |
| | YACEVTHQGLSSPVTKSFNRGECHHHHHH | |
| STEAP-1, | EVQLVESGGGLVQPGGSLRLSCAVSGYSITSDYAWNWVRQA | 241 |
| WO2015112909, | PGKGLEWVGYISNSGSTSYNPSLKSRFTISRDTSKNTLYLQMN | |
| B112-2_HC | SLRAEDTAVYYCARERNYDYDDYYYAMDYWGQGTLVTVSSA | |
| | STKGPSVFPLAPSSKSTSGGTAALGCLVKDYFPEPVTVSWNS | |
| | GALTSGVHTFPAVLQSSGLYSLSSVVTVPSSSLGTQTYICNVN | |
| | HKPSNTKVDKKVEPKSCHHHHHH | |

Constructs for binding to STEAP-2

| | | |
|---|---|---|
| E200 + STEAP-2 binder | GHNYTTRNILPGLNITSDIQMTQSPSTLSASVGDRVTITCRASQ | 242 |
| (nfP2X₇ epitope | SISSWLAWYQQKPGRAPNLLISKASSLKSGVPSRFSGSGSGT | |
| underlined), | EFTLTVSSLQPDDFATYYCQQYYSYSYTFGQGTKLEIKRTVAA | |
| US20180104357, | PSVFIFPPSDEQLKSGTASVVCLLNNFYPREAKVQWKVDNALQ | |
| B113-1 LC | SGNSQESVTEQDSKDSTYSLSSTLTLSKADYEKHKVYACEVTH | |
| | QGLSSPVTKSFNRGECHHHHHH | |
| STEAP-2, | QVQLVESGGGVVQPGRSLRLSCVASGFTISSYGMNWVRQAP | 243 |
| US20180104357, | GKGLEWVAVISYDGGNKYSVDSVKGRFTISRDNSKNTLYLQM | |
| B113-2_HC | NSLRAEDSAVYYCARGRYFDLWGRGTLVTVSSASTKGPSVFP | |
| | LAPSSKSTSGGTAALGCLVKDYFPEPVTVSWNSGALTSGVHT | |
| | FPAVLQSSGLYSLSSVVTVPSSSLGTQTYICNVNHKPSNTKVD | |
| | KKVEPKSCHHHHHH | |

Constructs for binding to IL11Ra

| | | |
|---|---|---|
| E200 + IL11Ra binder | GHNYTTRNILPGLNITSDIVLTQSPATLSMTPGDSVSLSCRASQ | 244 |
| (nfP2X₇ epitope | SISNNLHWYQQKSHESPRLLIKYASQSISGIPSRFSGSGSGTDF | |
| underlined), | TLSFNSVETEDFGVYFCQQRYSWPLTFGAGTKLEMKRTVAAP | |
| WO2018109170, | SVFIFPPSDEQLKSGTASVVCLLNNFYPREAKVQWKVDNALQS | |
| B114-1_LC | GNSQESVTEQDSKDSTYSLSSTLTLSKADYEKHKVYACEVTH | |
| | QGLSSPVTKSFNRGECHHHHHH | |
| IL11Ra, | QVQLQQPGAELVRPGSSVKLSCKASGYTFTNYWMHWLKQRP | 245 |
| WO2018109170, | VQGLEWIGNIGPSDSKTHYNQKFKDKATLTVDKSSSTAYMQLN | |
| B114-2_HC | SLTSEDSAVYYCARGDYVLFTYWGQGTLVTVSAASTKGPSVF | |
| | PLAPSSKSTSGGTAALGCLVKDYFPEPVTVSWNSGALTSGVH | |
| | TFPAVLQSSGLYSLSSVVTVPSSSLGTQTYICNVNHKPSNTKV | |
| | DKKVEPKSCHHHHHH | |

Constructs for binding to CD163

| | | |
|---|---|---|
| E200 + CD163 binder | GHNYTTRNILPGLNITSDIVMTQSPSSLSASVGDRVTITCRASQ | 246 |
| (nfP2X₇ epitope | SVSSDVAWFQQKPGKSPKPLIYYASNRYSGVPSRFSGSGSGT | |
| underlined), | DFTLTISSLQAEDFAVYFCGQDYTSPRTFGGGTKLEIKRTVAAP | |
| WO2011039510A2, | SVFIFPPSDEQLKSGTASVVCLLNNFYPREAKVQWKVDNALQS | |
| B115-1_LC | GNSQESVTEQDSKDSTYSLSSTLTLSKADYEKHKVYACEVTH | |
| | QGLSSPVTKSFNRGECHHHHHH | |
| CD163, | QVQLQESGPGLVKPSETLSLTCTVSGYSITSDYAWNWIRQFP | 247 |
| WO2011039510A2, | GNKLEWMGYITYSGSTYYNPSLKSRVTISVDTSKNQFSLKLSS | |
| B115-2_HC | VTAADTATYYCVSGTYYFDYWGQGTTLTVSSASTKGPSVFPL | |
| | APSSKSTSGGTAALGCLVKDYFPEPVTVSWNSGALTSGVHTF | |
| | PAVLQSSGLYSLSSVVTVPSSSLGTQTYICNVNHKPSNTKVDK | |
| | KVEPKSCHHHHHH | |

Constructs for binding to Chlorotoxin

| | | |
|---|---|---|
| E200 + CLTX binder | GHNYTTRNILPGLNITSMCMPCFTTDHQMARKCDDCCGGKGR | 248 |
| (nfP2X₇ epitope | GKCYGPQCLCRKRTVAAPSVFIFPPSDEQLKSGTASVVCLLNN | |
| underlined), | FYPREAKVQWKVDNALQSGNSQESVTEQDSKDSTYSLSSTLT | |
| WO2017066481A1, | LSKADYEKHKVYACEVTHQGLSSPVTKSFNRGECHHHHHH | |
| B116-1_LC | | |
| CLTX, | MCMPCFTTDHQMARKCDDCCGGKGRGKCYGPQCLCRASTK | 249 |
| WO2017066481A1, | GPSVFPLAPSSKSTSGGTAALGCLVKDYFPEPVTVSWNSGAL | |
| B116-2_HC | TSGVHTFPAVLQSSGLYSLSSVVTVPSSSLGTQTYICNVNHKP | |
| | SNTKVDKKVEPKSCHHHHHH | |

Constructs for binding to CD206 Nanobody VH

| | | |
|---|---|---|
| E200 + CD206 binder | GHNYTTRNILPGLNITSQVQLQESGGGLVQPGGSLRLSCAASG | 250 |
| (nfP2X₇ epitope | FSLDYYAIGWFRQAPGKEREGISCISYKGGSTTYADSVKGRFTI | |
| underlined), | SKDNAKNTAYLQMNSLKPEDTGIYSCAAGFVCYNYDYWGQGT | |
| WO2014140376A1, | QVTVSSASTKGPSVFPLAPSSKSTSGGTAALGCLVKDYFPEPV | |
| B117-2_HC | TVSWNSGALTSGVHTFPAVLQSSGLYSLSSVVTVPSSSLGTQT | |
| | YICNVNHKPSNTKVDKKVEPKSCHHHHHH | |

Constructs for binding to IL1RAP

| | | |
|---|---|---|
| E200 + IL1RAP binder | GHNYTTRNILPGLNITSDVQMTQSPSSLSASVGDRVTITCQAS | 251 |
| (nfP2X₇ epitope | QSIYSFLSWYQQKPGQAPKLLIYAASDLESGVPSRFSGSGSGT | |
| underlined), | DFTLTISSLQPEDFATYYCQCNYIIDYGAFGQGTKVVIKRTVAA | |
| WO2017191325A9, | PSVFIFPPSDEQLKSGTASVVCLLNNFYPREAKVQWKVDNALQ | |
| B118-1_LC | SGNSQESVTEQDSKDSTYSLSSTLTLSKADYEKHKVYACEVTH | |
| | QGLSSPVTKSFNRGECHHHHHH | |
| IL1RAP, | EVQLEESGGRLVQPGTSLRLSCAVSGFSLSSYDMSWVRQAP | 252 |
| WO2017191325A9, | GKGLEWVSTIYIGGTTAYASWPKGRFTISKTNSKNTLYLQMNS | |
| B118-2_HC | LRAEDTAVYFCARLQGANYYNSLALWGQGTLVTVSSASTKGP | |
| | SVFPLAPSSKSTSGGTAALGCLVKDYFPEPVTVSWNSGALTS | |
| | GVHTFPAVLQSSGLYSLSSVVTVPSSSLGTQTYICNVNHKPSN | |
| | TKVDKKVEPKSCHHHHHH | |

Constructs for binding to MICA

| | | |
|---|---|---|
| E200 + MICA binder | GHNYTTRNILPGLNITSAIQLTQSPSSLSASVGDRVTITCRASQ | 253 |
| (nfP2X₇ epitope | GISSALAWYQQKPGKVPKSLIYDASSLESGVPSRFSGSGSGTD | |
| underlined), | FTLTISSLOPEDFATYYCQQFNSYPITFGQGTRLEIKRTVAAPS | |
| WO2019183551, | VFIFPPSDEQLKSGTASVVCLLNNFYPREAKVQWKVDNALQS | |
| B119-1_LC | GNSQESVTEQDSKDSTYSLSSTLTLSKADYEKHKVYACEVTH | |
| | QGLSSPVTKSFNRGECHHHHHH | |
| MICA, | QVQLVESGGGVVQPGRSLRLSCAASGFTFSNYAMHWVRQAP | 254 |
| WO2019183551, | GEGLEWVALIWYDGSNKFYGDSVKGRFTISRDNSKNTLYLQM | |
| B119-2_HC | NSLSAEDTAVYYCAREGSGHYWGQGTLVTVSSASTKGPSVFP | |
| | LAPSSKSTSGGTAALGCLVKDYFPEPVTVSWNSGALTSGVHT | |
| | FPAVLQSSGLYSLSSVVTVPSSSLGTQTYICNVNHKPSNTKVD | |
| | KKVEPKSCHHHHHH | |

Constructs for binding to MAGE-A1 scTCR

| | | |
|---|---|---|
| E200 + MAGE-A1 | GHNYTTRNILPGLNITSMKPTLISVLVIIFILRGTRAQRVTQPEKL | 255 |
| binder (nfP2X₇ epitope | LSVFKGAPVELKCNYSYSGSPELFWYVQYSRQRLQLLLRHISR | |
| underlined), | ESIKGFTADLNKGETSFHLKKPFAQEEDSAMYYCALRSGGYQ | |
| AU2018234830A1, | KVTFGTGTKLQVIPGGGGSGGGGSGGGGSMGIRLLCRVAFCF | |
| B120-1_LC | LAVGLVDVKVTQSSRYLVKRTGEKVFLECVQDMDHENMFWY | |
| | RQDPGLGLRLIYFSYDVKMKEKGDIPEGYSVSREKKERFSLILE | |
| | SASTNQTSMYLCASNNRDSYNSPLHFGNGTRLTVTHHHHHH | |

Constructs for binding to MAGE-A1 STCR

| | | |
|---|---|---|
| E200 + MAGE-A1 | GHNYTTRNILPGLNITSMKPTLISVLVIIFILRGTRAQRVTQPEKL | 256 |
| binder (nfP2X₇ epitope | LSVFKGAPVELKCNYSYSGSPELFWYVQYSRQRLQLLLRHISR | |
| underlined), | ESIKGFTADLNKGETSFHLKKPFAQEEDSAMYYCALRSGGYQ | |
| AU2018234830A1, | KVTFGTGTKLQVIPIQNPDPAVYQLRDSKSSDKSVCLFTDFDS | |
| B121-1_alpha chain | QTNVSQSKDSDVYITDKCVLDMRSMDFKSNSAVAWSNKSDFA | |
| | CANAFNNSIIPEDTFFPSPESS | |
| MAGE-A1, | MGIRLLCRVAFCFLAVGLVDVKVTQSSRYLVKRTGEKVFLECV | 257 |
| AU2018234830A1, | QDMDHENMFWYRQDPGLGLRLIYFSYDVKMKEKGDIPEGYSV | |
| B121-2_beta chain | SREKKERFSLILESASTNQTSMYLCASNNRDSYNSPLHFGNGT | |
| | RLTVTDLKNVFPPEVAVFEPSEAEISHTQKATLVCLATGFYPDH | |
| | VELSWWVNGKEVHSGVCTDPQPLKEQPALNDSRYALSSRLR | |
| | VSATFWQDPRNHFRCQVQFYGLSENDEWTQDRAKPVTQIVS | |
| | AEAWGRADHHHHHH | |

Constructs for binding to MAGE-A1 STCR/2

| | | |
|---|---|---|
| E200 + MAGE-A1 | MKPTLISVLVIIFILRGTRAQRVTQPEKLLSVFKGAPVELKCNYS | 258 |
| binder (nfP2X₇ epitope | YSGSPELFWYVQYSRQRLQLLLRHISRESIKGFTADLNKGETS | |
| underlined), | FHLKKPFAQEEDSAMYYCALRSGGYQKVTFGTGTKLQVIPIQN | |
| AU2018234830A1, | PDPAVYQLRDSKSSDKSVCLFTDFDSQTNVSQSKDSDVYITDK | |
| B122-1_alpha chain | CVLDMRSMDFKSNSAVAWSNKSDFACANAFNNSIIPEDTFFPS | |
| | PESSGGGGSGHNYTTRNILPGLNITS | |
| MAGE-A1, | MGIRLLCRVAFCFLAVGLVDVKVTQSSRYLVKRTGEKVFLECV | 259 |
| AU2018234830A1, | QDMDHENMFWYRQDPGLGLRLIYFSYDVKMKEKGDIPEGYSV | |
| B122-2_beta chain | SREKKERFSLILESASTNQTSMYLCASNNRDSYNSPLHFGNGT | |
| | RLTVTDLKNVFPPEVAVFEPSEAEISHTQKATLVCLATGFYPDH | |
| | VELSWWVNGKEVHSGVCTDPQPLKEQPALNDSRYALSSRLR | |
| | VSATFWQDPRNHFRCQVQFYGLSENDEWTQDRAKPVTQIVS | |
| | AEAWGRADHHHHHH | |

Constructs for binding TRBC1

| | | |
|---|---|---|
| E200 + TRBC1 binder | GHNYTTRNILPGLNITSDWMTQSPLSLPVSLGDQASISCRSSQ | 260 |
| (nfP2X₇ epitope | RLVHSNGNTYLHWYLQKPGQSPKLLIYRVSNRFPGVPDRFSG | |
| underlined), Jovi-1, | SGSGTDFTLKISRVEAEDLGIYFCSQSTHVPYTFGGGTKLEIKR | |
| WO2015132598A1, | TVAAPSVFIFPPSDEQLKSGTASVVCLLNNFYPREAKVQWKVD | |
| B122-1_LC | NALQSGNSQESVTEQDSKDSTYSLSSTLTLSKADYEKHKVYA | |
| | CEVTHQGLSSPVTKSFNRGECHHHHHH | |
| TRBC1, Jovi-1, | EVRLQQSGPDLIKPGASVKMSCKASGYTFTGYVMHVWKQRP | 261 |
| WO2015132598A1, | GQGLEWIGFINPYNDDIQSNERFRGKATLTSDKSSTTAYMELS | |
| B122-2_HC | SLTSEDSAVYYCARGAGYNFDGAYRFFDFWGQGTTLTVSSAS | |
| | TKGPSVFPLAPSSKSTSGGTAALGCLVKDYFPEPVTVSWNSG | |
| | ALTSGVHTFPAVLQSSGLYSLSSVVTVPSSSLGTQTYICNVNH | |
| | KPSNTKVDKKVEPKSCHHHHHH | |

Constructs for binding TRBC2

| | | |
|---|---|---|
| E200 + TRBC2 binder | GHNYTTRNILPGLNITSQPQSVSESPGKTVTISCTRSSGNFASK | 262 |
| (nfP2X₇ epitope | YVQWYQQRPGSSPTTVIYENYQRPSGVPDRFSGSIDSSSNSA | |
| underlined), F09, | TLTISGLKTEDEADYYCQSYDEVSWFGGGTQLTVLGQPAAKR | |
| WO2015132598A1, | TVAAPSVFIFPPSDEQLKSGTASVVCLLNNFYPREAKVQWKVD | |
| B123-1_LC | NALQSGNSQESVTEQDSKDSTYSLSSTLTLSKADYEKHKVYA | |
| | CEVTHQGLSSPVTKSFNRGECHHHHHH | |
| TRBC2, F09, | QMQLVQSGAEVKKPGASVKVSCKASGYTFASYYHWVRQAPG | 263 |
| WO2015132598A1, | QGLEWGIINPSGGSTSYAQKFQGRVTMTRDTSTSTVYMELSR | |
| B123-2_HC | LRSDDTAVYYCASNRGGSYKSVGMDVWGQGTTVTVSSASTK | |
| | GPSVFPLAPSSKSTSGGTAALGCLVKDYFPEPVTVSWNSGAL | |
| | TSGVHTFPAVLQSSGLYSLSSVVTVPSSSLGTQTYICNVNHKP | |
| | SNTKVDKKVEPKSCHHHHHH | |

Constructs for binding urokinase-type
plasminogen activator receptor (uPAR)

| | | |
|---|---|---|
| E200 + uPAR binder | GHNYTTRNILPGLNITSDIVLTQSPASLAVSLGQRATISCRASKS | 264 |
| (nfP2X₇ epitope | VSTSGYSYMHWYQQKPGQPPKLLIYLASNLESGVPARFSGSG | |
| underlined), 7G1, | SGTDFTLDIHPVEEEDAATYYCQHSRELPYTFGGGTKLELKRT | |
| WO2006094828, | VAAPSVFIFPPSDEQLKSGTASVVCLLNNFYPREAKVQWKVDN | |
| B124-1_LC | ALQSGNSQESVTEQDSKDSTYSLSSTLTLSKADYEKHKVYACE | |
| | VTHQGLSSPVTKSFNRGECHHHHHH | |
| uPAR, 7G1, | VQLQESGPELKKPGETVKISCKASGYTFTDYSMHWVKQAPGK | 265 |
| WO2006094828, | GLKCMGWINTETTKSTYADDFKGRFALSLETSASTVYLQISNLK | |
| B124-2_HC | NEDTATYFCAREASYGEFDYWGQGTTVTVSSASTKGPSVFPL | |
| | APSSKSTSGGTAALGCLVKDYFPEPVTVSWNSGALTSGVHTF | |
| | PAVLQSSGLYSLSSVVTVPSSSLGTQTYICNVNHKPSNTKVDK | |
| | KVEPKSCHHHHHH | |

EGFRvIII targeted CAR

| | | |
|---|---|---|
| EGFRvIII targeted CAR | EVQVLESGGGLVQPGGSLRLSCAASGFTFSSYAMSWVRQAP | 266 |
| scFv_(HL- | GKGLEWVSAISGSGGSTNYADSVKGRFTISRDNSKNTLYLQM | |
| configuration_clone_139)_ | NSLRAEDTAVYYCAGSSGWSEYWGQGTLVTVSSGGGGSGG | |
| CD8a_CD8TM_CD | GGSGGGGSDIQMTQSPSSLSASVGDRVTITCRASQGIRNNLA | |
| 28_CD137_CD3zeta | WYQQKPGKAPKRLIYAASNLQSGVPSRFTGSGSGTEFTLIVSS | |
| | LQPEDFATYYCLQHHSYPLTSGGGTKVEIKTTTPAPRPPTPAP | |
| | TIASQPLSLRPEACRPAAGGAVHTRGLDFACDIYIWAPLAGTC | |
| | GVLLLSLVITLYCRSKRSRLLHSDYMNMTPRRPGPTRKHYQPY | |
| | APPRDFAAYRSKRGRKKLLYIFKQPFMRPVQTTQEEDGCSCR | |
| | FPEEEEGGCELRVKFSRSADAPAYKQGQNQLYNELNLGRREE | |
| | YDVLDKRRGRDPEMGGKPRRKNPQEGLYNELQKDKMAEAYS | |
| | EIGMKGERRRGKGHDGLYQGLSTATKDTYDALHMQALPPR | |
| EGFRvIII peptide | LEEKKGNYVVTDH | 267 |

Constructs for binding CD33

| | | |
|---|---|---|
| EGFRvIII- | LEEKKGNYVVTDHDIQLTQSPSTLSASVGDRVTITCRASESLD | 268 |
| peptide + CD33 binder | NYGIRFLTWFQQKPGKAPKLLMYAASNQGSGVPSRFSGSGS | |
| (EGFRvIll epitope | GTEFTLTISSLQPDDFATYYCQQTKEVPWSFGQGTKVEVKRTV | |
| underlined) | AAPSVFIFPPSDEQLKSGTASVVCLLNNFYPREAKVQWKVDNA | |
| CD33, Gemtuzumab, | LQSGNSQESVTEQDSKDSTYSLSSTLTLSKADYEKHKVYACEV | |
| B030-3_LC | THQGLSSPVTKSFNRGECHHHHHH | |
| CD33, Gemtuzumab, | EVQLVQSGAEVKKPGSSVKVSCKASGYTITDSNIHWVRQAPG | 269 |
| B030-2_HC | QSLEWIGYIYPYNGGTDYNQKFKNRATLTVDNPTNTAYMELSS | |
| | LRSEDTAFYYCVNGNPWLAYWGQGTLVTVSSASTKGPSVFPL | |
| | APSSKSTSGGTAALGCLVKDYFPEPVTVSWNSGALTSGVHTF | |
| | PAVLQSSGLYSLSSVVTVPSSSLGTQTYICNVNHKPSNTKVDK | |
| | KVEPKSCHHHHHH | |

Constructs for binding Her2

| | | |
|---|---|---|
| EGFRvIII- | LEEKKGNYVVTDHDIQMTQSPSSLSASVGDRVTITCRASQDVN | 270 |
| peptide + CD33 binder | TAVAWYQQKPGKAPKLLIYSASFLYSGVPSRFSGSRSGTDFTL | |
| (EGFRvIII epitope | TISSLOPEDFATYYCQQHYTTPPTFGQGTKVEIKRTVAAPSVFI | |
| underlined) | FPPSDEQLKSGTASVVCLLNNFYPREAKVQWKVDNALQSGNS | |
| Her2, Gemtuzumab, | QESVTEQDSKDSTYSLSSTLTLSKADYEKHKVYACEVTHQGLS | |
| B033-3_LC | SPVTKSFNRGECHHHHHH | |
| Her2, Trastuzumab, | EVQLVESGGGLVQPGGSLRLSCAASGFNIKDTYIHWVRQAPG | 271 |
| B033-2_HC | KGLEWVARIYPTNGYTRYADSVKGRFTISADTSKNTAYLQMNS | |
| | LRAEDTAVYYCSRWGGDGFYAMDYWGQGTLVTVSSASTKGP | |
| | SVFPLAPSSKSTSGGTAALGCLVKDYFPEPVTVSWNSGALTS | |
| | GVHTFPAVLQSSGLYSLSSVVTVPSSSLGTQTYICNVNHKPSN | |
| | TKVDKKVEPKSCHHHHHH | |

CLDN6 targeted CAR

| | | |
|---|---|---|
| CLDN6 targeted CAR | EVQLLESGGGLVQPGGSMRLSCAASGFTFSNYWMNWVRQA | 272 |
| scFv_(HL- | PGKGLEWVAQIRLKSDNYATHYADSVKGRFTISRDDSKNTVYL | |
| configuration_clone_Ab | QMNSLRAEDTGVYYCNDGPPSGYWGQGTLLTVSSGGGGSG | |
| 3-4)_CD8a_CD8TM_CD | GGGSGGGGSDIQMTQSPSSLSASVGDRVTITCRISENIYSYLA | |
| 28_CD137_CD3zeta | WYQQKPGKAPKLLIYNAKILVEGVPSRFSGSGSGTDFTLTISSL | |
| | QPEDFATYYCQHHYTVPWTFGQGTKLEIKTTTPAPRPPTPAPT | |
| | IASQPLSLRPEACRPAAGGAVHTRGLDFACDIYIWAPLAGTCG | |
| | VLLLSLVITLYCRSKRSRLLHSDYMNMTPRRPGPTRKHYQPYA | |
| | PPRDFAAYRSKRGRKKLLYIFKQPFMRPVQTTQEEDGCSCRF | |
| | PEEEEGGCELRVKFSRSADAPAYKQGQNQLYNELNLGRREEY | |
| | DVLDKRRGRDPEMGGKPRRKNPQEGLYNELQKDKMAEAYSE | |
| | IGMKGERRRGKGHDGLYQGLSTATKDTYDALHMQALPPR | |
| CLDN6 peptide 23-mer | WTAHAIIRDFYNPLVAEAQKREL | 273 |
| CLDN6 peptide 13-mer | TAHAIIRDFYNPL | 274 |
| CLDN6 peptide 10-mer | LVAEAQKREL | 275 |

Constructs for binding CD33

| | | |
|---|---|---|
| CLDN6-peptide + CD33 | WTAHAIIRDFYNPLVAEAQKRELDIQLTQSPSTLSASVGDRVTI | 276 |
| binder (CLDN6 epitope | TCRASESLDNYGIRFLTWFQQKPGKAPKLLMYAASNQGSGVP | |
| underlined) | SRFSGSGSGTEFTLTISSLQPDDFATYYCQQTKEVPWSFGQG | |
| CD33, Gemtuzumab, | TKVEVKRTVAAPSVFIFPPSDEQLKSGTASVVCLLNNFYPREA | |
| B030-4_LC | KVQWKVDNALQSGNSQESVTEQDSKDSTYSLSSTLTLSKADY | |
| | EKHKVYACEVTHQGLSSPVTKSFNRGECHHHHHH | |
| CD33, Gemtuzumab, | EVQLVQSGAEVKKPGSSVKVSCKASGYTITDSNIHWVRQAPG | 277 |
| B030-2_HC | QSLEWIGYIYPYNGGTDYNQKFKNRATLTVDNPTNTAYMELSS | |
| | LRSEDTAFYYCVNGNPWLAYWGQGTLVTVSSASTKGPSVFPL | |
| | APSSKSTSGGTAALGCLVKDYFPEPVTVSWNSGALTSGVHTF | |
| | PAVLQSSGLYSLSSVVTVPSSSLGTQTYICNVNHKPSNTKVDK | |
| | KVEPKSCHHHHHH | |

Constructs for binding Her2

| | | |
|---|---|---|
| CLDN6-peptide + Her2 | WTAHAIIRDFYNPLVAEAQKRELDIQMTQSPSSLSASVGDRVTI | 278 |
| binder (CLDN6 epitope | TCRASQDVNTAVAWYQQKPGKAPKLLIYSASFLYSGVPSRFS | |
| underlined) | GSRSGTDFTLTISSLOPEDFATYYCQQHYTTPPTFGQGTKVEI | |
| Her2, Trastuzumab, | KRTVAAPSVFIFPPSDEQLKSGTASVVCLLNNFYPREAKVQWK | |
| B033-4 LC | VDNALQSGNSQESVTEQDSKDSTYSLSSTLTLSKADYEKHKV | |
| | YACEVTHQGLSSPVTKSFNRGECHHHHHH | |
| Her2, Trastuzumab, | EVQLVESGGGLVQPGGSLRLSCAASGFNIKDTYIHWVRQAPG | 279 |
| B033-2_HC | KGLEWVARIYPTNGYTRYADSVKGRFTISADTSKNTAYLQMNS | |
| | LRAEDTAVYYCSRWGGDGFYAMDYWGQGTLVTVSSASTKGP | |
| | SVFPLAPSSKSTSGGTAALGCLVKDYFPEPVTVSWNSGALTS | |
| | GVHTFPAVLQSSGLYSLSSVVTVPSSSLGTQTYICNVNHKPSN | |
| | TKVDKKVEPKSCHHHHHH | |

Constructs for binding B7-H7 (HHLA2)

| | | |
|---|---|---|
| E200 + B7H7 binder | GHNYTTRNILPGLNITSDIVMTQSPSSLAVSAGEKVTISCLSSQ | 280 |
| (nfP2X₇ epitope | SLFSSNTKRNYLNWYLQKPGQSPKLLIYHASTRLTGVPGRFIG | |
| underlined), 4.5, | SGSGTDFTLTVSTVQAEDLGDYFCQQHYETPLTFGDGTRLEIK | |
| WO2014100823A1, | RTVAAPSVFIFPPSDEQLKSGTASVVCLLNNFYPREAKVQWKV | |
| B125-1_LC | DNALQSGNSQESVTEQDSKDSTYSLSSTLTLSKADYEKHKVY | |
| | ACEVTHQGLSSPVTKSFNRGECHHHHHH | |
| B7H7, 4.5, | QIQLQESGPGLVKPSQSLSLTCSVTGFSITTGGYYWNWIRQFP | 281 |
| WO2014100823A1, | GKKLEWMGYIYTSGRTSYNPSLKSRISITRDTSKNQFFLQLNS | |
| B125-2_HC | MTTEDTATYYCADMADKGGWFAYWGQGTLVTVSSASTKGPS | |
| | VFPLAPSSKSTSGGTAALGCLVKDYFPEPVTVSWNSGALTSG | |
| | VHTFPAVLQSSGLYSLSSVVTVPSSSLGTQTYICNVNHKPSNT | |
| | KVDKKVEPKSCHHHHHH | |

Constructs for binding CD34

| | | |
|---|---|---|
| E200 + CD34 binder | GHNYTTRNILPGLNITSDVLLTQSPLSLPVTLGQPASISCRSSQ | 282 |
| (nfP2X₇ epitope | TIVHSNGNTYLEWFQQRPGQSPRLLIYQVSNRFSGVPDRFSG | |
| underlined), h4C8b, | SGSGTDFTLKISRVEAEDVGVYYCFQGSHVPRTFGGGTKVEIK | |
| US20100311955A1, | RTVAAPSVFIFPPSDEQLKSGTASVVCLLNNFYPREAKVQWKV | |
| B126-1_LC | DNALQSGNSQESVTEQDSKDSTYSLSSTLTLSKADYEKHKVY | |
| | ACEVTHQGLSSPVTKSFNRGECHHHHHH | |
| CD34, h4C8b, | QIQLVQSGSELKKPGASVKVSCKASGYTFTNYGMNWVRQAP | 283 |
| US20100311955A1, | GQGLKWMGWINTNTGEPKYAEEFKGRFALSLDTSVSTAYLQI | |
| B126-2_HC | NSLKAEDTAVYFCARGYGNYARGAWLAYWGQGTLVTVSSAS | |
| | TKGPSVFPLAPSSKSTSGGTAALGCLVKDYFPEPVTVSWNSG | |
| | ALTSGVHTFPAVLQSSGLYSLSSVVTVPSSSLGTQTYICNVNH | |
| | KPSNTKVDKKVEPKSCHHHHHH | |

Constructs for binding CD7

| | | |
|---|---|---|
| E200 + CD7 binder | GHNYTTRNILPGLNITSDIQMTQTTSSLSASLGDRVTISCSASQ | 284 |
| (nfP2X₇ epitope | GISNYLNWYQQKPDGTVKLLIYYTSSLHSGVPSRFSGSGSGTD | |
| underlined), seq 1, | YSLTISNLEPEDIATYYCQQYSKLPYTFGGGTKLEIKRTVAAPS | |
| WO2003051926, | VFIFPPSDEQLKSGTASVVCLLNNFYPREAKVQWKVDNALQS | |
| B127-1_LC | GNSQESVTEQDSKDSTYSLSSTLTLSKADYEKHKVYACEVTH | |
| | QGLSSPVTKSFNRGECHHHHHH | |
| CD7, seq 1, | EVQLVESGGGLVKPGGSLKLSCAASGLTFSSYAMSWVRQTPE | 285 |
| WO2003051926, | KRLEWVASISSGGFTYYPDSVKGRFTISRDNARNILYLQMSSL | |
| B127-2_HC | RSEDTAMYYCARDEVRGYLDVWGAGTTVTVSSASTKGPSVF | |
| | PLAPSSKSTSGGTAALGCLVKDYFPEPVTVSWNSGALTSGVH | |
| | TFPAVLQSSGLYSLSSVVTVPSSSLGTQTYICNVNHKPSNTKV | |
| | DKKVEPKSCHHHHHH | |

Constructs for binding CD7

| | | |
|---|---|---|
| E200 + CD7 binder | GHNYTTRNILPGLNITSMDVQLQESGGGSVQAGGSLRLSCPA | 286 |
| (nfP2X₇ epitope | SGYTFSHYCMGWNRQAPGKEREEVATIDTDDTPTYADSVMG | |
| underlined), seq 32, | RFTISRDNANNALYLQMNDLKPEDTSMYYCAIWMKLRGSCHD | |
| US20170226204, | RRLEVRGQGTQVTVSINASTKGPSVFPLAPSSKSTSGGTAALG | |
| B128-1_HC | CLVKDYFPEPVTVSWNSGALTSGVHTFPAVLQSSGLYSLSSVV | |
| | TVPSSSLGTQTYICNVNHKPSNTKVDKKVEPKSCHHHHHH | |

Constructs for binding GPRC5D

| | | |
|---|---|---|
| E200 + GPRC5D binder | GHNYTTRNILPGLNITSQSVLTQPPSVSAAPGQKVTIPCSGSRS | 287 |
| (nfP2X₇ epitope | NVGNYYVSWYQQLPGTAPKLLIYDNNKRPSGIPDRFSGSKSG | |
| underlined), seq 1, | TSATLGITGLQTGDEADYFCGTWDGSLSAHVFGTGTKVTVLG | |
| SG10202007326Q, | RTVAAPSVFIFPPSDEQLKSGTASVVCLLNNFYPREAKVQWKV | |
| B129-1_LC | DNALQSGNSQESVTEQDSKDSTYSLSSTLTLSKADYEKHKVY | |
| | ACEVTHQGLSSPVTKSFNRGECHHHHHH | |
| GPRC5D, seq 1, | QVQLVQSGSELKKPGASVRVSCTASGYTFTSYYMHWVRQAP | 288 |
| SG10202007326Q, | GQGLEWMGVINPNAGSTRYAQKFQGRVTMSTDTSTSTAYMD | |
| B129-2_HC | LSSLRSEDTAVYYCARGMYRSLLFYDPWGQGTLVTVSSASTK | |
| | GPSVFPLAPSSKSTSGGTAALGCLVKDYFPEPVTVSWNSGAL | |
| | TSGVHTFPAVLQSSGLYSLSSVVTVPSSSLGTQTYICNVNHKP | |
| | SNTKVDKKVEPKSCHHHHHH | |

Constructs for binding TIM-3

| | | |
|---|---|---|
| E200 + TIM-3 binder | GHNYTTRNILPGLNITSDIQMTQSPSSLSASVGDRVTITCLASQ | 289 |
| (nfP2X₇ epitope | PIGIWLAWYQQKPGKAPKLLIYAATSLADGVPSRFSGSGSGTD | |
| underlined), h1701- | FTFTISSLQPEDIATYYCQQLYSSPWTFGGGTKVEIKRTVAAPS | |
| 009NKG, EP3587452, | VFIFPPSDEQLKSGTASVVCLLNNFYPREAKVQWKVDNALQS | |
| B130-1_LC | GNSQESVTEQDSKDSTYSLSSTLTLSKADYEKHKVYACEVTH | |
| | QGLSSPVTKSFNRGECHHHHHH | |
| TIM-3, h1701-009NKG, | QVQLVQSGAEVKKPGASVKVSCKASGYTFTDYYMNWVRQAP | 290 |
| EP3587452, B130- | GQGLEWMGDIIPNKGGSKYNQKFKDRVTMTTDTSTSTAYMEL | |
| 2_HC | RSLRSDDTAVYYCATWGYGSSYRWFDYWGQGTLVTVSSAST | |
| | KGPSVFPLAPSSKSTSGGTAALGCLVKDYFPEPVTVSWNSGA | |
| | LTSGVHTFPAVLQSSGLYSLSSVVTVPSSSLGTQTYICNVNHK | |
| | PSNTKVDKKVEPKSCHHHHHH | |

Constructs for binding to CD191 (CCR1)

| | | |
|---|---|---|
| E200 + CD191 binder | GHNYTTRNILPGLNITSDIVMTQSPLSLPVTLGEPASISCRSSQS | 291 |
| (nfP2X₇ epitope | LVHRNGITFFHWYLQKPGQSPKLLIYKISNRFSGVPDRFSGSG | |
| underlined), hzmAb5- | SGTDFTLKISRVEAEDVGVYFCSQGTHVPPTFGQGTKLEIKRT | |
| 06_LV5HV14, | VAAPSVFIFPPSDEQLKSGTASVVCLLNNFYPREAKVQWKVDN | |
| EP3656791, B131- | ALQSGNSQESVTEQDSKDSTYSLSSTLTLSKADYEKHKVYACE | |
| 1_LC | VTHQGLSSPVTKSFNRGECHHHHHH | |
| CD191, hzmAb5- | QVQLQQSGPGLVKPSQTLSITCTVSGFSLNNYGVHWVRQPPG | 292 |
| 06_LV5HV14, | KGLEWLGVIWSAGTTVYNAAAISRLTISKDTSKNQVSFKMSSLT | |
| EP3656791, B131- | AADTAVYYCAKDGSRYYTAMDYWGQGTLVTVSSASTKGPSV | |
| 2_HC | FPLAPSSKSTSGGTAALGCLVKDYFPEPVTVSWNSGALTSGV | |
| | HTFPAVLQSSGLYSLSSVVTVPSSSLGTQTYICNVNHKPSNTK | |
| | VDKKVEPKSCHHHHHH | |

Constructs for binding to CD66b (CEACAM8)

| | | |
|---|---|---|
| E200 + CD66b binder | GHNYTTRNILPGLNITSDIQMTQSPSSLSASVGDRVTITCRASS | 293 |
| (nfP2X₇ epitope | SVSYMHWYQQKPGKAPKPLIYATSNLASGVPSRFSGSGSGTD | |
| underlined), BW250- | FTFTISSLQPEDIATYYCQQWSSNPLTFGQGTKVEIKRTVAAPS | |
| 183, EP0585570A1, | VFIFPPSDEQLKSGTASVVCLLNNFYPREAKVQWKVDNALQS | |
| B132-1_LC | GNSQESVTEQDSKDSTYSLSSTLTLSKADYEKHKVYACEVTH | |
| | QGLSSPVTKSFNRGECHHHHHH | |
| CD66b, BW250-183, | QVQLQESGPGLVRPSQTLSLTCTVSGFSDYYMNWVRQPPGR | 294 |
| EP0585570A1, B132- | GLEWIGFISNKPNGHTTEYSASVKGRVTMLRDTSKNQFSLRLS | |
| 2_HC | SVTAADTAVYYCARDKGIRWYFDVWGQGSLVTVSSASTKGPS | |
| | VFPLAPSSKSTSGGTAALGCLVKDYFPEPVTVSWNSGALTSG | |
| | VHTFPAVLQSSGLYSLSSVVTVPSSSLGTQTYICNVNHKPSNT | |
| | KVDKKVEPKSCHHHHHH | |

Constructs for binding to CD11b (MAC-1)

| | | |
|---|---|---|
| E200 + CD66b binder | GHNYTTRNILPGLNITSDIQMTQSPSSLSASLGERVSLTCRASQ | 295 |
| (nfP2X₇ epitope | EISGYLSWHQQKPDGTIKRLLYSTSTLDSGVPKRFSGSRSGSD | |
| underlined), seq_1, | YSLTISSLESEDFADYYCLQYAISPPTFGGGTKLEIKRTVAAPSV | |
| WO2017220369A1, | FIFPPSDEQLKSGTASVVCLLNNFYPREAKVQWKVDNALQSG | |
| B133-1_LC | NSQESVTEQDSKDSTYSLSSTLTLSKADYEKHKVYACEVTHQ | |
| | GLSSPVTKSFNRGECHHHHHH | |
| CD11b, seq_1, | QVTLKESGPGILQTSQTLSLTCSFSGFSLSTSGMGVSWIRQPS | 296 |
| WO2017220369A1, | GKGLEWLAHIYWDDDKRYNPSLKSRLTISKDTSRNQVFLKITS | |
| B133-2_HC | VDTTDTATYYCALNYYNSTYNFDFWGQGTTLTVSSASTKGPS | |
| | VFPLAPSSKSTSGGTAALGCLVKDYFPEPVTVSWNSGALTSG | |
| | VHTFPAVLQSSGLYSLSSVVTVPSSSLGTQTYICNVNHKPSNT | |
| | KVDKKVEPKSCHHHHHH | |

Constructs for binding to EMR2 (ADGRE2)

| | | |
|---|---|---|
| E200 + EMR2 binder | GHNYTTRNILPGLNITSDIQMTQSPSSLSASVGDRVTITCKASQ | 297 |
| (nfP2X₇ epitope | NVRTTVDWYQQKPGKAPKLLIYLASNRHTGVPSRFSGSGSGT | |
| underlined), | DFTLTISSLQPEDFATYYCLQHRNYPLTFGGGTKVEIKRTVAAP | |
| hSC93.253, | SVFIFPPSDEQLKSGTASVVCLLNNFYPREAKVQWKVDNALQS | |
| WO2017087800A1, | GNSQESVTEQDSKDSTYSLSSTLTLSKADYEKHKVYACEVTH | |
| B134-1_LC | QGLSSPVTKSFNRGECHHHHHH | |
| EMR2, hSC93.253, | EVQLVESGGGLVKPGGSLRLSCAASGFTFSSYDMSWVRQAP | 298 |
| WO2017087800A1, | GKGLEWVSTISSGGNYNYYPDSVKGRFTISRDNAKNSLYLQM | |
| B134-2_HC | NSLRAEDTAVYYCARHYDYPDYAMDYWGQGTTVTVSSASTK | |
| | GPSVFPLAPSSKSTSGGTAALGCLVKDYFPEPVTVSWNSGAL | |
| | TSGVHTFPAVLQSSGLYSLSSVVTVPSSSLGTQTYICNVNHKP | |
| | SNTKVDKKVEPKSCHHHHHH | |

Constructs for binding to MUC16

| | | |
|---|---|---|
| E200 + MUC16 binder | GHNYTTRNILPGLNITSDIVMTQAAPSVPVTPGESVSISCRSSK | 299 |
| (nfP2X₇ epitope | SLLHSNGNTYLYWFLQRPGQSPQRLIYYMSNLASGVPDRFSG | |
| underlined), 18C6, | RGSGTDFTLRISRVEAEDVGVYYCMQSLEYPLTFGGGTKLEIK | |
| WO2016149368A1, | RTVAAPSVFIFPPSDEQLKSGTASVVCLLNNFYPREAKVQWKV | |
| B135-1_LC | DNALQSGNSQESVTEQDSKDSTYSLSSTLTLSKADYEKHKVY | |
| | ACEVTHQGLSSPVTKSFNRGECHHHHHH | |
| MUC16, 18C6, | QVTLKESGPGILQPSQTLSLTCSFSGFSLSTVGMGVGWSRQP | 300 |
| WO2016149368A1, | SGKGLEWLAHIWWDDEDKYYNPALKSRLTISKDTSKNQVFLKI | |
| B135-2_HC | ANVDTADTATYYCTRIGTAQATDALDYWGQGTSVTVSSASTK | |
| | GPSVFPLAPSSKSTSGGTAALGCLVKDYFPEPVTVSWNSGAL | |
| | TSGVHTFPAVLQSSGLYSLSSVVTVPSSSLGTQTYICNVNHKP | |
| | SNTKVDKKVEPKSCHHHHHH | |

Constructs for binding to NYESO-1 HLA-A2 (scTCR)

| | | |
|---|---|---|
| E200 + scTCR NYESO- | GHNYTTRNILPGLNITSAQSVAQPEDLVNVAEGNPLTVKCTYS | 301 |
| 1v1 binder (nfP2X₇ | VSGNPYLFWYVQYPNRGLQFLLKYLGDSALVKGSYGFEAEFN | |
| epitope underlined), | KSQTSFHLKKPSALVSDSALYFCAVRDINSGAGSYQLTFGKGT | |
| 18C6, | KLSVIPGGGGGGGGSGGGGSSAVISQKPSRDIKQRGTSLTIQ | |
| WO2017109496A1, | CQVDKRLALMFWYRQQPGQSPTLIATAWTGGEATYESGFVID | |
| B136-1_v1 | KFPISRPNLTFSTLTVSNMSPEDSSIYLCSVGGSGAADTQYFG | |
| | PGTRLTVLHHHHHH | |
| E200 + scTCR NYESO- | HHHHHHAQSVAQPEDLVNVAEGNPLTVKCTYSVSGNPYLFW | 302 |
| 1v2 binder (nfP2X₇ | YVQYPNRGLQFLLKYLGDSALVKGSYGFEAEFNKSQTSFHLKK | |
| epitope underlined), | PSALVSDSALYFCAVRDINSGAGSYQLTFGKGTKLSVIPGGGG | |
| WO2017109496A1, | SGGGGSGGGGSSAVISQKPSRDIKQRGTSLTIQCQVDKRLAL | |
| B136-2_v2 | MFWYRQQPGQSPTLIATAWTGGEATYESGFVIDKFPISRPNLT | |
| | FSTLTVSNMSPEDSSIYLCSVGGSGAADTQYFGPGTRLTVLGG | |
| | GGSGHNYTTRNILPGLNITS | |

Constructs for binding to SURVIVIN HLA-A2 (scTCR)

| | | |
|---|---|---|
| E200 + scTCR | GHNYTTRNILPGLNITSSQTIHQWPATLVQPVGSPLSLECTVEG | 303 |
| SURVIVINv1 binder | TSNPNLYWYRQAAGRGLELLFYSVGIGQISSEVPQNLFASRPQ | |
| (nfP2X₇ epitope | DRQFILSSKKLLLSDSGFYLCAWSIGAEQFFGPGTRLTVLEDLK | |
| underlined), 18C6, | NGSADDAKKDAAKKDGKSGGGGSGGGGSGGGGSQKEVEQN | |
| NZ719707, B137-1_v1 | SGPLSVPEGAIASLNCTYSDRGSQSFFWYRQYSGKSPELIMSI | |
| | YSNGDKEDGRFTAQLNKASQYVSLLIRDSQPSDSATYLCAVSK | |
| | GYKMFGDGTQLVVKPNIHHHHHH | |
| E200 + scTCR NYESO- | HHHHHHSQTIHQWPATLVQPVGSPLSLECTVEGTSNPNLYWY | 304 |
| 1v2 binder (nfP2X₇ | RQAAGRGLELLFYSVGIGQISSEVPQNLFASRPQDRQFILSSKK | |
| epitope underlined), | LLLSDSGFYLCAWSIGAEQFFGPGTRLTVLEDLKNGSADDAKK | |
| NZ719707, B137-2_v2 | DAAKKDGKSGGGGSGGGGSGGGGSQKEVEQNSGPLSVPEG | |
| | AIASLNCTYSDRGSQSFFWYRQYSGKSPELIMSIYSNGDKEDG | |
| | RFTAQLNKASQYVSLLIRDSQPSDSATYLCAVSKGYKMFGDGT | |
| | QLVVKPNIGGGGSGHNYTTRNILPGLNITS | |

Constructs for biding to BCMA (ligand)

| | | |
|---|---|---|
| E200 + dAPRIL binder | GHNYTTRNILPGLNITSSVLHLVPINATSKDDSDVTEVMWQPAL | 305 |
| (nfP2X₇ epitope | RRGRGLQAQGYGVRIQDAGVYLLYSQVLFQDVTFTMGQVVS | |
| underlined), | REGQGRQETLFRCIRSMPSHPDRAYNSCYSAGVFHLHQGDIL | |
| WO2015052538A1, | SVIIPRARAKLNLSPHGTFLGFVKLSGGGSDPHHHHHH | |
| B138-1_v1 | | |
| E200 + dAPRIL binder | HHHHHHSVLHLVPINATSKDDSDVTEVMWQPALRRGRGLQA | 306 |
| (nfP2X₇ epitope | QGYGVRIQDAGVYLLYSQVLFQDVTFTMGQVVSREGQGRQE | |
| underlined), | TLFRCIRSMPSHPDRAYNSCYSAGVFHLHQGDILSVIIPRARAK | |
| WO2015052538A1, | LNLSPHGTFLGFVKLSGGGSDPGHNYTTRNILPGLNITS | |
| B139-2_v2 | | |

Orchestration molecule OR1 (nfP2X₇),
single domain Fc fusion protein

| | | |
|---|---|---|
| OR1, HC, BIL03 | EVQLLESGGGLVQPGGSLRLSCAASGFTFRNHDMGWVRQAP | 307 |
| | GKGLEWVSAISGSGGSTYYANSVKGRFTISRDNSKNTLYLQM | |
| | NSLRAEDTAVYYCAEPKPMDTEFDYRSPGTLVTVSSEPKSCD | |
| | KTHTCPPCPAPELLGGPDVFLFPPKPKDTLMISRTPEVTCVVV | |
| | DVSHEDPEVKFNWYVDGVEVHNAKTKPREEQYNSTYRVVSVL | |
| | TVLHQDWLNGKEYKCKVSNKALPAPEEKTISKAKGQPREPQV | |
| | YTLPPSRDELTKNQVSLTCLVKGFYPSDIAVEWESNGQPENNY | |
| | KTTPPVLDSDGSFFLYSKLTVDKSRWQQGNVFSCSVMHEALH | |
| | NHYTQKSLSLSPGKHHHHHH | |

Orchestration molecules OR2, OR2-3, OR2-4,
OR2-5, OR2-6 (nfP2X₇), IgG1 format

| | | |
|---|---|---|
| OR2, HC, BIL03 | EVQLLESGGGLVQPGGSLRLSCAASGFTFRNHDMGWVRQAP | 308 |
| | GKGLEWVSAISGSGGSTYYANSVKGRFTISRDNSKNTLYLQM | |
| | NSLRAEDTAVYYCAEPKPMDTEFDYRSPGTLVTVSSASTKGP | |
| | SVFPLAPSSKSTSGGTAALGCLVKDYFPEPVTVSWNSGALTS | |
| | GVHTFPAVLQSSGLYSLSSVVTVPSSSLGTQTYICNVNHKPSN | |
| | TKVDKKVEPKSCDKTHTCPPCPAPELLGGPDVFLFPPKPKDTL | |
| | MISRTPEVTCVVVDVSHEDPEVKFNWYVDGVEVHNAKTKPRE | |
| | EQYNSTYRVVSVLTVLHQDWLNGKEYKCKVSNKALPAPEEKTI | |
| | SKAKGQPREPQVYTLPPSRDELTKNQVSLTCLVKGFYPSDIAV | |
| | EWESNGQPENNYKTTPPVLDSDGSFFLYSKLTVDKSRWQQG | |
| | NVFSCSVMHEALHNHYTQKSLSLSPGKHHHHHH | |
| OR3, LC, WTB1 | DIQMTQSPSSLSASVGDRVTITCRASQYIYDYLNWYQQKPGKA | 309 |
| | PKLLIYAASYLQSGVPSRFSGSGSGTDFTLTISSLOPEDFATYY | |
| | CQQYHHPSTFGQGTKVEIKRTVAAPSVFIFPPSDEQLKSGTAS | |
| | VVCLLNNFYPREAKVQWKVDNALQSGNSQESVTEQDSKDST | |
| | YSLSSTLTLSKADYEKHKVYACEVTHQGLSSPVTKSFNRGEC | |
| OR4, LC, WTF4 | DIQMTQSPSSLSASVGDRVTITCRASQSISSYLNWYQQKPGKA | 310 |
| | PKLLIYSASALQSGVPSRFSGSGSGTDFTLTISSLQPEDFVTYY | |
| | CQQLGQGPYTFGQGTKVEIKRTVAAPSVFIFPPSDEQLKSGTA | |
| | SVVCLLNNFYPREAKVQWKVDNALQSGNSQESVTEQDSKDS | |
| | TYSLSSTLTLSKADYEKHKVYACEVTHQGLSSPVTKSFNRGEC | |
| OR5, LC, WTF11 | DIQMTQSPSSLSASVGDRVTITCRASQSISSYLNWYQQKPGKA | 311 |
| | PKLLIYSASSLQSGVPSRFSGSGSGTDFTLTISSLQPEDFATYY | |
| | CQQYGYSPHTFGQGTKVEIKRTVAAPSVFIFPPSDEQLKSGTA | |
| | SVVCLLNNFYPREAKVQWKVDNALQSGNSQESVTEQDSKDS | |
| | TYSLSSTLTLSKADYEKHKVYACEVTHQGLSSPVTKSFNRGEC | |
| OR6, LC, WTG6 | DIQMTQSPSSLSASVGDRVTITCRASQSISSYLNWYQQKPGKA | 312 |
| | PKLLIYDSSALQSGVPSRFSGSGSGTDFTLTISSLOPEDFATYY | |
| | CQQSVALPYTFGQGTKVEIKRTVAAPSVFIFPPSDEQLKSGTA | |
| | SVVCLLNNFYPREAKVQWKVDNALQSGNSQESVTEQDSKDS | |
| | TYSLSSTLTLSKADYEKHKVYACEVTHQGLSSPVTKSFNRGEC | |

Orchestration molecule OR13, OR13-3,
OR13-4, OR13-5, OR13-6 (CD3 x nfP2X₇)

| | | |
|---|---|---|
| OR13, HC, BIL03 | EVQLLESGGGLVQPGGSLRLSCAASGFTFRNHDMGWVRQAP | 313 |
| | GKGLEWVSAISGSGGSTYYANSVKGRFTISRDNSKNTLYLQM | |
| | NSLRAEDTAVYYCAEPKPMDTEFDYRSPGTLVTVSSASTKGP | |
| | SVFPLAPSSKSTSGGTAALGCLVKDYFPEPVTVSWNSGALTS | |
| | GVHTFPAVLQSSGLYSLSSVVTVPSSSLGTQTYICNVNHKPSN | |
| | TKVDKKVEPKSCDKTHTCPPCPAPELLGGPDVFLFPPKPKDTL | |
| | MISRTPEVTCVVVDVSHEDPEVKFNWYVDGVEVHNAKTKPRE | |
| | EQYNSTYRVVSVLTVLHQDWLNGKEYKCKVSNKALPAPEEKTI | |
| | SKAKGQPSGDIKLQQSGAELARPGASVKMSCKTSGYTFTRYT | |
| | MHWVKQRPGQGLEWIGYINPSRGYTNYNQKFKDKATLTTDKS | |
| | SSTAYMQLSSLTSEDSAVYYCARYYDDHYCLDYWGQGTTLTV | |
| | SSVEGGSGGSGGSGGSGGVDDIQLTQSPAIMSASPGEKVTM | |
| | TCRASSSVSYMNWYQQKSGTSPKRWIYDTSKVASGVPYRFS | |
| | GSGSGTSYSLTISSMEAEDAATYYCQQWSSNPLTFGAGTKLE | |
| | LKHHHHHH | |
| OR3, LC, WTB1 | DIQMTQSPSSLSASVGDRVTITCRASQYIYDYLNWYQQKPGKA | 314 |
| | PKLLIYAASYLQSGVPSRFSGSGSGTDFTLTISSLOPEDFATYY | |
| | CQQYHHPSTFGQGTKVEIKRTVAAPSVFIFPPSDEQLKSGTAS | |
| | VVCLLNNFYPREAKVQWKVDNALQSGNSQESVTEQDSKDST | |
| | YSLSSTLTLSKADYEKHKVYACEVTHQGLSSPVTKSFNRGEC | |
| OR4, LC, WTF4 | DIQMTQSPSSLSASVGDRVTITCRASQSISSYLNWYQQKPGKA | 315 |
| | PKLLIYSASALQSGVPSRFSGSGSGTDFTLTISSLQPEDFVTYY | |
| | CQQLGQGPYTFGQGTKVEIKRTVAAPSVFIFPPSDEQLKSGTA | |
| | SVVCLLNNFYPREAKVQWKVDNALQSGNSQESVTEQDSKDS | |
| | TYSLSSTLTLSKADYEKHKVYACEVTHQGLSSPVTKSFNRGEC | |
| OR5, LC, WTF11 | DIQMTQSPSSLSASVGDRVTITCRASQSISSYLNWYQQKPGKA | 316 |
| | PKLLIYSASSLQSGVPSRFSGSGSGTDFTLTISSLQPEDFATYY | |
| | CQQYGYSPHTFGQGTKVEIKRTVAAPSVFIFPPSDEQLKSGTA | |
| | SVVCLLNNFYPREAKVQWKVDNALQSGNSQESVTEQDSKDS | |
| | TYSLSSTLTLSKADYEKHKVYACEVTHQGLSSPVTKSFNRGEC | |
| OR6, LC, WTG6 | DIQMTQSPSSLSASVGDRVTITCRASQSISSYLNWYQQKPGKA | 317 |
| | PKLLIYDSSALQSGVPSRFSGSGSGTDFTLTISSLQPEDFATYY | |
| | CQQSVALPYTFGQGTKVEIKRTVAAPSVFIFPPSDEQLKSGTA | |
| | SVVCLLNNFYPREAKVQWKVDNALQSGNSQESVTEQDSKDS | |
| | TYSLSSTLTLSKADYEKHKVYACEVTHQGLSSPVTKSFNRGEC | |

Orchestration molecule OR14, OR14-20 (CD3 x nfP2X₇)

| | | |
|---|---|---|
| OR14, HC, 4B11 | EVQLLESGGGLVQPGGSLRLSCAASGVRISDEDMGWVRQAP | 318 |
| | GKGLEWVSAIDDAGDSTYYADSVKGRFTISRDNSKNTLYLQM | |
| | NSLRAEDTAVYYCAKVYPYDTVFDYWGQGTLVTVSSASTKGP | |
| | SVFPLAPSSKSTSGGTAALGCLVKDYFPEPVTVSWNSGALTS | |
| | GVHTFPAVLQSSGLYSLSSVVTVPSSSLGTQTYICNVNHKPSN | |
| | TKVDKKVEPKSCDKTHTCPPCPAPELLGGPDVFLFPPKPKDTL | |
| | MISRTPEVTCVVVDVSHEDPEVKFNWYVDGVEVHNAKTKPRE | |
| | EQYNSTYRVVSVLTVLHQDWLNGKEYKCKVSNKALPAPEEKTI | |
| | SKAKGQPSGDIKLQQSGAELARPGASVKMSCKTSGYTFTRYT | |
| | MHWVKQRPGQGLEWIGYINPSRGYTNYNQKFKDKATLTTDKS | |
| | SSTAYMQLSSLTSEDSAVYYCARYYDDHYCLDYWGQGTTLTV | |
| | SSVEGGSGGSGGSGGSGGVDDIQLTQSPAIMSASPGEKVTM | |
| | TCRASSSVSYMNWYQQKSGTSPKRWIYDTSKVASGVPYRFS | |
| | GSGSGTSYSLTISSMEAEDAATYYCQQWSSNPLTFGAGTKLE | |
| | LKHHHHHH | |
| OR20, LC, Gr2B1 | DIQMTQSPSSLSASVGDRVTITCRASQSISSYLNWYQQKPGKA | 319 |
| | PKLLIYAASDLQSGVPSRFSGSGSGTDFTLTISSLOPEDFATYY | |
| | CQQSIWAPYTFGQGTKVEIKRTVAAPSVFIFPPSDEQLKSGTA | |
| | SVVCLLNNFYPREAKVQWKVDNALQSGNSQESVTEQDSKDS | |
| | TYSLSSTLTLSKADYEKHKVYACEVTHQGLSSPVTKSFNRGEC | |

Orchestration molecule OR15 (CD3 x nfP2X₇)

| | | |
|---|---|---|
| OR15, BITE, VH 4B11 | EVQLLESGGGLVQPGGSLRLSCAASGVRISDEDMGWVRQAP | 320 |
| | GKGLEWVSAIDDAGDSTYYADSVKGRFTISRDNSKNTLYLQM | |
| | NSLRAEDTAVYYCAKVYPYDTVFDYWGQGTLVTVSSGGGGS | |
| | DIKLQQSGAELARPGASVKMSCKTSGYTFTRYTMHWVKQRP | |
| | GQGLEWIGYINPSRGYTNYNQKFKDKATLTTDKSSSTAYMQLS | |
| | SLTSEDSAVYYCARYYDDHYCLDYWGQGTTLTVSSVEGGSG | |
| | GSGGSGGSGGVDDIQLTQSPAIMSASPGEKVTMTCRASSSVS | |
| | YMNWYQQKSGTSPKRWIYDTSKVASGVPYRFSGSGSGTSYS | |
| | LTISSMEAEDAATYYCQQWSSNPLTFGAGTKLELKHHHHHH | |

Orchestration molecule OR16 (CD3 x nfP2X₇)

| | | |
|---|---|---|
| OR16, BITE, VH BIL03 | EVQLLESGGGLVQPGGSLRLSCAASGFTFRNHDMGWVRQAP | 321 |
| | GKGLEWVSAISGSGGSTYYANSVKGRFTISRDNSKNTLYLQM | |
| | NSLRAEDTAVYYCAEPKPMDTEFDYRSPGTLVTVSSGGGGSD | |
| | IKLQQSGAELARPGASVKMSCKTSGYTFTRYTMHWVKQRPG | |
| | QGLEWIGYINPSRGYTNYNQKFKDKATLTTDKSSSTAYMQLSS | |
| | LTSEDSAVYYCARYYDDHYCLDYWGQGTTLTVSSVEGGSGG | |
| | SGGSGGSGGVDDIQLTQSPAIMSASPGEKVTMTCRASSSVSY | |
| | MNWYQQKSGTSPKRWIYDTSKVASGVPYRFSGSGSGTSYSL | |
| | TISSMEAEDAATYYCQQWSSNPLTFGAGTKLELKHHHHHH | |

Orchestration molecule OR17 (CD3 x nfP2X₇)

| | | |
|---|---|---|
| OR16, BITE, BIL03- | DIQMTQSPSSLSASVGDRVTITCRASQYIYDYLNWYQQKPGKA | 322 |
| WTB1 | PKLLIYAASYLQSGVPSRFSGSGSGTDFTLTISSLOPEDFATYY | |
| | CQQYHHPSTFGQGTKVEIKGGGGSGGGGSGGGGSEVQLLES | |
| | GGGLVQPGGSLRLSCAASGFTFRNHDMGWVRQAPGKGLEW | |
| | VSAISGSGGSTYYANSVKGRFTISRDNSKNTLYLQMNSLRAED | |
| | TAVYYCAEPKPMDTEFDYRSPGTLVTVSSGGGGSDIKLQQSG | |
| | AELARPGASVKMSCKTSGYTFTRYTMHWVKQRPGQGLEWIG | |
| | YINPSRGYTNYNQKFKDKATLTTDKSSSTAYMQLSSLTSEDSA | |
| | VYYCARYYDDHYCLDYWGQGTTLTVSSVEGGSGGSGGSGG | |
| | SGGVDDIQLTQSPAIMSASPGEKVTMTCRASSSVSYMNWYQQ | |
| | KSGTSPKRWIYDTSKVASGVPYRFSGSGSGTSYSLTISSMEAE | |
| | DAATYYCQQWSSNPLTFGAGTKLELKHHHHHH | |

Orchestration molecule OR19_6 (CD3 x CD19)

| | | |
|---|---|---|
| OR19_6, BITE, BIL03- | DIVMTQSPATLSLSPGERATLSCRSSKSLQNVNGNTYLYWFQ | 323 |
| WTB1 | QKPGQSPQLLIYRMSNLNSGVPDRFSGSGSGTEFTLTISSLEP | |
| | EDFAVYYCMQHLEYPITFGAGTKLEIKGGGGSGGGGSGGGGS | |
| | EVQLVESGGGLVKPGGSLKLSCAASGYTFTSYVMHWVRQAP | |
| | GKGLEWIGYINPYNDGTKYNEKFQGRVTISSDKSISTAYMELSS | |
| | LRSEDTAMYYCARGTYYYGTRVFDYWGQGTLVTVSSGGGGS | |
| | DIKLQQSGAELARPGASVKMSCKTSGYTFTRYTMHWVKQRP | |
| | GQGLEWIGYINPSRGYTNYNQKFKDKATLTTDKSSSTAYMQLS | |
| | SLTSEDSAVYYCARYYDDHYCLDYWGQGTTLTVSSVEGGSG | |
| | GSGGSGGSGGVDDIQLTQSPAIMSASPGEKVTMTCRASSSVS | |
| | YMNWYQQKSGTSPKRWIYDTSKVASGVPYRFSGSGSGTSYS | |
| | LTISSMEAEDAATYYCQQWSSNPLTFGAGTKLELKHHHHHH | |

Orchestration molecule OR30 (BITE, CD3 x nfP2X₇) = HL HL

| | | |
|---|---|---|
| OR30, BITE, 4A7- | EVQLLESGGGLVQPGGSLRLSCAASGFRISDEDMGWVRQAP | 324 |
| Gr2B1 | GKGLEWVSAIDDAGDSTYYADSVKGRFTISRDNSKNTLYLQM | |
| | NSLRAEDTAVYYCAKVYPYDTVFDYWGQGTLVTVSSGGGGS | |
| | GGGGSGGGGSTDIQMTQSPSSLSASVGDRVTITCRASQSISS | |
| | YLNWYQQKPGKAPKLLIYAASDLQSGVPSRFSGSGSGTDFTL | |
| | TISSLQPEDFATYYCQQSIWAPYTFGQGTKVEIKRGGGGSDIK | |
| | LQQSGAELARPGASVKMSCKTSGYTFTRYTMHWVKQRPGQG | |
| | LEWIGYINPSRGYTNYNQKFKDKATLTTDKSSSTAYMQLSSLT | |
| | SEDSAVYYCARYYDDHYCLDYWGQGTTLTVSSVEGGSGGSG | |
| | GSGGSGGVDDIQLTQSPAIMSASPGEKVTMTCRASSSVSYMN | |
| | WYQQKSGTSPKRWIYDTSKVASGVPYRFSGSGSGTSYSLTIS | |
| | SMEAEDAATYYCQQWSSNPLTFGAGTKLELKHHHHHH | |

Orchestration molecule OR31 (BITE, CD3 x nfP2X₇) = LH HL

| | | |
|---|---|---|
| OR31, BITE, 4A7- | DIQMTQSPSSLSASVGDRVTITCRASQSISSYLNWYQQKPGKA | 325 |
| Gr2B1 | PKLLIYAASDLQSGVPSRFSGSGSGTDFTLTISSLQPEDFATYY | |
| | CQQSIWAPYTFGQGTKVEIKRGGGGSGGGGSGGGGSEVQLL | |
| | ESGGGLVQPGGSLRLSCAASGFRISDEDMGWVRQAPGKGLE | |
| | WVSAIDDAGDSTYYADSVKGRFTISRDNSKNTLYLQMNSLRAE | |
| | DTAVYYCAKVYPYDTVFDYWGQGTLVTVSSGGGGSDIKLQQS | |
| | GAELARPGASVKMSCKTSGYTFTRYTMHWVKQRPGQGLEWI | |
| | GYINPSRGYTNYNQKFKDKATLTTDKSSSTAYMQLSSLTSEDS | |
| | AVYYCARYYDDHYCLDYWGQGTTLTVSSVEGGSGGSGGSG | |
| | GSGGVDDIQLTQSPAIMSASPGEKVTMTCRASSSVSYMNWYQ | |
| | QKSGTSPKRWIYDTSKVASGVPYRFSGSGSGTSYSLTISSMEA | |
| | EDAATYYCQQWSSNPLTFGAGTKLELKHHHHHH | |

Orchestration molecule OR32 (BITE, CD16 x nfP2X₇) = HL LH

| | | |
|---|---|---|
| OR32, BIKE, 4A7- | EVQLLESGGGLVQPGGSLRLSCAASGFRISDEDMGWVRQAP | 326 |
| Gr2B1 | GKGLEWVSAIDDAGDSTYYADSVKGRFTISRDNSKNTLYLQM | |
| | NSLRAEDTAVYYCAKVYPYDTVFDYWGQGTLVTVSSGGGGS | |
| | GGGGSGGGGSTDIQMTQSPSSLSASVGDRVTITCRASQSISS | |
| | YLNWYQQKPGKAPKLLIYAASDLQSGVPSRFSGSGSGTDFTL | |
| | TISSLOPEDFATYYCQQSIWAPYTFGQGTKVEIKRGGGGSQVT | |
| | LKESGPGILQPSQTLSLTCSFSGFSLRTSGMGVGWIRQPSGK | |
| | GLEWLAHIWWDDDKRYNPALKSRLTISKDTSSNQVFLKIASVD | |
| | TADTATYYCAQINPAWFAYWGQGTLVTVSSGGGGSGGGGSG | |
| | GGGSDIVLTQSPASLAVSLGQRATISCKASQSVDFDGDSFMN | |
| | WYQQKPGQPPKLLIYTTSNLESGIPARFSASGSGTDFTLNIHPV | |
| | EEEDTATYYCQQSNEDPYTFGGGTKLEIKHHHHHH | |

Orchestration molecule OR33 (BITE, CD16 x nfP2X₇) = LH HL

| | | |
|---|---|---|
| OR33, BİKE, 4A7- | DIQMTQSPSSLSASVGDRVTITCRASQSISSYLNWYQQKPGKA | 327 |
| Gr2B1 | PKLLIYAASDLQSGVPSRFSGSGSGTDFTLTISSLQPEDFATYY | |
| | CQQSIWAPYTFGQGTKVEIKRGGGGSGGGGSGGGGSEVQLL | |
| | ESGGGLVQPGGSLRLSCAASGFRISDEDMGWVRQAPGKGLE | |
| | WVSAIDDAGDSTYYADSVKGRFTISRDNSKNTLYLQMNSLRAE | |
| | DTAVYYCAKVYPYDTVFDYWGQGTLVTVSSGGGGSQVTLKE | |
| | SGPGILQPSQTLSLTCSFSGFSLRTSGMGVGWIRQPSGKGLE | |
| | WLAHIWWDDDKRYNPALKSRLTISKDTSSNQVFLKIASVDTAD | |
| | TATYYCAQINPAWFAYWGQGTLVTVSSGGGGSGGGGSGGG | |
| | GSDIVLTQSPASLAVSLGQRATISCKASQSVDFDGDSFMNWY | |
| | QQKPGQPPKLLIYTTSNLESGIPARFSASGSGTDFTLNIHPVEE | |
| | EDTATYYCQQSNEDPYTFGGGTKLEIKHHHHHH | |

Orchestration molecule OR34 (CD3 x nfP2X₇)

| | | |
|---|---|---|
| OR34, HC, 4A7 | EVQLLESGGGLVQPGGSLRLSCAASGFRISDEDMGWVRQAP | 328 |
| | GKGLEWVSAIDDAGDSTYYADSVKGRFTISRDNSKNTLYLQM | |
| | NSLRAEDTAVYYCAKVYPYDTVFDYWGQGTLVTVSSASTKGP | |
| | SVFPLAPSSKSTSGGTAALGCLVKDYFPEPVTVSWNSGALTS | |
| | GVHTFPAVLQSSGLYSLSSVVTVPSSSLGTQTYICNVNHKPSN | |
| | TKVDKKVEPKSCDKTHTSPGQPSGDIKLQQSGAELARPGASV | |
| | KMSCKTSGYTFTRYTMHWVKQRPGQGLEWIGYINPSRGYTN | |
| | YNQKFKDKATLTTDKSSSTAYMQLSSLTSEDSAVYYCARYYD | |
| | DHYCLDYWGQGTTLTVSSVEGGSGGSGGSGGSGGVDDIQLT | |
| | QSPAIMSASPGEKVTMTCRASSSVSYMNWYQQKSGTSPKRW | |
| | IYDTSKVASGVPYRFSGSGSGTSYSLTISSMEAEDAATYYCQQ | |
| | WSSNPLTFGAGTKLELKHHHHHH | |
| OR20, LC, Gr2B1 | DIQMTQSPSSLSASVGDRVTITCRASQSISSYLNWYQQKPGKA | 329 |
| | PKLLIYAASDLQSGVPSRFSGSGSGTDFTLTISSLOPEDFATYY | |
| | CQQSIWAPYTFGQGTKVEIKRTVAAPSVFIFPPSDEQLKSGTA | |
| | SVVCLLNNFYPREAKVQWKVDNALQSGNSQESVTEQDSKDS | |
| | TYSLSSTLTLSKADYEKHKVYACEVTHQGLSSPVTKSFNRGEC | |
| OR21, LC, Gr2C12 | DIQMTQSPSSLSASVGDRVTITCRASQSISSYLNWYQQKPGKA | 330 |
| | PKLLIYDASSLQSGVPSRFSGSGSGTDFTLTISSLOPEDFATYY | |
| | CQQGYYLPGTFGQGTKVEIKRTVAAPSVFIFPPSDEQLKSGTA | |
| | SVVCLLNNFYPREAKVQWKVDNALQSGNSQESVTEQDSKDS | |
| | TYSLSSTLTLSKADYEKHKVYACEVTHQGLSSPVTKSFNRGEC | |
| OR22, LC, Gr2F10 | DIQMTQSPSSLSASVGDRVTITCRASQSISSYLNWYQQKPGKA | 331 |
| | PKLLIYAASSLQSGVPSRFSGSGSGTDFTLTISSLQPEDFATYY | |
| | CQQSSSHPHTFGQGTKVEIKRTVAAPSVFIFPPSDEQLKSGTA | |
| | SVVCLLNNFYPREAKVQWKVDNALQSGNSQESVTEQDSKDS | |
| | TYSLSSTLTLSKADYEKHKVYACEVTHQGLSSPVTKSFNRGEC | |
| OR23, LC, Gr2F11 | DIQMTQSPSSLSASVGDRVTITCRASQSISSYLNWYQQKPGKA | 332 |
| | PKLLIYAASSLQSGVPSRFSGSGSGTDFTLTISSLOPEDFATYY | |
| | CQQTYEHPYTFGQGTKVEIKRTVAAPSVFIFPPSDEQLKSGTA | |
| | SVVCLLNNFYPREAKVQWKVDNALQSGNSQESVTEQDSKDS | |
| | TYSLSSTLTLSKADYEKHKVYACEVTHQGLSSPVTKSFNRGEC | |
| | * | |
| OR24, LC, Gr2F12 | DIQMTQSPSSLSASVGDRVTITCRASQSISSYLNWYQQKPGKA | 333 |
| | PKLLIYSASNLQSGVPSRFSGSGSGTDFTLTISSLOPEDFATYY | |
| | CQQVDGSPQTFGQGTKVEIKRTVAAPSVFIFPPSDEQLKSGTA | |
| | SVVCLLNNFYPREAKVQWKVDNALQSGNSQESVTEQDSKDS | |
| | TYSLSSTLTLSKADYEKHKVYACEVTHQGLSSPVTKSFNRGEC | |

Orchestration molecule OR35 (CD16 x nfP2X₇)

| | | |
|---|---|---|
| OR35, HC, 4A7 | EVQLLESGGGLVQPGGSLRLSCAASGFRISDEDMGWVRQAP | 334 |
| | GKGLEWVSAIDDAGDSTYYADSVKGRFTISRDNSKNTLYLQM | |
| | NSLRAEDTAVYYCAKVYPYDTVFDYWGQGTLVTVSSASTKGP | |
| | SVFPLAPSSKSTSGGTAALGCLVKDYFPEPVTVSWNSGALTS | |
| | GVHTFPAVLQSSGLYSLSSVVTVPSSSLGTQTYICNVNHKPSN | |
| | TKVDKKVEPKSCDKTHTSPGQPSGQVTLKESGPGILQPSQTLS | |
| | LTCSFSGFSLRTSGMGVGWIRQPSGKGLEWLAHIWWDDDKR | |
| | YNPALKSRLTISKDTSSNQVFLKIASVDTADTATYYCAQINPAW | |
| | FAYWGQGTLVTVSSGGGGSGGGGSGGGGSDIVLTQSPASLA | |
| | VSLGQRATISCKASQSVDFDGDSFMNWYQQKPGQPPKLLIYT | |
| | TSNLESGIPARFSASGSGTDFTLNIHPVEEEDTATYYCQQSNE | |
| | DPYTFGGGTKLEIKHHHHHH | |
| OR20, LC, Gr2B1 | DIQMTQSPSSLSASVGDRVTITCRASQSISSYLNWYQQKPGKA | 335 |
| | PKLLIYAASDLQSGVPSRFSGSGSGTDFTLTISSLOPEDFATYY | |
| | CQQSIWAPYTFGQGTKVEIKRTVAAPSVFIFPPSDEQLKSGTA | |
| | SVVCLLNNFYPREAKVQWKVDNALQSGNSQESVTEQDSKDS | |
| | TYSLSSTLTLSKADYEKHKVYACEVTHQGLSSPVTKSFNRGEC | |
| OR21, LC, Gr2C12 | DIQMTQSPSSLSASVGDRVTITCRASQSISSYLNWYQQKPGKA | 336 |
| | PKLLIYDASSLQSGVPSRFSGSGSGTDFTLTISSLQPEDFATYY | |
| | CQQGYYLPGTFGQGTKVEIKRTVAAPSVFIFPPSDEQLKSGTA | |
| | SVVCLLNNFYPREAKVQWKVDNALQSGNSQESVTEQDSKDS | |
| | TYSLSSTLTLSKADYEKHKVYACEVTHQGLSSPVTKSFNRGEC | |
| OR22, LC, Gr2F10 | DIQMTQSPSSLSASVGDRVTITCRASQSISSYLNWYQQKPGKA | 337 |
| | PKLLIYAASSLQSGVPSRFSGSGSGTDFTLTISSLOPEDFATYY | |
| | CQQSSSHPHTFGQGTKVEIKRTVAAPSVFIFPPSDEQLKSGTA | |
| | SVVCLLNNFYPREAKVQWKVDNALQSGNSQESVTEQDSKDS | |
| | TYSLSSTLTLSKADYEKHKVYACEVTHQGLSSPVTKSFNRGEC | |
| OR23, LC, Gr2F11 | DIQMTQSPSSLSASVGDRVTITCRASQSISSYLNWYQQKPGKA | 338 |
| | PKLLIYAASSLQSGVPSRFSGSGSGTDFTLTISSLOPEDFATYY | |
| | CQQTYEHPYTFGQGTKVEIKRTVAAPSVFIFPPSDEQLKSGTA | |
| | SVVCLLNNFYPREAKVQWKVDNALQSGNSQESVTEQDSKDS | |
| | TYSLSSTLTLSKADYEKHKVYACEVTHQGLSSPVTKSFNRGEC | |
| | * | |
| OR24, LC, Gr2F12 | DIQMTQSPSSLSASVGDRVTITCRASQSISSYLNWYQQKPGKA | 339 |
| | PKLLIYSASNLQSGVPSRFSGSGSGTDFTLTISSLOPEDFATYY | |
| | CQQVDGSPQTFGQGTKVEIKRTVAAPSVFIFPPSDEQLKSGTA | |
| | SVVCLLNNFYPREAKVQWKVDNALQSGNSQESVTEQDSKDS | |
| | TYSLSSTLTLSKADYEKHKVYACEVTHQGLSSPVTKSFNRGEC | |

Orchestration molecule OR36 (CD3 x CLDN6), IgG1 format

| | | |
|---|---|---|
| OR36, HC, CLDN6 | EVQLLESGGGLVQPGGSMRLSCAASGFTFSNYWMNWVRQA | 340 |
| clone_Ab3-4_seq, | PGKGLEWVAQIRLKSDNYATHYADSVKGRFTISRDDSKNTVYL | |
| WO2019056023 | QMNSLRAEDTGVYYCNDGPPSGYWGQGTLLTVSSASTKGPS | |
| | VFPLAPSSKSTSGGTAALGCLVKDYFPEPVTVSWNSGALTSG | |
| | VHTFPAVLQSSGLYSLSSVVTVPSSSLGTQTYICNVNHKPSNT | |
| | KVDKKVEPKSCDKTHTCPPCPAPELLGGPDVFLFPPKPKDTLM | |
| | ISRTPEVTCVVVDVSHEDPEVKFNWYVDGVEVHNAKTKPREE | |
| | QYNSTYRVVSVLTVLHQDWLNGKEYKCKVSNKALPAPEEKTIS | |
| | KAKGQPREPQVYTLPPSRDELTKNQVSLTCLVKGFYPSDIAVE | |
| | WESNGQPENNYKTTPPVLDSDGSFFLYSKLTVDKSRWQQGN | |
| | VFSCSVMHEALHNHYTQKSLSLSPGKHHHHHH | |
| OR36, LC, CLDN6 | DIQMTQSPSSLSASVGDRVTITCRISENIYSYLAWYQQKPGKA | 341 |
| clone_Ab3-4_seq, | PKLLIYNAKILVEGVPSRFSGSGSGTDFTLTISSLOPEDFATYYC | |
| WO2019056023 | QHHYTVPWTFGQGTKLEIKRTVAAPSVFIFPPSDEQLKSGTAS | |
| | VVCLLNNFYPREAKVQWKVDNALQSGNSQESVTEQDSKDST | |
| | YSLSSTLTLSKADYEKHKVYACEVTHQGLSSPVTKSFNRGEC | |

Orchestration molecule OR37, (CD3 x CLDN6), BITE

| | | |
|---|---|---|
| OR37, BITE, CD3 | DIQMTQSPSSLSASVGDRVTITCRISENIYSYLAWYQQKPGKA | 342 |
| (OKT3) x CLDN6 | PKLLIYNAKILVEGVPSRFSGSGSGTDFTLTISSLOPEDFATYYC | |
| clone_Ab3-4_seq, | QHHYTVPWTFGQGTKLEIKGGGGSGGGGSGGGGSEVQLLES | |
| WO2019056023 | GGGLVQPGGSMRLSCAASGFTFSNYWMNWVRQAPGKGLEW | |
| | VAQIRLKSDNYATHYADSVKGRFTISRDDSKNTVYLQMNSLRA | |
| | EDTGVYYCNDGPPSGYWGQGTLLTVSSGGGGSDIKLQQSGA | |
| | ELARPGASVKMSCKTSGYTFTRYTMHWVKQRPGQGLEWIGYI | |
| | NPSRGYTNYNQKFKDKATLTTDKSSSTAYMQLSSLTSEDSAV | |
| | YYCARYYDDHYCLDYWGQGTTLTVSSVEGGSGGSGGSGGS | |
| | GGVDDIQLTQSPAIMSASPGEKVTMTCRASSSVSYMNWYQQK | |
| | SGTSPKRWIYDTSKVASGVPYRFSGSGSGTSYSLTISSMEAED | |
| | AATYYCQQWSSNPLTFGAGTKLELKHHHHHH | |

Orchestration molecule OR38 (CD16 x CLDN6), BIKE

| | | |
|---|---|---|
| OR38, BITE, CD16 | DIQMTQSPSSLSASVGDRVTITCRISENIYSYLAWYQQKPGKA | 343 |
| (3G8) x CLDN6 | PKLLIYNAKILVEGVPSRFSGSGSGTDFTLTISSLOPEDFATYYC | |
| clone_Ab3-4_seq, | QHHYTVPWTFGQGTKLEIKGGGGSGGGGSGGGGSEVQLLES | |
| WO2019056023. | GGGLVQPGGSMRLSCAASGFTFSNYWMNWVRQAPGKGLEW | |
| | VAQIRLKSDNYATHYADSVKGRFTISRDDSKNTVYLQMNSLRA | |
| | EDTGVYYCNDGPPSGYWGQGTLLTVSSGGGGSQVTLKESGP | |
| | GILQPSQTLSLTCSFSGFSLRTSGMGVGWIRQPSGKGLEWLA | |
| | HIWWDDDKRYNPALKSRLTISKDTSSNQVFLKIASVDTADTATY | |
| | YCAQINPAWFAYWGQGTLVTVSSGGGGSGGGGSGGGGSDIV | |
| | LTQSPASLAVSLGQRATISCKASQSVDFDGDSFMNWYQQKPG | |
| | QPPKLLIYTTSNLESGIPARFSASGSGTDFTLNIHPVEEEDTATY | |
| | YCQQSNEDPYTFGGGTKLEIKHHHHHH | |

Orchestration molecule OR39 (CD3 x EGFRvIII), IgG1 format

| | | |
|---|---|---|
| OR39, HC, EGFRvIII | EVQVLESGGGLVQPGGSLRLSCAASGFTFSSYAMSWVRQAP | 344 |
| (clone 139), | GKGLEWVSAISGSGGSTNYADSVKGRFTISRDNSKNTLYLQM | |
| WO2013185010 | NSLRAEDTAVYYCAGSSGWSEYWGQGTLVTVSSASTKGPSV | |
| | FPLAPSSKSTSGGTAALGCLVKDYFPEPVTVSWNSGALTSGV | |
| | HTFPAVLQSSGLYSLSSVVTVPSSSLGTQTYICNVNHKPSNTK | |
| | VDKKVEPKSCDKTHTCPPCPAPELLGGPDVFLFPPKPKDTLMI | |
| | SRTPEVTCVVVDVSHEDPEVKFNWYVDGVEVHNAKTKPREE | |
| | QYNSTYRVVSVLTVLHQDWLNGKEYKCKVSNKALPAPEEKTIS | |
| | KAKGQPREPQVYTLPPSRDELTKNQVSLTCLVKGFYPSDIAVE | |
| | WESNGQPENNYKTTPPVLDSDGSFFLYSKLTVDKSRWQQGN | |
| | VFSCSVMHEALHNHYTQKSLSLSPGKHHHHHH | |
| OR39, LC, EGFRvIII | DIQMTQSPSSLSASVGDRVTITCRASQGIRNNLAWYQQKPGK | 345 |
| (clone 139), | APKRLIYAASNLQSGVPSRFTGSGSGTEFTLIVSSLQPEDFATY | |
| WO2013185010 | YCLQHHSYPLTSGGGTKVEIKRTVAAPSVFIFPPSDEQLKSGT | |
| | ASVVCLLNNFYPREAKVQWKVDNALQSGNSQESVTEQDSKD | |
| | STYSLSSTLTLSKADYEKHKVYACEVTHQGLSSPVTKSFNRGE | |
| | C | |

Orchestration molecule OR40 (CD3 x EGFRvIII), BITE

| | | |
|---|---|---|
| OR39, BITE, CD3 | DIQMTQSPSSLSASVGDRVTITCRASQGIRNNLAWYQQKPGK | 346 |
| (OKT3) x EGFRvIII | APKRLIYAASNLQSGVPSRFTGSGSGTEFTLIVSSLOPEDFATY | |
| (clone 139), | YCLQHHSYPLTSGGGTKVEIKRGGGGSGGGGSGGGGSEVQV | |
| WO2013185010 | LESGGGLVQPGGSLRLSCAASGFTFSSYAMSWVRQAPGKGL | |
| | EWVSAISGSGGSTNYADSVKGRFTISRDNSKNTLYLQMNSLR | |
| | AEDTAVYYCAGSSGWSEYWGQGTLVTVSSGGGGSDIKLQQS | |
| | GAELARPGASVKMSCKTSGYTFTRYTMHWVKQRPGQGLEWI | |
| | GYINPSRGYTNYNQKFKDKATLTTDKSSSTAYMQLSSLTSEDS | |
| | AVYYCARYYDDHYCLDYWGQGTTLTVSSVEGGSGGSGGSG | |
| | GSGGVDDIQLTQSPAIMSASPGEKVTMTCRASSSVSYMNWYQ | |
| | QKSGTSPKRWIYDTSKVASGVPYRFSGSGSGTSYSLTISSMEA | |
| | EDAATYYCQQWSSNPLTFGAGTKLELKHHHHHH | |

Orchestration molecule OR41 (CD3 x EGFRvIII), BIKE

| | | |
|---|---|---|
| OR41, BITE, CD16 | DIQMTQSPSSLSASVGDRVTITCRASQGIRNNLAWYQQKPGK | 347 |
| (3G8) x EGFRvIII | APKRLIYAASNLQSGVPSRFTGSGSGTEFTLIVSSLOPEDFATY | |
| (clone 139), | YCLQHHSYPLTSGGGTKVEIKGGGGSGGGGSGGGGSEVQVL | |
| WO2013185010 | ESGGGLVQPGGSLRLSCAASGFTFSSYAMSWVRQAPGKGLE | |
| | WVSAISGSGGSTNYADSVKGRFTISRDNSKNTLYLQMNSLRA | |
| | EDTAVYYCAGSSGWSEYWGQGTLVTVSSGGGGSQVTLKESG | |
| | PGILQPSQTLSLTCSFSGFSLRTSGMGVGWIRQPSGKGLEWL | |
| | AHIWWDDDKRYNPALKSRLTISKDTSSNQVFLKIASVDTADTAT | |
| | YYCAQINPAWFAYWGQGTLVTVSSGGGGSGGGGSGGGGSDI | |
| | VLTQSPASLAVSLGQRATISCKASQSVDFDGDSFMNWYQQKP | |
| | GQPPKLLIYTTSNLESGIPARFSASGSGTDFTLNIHPVEEEDTA | |
| | TYYCQQSNEDPYTFGGGTKLEIKHHHHHH | |

Exemplary bridging molecule constructs for binding to CD200

| | | |
|---|---|---|
| E200 + CD200, | QVQLQQSGSELKKPGASVKISCKASGYSFTDYIILWVRQNPGK | 348 |
| samalizumab, B140-1 | GLEWIGHIDPYYGSSNYNLKFKGRVTITADQSTTTAYMELSSLR | |
| | SEDTAVYYCGRSKRDYFDYWGQGTTLTVSSASTKGPSVFPLA | |
| | PSSKSTSGGTAALGCLVKDYFPEPVTVSWNSGALTSGVHTFP | |
| | AVLQSSGLYSLSSVVTVPSSSLGTQTYICNVNHKPSNTKVDKK | |
| | VEPKSCHHHHHH | |
| CD200, LC, | DIQMTQSPSSLSASIGDRVTITCKASQDINSYLSWFQQKPGKA | 349 |
| samalizumab, B140-2 | PKLLIYRANRLVDGVPSRFSGSGSGTDYTLTISSLQPEDFAVYY | |
| | CLQYDEFPYTFGGGTKLEIKRTVAAPSVFIFPPSDEQLKSGTAS | |
| | VVCLLNNFYPREAKVQWKVDNALQSGNSQESVTEQDSKDST | |
| | YSLSSTLTLSKADYEKHKVYACEVTHQGLSSPVTKSFNRGECH | |
| | HHHHH | |

Exemplary orchestration molecules (OR molecules)
for binding innate immune cells

| | | |
|---|---|---|
| OR_BİKE1 | DIQMTQSPSSLSASVGDRVTITCRASQYIYDYLNWYQQKPGKA | 350 |
| OR105 | PKLLIYAASYLQSGVPSRFSGSGSGTDFTLTISSLOPEDFATYY | |
| | CQQYHHPSTFGQGTKVEIKRGGGGSGGGGSGGGGSEVQLLE | |
| | SGGGLVQPGGSLRLSCAASGFTFRNHDMGWVRQAPGKGLE | |
| | WVSAISGSGGSTYYANSVKGRFTISRDNSKNTLYLQMNSLRAE | |
| | DTAVYYCAEPKPMDTEFDYRSPGTLVTVSSVEGGSGGSGGS | |
| | GGSGGVDQVTLKESGPGILQPSQTLSLTCSFSGFSLRTSGMG | |
| | VGWIRQPSGKGLEWLAHIWWDDDKRYNPALKSRLTISKDTSS | |
| | NQVFLKIASVDTADTATYYCAQINPAWFAYWGQGTLVTVSSG | |
| | GGGSGGGGSGGGGSDIVLTQSPASLAVSLGQRATISCKASQS | |
| | VDFDGDSFMNWYQQKPGQPPKLLIYTTSNLESGIPARFSASG | |
| | SGTDFTLNIHPVEEEDTATYYCQQSNEDPYTFGGGTKLEIKHH | |
| | HHHH | |
| OR_BİKE2 | DIVLTQSPASLAVSLGQRATISCKASQSVDFDGDSFMNWYQQ | 351 |
| OR106 | KPGQPPKLLIYTTSNLESGIPARFSASGSGTDFTLNIHPVEEED | |
| | TATYYCQQSNEDPYTFGGGTKLEIKGGGGSGGGGSGGGGSQ | |
| | VTLKESGPGILQPSQTLSLTCSFSGFSLRTSGMGVGWIRQPSG | |
| | KGLEWLAHIWWDDDKRYNPALKSRLTISKDTSSNQVFLKIASV | |
| | DTADTATYYCAQINPAWFAYWGQGTLVTVSSVEGGSGGSGG | |
| | SGGSGGVDEVQLLESGGGLVQPGGSLRLSCAASGFTFRNHD | |
| | MGWVRQAPGKGLEWVSAISGSGGSTYYANSVKGRFTISRDN | |
| | SKNTLYLQMNSLRAEDTAVYYCAEPKPMDTEFDYRSPGTLVT | |
| | VSSGGGGSGGGGSGGGGSDIQMTQSPSSLSASVGDRVTITC | |
| | RASQYIYDYLNWYQQKPGKAPKLLIYAASYLQSGVPSRFSGSG | |
| | SGTDFTLTISSLQPEDFATYYCQQYHHPSTFGQGTKVEIKRHH | |
| | HHHH | |
| Heavy chain | QVTLKESGPGILQPSQTLSLTCSFSGFSLRTSGMGVGWIRQPS | 352 |
| tetravalent molecule | GKGLEWLAHIWWDDDKRYNPALKSRLTISKDTSSNQVFLKIAS | |
| 1 | VDTADTATYYCAQINPAWFAYWGQGTLVTVSSASTKGPSVFP | |
| OR100 | LAPSSKSTSGGTAALGCLVKDYFPEPVTVSWNSGALTSGVHT | |
| | FPAVLQSSGLYSLSSVVTVPSSSLGTQTYICNVNHKPSNTKVD | |
| | KKVEPKSCDKTHTCPPCPAPPVAGPSVFLFPPKPKDTLMISRT | |
| | PEVTCVVVGVSHEDPEVKFNWYVDGVEVHNAKTKPREEQYQ | |
| | STYRVVSVLTVLHQDWLNGKEYKCKVSNKQLPSPIEKTISKAK | |
| | GQPREPQVYTLPPSRDELTKNQVSLTCLVKGFYPSDIAVEWES | |
| | NGQPENNYKTTPPVLDSDGSFFLYSKLTVDKSRWQQGNVFSC | |
| | SVMHEALHNHYTQKSLSLSPGKGGGGSDIQMTQSPSSLSASV | |
| | GDRVTITCRASQYIYDYLNWYQQKPGKAPKLLIYAASYLQSGV | |
| | PSRFSGSGSGTDFTLTISSLQPEDFATYYCQQYHHPSTFGQG | |
| | TKVEIKRGGGGSGGGGSGGGGSEVQLLESGGGLVQPGGSLR | |
| | LSCAASGFTFRNHDMGWVRQAPGKGLEWVSAISGSGGSTYY | |
| | ANSVKGRFTISRDNSKNTLYLQMNSLRAEDTAVYYCAEPKPM | |
| | DTEFDYRSPGTLVTVSSHHHHHH | |
| Heavy chain bivalent | QVTLKESGPGILQPSQTLSLTCSFSGFSLRTSGMGVGWIRQPS | 353 |
| molecule 1 | GKGLEWLAHIWWDDDKRYNPALKSRLTISKDTSSNQVFLKIAS | |
| OR108 | VDTADTATYYCAQINPAWFAYWGQGTLVTVSSASTKGPSVFP | |
| | LAPSSKSTSGGTAALGCLVKDYFPEPVTVSWNSGALTSGVHT | |
| | FPAVLQSSGLYSLSSVVTVPSSSLGTQTYICNVNHKPSNTKVD | |
| | KKVEPKSCDKTHTSPPSPAPPVAGPSVFLFPPKPKDTLMISRT | |
| | PEVTCVVVGVSHEDPEVKFNWYVDGVEVHNAKTKPREEQYQ | |
| | STYRVVSVLTVLHQDWLNGKEYKCKVSNKQLPSPIEKTISKAK | |
| | GQPREPQVYTLPPSRDELTKNQVSLTCLVKGFYPSDIAVEWES | |
| | NGQPENNYKTTPPVLDSDGSFFLYSKLTVDKSRWQQGNVFSC | |
| | SVMHEALHNHYTQKSLSLSPGKGGGGSDIQMTQSPSSLSASV | |
| | GDRVTITCRASQYIYDYLNWYQQKPGKAPKLLIYAASYLQSGV | |
| | PSRFSGSGSGTDFTLTISSLOPEDFATYYCQQYHHPSTFGQG | |
| | TKVEIKRGGGGSGGGGSGGGGSEVQLLESGGGLVQPGGSLR | |
| | LSCAASGFTFRNHDMGWVRQAPGKGLEWVSAISGSGGSTYY | |
| | ANSVKGRFTISRDNSKNTLYLQMNSLRAEDTAVYYCAEPKPM | |
| | DTEFDYRSPGTLVTVSSHHHHHH | |
| Heavy chain | QVTLKESGPGILQPSQTLSLTCSFSGFSLRTSGMGVGWIRQPS | 354 |
| tetravalent molecule | GKGLEWLAHIWWDDDKRYNPALKSRLTISKDTSSNQVFLKIAS | |
| 2 | VDTADTATYYCAQINPAWFAYWGQGTLVTVSSASTKGPSVFP | |
| OR101 | LAPSSKSTSGGTAALGCLVKDYFPEPVTVSWNSGALTSGVHT | |
| | FPAVLQSSGLYSLSSVVTVPSSSLGTQTYICNVNHKPSNTKVD | |
| | KKVEPKSCDKTHTCPPCPAPPVAGPSVFLFPPKPKDTLMISRT | |
| | PEVTCVVVGVSHEDPEVKFNWYVDGVEVHNAKTKPREEQYQ | |
| | STYRVVSVLTVLHQDWLNGKEYKCKVSNKQLPSPIEKTISKAK | |
| | GGGGSDIQMTQSPSSLSASVGDRVTITCRASQYIYDYLNWYQ | |
| | QKPGKAPKLLIYAASYLQSGVPSRFSGSGSGTDFTLTISSLOPE | |
| | DFATYYCQQYHHPSTFGQGTKVEIKRGGGGSGGGGSGGGG | |
| | SEVQLLESGGGLVQPGGSLRLSCAASGFTFRNHDMGWVRQA | |
| | PGKGLEWVSAISGSGGSTYYANSVKGRFTISRDNSKNTLYLQ | |
| | MNSLRAEDTAVYYCAEPKPMDTEFDYRSPGTLVTVSSHHHHH | |
| | H | |
| Heavy chain bivalent | QVTLKESGPGILQPSQTLSLTCSFSGFSLRTSGMGVGWIRQPS | 355 |
| molecule 2 | GKGLEWLAHIWWDDDKRYNPALKSRLTISKDTSSNQVFLKIAS | |
| OR109 | VDTADTATYYCAQINPAWFAYWGQGTLVTVSSASTKGPSVFP | |
| | LAPSSKSTSGGTAALGCLVKDYFPEPVTVSWNSGALTSGVHT | |
| | FPAVLQSSGLYSLSSVVTVPSSSLGTQTYICNVNHKPSNTKVD | |
| | KKVEPKSCDKTHTSPPSPAPPVAGPSVFLFPPKPKDTLMISRT | |
| | PEVTCVVVGVSHEDPEVKFNWYVDGVEVHNAKTKPREEQYQ | |
| | STYRVVSVLTVLHQDWLNGKEYKCKVSNKQLPSPIEKTISKAK | |
| | GGGGSDIQMTQSPSSLSASVGDRVTITCRASQYIYDYLNWYQ | |
| | QKPGKAPKLLIYAASYLQSGVPSRFSGSGSGTDFTLTISSLQPE | |
| | DFATYYCQQYHHPSTFGQGTKVEIKRGGGGSGGGGGGGG | |
| | SEVQLLESGGGLVQPGGSLRLSCAASGFTFRNHDMGWVRQA | |
| | PGKGLEWVSAISGSGGSTYYANSVKGRFTISRDNSKNTLYLQ | |
| | MNSLRAEDTAVYYCAEPKPMDTEFDYRSPGTLVTVSSHHHHH | |
| | H | |
| Heavy chain | QVTLKESGPGILQPSQTLSLTCSFSGFSLRTSGMGVGWIRQPS | 356 |
| tetravalent molecule | GKGLEWLAHIWWDDDKRYNPALKSRLTISKDTSSNQVFLKIAS | |
| 3 | VDTADTATYYCAQINPAWFAYWGQGTLVTVSSASTKGPSVFP | |
| OR102 | LAPSSKSTSGGTAALGCLVKDYFPEPVTVSWNSGALTSGVHT | |
| | FPAVLQSSGLYSLSSVVTVPSSSLGTQTYICNVNHKPSNTKVD | |
| | KKVEPKSCDKTHTCPPCPAPPVAGPSVFLFPPKGQPREPQVY | |
| | TLPPSRDELTKNQVSLTCLVKGFYPSDIAVEWESNGQPENNYK | |
| | TTPPVLDSDGSFFLYSKLTVDKSRWQQGNVFSCSVMHEALHN | |
| | HYTQKSLSLSPGKGGGGSDIQMTQSPSSLSASVGDRVTITCR | |
| | ASQYIYDYLNWYQQKPGKAPKLLIYAASYLQSGVPSRFSGSGS | |
| | GTDFTLTISSLQPEDFATYYCQQYHHPSTFGQGTKVEIKRGGG | |
| | GSGGGGSGGGGSEVQLLESGGGLVQPGGSLRLSCAASGFTF | |
| | RNHDMGWVRQAPGKGLEWVSAISGSGGSTYYANSVKGRFTI | |
| | SRDNSKNTLYLQMNSLRAEDTAVYYCAEPKPMDTEFDYRSPG | |
| | TLVTVSSHHHHH | |
| Heavy chain bivalent | QVTLKESGPGILQPSQTLSLTCSFSGFSLRTSGMGVGWIRQPS | 357 |
| molecule 3 | GKGLEWLAHIWWDDDKRYNPALKSRLTISKDTSSNQVFLKIAS | |
| OR104 | VDTADTATYYCAQINPAWFAYWGQGTLVTVSSASTKGPSVFP | |
| | LAPSSKSTSGGTAALGCLVKDYFPEPVTVSWNSGALTSGVHT | |
| | FPAVLQSSGLYSLSSVVTVPSSSLGTQTYICNVNHKPSNTKVD | |
| | KKVEPKSCDKTHTSPPSPAPPVAGPSVFLFPPKGQPREPQVY | |
| | TLPPSRDELTKNQVSLTCLVKGFYPSDIAVEWESNGQPENNYK | |
| | TTPPVLDSDGSFFLYSKLTVDKSRWQQGNVFSCSVMHEALHN | |
| | HYTQKSLSLSPGKGGGGSDIQMTQSPSSLSASVGDRVTITCR | |
| | ASQYIYDYLNWYQQKPGKAPKLLIYAASYLQSGVPSRFSGSGS | |
| | GTDFTLTISSLOPEDFATYYCQQYHHPSTFGQGTKVEIKRGGG | |
| | GSGGGGSGGGGSEVQLLESGGGLVQPGGSLRLSCAASGFTF | |
| | RNHDMGWVRQAPGKGLEWVSAISGSGGSTYYANSVKGRFTI | |
| | SRDNSKNTLYLQMNSLRAEDTAVYYCAEPKPMDTEFDYRSPG | |
| | TLVTVSSHHHHHH | |
| Heavy chain | QVTLKESGPGILQPSQTLSLTCSFSGFSLRTSGMGVGWIRQPS | 358 |
| tetravalent molecule | GKGLEWLAHIWWDDDKRYNPALKSRLTISKDTSSNQVFLKIAS | |
| 4 | VDTADTATYYCAQINPAWFAYWGQGTLVTVSSASTKGPSVFP | |
| OR103 | LAPSSKSTSGGTAALGCLVKDYFPEPVTVSWNSGALTSGVHT | |
| | FPAVLQSSGLYSLSSVVTVPSSSLGTQTYICNVNHKPSNTKVD | |
| | KKVEPKSCDKTHTCPPCPAPPVAGPSVFLFPPKDIQMTQSPSS | |
| | LSASVGDRVTITCRASQYIYDYLNWYQQKPGKAPKLLIYAASYL | |
| | QSGVPSRFSGSGSGTDFTLTISSLQPEDFATYYCQQYHHPSTF | |
| | GQGTKVEIKRGGGGSGGGGSGGGGSEVQLLESGGGLVQPG | |
| | GSLRLSCAASGFTFRNHDMGWVRQAPGKGLEWVSAISGSGG | |
| | STYYANSVKGRFTISRDNSKNTLYLQMNSLRAEDTAVYYCAEP | |
| | KPMDTEFDYRSPGTLVTVSSHHHHHH | |
| Heavy chain bivalent | QVTLKESGPGILQPSQTLSLTCSFSGFSLRTSGMGVGWIRQPS | 359 |
| molecule 4 | GKGLEWLAHIWWDDDKRYNPALKSRLTISKDTSSNQVFLKIAS | |
| OR107 | VDTADTATYYCAQINPAWFAYWGQGTLVTVSSASTKGPSVFP | |
| | LAPSSKSTSGGTAALGCLVKDYFPEPVTVSWNSGALTSGVHT | |
| | FPAVLQSSGLYSLSSVVTVPSSSLGTQTYICNVNHKPSNTKVD | |
| | KKVEPKSCDKTHTGGGGSDIQMTQSPSSLSASVGDRVTITCR | |
| | ASQYIYDYLNWYQQKPGKAPKLLIYAASYLQSGVPSRFSGSGS | |
| | GTDFTLTISSLQPEDFATYYCQQYHHPSTFGQGTKVEIKRGGG | |
| | GSGGGGSGGGGSEVQLLESGGGLVQPGGSLRLSCAASGFTF | |
| | RNHDMGWVRQAPGKGLEWVSAISGSGGSTYYANSVKGRFTI | |
| | SRDNSKNTLYLQMNSLRAEDTAVYYCAEPKPMDTEFDYRSPG | |
| | TLVTVSSHHHHHH | |

Further exemplary E200 peptides derived from nfP2X₇ receptor

| | | |
|---|---|---|
| E200 + IgG hinge (E200 | GHNYTTRNILPGLNITSEPKSSDKTHT | 361 |
| underlined) | | |
| E200 + GS linker_IgG | GHNYTTRNILPGLNITSGSEPKSSDKTHT | 362 |
| hinge | | |
| E200 + G4S linker + IgG | GHNYTTRNILPGLNITSGGGGSEPKSSDKTHT | 363 |
| hinge | | |
| Extended E200 + _IgG | GHNYTTRNILPGLNITSTFHKTSGSGKEPKSSDKTHT | 364 |
| hinge | | |
| Extended E200 + GS | GHNYTTRNILPGLNITSTFHKTSGSGKGSEPKSSDKTHT | 365 |
| linker + _IgG hinge | | |
| Extended E200 + G4S | GHNYTTRNILPGLNITSTFHKTSGSGKGGGGSEPKSSDKTHT | 366 |
| linker + IgG hinge | | |
| E200 + IgG hinge + GS | GHNYTTRNILPGLNITSEPKSSDKTHTGS | 367 |
| linker | | |
| E200 + GS linker + IgG | GHNYTTRNILPGLNITSGSEPKSSDKTHTGS | 368 |
| hinge + GS linker | | |
| E200 + G4Slinker + IgG | GHNYTTRNILPGLNITSGGGGSEPKSSDKTHTGS | 369 |
| hinge + GS linker | | |
| Extended E200 + _IgG | GHNYTTRNILPGLNITSTFHKTSGSGKEPKSSDKTHTGS | 370 |
| hinge + GSlinker | | |
| Extended E200 + GS | GHNYTTRNILPGLNITSTFHKTSGSGKGSEPKSSDKTHTGS | 371 |
| linker + _IgG | | |
| hinge + GSlinker | | |
| Extended E200 + G4S | GHNYTTRNILPGLNITSTFHKTSGSGKGGGGSEPKSSDKTHTG | 372 |
| linker + _IgG | S | |
| hinge + GSlinker | | |
| E200 + IgG hinge + G4S | GHNYTTRNILPGLNITSEPKSSDKTHTGGGGS | 373 |
| linker | | |
| E200 + GS linker + IgG | GHNYTTRNILPGLNITSGSEPKSSDKTHTGGGGS | 374 |
| hinge + G4S linker | | |
| E200 + G4S linker + IgG | GHNYTTRNILPGLNITSGGGGSEPKSSDKTHTGGGGS | 375 |
| hinge + G4S linker | | |
| Extended E200 + IgG | GHNYTTRNILPGLNITSTFHKTSGSGKEPKSSDKTHTGGGGS | 376 |
| hinge + G4S linker | | |
| Extended E200 + GS | GHNYTTRNILPGLNITSTFHKTSGSGKGSEPKSSDKTHTGGGG | 377 |
| linker + IgG hinge + G4S | S | |
| linker | | |
| Extended E200 + G4S | GHNYTTRNILPGLNITSTFHKTSGSGKGGGGSEPKSSDKTHTG | 378 |
| linker + IgG hinge + G4S | GGGS | |
| linker | | |
| N-term extended E200 | DFPGHNYTTRNILPGLNITSEPKSSDKTHT | 379 |
| + IgG hinge | | |
| N-term extended E200 | DFPGHNYTTRNILPGLNITSGSEPKSSDKTHT | 380 |
| + GS linker + IgG hinge | | |
| N-term extended E200 | DFPGHNYTTRNILPGLNITSGGGGSEPKSSDKTHT | 381 |
| + G4S linker + IgG hinge | | |
| N and C term extended | DFPGHNYTTRNILPGLNITSTFHKTSGSGKEPKSSDKTHT | 382 |
| E200 + IgG hinge | | |
| N and C term extended | DFPGHNYTTRNILPGLNITSTFHKTSGSGKGSEPKSSDKTHT | 383 |
| E200 + GS linker + IgG | | |
| hinge | | |
| N and C term extended | DFPGHNYTTRNILPGLNITSTFHKTSGSGKGGGGSEPKSSDKT | 384 |
| E200 + G4S linker + IgG | HT | |
| hinge | | |
| N-term extended | DFPGHNYTTRNILPGLNITSEPKSSDKTHTGS | 385 |
| E200 + IgG hinge + GS | | |
| linker | | |
| N-term extended | DFPGHNYTTRNILPGLNITSGSEPKSSDKTHTGS | 386 |
| E200 + GS linker + IgG | | |
| hinge + GS linker | | |
| N-term extended | DFPGHNYTTRNILPGLNITSGGGGSEPKSSDKTHTGS | 387 |
| E200 + G4S linker + IgG | | |
| hinge + GS linker | | |
| N-term and C term | DFPGHNYTTRNILPGLNITSTFHKTSGSGKEPKSSDKTHTGS | 388 |
| extended E200 + IgG | | |
| hinge + GS linker | | |
| N-term and C term | DFPGHNYTTRNILPGLNITSTFHKTSGSGKGSEPKSSDKTHTG | 389 |
| extended E200 + GS | S | |
| linker + IgG hinge + GS | | |
| linker | | |
| N-term and C term | DFPGHNYTTRNILPGLNITSTFHKTSGSGKGGGGSEPKSSDKT | 390 |
| extended E200 + G4S | HTGS | |
| linker + IgG hinge + GS | | |
| linker | | |
| N-term extended | DFPGHNYTTRNILPGLNITSEPKSSDKTHTGGGGS | 391 |
| E200 + IgG hinge + G4S | | |
| linker | | |
| N-term extended | DFPGHNYTTRNILPGLNITSGSEPKSSDKTHTGGGGS | 392 |
| E200 + GS linker + IgG | | |
| hinge + G4S linker | | |
| N-term extended | DFPGHNYTTRNILPGLNITSGGGGSEPKSSDKTHTGGGGS | 393 |
| E200 + G4S linker + IgG | | |
| hinge + G4S linker | | |
| N-term and C term | DFPGHNYTTRNILPGLNITSTFHKTSGSGKEPKSSDKTHTGGG | 394 |
| extended E200 + IgG | GS | |
| hinge + G4S linker | | |
| N-term and C term | DFPGHNYTTRNILPGLNITSTFHKTSGSGKGSEPKSSDKTHTG | 395 |
| extended E200 + GS | GGGS | |
| linker + IgG hinge + G4S | | |
| linker | | |
| N-term and C term | DFPGHNYTTRNILPGLNITSTFHKTSGSGKGGGGSEPKSSDKT | 396 |
| extended E200 + G4S | HTGGGGS | |
| linker + IgG hinge + G4S | | |
| linker | | |
| IgG hinge + G4S linker | DKTHTSPPSPAPELLGGGGSDFPGHNYTTRNILPGLNITS | 437 |
| N-term extended E200 | | |
| (underlined) | | |
| G4S linker + IgG | GGGGSEPKSSDKTHTSPPSPAPELLGGGGS | 438 |
| hinge + N-term extended | DFPGHNYTTRNILPGLNITS | |
| E200 (underlined) | | |
| Example of CD22- | QVQLQQSGPGMVKPSQTLSLTCAISGDSVSSNSVAWNWIRQS | 439 |
| binding bridge with | PSRGLEWLGRTYYRSTWYNDYAVSMKSRITINPDTNKNQFSL | |
| E200 epitope | QLNSVTPEDTAVYYCAREVTGDLEDAFDIWGQGTMVTVSSAS | |
| (underlined) at C | TKGPSVFPLAPSSKSTSGGTAALGCLVKDYFPEPVTVSWNSG | |
| terminus of heavy | ALTSGVHTFPAVLQSSGLYSLSSVVTVPSSSLGTQTYICNVNH | |
| chain (CD22_m971-L7 | KPSNTKVDKKVEPKSCDKTHTSPPSPAPELLGGGGS | |
| clone) | DFPGHNYTTRNILPGLNITS | |
| Example of CD79B- | DIVLTQSPDSLAVSLGERATINCKASQSVDYDGDSFLNWYQQK | 440 |
| binding bridge with | PGQPPKLLIYAASNLESGVPDRFSGSGSGTDFTLTISSLQAEDV | |
| E200 epitope | AVYYCQQSNEDPLTFGGGTKVEIKRTVAAPSVFIFPPSDEQLK | |
| (underlined) at C | SGTASVVCLLNNFYPREAKVQWKVDNALQSGNSQESVTEQD | |
| terminus of light chain | SKDSTYSLSSTLTLSKADYEKHKVYACEVTHQGLSSPVTKSFN | |
| (CD79B_VL_US20210 | RGECGGGGSEPKSSDKTHTSPPSPAPELLGGGGS | |
| 145878A1_SEQ_75_C | DFPGHNYTTRNILPGLNITS | |
| D9W23) | | |

TABLE 2
Antigen binding proteins for binding dysfunctional P2X₇ receptor

| | | SEQ ID | |
|---|---|---|---|
| Clone | Region | NO: | Amino acid sequence |
| V4B11 | HCDR1 | 397 | DEDMG |
| V4A7 | | | |
| V4B11 | HCDR2 | 398 | AIDDAGDSTYYADSVKG |
| V4A7 | | | |
| V4B11 | HCDR3 | 399 | VYPYDTVFDY |
| | VH | 400 | EVQLLESGGGLVQPGGSLRLSCAASGVRISDEDMGW |
| | | | VRQAPGKGLEWVSAIDDAGDSTYYADSVKGRFTISRD |
| | | | NSKNTLYLQMNSLRAEDTAVYYCAKVYPYDTVFDYW |
| | | | GQGTLVTVSS |
| V4A7 | HCDR3 | 401 | VYPYDTVFDY |
| | VH | 402 | EVQLLESGGGLVQPGGSLRLSCAASGFRISDEDMGW |
| | | | VRQAPGKGLEWVSAIDDAGDSTYYADSVKGRFTISRD |
| | | | NSKNTLYLQMNSLRAEDTAVYYCAKVYPYDT |
| | | | VFDYWGQGTLVTVSS |
| V4A7 | HFR1 | 403 | EVQLLESGGGLVQPGGSLRLSCAASGERIS |
| V4B11 | HFR1 | 404 | EVQLLESGGGLVQPGGSLRLSCAASGVRIS |
| V4B11 | HFR2 | 405 | WVRQAPGKGLEWVS |
| V4A7 | | | |
| V4B11 | HFR3 | 406 | RFTISRDNSKNTLYLQMNSLRAEDTAVYYCAK |
| V4A7 | | | |
| V4B11 | HFR4 | 407 | WGQGTLVTVSS |
| V4A7 | | | |
| BIL03 | HCDR1 | 408 | NHDMG |
| | (Kabat) | | |
| | HCDR2 | 409 | AISGSGGSTYYANSVKG |
| | (Kabat) | | |
| | HCDR3 | 410 | PKPMDTEFDY |
| | (Kabat) | | |
| | VH | 411 | EVQLLESGGGLVQPGGSLRLSCAASGFTFRNHDMG |
| | | | WVRQAPGKGLEWVSAISGSGGSTYYANSVKGRFTIS |
| | | | RDNSKNTLYLQMNSLRAEDTAVYYCAEPKPMDTEFD |
| | | | YRSPGTLVTVSS |
| | HFR1 | 412 | EVQLLESGGGLVQPGGSLRLSCAASGFTFR |
| | (Kabat) | | |
| | HFR2 | 413 | WVRQAPGKGLEWVS |
| | (Kabat) | | |
| | HFR3 | 414 | RFTISRDNSKNTLYLQMNSLRAEDTAVYYCAE |
| | (Kabat) | | |
| | HFR4 | 415 | RSPGTLVTVSS |
| | (Kabat) | | |
| | HCDR1 | 416 | GFTFRNH |
| | (Chothia) | | |
| | HCDR2 | 417 | SGSGGS |
| | (Chothia) | | |
| | HCDR3 | 418 | PKPMDTEFDY |
| | (Chothia) | | |
| | HFR1 | 419 | EVQLLESGGGLVQPGGSLRLSCAAS |
| | (Chothia) | | |
| | HFR2 | 420 | DMGWVRQAPGKGLEWVSAI |
| | (Chothia) | | |
| | HFR3 | 421 | TYYANSVKGRFTISRDNSKNTLYLQMNSLRAEDTAVY |
| | (Chothia) | | YCAE |
| | HFR4 | 422 | RSPGTLVTVSS |
| | (Chothia) | | |
| | HCDR1 | 423 | GFTFRNHD |
| | (IMGT) | | |
| | HCDR2 | 424 | ISGSGGST |
| | (IMGT) | | |
| | HCDR3 | 425 | AEPKPMDTEFDY |
| | (IMGT) | | |
| | HFR1 | 426 | EVQLLESGGGLVQPGGSLRLSCAAS |
| | (IMGT) | | |
| | HFR2 | 427 | MGWVRQAPGKGLEWVSA |
| | (IMGT) | | |
| | HFR3 | 428 | YYANSVKGRFTISRDNSKNTLYLQMNSLRAEDTAVYY |
| | (IMGT) | | C |
| | HFR4 | 429 | RSPGTLVTVSS |
| | (IMGT) | | |
| | HCDR1 | 430 | NHDMG |
| | (Martin) | | |
| | HCDR2 | 431 | AISGSGGSTYYANSVKG |
| | (Martin) | | |
| | HCDR3 | 432 | PKPMDTEFDY |
| | (Martin) | | |
| | HFR1 | 433 | EVQLLESGGGLVQPGGSLRLSCAASGFTFR |
| | (Martin) | | |
| | HFR2 | 434 | WVRQAPGKGLEWVS |
| | (Martin) | | |
| | HFR3 | 435 | RFTISRDNSKNTLYLQMNSLRAEDTAVYYCAE |
| | (v) | | |
| | HFR4 | 436 | RSPGTLVTVSS |
| | (Martin) | | |

### DETAILED DESCRIPTION OF THE EMBODIMENTS

Reference will now be made in detail to certain embodiments of the invention. While the invention will be described in conjunction with the embodiments, it will be understood that the intention is not to limit the invention to those embodiments. On the contrary, the invention is intended to cover all alternatives, modifications, and equivalents, which may be included within the scope of the present invention as defined by the claims.

One skilled in the art will recognise many methods and materials similar or equivalent to those described herein, which could be used in the practice of the present invention. The present invention is in no way limited to the methods and materials described.

It will be understood that the invention disclosed and defined in this specification extends to all alternative combinations of two or more of the individual features mentioned or evident from the text or drawings. All of these different combinations constitute various alternative aspects of the invention.

All of the patents and publications referred to herein are incorporated by reference in their entirety.

The present invention seeks to address one or more of the deficiencies of the prior art and is based on the recognition by the inventors that it is possible to exploit the cancer-specific expression of tumour-specific antigens, such as a dysfunctional P2X₇ receptor, to:
a) redirect native immune cells, for example adaptive or innate immune cells such as T cells, NK cells, macrophages, monocytes and granulocytes which are redirected to a tumour-specific antigen, such as a dysfunctional P2X₇ receptor; immune cells are redirected to cancerous cells by modified proteins such as antibodies like IgG1 antibodies with enhanced Fc receptor binding capabilities, e.g. SDIE modification. Further, bispecific or trispecific fusion proteins also regarded as polyfunctional fusion proteins, recruiting immune cells via immune receptors such as CD3 on T cells, CD16 on NK cells or CD32 or CD64 on macrophages/monocytes and granulocytes. The format of the fusion may vary from full-size antibodies to bispecific T cell engagers and variations thereof.
b) provide a tunable, “switchable” approach to targeted cell killing in a variety of settings that minimise on-target, off-tumour effects;
c) minimise aberrant immune responses to adaptor molecules; and
d) amplify the immune response by bringing a target cell and immune cell into proximity and activate the immune cell for the immune cell to act on the target cell.

The invention provides a new treatment modality comprising a first component and second component.

The first component is the administration (or expression) of an antigen binding protein (also referred to herein as “ORCHESTRATION” (OR) molecule) e.g. a bispecific fusion protein that binds to a tumour-specific antigen (preferably nfP2X₇ receptor, more preferably the nfP2X₇ E200 epitope) and a second antigen binding domain that binds to an antigen on an immune cell, preferably that antigen is an activating receptor, thereby recruiting immune cells to the tumour cell. Examples of suitable immune antigens for binding by the OR molecules are provided further herein. The use of the OR molecules on their own has a potent antitumoural effect mediated by the engaged immune cell, e.g. a T cell via a bispecific T cell recruiting fusion protein or an NK cell via a bispecific NK cell recruiting fusion protein. As used herein the antigen binding protein defined herein may also be referred to as an OR molecule.

The second component is the administration (or expression) of a bridging molecule that can redirect immune cells in the context of OR molecules. The second component, the bridging molecule, might bind to other cell surface molecules, such as tumour-associated antigens, e.g. CD19, CD20, CD33, intracellularly processed proteins that are presented as peptides of various length via MHC I and II or via any other mechanism of accessible surface antigen exposure. The design of the bridging molecule incorporates a tumour-specific epitope moiety, e.g. a nfP2X₇ derived peptide antigen (referred to herein as dysfunctional P2X₇ receptor epitope moiety). The enrichment of bridging molecules on cancer cells leads to the increase of nfP2X₇ target antigens on all cells that are targeted by the bridging molecule, e.g. a CD19 Fab based bridging molecule may bind to CD19 positive cancer cells and physiological CD19 expressing B-lineage derived cells. The OR molecules may then in the following step enrich at cancer cells via binding specifically to cancer cells expressing nfP2X₇ as well as to the dysfunctional P2X₇ receptor epitope moiety (e.g. nfP2X₇ E200 derived peptide antigen) that is part of the bridging molecule.

The bridging molecules in general may redirect natively present immune cells, e.g. T cells, NK cells, macrophages, monocytes etc, towards cancer cells via targeting of tumour-associated antigens and as well redirect CAR expressing cells with the purpose of recruitment of the native immune system and, if present, CAR expressing cells at the same time. By the combination of the OR molecules, the CAR expressing cells and the bridging molecules, all different sorts of effector cells are redirected to target the cancer cells specifically and in targeting tumour-associated and/or tumour-specific antigens. The orchestration of the native present immune system and the artificial transgenic CAR expressing cell subsets, will significantly amplify the anti-cancer response and result in improved cancer control.

The cell surface molecules targeted by the bridging molecules may be associated with cancer/a tumour (such as tumour-associated antigens expressed on the surface of cancer cells). In further embodiments, the targeted cell surface molecules may be associated with an infection, or associated with any other disease (including autoimmune diseases). In such embodiments, the molecules may be cell surface antigens associated with the disease or may include peptide/HLA complexes presented on cells. In certain examples the molecule may comprise CD19 in B-lineage malignancy. The molecule may comprise targeted peptides related to cancer-specific proteins (genetic aberrations such as cancer testis antigens and others which are specific to the cancer patient). The molecule may be a peptide/HLA complex comprising peptides derived from an infectious agent e.g. of viral, bacterial, protozoan, virion, prion and fungal origin. The molecule may be a peptide/HLA complex comprising peptides associated with an autoimmune disease (e.g., Sm peptides associated with lupus). Further the targeted antigens may be processed sugar molecules (GD2) or lipids.

Further, the present invention provides several advantages over existing antibody or bispecific fusion protein-based therapies as the primary targeting is tumour specific (the first antigen binding domain of the antigen binding protein or ORN molecule binds tumour-specific antigens) and not tumour associated antigens. Only by introduction of the bridging molecules does tumour-associated antigen targeting would come into play.

While antibody therapy today has been limited to directly target tumour-associated antigens or to use a non-functional antibody redirecting antibodies to tumour-associated antigens, the dual principle the Orchestration technology provides cancer specific targeting as a maintenance therapy with no direct toxicity to healthy tissue plus the ability to boost the effector function of the OR molecule function by introducing bridging molecules targeted to alternative target antigens. The nature and origin of potential target antigens is outlined above.

In another example, adaptor molecules include small peptide tags to redirect standard scFv-based adaptor CARs. However, concerns again have been raised with respect to the immunogenic potential of the small peptide tags included in the adaptor molecules, particularly in the case where the tag comprises non-human sequences, or sequences derived from human nuclear proteins.

In contrast to the approaches of the prior art, the present invention takes advantage of both the specificity of the OR molecules that bind dysfunctional P2X₇ and of the unique properties of epitopes derived from dysfunctional P2X₇ receptor.

The present invention provides an advantage over existing mono-antigen directed antibody and fusion protein based therapy in that it:
makes use of OR molecules that are functional (i.e., there is no requirement for the use of a switching molecule to activate the native present immune cells);
utilises cancer specific immune cell recruitment and thus will not bind to healthy cells since dysfunctional P2X₇ receptor is only exposed on the surface of cancer cells;
comprises non-immunogenic, and naturally occurring human epitopes on the bridging molecule in the form of epitopes derived from dysfunctional P2X₇ receptor.

Thus the present invention provides a new concept and approach in the use of OR molecules alongside with adaptor/bridging molecules for cancer specific targeting as well as cancer associated targeting.

In more detail, the specificity of the OR molecules that bind dysfunctional nfP2X₇ receptor (also referred to herein as nfP2X₇ CAR) results from the fact that dysfunctional P2X₇ is only exposed on the surface of transformed cells. Further, by including an epitope from nfP2X₇ in a bridging molecule, OR molecule-mediated recognition can be broadened to include any target antigen of interest via the corresponding bridging molecule. The nfP2X₇ targeted OR molecules solely recognise the dysfunctional P2X₇ receptor, e.g. the E200 (or E300 or E200-300 composite) epitope, however the use of a bridging molecule facilitates unlimited targeting by means of any accessible recognition site expressed on the cell surface, e.g. an nfP2X₇ recruiting OR molecule can be additionally directed (or redirected) to bind to CD19 positive cells through the use of a bridging molecule that comprises a targeting moiety for binding CD19 on a cell surface, and an E200 epitope moiety from nfP2X₇.

P2X₇ is a human receptor protein that is commonly expressed in human tissue, particularly immune and neural cells. There is no reported or registered case of autoimmune response raised against P2X₇ receptors. Exemplary targeted epitopes such as E200, E300 and E200/E300 are not genetically defined but only result from a conformational change of the tertiary structure of P2X₇. Thus, these are non-immunogenic peptide sequences that are an unaltered part of the P2X₇ sequence. Only under artificial conditions using adjuvants and conjugates can immune responses be produced against the target.

An advantage of the non-immunogenic recognition sites, e.g. the peptide sequence of E200 or E300 or the composite peptide E200-300, in the bridging molecule facilitates long-term application of bridging molecules with various specificities without induction of neutralising antibodies and/or T cell mediated rejection of cells. This represents a significant advantage over the design of bridging/adaptor molecules described in the prior art.

Moreover, nfP2X₇ CARs only target cancer tissue specifically, and therefore the approach of the present invention presents minimal risk of “on-target, off-tumour” effects and damage to healthy tissue through off-target binding of the OR molecules which are cancer specific. The present invention therefore exploits the specificity of nfP2X₇ targeted OR molecules in two ways: firstly by relying on the fact that nfP2X₇ OR molecules only target nfP2X₇ expressing cells (cancer cells only) and secondly, by relying on the fact that bridging molecules, engineered to express the nfP2X₇ E200 derived peptide epitope moiety, can be used to redirect immune cells via OR molecules towards other tumour-associated and/or specific target antigens in a switchable, tunable manner.

Thus, the use of the bridging molecules of the invention allows for the redirection of immune cells recruited by OR molecules for the targeting of any surface expressed target antigen.

A particular advantage of the present approach is that the targeting is limited to the time period during which the bridging molecule therapeutic in vivo is persistent in circulation. This means that any toxicity arising from the “on-target, off-tumour” expression of the target antigen in healthy tissue is minimised. This is because once the bridging molecule has been cleared from the body, nfP2X₇ targeted OR molecules recruited cells are again only capable of tumour-specifically targeting nfP2X₇. Further, the administration of OR molecules can be initiated, terminated and reinitiated at any time. In other words, as the targeting of target antigens other than nfP2X₇ is bridging molecule-dependent, the targeting can be regulated by the application of bridging molecules. This facilitates an easy to implement approach for “switching-on” and “switching-off” the targeting of cancer cells via the antigen bound by the targeting moiety of the bridging molecules such that the OR molecule redirected immune cells may be transiently directed to cancer cells via antigens other than nfP2X₇. Further, the length of time during which the OR molecule redirected immune cells are redirected to other cancer antigens can be modulated by the time for which the bridging molecules are administered to a patient in need. Once the OR molecule application has been terminated the cancer-specific targeting of OR molecule engaged immune cells would be terminated as well according to the pharmacokinetics and dynamics of this particular OR molecule or the OR molecules applied to the patient.

It should therefore be clear that the present invention finds application in a variety of settings. For example, in the context of oncology treatment, the present invention allows for the use of a single class of immune effector cell recruiting OR molecule (i.e., for binding dysfunctional P2X₇ receptor present on cancer cells) or in combination with bridging molecules to target multiple antigens present on the cancer cells. More specifically, using a bridging molecule that comprises, for example a targeting moiety for binding CD19, the OR molecule recruited immune cells can be targeted to the cancer cells at both the tumour-specific antigen (e.g. dysfunctional P2X₇ receptor) and the tumour-associated antigen CD19. This maximises the likelihood of the cancer cell being recognized and eliminated because it is being targeted at multiple sites. Moreover, it should be evident that the use of multiple bridging molecules, or bridging molecules comprising more than one targeting moiety, facilitates the “painting” of the cancer cell surface with CAR T cells. In other words, the invention provides for the use of a variety of different bridging molecules, each comprising epitopes for being bound by one OR molecule or a panel of OR molecules with the capability of recruiting different immune effector cells specifically to cancer directly via nfP2X₇ targeting as well as by redirecting via the additionally introduced BRiDGE molecules. Thus the OR molecules comprise the ability to engage immune effector cells to be directed or redirected to bind to cancer cells via multiple cancer antigens (e.g. nfP2X₇, CD19, CD20, CD22 etc.) at the same time. In this way, the cancer cells can be targeted and bound by the OR molecule recruited immune effector cells by multiple sites, in consequence increasing the anticancer effector function and in the latest instance elimination of cancer cells.

This approach is also particularly useful in the case of cancers that express low levels of dysfunctional P2X₇ receptor, such as Burkitt's lymphoma or subcategories of solid tumours arising from various epithelial, mesenchymal, neural or germinal origins. Another example of a low-expressing cancer cell type may be the triple negative breast cancer (e.g. MDA-MB-231 cell line). Other examples include solid tumour tissues tested in tissue arrays from PDX models, several of which show lower receptor expression than other cancers. Such examples include but are not limited to neuroblastoma, colorectal cancer, lung cancer, breast cancer or brain cancer.

In further examples, the invention finds application in the context of preventing or minimising the severity of an infection with a pathogen (preferably an intracellular pathogen). While not limited to an oncology setting, this may be particularly useful in the treatment of patients receiving cancer therapy and who are immunocompromised (and therefore susceptible to infection with opportunistic or other pathogens). Thus, a patient who has received (or is continuing to receive) a treatment with OR molecules that bind dysfunctional P2X₇ receptor, can simultaneously be administered a bridging molecule that facilitates the redirection of the immune effector cells to cells that present peptides from an infectious agent on MHC molecules on their cell surface. In other words, the invention provides a platform for simultaneous or sequential treatment of cancer and an infectious disease as well as autoimmune disease.

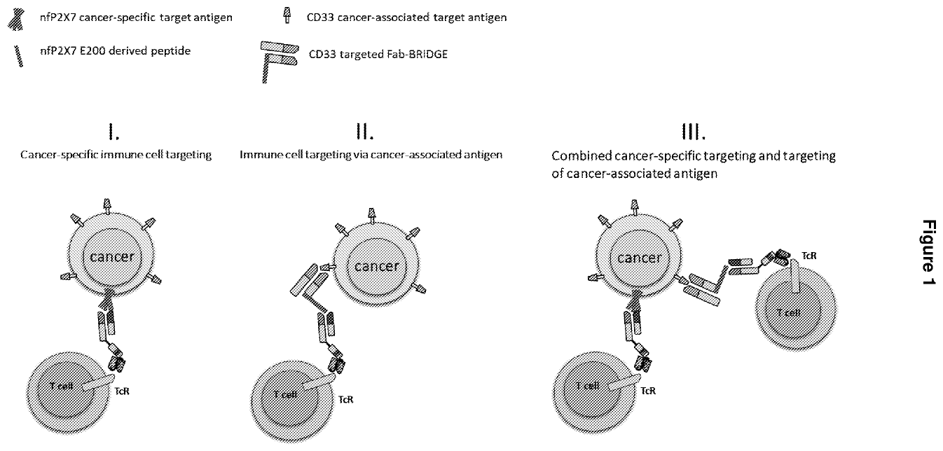

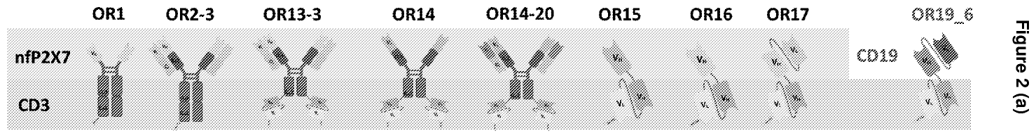

The basic principle as well as the engagement of nfPX7 CAR expressing effector cells via nfP2X₇ E200-derived peptide tagged bridging molecules and the different formats of bridging molecules is illustrated and outlined in FIGS. 1 to 3.

Using nfP2X₇R OR molecules without the presence of bridging molecules, recruited effector cells exhibit cancer-specific targeting (FIG. 1: scenario I).

In order to broaden the applicability to nfP2X₇ functionally negative cancers (very low or negative for nfP2X₇) OR molecule recruited immune effector cells may be redirected to cancer cells via bridging molecules targeting cancer-associated antigens as illustrated for CD33 or cancer-specific antigens via TcR-like mAbs. The specificity of the bridging molecules is unlimited meaning any surface expressed target antigen or presented antigen in the context of MHC peptide presentation (class I and II) via TcR-like mAb or ligands may engage the nfP2X₇ CAR expressing effector cells in the same mode of action (FIG. 1: scenario II).

In most cases, the dual-function of the OR molecule recruiting immune effector cells is utilised (FIG. 1: scenario III). It is a combination of scenario I. and II, which means that OR molecules recruit and engage immune effector cells directly to cancer cells via nfP2X₇ expressed on the cancer cells and additionally get recruited to the cancer cells via bridging molecules targeting cancer-associated antigens as illustrated for CD33 or cancer-specific antigens via TcR-like mAbs.

### Definitions

Unless defined otherwise, technical and scientific terms used herein have the same meaning as commonly understood by one of ordinary skill in the art to which this invention belongs.

For purposes of interpreting this specification, the following definitions will generally apply and whenever appropriate, terms used in the singular will also include the plural and vice versa.

“Purinergic receptor” generally refers to a receptor that uses a purine (such as ATP) as a ligand.

“P2X₇ receptor” generally refers to a purinergic receptor formed from three protein subunits or monomers, with at least one of the monomers having an amino acid sequence substantially as shown in SEQ ID NO: 1 below:

SEQ ID NO: 1

MPACCSCSDVFQYETNKVTRIQSMNYGTIKWFFHVIIFSYVCFALVSDKLYQRKEPVIS SVHTKVKGIAEVKEEIVENGVKKLVHSVFDTADYTFPLQGNSFFVMTNFLKTEGQEQRL CPEYPTRRTLCSSDRGCKKGWMDPQSKGIQTGRCVVYEGNQKTCEVSAWCPIEAVE EAPRPALLNSAENFTVLIKNNIDFPGHNYTTRNILPGLNITCTFHKTQNPQCPIFRLGDIF RETGDNFSDVAIQGGIMGIEIYWDCNLDRWFHHCRPKYSFRRLDDKTTNVSLYPGYNF RYAKYYKENNVEKRTLIKVFGIRFDILVFGTGGKFDIIQLVVYIGSTLSYFGLAAVFIDFLID TYSSNCCRSHIYPWCKCCQPCVVNEYYYRKKCESIVEPKPTLKYVSFVDESHIRMVNQ QLLGRSLQDVKGQEVPRPAMDFTDLSRLPLALHDTPPIPGQPEEIQLLRKEATPRSRD SPVWCQCGSCLPSQLPESHRCLEELCCRKKPGACITTSELFRKLVLSRHVLQFLLLYQ EPLLALDVDSTNSRLRHCAYRCYATWRFGSQDMADFAILPSCCRWRIRKEFPKSEGQ YSGFKSPY

To the extent that P2X₇ receptor is formed from three monomers, it is a “trimer” or “trimeric”. “P2X₇ receptor” encompasses naturally occurring variants of P2X₇ receptor, e.g., wherein the P2X₇ monomers are splice variants, allelic variants, SNPs and isoforms including naturally-occurring truncated or secreted forms of the monomers forming the P2X₇ receptor (e.g., a form consisting of the extracellular domain sequence or truncated form of it), naturally-occurring variant forms (e.g., alternatively spliced forms) and naturally-occurring allelic variants. In certain embodiments of the invention, the native sequence P2X₇ monomeric polypeptides disclosed herein are mature or full-length native sequence polypeptides comprising the full-length amino acids sequence shown in SEQ ID NO: 1. In certain embodiments the P2X₇ receptor may have an amino acid sequence that is modified, for example various of the amino acids in the sequence shown in SEQ ID NO: 1 may be substituted, deleted, or a residue may be inserted.

“Functional P2X₇ receptor” generally refers to a form of the P2X₇ receptor having three intact binding sites or clefts for binding to ATP. When bound to ATP, the functional receptor forms a non-selective sodium/calcium channel that converts to a pore-like structure that enables the ingress of calcium ions and molecules of up to 900 Da into the cytosol, one consequence of which may be induction of programmed cell death. In normal homeostasis, expression of functional P2X₇ receptors is generally limited to cells that undergo programmed cell death such as thymocytes, dendritic cells, lymphocytes, macrophages and monocytes. There may also be some expression of functional P2X₇ receptors on erythrocytes and other cell types.

“Dysfunctional P2X₇ receptor” (also called “non-functional” or (nf) P2X₇) is a P2X₇ receptor that has an impaired response to ATP such that it is unable to form an apoptotic pore under physiological conditions. A dysfunctional P2X₇ receptor or (nfP2X₇ receptor) generally refers to a form of a P2X₇ receptor having a conformation, distinct from functional P2X₇, whereby the receptor is unable to form an apoptotic pore, but which is still able to operate as a non-selective channel through the maintenance of a single functional ATP binding site located between adjacent monomers. One example arises where one or more of the monomers has a cis isomerisation at Pro210 (according to SEQ ID NO: 1). The isomerisation may arise from any molecular event that leads to misfolding of the monomer, including for example, mutation of monomer primary sequence or abnormal post translational processing. One consequence of the isomerisation is that the receptor is unable to bind to ATP at one, or more particularly two, ATP binding sites on the trimer and as a consequence not be able to extend the opening of the channel. In the circumstances, the receptor cannot form a pore and this limits the extent to which calcium ions may enter the cytosol. Dysfunctional P2X₇ receptors are expressed on a wide range of epithelial and haematopoietic cancers. As used herein, the term “dysfunctional P2X₇ receptors” may be used interchangeably with the term “non-functional P2X₇ receptors” or “nfP2X₇ receptors”.

“Cancer associated-P2X₇ receptors” are generally P2X₇ receptors that are found on cancer cells (including, pre-neoplastic, neoplastic, malignant, benign or metastatic cells), but not on non-cancer or normal cells.

“E200 epitope” generally refers to an epitope having the sequence GHNYTTNILPGLNITC and variants thereof (e.g. SEQ ID NOs: 2-11, 15-30, 168, 361-396, 437 and 438).

“E300 epitope” generally refers to an epitope having the sequence KYYKENNVEKRTLIK and variants thereof (SEQ ID NOs: 12 and 13).

A “composite epitope” generally refers to an epitope that is formed from the juxtaposition of the E200 and E300 epitopes or parts of these epitopes. An example of a composite epitope comprising E200 and E300 epitopes is GHNYTTRNILPGAGAKYYKENNVEK (SEQ ID NO: 14).

“Antibodies” or “immunoglobulins” or “Igs” are gamma globulin proteins that are found in blood, or other bodily fluids of vertebrates that function in the immune system to bind antigen, hence identifying and/or neutralising foreign objects.

Antibodies are generally a heterotetrameric glycoprotein composed of two identical light (L) chains and two identical heavy (H) chains. Each L chain is linked to a H chain by one covalent disulfide bond. The two H chains are linked to each other by one or more disulfide bonds depending on the H chain isotype. Each H and L chain also has regularly spaced intrachain disulfide BRiDGEs.

H and L chains define specific Ig domains. More particularly, each H chain has at the N-terminus, a variable domain (V_{H}) followed by three constant domains (CH) for each of the α and γ chains and four CH domains for μ and ε isotypes. Each L chain has at the N-terminus, a variable domain (V_{L}) followed by a constant domain (CL) at its other end. The V_{L} is aligned with the V_{H} and the C_{L} is aligned with the first constant domain of the heavy chain (CH1).

Antibodies can be assigned to different classes or isotypes. There are five classes of immunoglobulins: IgA, IgD, IgE, IgG, and IgM, having heavy chains designated α, δ, ε, γ, and μ, respectively. The γ and α classes are further divided into subclasses on the basis of relatively minor differences in ¾ sequence and function, e.g., humans express the following subclasses: IgG1, IgG2, IgG3, IgG4, IgAI, and IgA2. The L chain from any vertebrate species can be assigned to one of two clearly distinct types, called kappa and lambda, based on the amino acid sequences of their constant domains.

The constant domain includes the Fc portion that comprises the carboxy-terminal portions of both H chains held together by disulfides. The effector functions of antibodies such as ADCC are determined by sequences in the Fc region, which region is also the part recognised by Fc receptors (FcR) found on certain types of cells.

The pairing of a V_{H} and V_{L} together forms a “variable region” or “variable domain” including the amino-terminal domains of the heavy or light chain of the antibody. The variable domain of the heavy chain may be referred to as “V_{H}.” The variable domain of the light chain may be referred to as “V_{L}.” The V domain contains an “antigen binding site” that affects antigen binding and defines specificity of a particular antibody for its particular antigen. V regions span about 110 amino acid residues and consist of relatively invariant stretches called framework regions (FRs) (generally about 4) of 15-30 amino acids separated by shorter regions of extreme variability called “hypervariable regions” (generally about 3) that are each generally 9-12 amino acids long. The FRs largely adopt a β-sheet configuration and the hypervariable regions form loops connecting, and in some cases forming part of, the β-sheet structure.

“Hypervariable region” refers to the regions of an antibody variable domain that are hypervariable in sequence and/or form structurally defined loops. Generally, antibodies comprise six hypervariable regions; three in the V_{H} (H1, H2, H3), and three in the V_{L} (L1, L2, L3).

For the purposes for the present disclosure, the term “antibody” includes a protein capable of specifically binding to one or a few closely related antigens by virtue of an antigen binding domain contained within a Fv. This term includes four chain antibodies (e.g., two light chains and two heavy chains), recombinant or modified antibodies (e.g., chimeric antibodies, humanised antibodies, human antibodies, CDR-grafted antibodies, primatised antibodies, de-immunised antibodies, synhumanised antibodies, half-antibodies, bispecific antibodies).

An antibody generally comprises constant domains, which can be arranged into a constant region or constant fragment or fragment crystallisable (Fc). Exemplary forms of antibodies comprise a four-chain structure as their basic unit. Full-length antibodies comprise two heavy chains (˜50 to 70 kD) covalently linked and two light chains (˜23 kDa each). A light chain generally comprises a variable region (if present) and a constant domain and in mammals is either a κ light chain or a λ light chain. A heavy chain generally comprises a variable region and one or two constant domain(s) linked by a hinge region to additional constant domain(s). Heavy chains of mammals are of one of the following types α, δ, ε, γ, or μ. Each light chain is also covalently linked to one of the heavy chains. For example, the two heavy chains and the heavy and light chains are held together by inter-chain disulfide bonds and by non-covalent interactions. The number of inter-chain disulfide bonds can vary among different types of antibodies. Each chain has an N-terminal variable region (V_{H} or V_{L} wherein each are ˜110 amino acids in length) and one or more constant domains at the C-terminus. The constant domain of the light chain (C_{L} which is ˜110 amino acids in length) is aligned with and disulfide bonded to the first constant domain of the heavy chain (C_{H1} which is 330 to 440 amino acids in length). The light chain variable region is aligned with the variable region of the heavy chain. The antibody heavy chain can comprise 2 or more additional C_{H} domains (such as, C_{H2}, C_{H3} and the like) and can comprise a hinge region between the C_{H1} and C_{H2} constant domains. Antibodies can be of any type (e.g., IgG, IgE, IgM, IgD, IgA, and IgY), class (e.g., IgG₁, IgG₂, IgG₃, IgG₄, IgA₁ and IgA₂) or subclass. In one example, the antibody is a murine (mouse or rat) antibody or a primate (such as, human) antibody. In one example the antibody heavy chain is missing a C-terminal lysine residue. In one example, the antibody is humanised, synhumanised, chimeric, CDR-grafted or deimmunised.

The terms “full-length antibody”, “intact antibody” or “whole antibody” are used interchangeably to refer to an antibody in its substantially intact form, as opposed to an antigen binding fragment of an antibody. Specifically, whole antibodies include those with heavy and light chains including an Fc region. The constant domains may be wild-type sequence constant domains (e.g., human wild-type sequence constant domains) or amino acid sequence variants thereof.

As used herein, “variable region” refers to the portions of the light and/or heavy chains of an antibody as defined herein that is capable of specifically binding to an antigen and, includes amino acid sequences of complementarity determining regions (CDRs); i.e., CDR₁, CDR₂, and CDR₃, and framework regions (FRs). For example, the variable region comprises three or four FRs (e.g., FR₁, FR₂, FR₃ and optionally FR₄) together with three CDRs. VH refers to the variable region of the heavy chain. V_{L} refers to the variable region of the light chain.

As used herein, the term “complementarity determining regions” (syn. CDRs; i.e., CDR₁, CDR₂, and CDR₃) refers to the amino acid residues of an antibody variable region the presence of which are major contributors to specific antigen binding. Each variable region domain (V_{H} or V_{L}) typically has three CDRs identified as CDR₁, CDR₂ and CDR₃. The CDRs of V_{H} are also referred to herein as CDR H₁, CDR H₂ and CDR H₃, respectively, wherein CDR H₁ corresponds to CDR 1 of V_{H}, CDR H₂ corresponds to CDR 2 of V_{H} and CDR H₃ corresponds to CDR 3 of V_{H}. Likewise, the CDRs of V_{L} are referred to herein as CDR L₁, CDR L₂ and CDR L₃, respectively, wherein CDR L₁ corresponds to CDR 1 of V_{L}, CDR L₂ corresponds to CDR 2 of V_{L} and CDR L₃ corresponds to CDR 3 of V_{L}. In one example, the amino acid positions assigned to CDRs and FRs are defined according to Kabat Sequences of Proteins of Immunological Interest, National Institutes of Health, Bethesda, Md., 1987 and 1991 (also referred to herein as “the Kabat numbering system”). In another example, the amino acid positions assigned to CDRs and FRs are defined according to the Enhanced Chothia Numbering Scheme (http://www.bioinfo.org.uk/mdex.html). The present invention is not limited to FRs and CDRs as defined by the Kabat numbering system, but includes all numbering systems, including the canonical numbering system or of Chothia and Lesk J. Mol. Biol. 196: 901-917, 1987; Chothia et al., Nature 342: 877-883, 1989; and/or Al-Lazikani et al., J. Mol. Biol. 273: 927-948, 1997; the numbering system of Honnegher and Plükthun J. Mol. Biol. 309: 657-670, 2001; or the IMGT system discussed in Giudicelli et al., Nucleic Acids Res. 25: 206-211 1997.

In one example, the CDRs are defined according to the Kabat numbering system. Optionally, heavy chain CDR₂ according to the Kabat numbering system does not comprise the five C-terminal amino acids listed herein or any one or more of those amino acids are substituted with another naturally-occurring amino acid. In this regard, Padlan et al., FASEB J., 9: 133-139, 1995 established that the five C-terminal amino acids of heavy chain CDR₂ are not generally involved in antigen binding.

“Framework regions” (FRs) are those variable region residues other than the CDR residues. The FRs of VH are also referred to herein as FR H₁, FR H₂, FR H₃ and FR H₄, respectively, wherein FR H₁ corresponds to FR 1 of V_{H}, FR H₂ corresponds to FR 2 of V_{H}, FR H₃ corresponds to FR 3 of V_{H} and FR H₄ corresponds to FR 4 of V_{H}. Likewise, the FRs of V_{L} are referred to herein as FR L₁, FR L₂, FR L₃ and FR L₄, respectively, wherein FR L₁ corresponds to FR 1 of V_{L}, FR L₂ corresponds to FR 2 of V_{L}, FR L₃ corresponds to FR 3 of V_{L} and FR L₄ corresponds to FR 4 of V_{L}.

“Framework” or “FR” residues are those variable domain residues other than the hypervariable region residues herein defined.

An “antigen binding domain” generally refers to a molecule that includes at least the hypervariable and framework regions that are required for imparting antigen binding function to a V domain. An antigen binding protein or antigen binding domain may be in the form of an antibody or an antibody fragment, such as a mAb, single domain (SD)-mAb, dAb, Fab, SD-Fab, Fd, SD-Fv, Fv, F(ab′)2 or scFv.

An “intact” or “whole” antibody is one that comprises an antigen-binding site as well as a C_{L} and at least heavy chain constant domains, CH1, CH2 and CH3. The constant domains may be native sequence constant domains (e.g. human native sequence constant domains) or amino acid sequence variant thereof.

“Whole antibody fragments including a variable domain” include SD-mAb, Fab, Fab′, F(ab′)2, and Fv fragments; diabodies; linear antibodies, single-chain antibody molecules; and multi-specific antibodies formed from antibody fragments.

The “Fab fragment” consists of an entire L chain along with the variable region domain of the H chain (V_{H}), and the first constant domain of one heavy chain (CH1). Each Fab fragment is monovalent with respect to antigen binding, i.e., it has a single antigen-binding site.

A “Fab′ fragment” differs from Fab fragments by having additional few residues at the carboxy terminus of the CH1 domain including one or more cysteines from the antibody hinge region. Fab′-SH is the designation herein for Fab′ in which the cysteine residue(s) of the constant domains bear a free thiol group.

A “F(ab′)2 fragment” roughly corresponds to two disulphide linked Fab fragments having divalent antigen-binding activity and is still capable of cross-linking antigen.

An “Fv” is the minimum antibody fragment that contains a complete antigen-recognition and binding site. This fragment consists of a dimer of one heavy and one light chain variable region domain in tight, non-covalent association.

In a single-chain Fv (scFv) species, one heavy and one light chain variable domain can be covalently linked by a flexible peptide linker such that the light and heavy chains can associate in a “dimeric” structure analogous to that in a two-chain Fv species. From the folding of these two domains emanate six hypervariable loops (3 loops each from the H and L chain) that contribute the amino acid residues for antigen binding and confer antigen binding specificity to the antibody.

“Single-chain Fv” also abbreviated as “sFv” or “scFv” are antibody fragments that comprise the V_{H} and V_{L} antibody domains connected to form a single polypeptide chain. Preferably, the scFv polypeptide further comprises a polypeptide linker between the V_{H} and V_{L} domains that enables the scFv to form the desired structure for antigen binding.

A “single variable domain” is half of an Fv (comprising only three CDRs specific for an antigen) that has the ability to recognise and bind antigen, although generally at a lower affinity than the entire binding site.

“Diabodies” refers to antibody fragments with two antigen-binding sites, which fragments comprise a heavy-chain variable domain (V_{H}) connected to a light-chain variable domain (V_{L}) in the same polypeptide chain (V_{H}-V_{L}). The small antibody fragments are prepared by constructing sFv fragments (see preceding paragraph) with short linkers (about 5-10 residues) between the V_{H} and V_{L} domains such that interchain but not intra-chain pairing of the V domains is achieved, resulting in a bivalent fragment, i.e., a fragment having two antigen-binding sites.

Diabodies may be bivalent or bispecific. Bispecific diabodies are heterodimers of two “crossover” sFv fragments in which the V_{H} and V_{L} domains of the two antibodies are present on different polypeptide chains. Triabodies and tetrabodies are also generally known in the art.

An “isolated antibody” is one that has been identified and separated and/or recovered from a component of its pre-existing environment. Contaminant components are materials that would interfere with therapeutic uses for the antibody, and may include enzymes, hormones, and other proteinaceous or non-proteinaceous solutes.

A “human antibody” refers to an antibody that possesses an amino acid sequence that corresponds to that of an antibody produced by a human. Human antibodies can be produced using various techniques known in the art, including phage—display libraries. Human antibodies can be prepared by administering the antigen to a transgenic animal that has been modified to produce such antibodies in response to antigenic challenge, but whose endogenous loci have been disabled.

“Humanised” forms of non-human (e.g., rodent) antibodies are chimeric antibodies that contain minimal sequence derived from the non-human antibody. For the most part, humanised antibodies are human immunoglobulins (recipient antibody) in which residues from a hypervariable region of the recipient are replaced by residues from a hypervariable region of a non-human species (donor antibody) such as mouse, rat, rabbit or non-human primate having the desired antibody specificity, affinity, and capability. In some instances, framework region (FR) residues of the human immunoglobulin are replaced by corresponding non-human residues. Furthermore, humanised antibodies may comprise residues that are not found in the recipient antibody or in the donor antibody. These modifications are made to further refine antibody performance. In general, the humanised antibody will comprise substantially all of at least one, and typically two, variable domains, in which all or substantially all of the hypervariable loops correspond to those of a non-human immunoglobulin and all or substantially all of the FRs are those of a human immunoglobulin sequence. The humanised antibody optionally also will comprise at least a portion of an immunoglobulin constant region (Fc), typically that of a human immunoglobulin.

“Monoclonal antibody” refers to an antibody obtained from a population of substantially homogeneous antibodies, i.e., the individual antibodies comprising the population are identical except for possible naturally occurring mutations that may be present in minor amounts. Monoclonal antibodies are highly specific, being directed against a single antigenic site or determinant on the antigen. In addition to their specificity, the monoclonal antibodies are advantageous in that they may be synthesised uncontaminated by other antibodies. Monoclonal antibodies may be prepared by the hybridoma methodology. The “monoclonal antibodies” may also be isolated from phage antibody libraries using molecular engineering techniques.

The term “anti-P2X₇ receptor antibody” or “an antibody that binds to P2X₇ receptor” refers to an antibody that is capable of binding P2X₇ receptor with sufficient affinity such that the antibody is useful as a diagnostic and/or therapeutic agent in targeting P2X₇ receptor, typically non-functional P2X₇ receptor or a cancer associated P2X₇ receptor. Preferably, the extent of binding of a P2X₇ receptor antibody to an unrelated protein is less than about 10% of the binding of the antibody to P2X₇ receptor as measured, e.g., by a radioimmunoassay (RIA), Enzyme-Linked Immunosorbent Assay (ELISA), Biacore or Flow Cytometry. In certain embodiments, an antibody that binds to P2X₇ receptor has a dissociation constant (Kd) of <1 μM, <100 nM, <10 nM, <1 nM, or <0.1 nM. An anti nfP2X₇ receptor antibody is generally one having some or all of these serological characteristics and that binds to dysfunctional P2X₇ receptors but not to functional P2X₇ receptors.

An “affinity matured” antibody is one with one or more alterations in one or more hypervariable regions thereof that result in an improvement in the affinity of the antibody for the antigen, compared to a parent antibody that does not possess those alteration(s). Preferred affinity matured antibodies will have nanomolar or even picomolar affinities for the target antigen. Affinity matured antibodies are produced by procedures known in the art.

A “blocking” antibody” or an “antagonist” antibody is one that inhibits or reduces biological activity of the antigen it binds. Preferred blocking antibodies or antagonist antibodies substantially or completely inhibit the biological activity of the antigen.

An “agonist antibody”, as used herein, is an antibody, which mimics at least one of the functional activities of a polypeptide of interest.

“Binding affinity” generally refers to the strength of the sum total of non-covalent interactions between a single binding site of a molecule (e.g., an antibody) and its binding partner (e.g., an antigen). Unless indicated otherwise, as used herein, “binding affinity” refers to intrinsic binding affinity, which reflects a 1:1 interaction between members of a binding pair (e.g., antibody and antigen). The affinity of a molecule X for its partner Y can generally be represented by the dissociation constant (Kd). Affinity can be measured by common methods known in the art, including those described herein. Low-affinity antibodies generally bind antigen slowly and tend to dissociate readily, whereas high-affinity antibodies generally bind antigen faster and tend to remain bound longer. A variety of methods of measuring binding affinity are known in the art, any of which can be used for purposes of the present invention.

As used herein, the term “antigen” is intended to include substances that bind to or evoke the production of one or more antibodies and may comprise, but is not limited to, proteins, peptides, polypeptides, oligopeptides, lipids, carbohydrates, and combinations thereof, for example a glycosylated protein or a glycolipid. The term “antigen” as used herein refers to a molecular entity that may be expressed on a target cell and that can be recognised by means of the adaptive immune system including but not restricted to antibodies or TCRs, or engineered molecules including but not restricted to transgenic TCRs, CARs, scFvs or multimers thereof, Fab-fragments or multimers thereof, antibodies or multimers thereof, single chain antibodies or multimers thereof, or any other molecule that can execute binding to a structure with high affinity.

“Epitope” generally refers to that part of an antigen that is bound by the antigen binding site of an antibody. An epitope may be “linear” in the sense that the hypervariable loops of the antibody CDRs that form the antigen binding site bind to a sequence of amino acids as in a primary protein structure. In certain embodiments, the epitope is a “conformational epitope” i.e. one in which the hypervariable loops of the CDRs bind to residues as they are presented in the tertiary or quaternary protein structure.

As used herein, the term “antigen binding domain” and shall be taken to mean a region of an antibody that is capable of specifically binding to an antigen, i.e., a V_{H} or a V_{L} or an Fv comprising both a VH and a VL. The antigen binding domain need not be in the context of an entire antibody, e.g., it can be in isolation (e.g., a domain antibody) or in another form, e.g., as described herein, such as a scFv.

The term “target cell” as used herein refers to a cell that expresses a dysfunctional P2X₇ receptor (e.g. nfP2X₇ receptor) or a cell surface molecule to which the targeting moiety of the bridging molecule binds. The target cell may be a cancer cell or any other diseased cell.

The term “disorder” or “condition” means a functional abnormality or disturbance in a subject such as a cancer, an autoimmune disorder, or an infection by virus, bacteria, parasite, or others.

For example, a nucleic acid or a peptide naturally present in a living animal is not “isolated”, but the same nucleic acid or peptide partially or completely separated from the coexisting materials of its natural state is “isolated”. An isolated nucleic acid or protein can also exist in a non-native environment such as, for example, in a host cell.

As used herein, the term “subject” refers to a mammal such as mouse, rat, cow, pig, goat, chicken, dog, monkey or human. Preferentially, the subject is a human. The subject may be a subject suffering from a disorder such as cancer (a patient), but the subject also may be a healthy subject.

The term “autologous” as used herein refers to any material derived from the same subject to whom it is later re-introduced.

The term “allogeneic” as used herein refers to any material derived from a different subject of the same species as the subject to whom the material is re-introduced.

The terms “therapeutically effective amount” or “therapeutically effective population” mean an amount of, for example, a cell population that provides a therapeutic benefit in a subject.

The terms “binds to”, “specifically binds to” or “specific for” with respect to a targeting moiety, as used e.g. in the bridging molecule as disclosed herein, or of a CAR referring to an antigen-binding domain that recognises and binds to a specific antigen, does not substantially recognise or bind to other molecules in a sample. An antigen-binding domain or targeting moiety that binds specifically to an antigen from one species also may bind to that antigen from another species. This cross-species reactivity is typical of many antibodies and therefore not contrary to the definition that the antigen-binding domain is specific. An antigen-binding domain that specifically binds to an antigen may bind also to different allelic forms of the antigen (allelic variants, splice variants, isoforms etc.) or homologous variants of this antigen from the same gene family. This cross reactivity is typical of many antibodies and therefore not contrary to the definition that the antigen-binding domain is specific.

The terms “engineered cell” and “genetically modified cell” as used herein can be used interchangeably. The terms mean containing and/or expressing a foreign gene or nucleic acid sequence that in turn modifies the genotype or phenotype of the cell or its progeny. Especially, the terms refer to the fact that cells, preferentially immune cells, can be manipulated by recombinant methods well known in the art to express stably or transiently peptides or proteins that are not expressed in these cells in the natural state. For example, immune cells are engineered to express an artificial construct such as a chimeric antigen receptor on their cell surface. For example, the CAR sequences may be delivered into cells using an adenoviral, adeno-associated viral (AAV)-based, retroviral or lentiviral vector or any other pseudotyped variations thereof or any other gene delivery mechanism such as electroporation or lipofection with CRISPR/Cas9, transposons (e.g. sleeping-beauty) or variations thereof. The gene delivery may be in the form of mRNA (transient) or DNA (transient or permanent).

The terms “immune cell” or “immune effector cell” refer to a cell that may be part of the immune system, either the adaptive (i.e. cellular or humoral) or innate immune system, and executes a particular effector function such as alpha-beta T cells, NK cells, NKT cells, B cells, Breg cells, Treg cells, innate lymphoid cells (ILC), cytokine induced killer (CIK) cells, lymphokine activated killer (LAK) cells, gamma-delta T cells, mesenchymal stem cells or mesenchymal stromal cells (MSC), monocytes or macrophages or any hematopoietic progenitor cells such as pluripotent stem cells and early progenitor subsets that may mature or differentiate into somatic cells. The cells may be naturally occurring or generated by cytokine exposure, artificial/genetically modified cells (such as iPSCs and other artificial cell types). The immune cell may be an artificial cell subset including induced pluripotent stem cells and cells maturated therefrom. Preferred immune cells are cells with cytotoxic effector function such as alpha-beta T cells, NK cells, NKT cells, ILC, CIK cells, LAK cells or gamma-delta T cells. “Effector function” means a specialised function of a cell, e.g. in a T cell an effector function may be cytolytic activity or helper cell activity including the secretion of cytokines.

The term “treat” (treatment of) a disorder as used herein means to reduce the frequency or severity of at least one sign or symptom of a disease or disorder experienced by a subject.

The term “expression” as used herein is defined as the transcription and/or translation of a particular nucleotide sequence driven by its promoter in a cell.

### Antigen Binding Protein

The present invention includes an antigen binding protein comprising:
(i) a first antigen binding domain that binds to a tumour-specific antigen; and
(ii) a second antigen binding domain that binds to a cell surface molecule on an immune cell.

In any aspect, the tumour-specific antigen is an antigen expressed on a solid tumour. In one embodiment, the tumour-specific antigen is any one of dysfunctional P2X₇ receptor, EGFRvIII or CLDN6.

In any aspect, the antigen binding protein may be any binding molecule, for example, a full-size antibody, or fragment thereof, or any antibody or fragment thereof described herein, an immunocytokine (antibody linked to a cytokine, or fragments thereof), a ligand (protein related, peptides, processed molecules, cytokines, hormones), a soluble T cell receptor (TcR), a single chain (sc) TcR, single chain T cell receptor binding motifs, a T cell receptor like mAb or a D domain (for example a D domain derived from the de novo-designed α-helical bundle, α3D).

In any aspect, the first antigen binding domain binds to, or specifically binds to, a dysfunctional P2X₇ receptor, EGFRvIII or CLDN6.

In any embodiment, the first antigen binding domain binds to an epitope associated with an adenosine triphosphate (ATP)-binding site of the dysfunctional P2X₇ receptor. In some embodiments, the dysfunctional P2X₇ receptor has a reduced capacity to bind ATP at the ATP-binding site compared to an ATP-binding capacity of a functional P2X₇ receptor (e.g., a receptor having wild-type sequence and having a conformation or fold of an ATP-binding receptor). In some embodiments the dysfunctional P2X₇ receptor cannot bind ATP at the ATP-binding site.

In any embodiment, the dysfunctional P2X₇ receptor has a conformational change that renders the receptor dysfunctional. In some embodiments, the conformational change is a change of an amino acid from the trans-conformation to the cis-conformation. In some embodiments, the amino acid that has changed from a trans-conformation to a cis-conformation is proline at amino acid position 210 of the dysfunctional P2X₇ receptor.

In any embodiment, the first antigen binding domain binds to an epitope that includes the proline at amino acid position 210 of the dysfunctional P2X₇ receptor. In some embodiments, the first antigen binding site binds to an epitope that includes one or more amino acid residues spanning from glycine at amino acid position 200 to cysteine at amino acid position 216, inclusive, of the dysfunctional P2X₇ receptor.

The first antigen binding domain present can be any suitable molecule that can interact with and specifically binds to a dysfunctional P2X₇ receptor. However, in some embodiments, the first antigen binding domain includes amino acid sequence homology to the amino acid sequence of an antibody, or a fragment thereof, which binds to the dysfunctional P2X₇ receptor. In some embodiments, the first antigen binding domain includes amino acid sequence homology to the amino acid sequence of a fragment-antigen binding (Fab) portion of an antibody that binds to a dysfunctional P2X₇ receptor. In some embodiments, the antibody is a humanised antibody.

In any embodiment, the first antigen binding domain includes amino acid sequence homology to the amino acid sequence of a single-chain variable fragment (scFv) or a multivalent scFv that binds to a dysfunctional P2X₇ receptor. In some embodiments, the multivalent scFv is a divalent or trivalent scFv.

In any embodiment, the first antigen binding domain includes amino acid sequence homology to a single-antibody domain (sdAb) that binds to a dysfunctional P2X₇ receptor.

In any embodiment, the first antigen binding domain includes a binding polypeptide that includes amino acid sequence homology to one or more complementarity determining regions (CDRs) of an antibody that binds to a dysfunctional P2X₇ receptor. In any embodiment, the binding polypeptide includes amino acid sequence homology to the CDR1, 2 and 3 domains of the V_{H} and/or V_{L} chain of an antibody that binds to a dysfunctional P2X₇ receptor. In preferred embodiments, the binding polypeptide comprises the amino acid sequence of the CDRs of the V_{H} and/or V_{L} chain of an antibody, or the amino acid sequence of the V_{H} and/or V_{L} chains of an antibody, or the amino acid sequence of an antibody or fragment thereof, wherein the antibody or fragment thereof comprises the amino acid sequences of any antibody described in PCT/AU2002/000061 or PCT/AU2002/001204 (or in any one of the corresponding U.S. Pat. Nos. 7,326,415, 7,888,473, 7,531,171, 8,080,635, 8,399,617, 8,709,425, 9,663,584, or U.S. Pat. No. 10,450,380), PCT/AU2007/001540 (or in corresponding U.S. Pat. No. 8,067,550), PCT/AU2007/001541 (or in corresponding US publication US 2010-0036101), PCT/AU2008/001364 (or in any one of the corresponding U.S. Pat. Nos. 8,440,186, 9,181,320, 9,944,701 or U.S. Pat. No. 10,597,451), PCT/AU2008/001365 (or in any one of the corresponding U.S. Pat. No. 8,293,491 or U.S. Pat. No. 8,658,385), PCT/AU2009/000869 (or in any one of the corresponding U.S. Pat. No. 8,597,643, 9,328,155 or 10,238,716), PCT/AU2010/001070 (or in any one of the corresponding publications WO/2011/020155, U.S. Pat. Nos. 9,127,059, 9,688,771, or U.S. Pat. No. 10,053,508), and PCT/AU2010/001741 (or in any one of the corresponding publications WO 2011/075789 or U.S. Pat. No. 8,835,609) the entire contents of which are hereby incorporated by reference. Preferably the antibody comprises the CDR amino acid sequences of 2-2-1 described in PCT/AU2010/001070 (or in any one of the corresponding U.S. Pat. Nos. 9,127,059, 9,688,771, or U.S. Pat. No. 10,053,508) or BPM09 described in PCT/AU2007/001541 (or in corresponding US publication US 2010-0036101) and produced by the hybridoma AB253 deposited with the European Collection of Cultures (ECACC) under Accession no. 06080101.

In any aspect, the cell surface molecule on an immune cell is present on the surface of a lymphoid or myeloid lineage cell. The lymphoid lineage cell may be a natural killer cell or lymphocyte. The lymphocyte may be a T lymphocyte (eg cytotoxic T cell, gd T cell, or NKT cell) or a B lymphocyte. The myeloid lineage cell may be a monocyte, such as a macrophage. The cell surface molecule on an immune cell may be any molecule that is present on an immune cell that can be bound by or detected by an antigen binding domain. Preferably, the cell surface molecule is only present on an immune cell and not present on a non-immune cell. Preferably the cell surface molecule is a receptor that directly or indirectly causes activation of the immune cell. Typically activation of the immune cell results in an increased ability to reduce the viability of a cancer cell.

In any aspect, the second antigen binding domain binds to, or specifically binds to, a cell surface molecule on an immune cell as described herein. In one embodiment, the cell surface molecule is a T cell receptor or a molecule associated with a T cell receptor, such as a TCR-alpha or beta chain, or CD3. In another embodiment, the cell surface molecule is a costimulatory receptor, such as CD27, CD28, CD30, CD40, DAP10, OX40, 4-1BB (CD137) and ICOS. In another embodiment, the cell surface molecule may be an Fc receptor, or portion thereof, such as FcγRI (CD64), FcγRIIa (CD32), FcγRIIb (CD32), FcγRIIIa (CD16a), FcγRIIIb (CD16b).

The second antigen binding domain may be any molecule which binds to a cell surface molecule on an immune cell. For example, the second antigen binding domain may comprise, or be part of, an antibody or antigen binding fragment thereof. Alternatively, the second antigen binding domain may be an Fc region or part thereof capable of binding to an Fc receptor such as FcRI or FcRIIIa. In any aspect or embodiment, the second antigen binding domain may be an Fc region of an antibody or a polypeptide comprising an Fc receptor binding domain.

In any aspect, the Fc region of an antibody is an Fc region of an IgG, more preferably IgG1, more preferably a human IgG1.

Preferably, the Fc region comprises two heavy chain fragments, more preferably the CH2 and CH3 domains of said heavy chain. In one embodiment, the heavy chain fragments are linked via disulphide linkages. In another embodiment, the fragments are not linked. In another embodiment, the Fc region includes one or more modifications that inhibit or prevent homo or heterodimerisation, for example prevent assembly or dimerization at a hinge region.

The term “Fc region” herein is used to define a C-terminal region of an immunoglobulin heavy chain that contains at least a portion of the constant region. In other words, the Fc region contains two heavy chain fragments comprising the CH2 and CH3 domains of an antibody. In the context of the present invention, the Fc region comprises two heavy chain fragments, preferably the CH2 and CH3 domains of said heavy chain. The two heavy chain fragments may be held together by two or more disulfide bonds and by hydrophobic interactions of the CH3 domains. The heavy chain constant domains that correspond to the different classes of immunoglobulins are called α, δ, ε, γ, and μ.

The term “Fc region” also includes native sequence Fc regions and variant Fc regions. The Fc region may include the carboxyl-terminus of the heavy chain. Antibodies produced by host cells may undergo post-translational cleavage of one or more, particularly one or two, amino acids from the C-terminus of the heavy chain. Therefore, an antibody produced by a host cell by expression of a specific nucleic acid molecule encoding a full-length heavy chain may include the full-length heavy chain, or it may include a cleaved variant of the full-length heavy chain. Unless otherwise specified herein, numbering of amino acid residues in the Fc region or constant region is according to the EU numbering system, also called the EU index, as described in Kabat et al., Sequences of Proteins of Immunological Interest, 5th Ed. Public Health Service, National Institutes of Health, Bethesda, MD, 1991. Amino acid sequence variants of the Fc region of an antibody may be contemplated. Amino acid sequence variants of an Fc region of an antibody may be prepared by introducing appropriate modifications into the nucleotide sequence encoding the antibody, or by peptide synthesis. Such modifications include, for example, deletions from, and/or insertions into and/or substitutions of residues within the amino acid sequences of the Fc region of the antibody. Any combination of deletion, insertion, and substitution can be made to arrive at the final construct, provided that the final construct possesses the desired characteristics, e.g., inducing or supporting an anti-inflammatory response.

The Fc region of the antibody may be an Fc region of any of the classes of antibody, such as IgA, IgD, IgE, IgG, and IgM. The “class” of an antibody refers to the type of constant domain or constant region possessed by its heavy chain. There are five major classes of antibodies: IgA, IgD, IgE, IgG, and IgM, and several of these may be further divided into subclasses (isotypes), e.g., IgG1, IgG2, IgG3, IgG4, IgA1, and IgA2. Accordingly, as used in the context of the present invention, the antibody may be an Fc region of an IgG. For example, the Fc region of the antibody may be an Fc region of an IgG1, an IgG2, an IgG2b, an IgG3 or an IgG4. In some aspects, the fusion protein of the present invention comprises an IgG of an Fc region of an antibody. In the context of the present invention, the Fc region of the antibody is an Fc region of an IgG, preferably IgG1.

The Fc region is composed of two heavy chains that contribute two or three constant domains depending on the class of the antibody. By binding to specific proteins, the Fc region ensures that each antibody generates an appropriate immune response for a given antigen.

An Fc receptor binding domain is any protein or polypeptide that binds to the Fc receptor on the surface of a cell. The Fc receptor binding domain may be an antigen binding domain of an antibody. The Fc receptor binding domain also binds to various cell receptors, such as Fc receptors, and other immune molecules, such as complement proteins.

The Fc region may include one or more mutations or modifications that increases affinity for binding an Fc receptor.

The Fc region may include one or more mutations or modifications that decreases affinity for binding an Fc receptor. For example, the Fc region may have one or more mutations or modifications that attenuate binding to an Fc receptor.

In any aspect or embodiment, the antigen binding protein may have a first antigen binding domain for binding to a tumour-specific antigen and a second antigen binding domain for binding to one of CD3 or CD16. Preferably, the antigen binding protein comprises a first antigen binding domain for binding to a dysfunctional P2X₇ receptor and a second antigen binding domain for binding to CD3 or CD16.

In any embodiment, an antigen binding protein for binding to a dysfunctional P2X₇ receptor and CD3 may comprise or consist of amino acid sequences set forth in SEQ ID NOs: 313 and 314, SEQ ID NOs: 313 and 315, SEQ ID NOs: 313 and 316, SEQ ID NOs: 313 and 317 or a sequence at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, identical thereto.

In any embodiment, an antigen binding protein for binding to a dysfunctional P2X₇ receptor and CD3 may comprise or consist of amino acid sequences set forth in SEQ ID NOs: 318 and 319 or a sequence at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, identical thereto.

In any embodiment, an antigen binding protein for binding to a dysfunctional P2X₇ receptor and CD3 may comprise or consist of an amino acid sequence set forth in SEQ ID NO: 320 or a sequence at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, identical thereto.

In any embodiment, an antigen binding protein for binding to a dysfunctional P2X₇ receptor and CD3 may comprise or consist of an amino acid sequence set forth in SEQ ID NO: 321 or a sequence at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, identical thereto.

In any embodiment, an antigen binding protein for binding to a dysfunctional P2X₇ receptor and CD3 may comprise or consist of an amino acid sequence set forth in SEQ ID NO: 322 or a sequence at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, identical thereto.

In any embodiment, an antigen binding protein for binding to a dysfunctional P2X₇ receptor and CD3 may comprise or consist of an amino acid sequence set forth in SEQ ID NO: 324 or a sequence at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, identical thereto.

In any embodiment, an antigen binding protein for binding to a dysfunctional P2X₇ receptor and CD3 may comprise or consist of an amino acid sequence set forth in SEQ ID NO: 325 or a sequence at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, identical thereto.

In any embodiment, an antigen binding protein for binding to a dysfunctional P2X₇ receptor and CD16 may comprise or consist of an amino acid sequence set forth in SEQ ID NO: 326 or a sequence at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, identical thereto.

In any embodiment, an antigen binding protein for binding to a dysfunctional P2X₇ receptor and CD16 may comprise or consist of an amino acid sequence set forth in SEQ ID NO: 327 or a sequence at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, identical thereto.

In any embodiment, an antigen binding protein for binding to a dysfunctional P2X₇ receptor and CD3 may comprise or consist of an amino acid sequence set forth in SEQ ID NO: 328 and 329, SEQ ID NO: 328 and 330, SEQ ID NO: 328 and 331, SEQ ID NO: 328 and 332, or SEQ ID NO: 328 and 333, or a sequence at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, identical thereto.

In any embodiment, an antigen binding protein for binding to a dysfunctional P2X₇ receptor and CD16 may comprise or consist of an amino acid sequence set forth in SEQ ID NO: 334 and 335, SEQ ID NO: 334 and 336, SEQ ID NO: 334 and 337, SEQ ID NO: 334 and 338, or SEQ ID NO: 334 and 339, or a sequence at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, identical thereto.

In any embodiment, an antigen binding protein for binding to CLDN6 and CD3 may comprise or consist of an amino acid sequence set forth in SEQ ID NO: 340 and 341, or a sequence at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, identical thereto.

In any embodiment, an antigen binding protein for binding to CLDN6 and CD3 may comprise or consist of an amino acid sequence set forth in SEQ ID NO: 342, or a sequence at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, identical thereto.

In any embodiment, an antigen binding protein for binding to CLDN6 and CD16 may comprise or consist of an amino acid sequence set forth in SEQ ID NO: 343, or a sequence at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, identical thereto.

In any embodiment, an antigen binding protein for binding to EGFRvIII and CD3 may comprise or consist of an amino acid sequence set forth in SEQ ID NO: 344 and 345, or a sequence at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, identical thereto.

In any embodiment, an antigen binding protein for binding to EGFRvIII and CD3 may comprise or consist of an amino acid sequence set forth in SEQ ID NO: 346 or SEQ ID NO: 347 or a sequence at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, identical thereto.

In any embodiment, the antigen binding protein (or OR molecule) may comprise or consist of an amino acid sequence specified in the Sequence information table above, for example any one or more of SEQ ID Nos: 307 to 347 or a sequence at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, identical thereto.

In any aspect, the antigen binding protein (or OR molecule) described herein does not have a HIS tag. Also contemplated, is an OR molecule that comprises an amino acid sequence specified in the Sequence information table above, for example any one or more of SEQ ID Nos: 307 to 347 but without a HIS tag specified in the sequence, or a sequence at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, identical thereto. Further, in one embodiment, the OR molecule may comprise a tag other than a HIS tag, or may comprise an amino acid sequence specified in the Sequence information table above, for example any one or more of SEQ ID Nos: 307 to 347 but with a different tag in the position of the HIS tag specified in the sequence.

### Chimeric Antigen Receptor (CAR)

The term cancer-specific CAR (T cell) targeting refers to the use of a CAR T cell for binding to a target antigen that is presented on the cell surface of tumour cells, but is not typically found on the surface of a healthy cell. In other words, normal cells under normal circumstances may be characterised by the absence of the target antigen on the extracellular membrane (and therefore the presence of the antigen on the cell surface cannot be detected). However, such cells may express mRNA encoding the antigen at an intracellular level. As CAR T cells only recognize surface-expressed antigens, the intracellular expression of the targeted proteins will not lead to CAR engagement.

The targeted epitopes E200 and E300 of the P2X₇ receptor are not exposed on the form of the receptor found in healthy tissue and thus these epitopes can be regarded as cancer specific. In other words, the E200 and E300 epitopes are only exposed, and available for binding when the P2X₇ receptor has an altered non-functional conformation, such as occurs in the context of cancer (in which case the receptor is referred to as nfP2X₇ receptor). Another example of a cancer-specific targeted epitope may be derived from the splice variant EGFRvIII. Still another example is the antigen CLDN6 which is mostly restricted to embryonic and foetal life and has very limited expression in healthy cells after the early phase in life and may be regarded as highly restricted and relatively overexpressed in cancer. The present invention contemplates the binding any such tumour-specific antigen, including nfP2X₇, EGFRvIII and CLDN6 for cancer-specific targeting and engaging CAR T cells via the bridging molecules described herein to cancer-associated antigens.

In general, a CAR may comprise an extracellular domain (extracellular part) comprising the antigen binding domain, a transmembrane domain and an intracellular signaling domain. The extracellular domain may be linked to the transmembrane domain by a linker. The extracellular domain may also comprise a signal peptide. The extracellular part of the CAR of the present invention comprises a tumour-specific antigen binding domain. For example, the tumour-specific antigen may be any one described herein, including nfP2X₇, EGFRvIII or CLDN6.

The tumour-specific antigen binding domain may be a nfP2X₇ binding domain that recognises the E200 (or E300 or E200-300 composite) epitope as disclosed herein. Specifically, the CAR as disclosed herein has an extracellular nfP2X₇ E200 binding domain as an antigen binding domain. Alternatively, the tumour-specific antigen binding domain may be an EGFRvIII binding domain that recognises an epitope resulting out of the fusion of the amino acid sequence starting at position 25-29 LEEKK, followed by the insertion of G and the subsequent amino acid sequence 298-304 NYVVTDH, the total epitope is a 13-mer comprised of the sequence LEEKKGNYVVTDH (SEQ ID NO: 267). Alternatively, the tumour-specific antigen binding domain may be a CLDN6 binding domain that recognises an epitope in the second extracellular domain of CLDN6 [UniProtKB—P56747 (CLDN6_HUMAN)] via the amino acid sequence of SEQ ID NO: 273, 274 or 275.

Typically, the antigen-recognition domain includes a binding polypeptide that includes amino acid sequence homology to one or more complementarity determining regions (CDRs) of an antibody that binds to a tumour-specific antigen (such as a dysfunctional P2X₇ receptor, EGFRvIII or CLDN6). In any embodiment, the binding polypeptide includes amino acid sequence homology to the CDR1, 2 and 3 domains of the V_{H} and/or V_{L} chain of an antibody that binds to a tumour-specific antigen (such as a dysfunctional P2X₇ receptor, EGFRvIII or CLDN6).

In particularly preferred embodiments of the invention, the antigen-recognition domain of the CAR binds to an epitope of the tumour-specific antigen nfP2X₇.

In such embodiments, the binding polypeptide comprises the amino acid sequence of the CDRs of the V_{H} and/or V_{L} chain of an antibody described in PCT/AU2002/000061 or PCT/AU2002/001204 (or in any one of the corresponding U.S. Pat. Nos. 7,326,415, 7,888,473, 7,531,171, 8,080,635, 8,399,617, 8,709,425, 9,663,584, or U.S. Pat. No. 10,450,380), PCT/AU2007/001540 (or in corresponding U.S. Pat. No. 8,067,550), PCT/AU2007/001541 (or in corresponding US publication US 2010-0036101), PCT/AU2008/001364 (or in any one of the corresponding U.S. Pat. Nos. 8,440,186, 9,181,320, 9,944,701 or U.S. Pat. No. 10,597,451), PCT/AU2008/001365 (or in any one of the corresponding U.S. Pat. No. 8,293,491 or U.S. Pat. No. 8,658,385), PCT/AU2009/000869 (or in any one of the corresponding U.S. Pat. No. 8,597,643, 9,328,155 or 10,238,716), PCT/AU2010/001070 (or in any one of the corresponding publications WO/2011/020155, U.S. Pat. Nos. 9,127,059, 9,688,771, or U.S. Pat. No. 10,053,508), and PCT/AU2010/001741 (or in any one of the corresponding publications WO 2011/075789 or U.S. Pat. No. 8,835,609) the entire contents of which are hereby incorporated by reference. Preferably the antibody comprises the CDR amino acid sequences of 2-2-1 described in PCT/AU2010/001070 (or in any one of the corresponding U.S. Pat. Nos. 9,127,059, 9,688,771, or U.S. Pat. No. 10,053,508) or BPM09 described in PCT/AU2007/001541 (or in corresponding US publication US 2010-0036101) and produced by the hybridoma AB253 deposited with the European Collection of Cultures (ECACC) under Accession no. 06080101, WO2013185010A1 or WO2019056023.

The binding polypeptide of the CAR may comprise the amino acid sequence of the V_{H} and/or V_{L} chains of an antibody described in PCT/AU2002/000061 or PCT/AU2002/001204 (or in any one of the corresponding U.S. Pat. Nos. 7,326,415, 7,888,473, 7,531,171, 8,080,635, 8,399,617, 8,709,425, 9,663,584, or U.S. Pat. No. 10,450,380), PCT/AU2007/001540 (or in corresponding U.S. Pat. No. 8,067,550), PCT/AU2007/001541 (or in corresponding US publication US 2010-0036101), PCT/AU2008/001364 (or in any one of the corresponding U.S. Pat. Nos. 8,440,186, 9,181,320, 9,944,701 or U.S. Pat. No. 10,597,451), PCT/AU2008/001365 (or in any one of the corresponding U.S. Pat. No. 8,293,491 or U.S. Pat. No. 8,658,385), PCT/AU2009/000869 (or in any one of the corresponding U.S. Pat. No. 8,597,643, 9,328,155 or 10,238,716), PCT/AU2010/001070 (or in any one of the corresponding publications WO/2011/020155, U.S. Pat. Nos. 9,127,059, 9,688,771, or U.S. Pat. No. 10,053,508), and PCT/AU2010/001741 (or in any one of the corresponding publications WO 2011/075789 or U.S. Pat. No. 8,835,609) the entire contents of which are hereby incorporated by reference. Preferably the antibody comprises the CDR amino acid sequences of 2-2-1 described in PCT/AU2010/001070 (or in any one of the corresponding U.S. Pat. Nos. 9,127,059, 9,688,771, or U.S. Pat. No. 10,053,508) or BPM09 described in PCT/AU2007/001541 (or in corresponding US publication US 2010-0036101) and produced by the hybridoma AB253 deposited with the European Collection of Cultures (ECACC) under Accession no. 06080101, WO2013185010A1 or WO2019056023.

The binding polypeptide of the CAR may comprise the amino acid sequence of an antibody or fragment thereof described in PCT/AU2002/000061 or PCT/AU2002/001204 (or in any one of the corresponding U.S. Pat. Nos. 7,326,415, 7,888,473, 7,531,171, 8,080,635, 8,399,617, 8,709,425, 9,663,584, or U.S. Pat. No. 10,450,380), PCT/AU2007/001540 (or in corresponding U.S. Pat. No. 8,067,550), PCT/AU2007/001541 (or in corresponding US publication US 2010-0036101), PCT/AU2008/001364 (or in any one of the corresponding U.S. Pat. Nos. 8,440,186, 9,181,320, 9,944,701 or U.S. Pat. No. 10,597,451), PCT/AU2008/001365 (or in any one of the corresponding U.S. Pat. No. 8,293,491 or U.S. Pat. No. 8,658,385), PCT/AU2009/000869 (or in any one of the corresponding U.S. Pat. No. 8,597,643, 9,328,155 or 10,238,716), PCT/AU2010/001070 (or in any one of the corresponding publications WO/2011/020155, U.S. Pat. Nos. 9,127,059, 9,688,771, or U.S. Pat. No. 10,053,508), and PCT/AU2010/001741 (or in any one of the corresponding publications WO 2011/075789 or U.S. Pat. No. 8,835,609) the entire contents of which are hereby incorporated by reference. Preferably the antibody comprises the CDR amino acid sequences of 2-2-1 described in PCT/AU2010/001070 (or in any one of the corresponding U.S. Pat. Nos. 9,127,059, 9,688,771, or U.S. Pat. No. 10,053,508) or BPM09 described in PCT/AU2007/001541 (or in corresponding US publication US 2010-0036101) and produced by the hybridoma AB253 deposited with the European Collection of Cultures (ECACC) under Accession no. 06080101, WO2013185010A1 or WO2019056023.

A “signal peptide” refers to a peptide sequence that directs the transport and localisation of the protein within a cell, e.g. to a certain cell organelle (such as the endoplasmic reticulum) and/or the cell surface.

Generally, an “antigen binding domain” refers to the region of the CAR that specifically binds to an antigen (and thereby is able to target a cell containing the antigen). The CARs of the invention may comprise one or more antigen binding domains. Generally, the targeting regions on the CAR are extracellular. The antigen binding domain may comprise an antibody or an antibody binding fragment thereof. The antigen binding domain may comprise, for example, full length heavy chain, Fab fragments, single chain Fv (scFv) fragments, divalent single chain antibodies or diabodies. Any molecule that binds specifically to a given antigen such as affibodies or ligand binding domains from naturally occurring receptors may be used as an antigen binding domain. Often the antigen binding domain is a scFv. Normally, in a scFv the variable regions of an immunoglobulin heavy chain and light chain are fused by a flexible linker to form a scFv. Such a linker may be for example the “(G₄/S₁)₃-linker” and variations thereof but the skilled person will appreciate that various linker sequences and formats may be used.

In some instances, it is beneficial for the antigen binding domain to be derived from the same species in which the CAR will be used in. For example, when it is planned to use it therapeutically in humans, it may be beneficial for the antigen binding domain of the CAR to comprise a human or humanised antibody or antigen binding fragment thereof. Human or humanised antibodies or antigen binding fragments thereof can be made by a variety of methods well known in the art. The CAR as disclosed herein has an extracellular linker/label epitope binding domain as an antigen binding domain allowing it to bind indirectly via a target cell binding molecule as disclosed herein to an antigen expressed on a target cell.

“Spacer” or “hinge” as used herein refers to the hydrophilic region that is between the antigen binding domain and the transmembrane domain. The CARs of the invention may comprise an extracellular spacer domain but it is also possible to leave out such a spacer. The spacer may include e.g. Fc fragments of antibodies or fragments thereof, hinge regions of antibodies or fragments thereof, CH2 or CH3 regions of antibodies, accessory proteins, artificial spacer sequences or combinations thereof. A prominent example of a spacer is the CD8alpha hinge.

The transmembrane domain of the CAR may be derived from any desired natural or synthetic source for such a domain. When the source is natural, the domain may be derived from any membrane-bound or transmembrane protein. The transmembrane domain may be derived for example from CD8alpha or CD28. When the key signalling and antigen recognition modules (domains) are on two (or even more) polypeptides, then the CAR may have two (or more) transmembrane domains. The splitting of key signalling and antigen recognition modules enables small molecule-dependent, titratable and reversible control over CAR cell expression (Wu et al, 2015, Science 350: 293-303) due to small molecule-dependent heterodimerising domains in each polypeptide of the CAR.

The cytoplasmic domain (or the intracellular signaling domain) of the CAR is responsible for activation of at least one of the normal effector functions of the immune cell in which the CAR is expressed. “Effector function” means a specialised function of a cell, e.g. in a T cell an effector function may be cytolytic activity or helper cell activity including the secretion of cytokines. The intracellular signalling domain refers to the part of a protein that transduces the effector function signal and directs the cell expressing the CAR to perform a specialised function. The intracellular signalling domain may include any complete, mutated or truncated part of the intracellular signalling domain of a given protein sufficient to transduce a signal that initiates or blocks immune cell effector functions.

The function of the intracellular domains may be pro- or anti-inflammatory and/or immunomodulatory, or a combination of such.

Prominent examples of intracellular signalling domains for use in the CARs include the cytoplasmic signaling sequences of the T cell receptor (TCR) and co-receptors that initiate signal transduction following antigen receptor engagement.

Generally, T cell activation can be mediated by two distinct classes of cytoplasmic signalling sequences, firstly those that initiate antigen-dependent primary activation through the TCR (primary cytoplasmic signalling sequences) and secondly those that act in an antigen-independent manner to provide a secondary or co-stimulatory signal (secondary cytoplasmic signalling sequences, co-stimulatory signalling domain). Therefore, an intracellular signalling domain of a CAR may comprise one or more primary cytoplasmic signalling domains and/or one or more secondary cytoplasmic signalling domains.

Primary cytoplasmic signalling sequences that act in a stimulatory manner may contain ITAMs (immunoreceptor tyrosine-based activation motifs) signalling motifs.

Examples of ITAM containing primary cytoplasmic signalling sequences often used in CARs are those derived from TCR zeta (CD3 zeta), FcR gamma, FcR beta, CD3 gamma, CD3 delta, CD3 epsilon, CD5, CD22, CD79a, CD79b and CD66d. Most prominent is the sequence derived from CD3 zeta.

The cytoplasmic domain of the CAR may be designed to comprise the CD3-zeta signaling domain by itself or combined with any other desired cytoplasmic domain(s). The cytoplasmic domain of the CAR can comprise a CD3 zeta chain portion and a co-stimulatory signalling region. The co-stimulatory signalling region refers to a part of the CAR comprising the intracellular domain of a co-stimulatory molecule. A co-stimulatory molecule is a cell surface molecule other than an antigen receptor or their ligands that is required for an efficient response of lymphocytes to an antigen. Examples for a co-stimulatory molecule are CD27, CD28, 4-1BB (CD137), OX40, CD30, CD40, PD-1, ICOS, lymphocyte function-associated antigen-1 (LFA-1), CD2, CD7, LIGHT, NKG2C and B7-H₃.

The cytoplasmic signalling sequences within the cytoplasmic signalling part of the CAR may be linked to each other with or without a linker in a random or specified order. A short oligo-or polypeptide linker, which is preferably between 2 and 10 amino acids in length, may form the linkage. A prominent linker is the glycine-serine doublet.

As an example, the cytoplasmic domain may comprise the signalling domain of CD3-zeta and the signalling domain of CD28. In another example the cytoplasmic domain may comprise the signalling domain of CD3-zeta and the signalling domain of CD27. In a further example, the cytoplasmic domain may comprise the signalling domain of CD3-zeta, the signalling domain of CD28, and the signalling domain of CD27.

As aforementioned, either the extracellular part or the transmembrane domain or the cytoplasmic domain of a CAR may also comprise a heterodimerising domain for the aim of splitting key signalling and antigen recognition modules of the CAR.

Non-limiting examples of CARs that may be used in accordance with the present invention are set forth in SEQ ID NOs: 165-167, 266 or 272. An example of the architecture of various CAR molecules is also provided herein in FIG. 35.

A CAR for use in accordance with the present invention, i.e. a CAR comprising an nfP2X₇ E200 binding domain, may be designed to comprise any portion or part of the above-mentioned domains as described herein in any order and/or combination resulting in a functional CAR.

The CARs as disclosed herein, or polypeptide(s) derived therefrom, nucleic acid molecule(s) or recombinant expression vectors cells encoding said CARs, or populations of cells expressing said CARs, may be isolated and/or purified. The term “isolated” means altered or removed from the natural state. For example, an isolated population of cells means an enrichment of such cells and separation from other cells that are normally associated in their naturally occurring state with said isolated cells. An isolated population of cells means a population of substantially purified cells that are a more homogenous population of cells than found in nature. Preferably, the enriched cell population comprises at least about 90% of the selected cell type. In particular aspects, the cell population comprises at least about 90%, 91%, 92%, 93%, 94%, 95%, 96%, 97%, 98%, 99%, or even 100% of the selected cell type.

The affinity at which the dysfunctional P2X₇ receptor binding domain of the CAR binds to the nfP2X₇ recognition site E200 of the bridging molecule can vary, but generally the binding affinity may be in the range of 100 μM, 1 nM, 10 nM, or 100 nM, preferably at least about 1 μM or 10 μM, even more preferably at least about 100 μM.

CAR T cells targeted to EGFRvIII may be used to treat solid cancers. EGFRvIII is a frequent splice variant of EGFR skipping exons 2-7. EGFRvIII is tumour specific and does not occur in healthy cells as EGFR is tightly regulated in normal cells. EGFRvIII is commonly expressed in glioblastoma but also in breast cancer and head and neck cancer. The EGFRvIII-CAR T in this context may have the sequence (SEQ ID NO: 266) and is targeted to the epitope resulting out of the fusion of the amino acid sequence starting at position 25-29 LEEKK, followed by the insertion of G and the subsequent amino acid sequence 298-304 NYVVTDH, the total epitope comprises or consists of the sequence LEEKKGNYVVTDH (SEQ ID NO: 267). The complete EGFR sequence is found at UniProtKB—P00533 (EGFR_HUMAN) and the complete protein counts 1210 amino acids in isoform 1.

EGFRvIII targeted CAR T cells may be used to treat glioblastoma in a conventional way to target EGFRvIII on cancer cells, but may also be redirected to other cancer-associated target antigens via the bridging molecules described herein if the EGFRvIII epitope moiety is integrated into the sequence of bridging molecules. The EGFRvIII CAR T cells then can be used in the same manner as outlined for the nfP2X₇ CAR targeted approach described herein. The peptide tag may be the 13-mer peptide LEEKKGNYVVTDH or a shortened or extended natural or artificial variant thereof, of SEQ ID NO: 267.

The amino acid sequence of EGFRvIII CAR compatible bridging molecules targeted to CD33 and Her2 are described in Table 1 as SEQ ID NO: 268 and 269, and SEQ ID NO: 270 and 271, respectively.

CLDN6 targeted CAR T cells may be used to treat solid cancers e.g. ovarian cancer. The CLDN6-CAR T in this context may have the sequence (SEQ ID NO: 272) and is targeted to the second extracellular domain of CLDN6 [UniProtKB—P56747 (CLD6_HUMAN] cells directly via the amino acid sequence [ECD2, >sp|P56747|138-160 WTAHAIIRDFYNPLVAEAQKREL (SEQ ID NO: 273)] but may also be redirected to other cancer-associated target antigens, e.g. CD33 or Her2 via the bridging molecules described herein, if the CLDN6 epitope moiety is integrated into the sequence of bridging molecules. The CLDN6 CAR T cells then can be used in the same manner as outlined for the nfP2X₇ CAR targeted approach described herein. The peptide tag may be the 23-mer peptide WTAHAIIRDFYNPLVAEAQKREL or a shortened or extended natural or artificial variant thereof, such as SEQ ID NO: 274 or 275.

### Bridging Molecules

It will be appreciated that the bridging molecule may be in any form, provided that it comprises a) a targeting moiety for binding a target cell and b) a tumour-specific antigen epitope moiety. Preferably, the tumour-specific antigen epitope moiety is a dysfunctional P2X₇ receptor epitope moiety, a EGFRvIII epitope moiety or a CLDN6 epitope moiety.

As used herein, reference to a bridging molecule may also be by use of the term “BRiDGE”.

Typically, the targeting moiety is in the form of a fusion protein in which the targeting moiety is linked to the tumour-specific antigen epitope moiety, preferably dysfunctional P2X₇ receptor epitope moiety, directly or via a linker.

Any suitable linker may be used. The linker may comprise a polypeptide, a peptide or a chemical group.

A linker may be a peptide having a length of up to 20, 30, 40 or 50 amino acids. The term “linked to” or “fused to” refers to a covalent bond, e.g., a peptide bond, formed between two moieties. Accordingly, in the context of the present invention the linker may have a length of 1, 2, 3, 4, 5, 6, 7, 8, 9, 10, 11, 12, 13, 14, 15, 16, 17, 18, 19, 20, 21, 22 or more amino acids. For example, the herein provided bridging molecule may comprise a linker between the targeting moiety and tumour-specific antigen epitope moiety, preferably the dysfunctional P2X₇ receptor epitope moiety. Such linkers have the advantage that they can make it more likely that the different polypeptides of the fusion protein fold independently and behave as expected.

The skilled person will be familiar with the design and use of various peptide linkers comprised of various amino acids, and of various lengths, which would be suitable for use as linkers in accordance with the present invention. The linker may comprise various combinations of repeated amino acid sequences. The linker may be a flexible linker (such as those comprising repeats of glycine and serine residues), a rigid linker (such as those comprising glutamic acid and lysine residues, flanking alanine repeats) and/or a cleavable linker (such as sequences that are susceptible by protease cleavage).

The peptide linker may be any one or more repeats of Gly-Gly-Ser (GGS), Gly-Gly-Gly-Ser (GGGS) or Gly-Gly-Gly-Gly-Ser (GGGGS) or variations thereof. In one embodiment, the linker may comprise or consist of the sequence GGGGSGGGGSGGGGS, i.e. (G4S)₃.

In one embodiment, the peptide linker can include the amino acid sequence GGGGGS (a linker of 6 amino acids in length) or even longer. The linker may be a series of repeating glycine and serine residues (GS) of different lengths, i.e., (GS)n where n is any number from 1 to 15 or more. For example, the linker may be (GS)₃ (i.e., GSGSGS) or longer (GS)₁₁ or longer. It will be appreciated that n can be any number including 1, 2, 3, 4, 5, 6, 7, 8, 9, 10, 11 or more.

The peptide linker may consist of a series of repeats of Thr-Pro (TP) comprising one or more additional amino acids N and C terminal to the repeat sequence. For example, the linker may comprise or consist of the sequence GTPTPTPTPTGEF (also known as the TP5 linker). In further aspects, the linker may be a short and/or alpha-helical rigid linker (e.g. A(EAAAK)3A, PAPAP or a dipeptide such as LE or CC).

The linker may also be comprised of a glycine-serine based linker and a linker derived from an immunoglobulin hinge region. Examples of such linker combinations are provided in Table 1 (eg: G4S+IgG-derived hinged+G4S+E200 sequence). It will also be appreciated that the targeting moiety and tumour-specific antigen epitope moiety, preferably the dysfunctional P2X₇ receptor epitope moiety may be linked via their C or N terminal regions. For example, the E200 epitope sequence may be linked via its C terminus to the N terminal region of the targeting moiety (including to either the heavy or light chain of the targeting moiety). Similarly, the E200 epitope sequence may be linked via its N terminus to the C terminal region of the targeting moiety (including to either the heavy or light chain of the targeting moiety). It is well within the purview of the skilled person to be able to design and generate suitable bridging molecules are herein described for use in accordance with the invention.

### Targeting Moiety of the Bridging Molecule

The targeting moiety of the bridging molecule may bind to a cell surface molecule on a target cell. The cell surface molecule may comprise an antigen. The cell surface molecule may be selected from a protein, a lipid moiety, a glycoprotein, a glycolipid, a carbohydrate, a polysaccharide, a nucleic acid, an MHC-bound peptide, or a combination thereof. The cell surface molecule may comprise parts (e.g., coats, capsules, cell walls, flagella, fimbriae, and toxins) of bacteria, viruses, and other microorganisms. The cell surface molecule may be expressed by the target cell. The cell surface molecule may not be expressed by the target cell. By way of non-limiting example, the cell surface molecule may be a ligand expressed by a cell that is not the target cell and that is bound to the target cell or a cell surface molecule of the target cell. Also, by non-limiting example, the cell surface molecule may be a toxin, exogenous molecule or viral protein that is bound to a cell surface or cell surface receptor of the target cell.

The bridging molecules may interact with a plurality of target cells. The target cell may be an infected cell. The target cell may be a pathogenically infected cell. The target cell may be a diseased cell. The target cell may be a genetically modified cell. The target cell may not be a host cell. The target cell may come from an invading organism (e.g. yeast, worm, bacteria, fungus). Further disclosed herein are bridging molecules that interact with a molecule on a non-cell target. The non-cell target may be a virus or a portion thereof. The non-cell target may be a fragment of a cell. The non-cell target may be an extracellular matrix component or protein.

The target cell may be derived from a tissue. The tissue may be selected from brain, oesophagus, breast, gut, intestine, colon, lung, glia, ovary, uterus, testes, prostate, gastrointestinal tract, bladder, liver, spleen, thymus, bone, fat and skin. The target cell may be derived from one or more endocrine glands. Alternatively, or additionally, the target cell may be derived from one or more endocrine glands. The endocrine gland may be a lymph gland, pituitary gland, thyroid gland, parathyroid gland, pancreas, gonad or pineal gland.

The target cell may be selected from a stem cell, a pluripotent cell, a hematopoietic stem cell or a progenitor cell. The target cell may be a circulating cell. The target cell may be an immune cell.

The target cell may be a cancer stem cell. The target cell may be a cancer cell. The cancer cell may be derived from a tissue. The tissue may be selected from, by way of non-limiting example, a brain, an oesophagus, a breast, a colon, a lung, a glia, an ovary, a uterus, a testicle, a prostate, a gastrointestinal tract, a bladder, a liver, a thyroid and skin. The cancer cell may be derived from bone. The cancer cell may be derived from blood. The cancer cell may be derived from a B cell, a T cell, a monocyte, a thrombocyte, a leukocyte, a neutrophil, an eosinophil, a basophil, a lymphocyte, a hematopoietic stem cell or an endothelial cell progenitor. The cancer cell may be derived from a CD19-positive B lymphocyte. The cancer cell may be derived from a stem cell. The cancer cell may be derived from a pluripotent cell. The cancer cell may be derived from one or more endocrine glands. The endocrine gland may be a lymph gland, pituitary gland, thyroid gland, parathyroid gland, pancreas, gonad or pineal gland.

The cell surface molecule of the target cell may be a receptor. The receptor may be an extracellular receptor. The receptor may be a cell surface receptor. By way of non-limiting example, the receptor may bind a hormone, a neurotransmitter, a cytokine, a growth factor or a cell recognition molecule. The receptor may be a transmembrane receptor. The receptor may be an enzyme-linked receptor. The receptor may be a G-protein couple receptor (GPCR). The receptor may be a growth factor receptor. By way of non-limiting example, the growth factor receptor may be selected from an epidermal growth factor receptor, a fibroblast growth factor receptor, a platelet derived growth factor receptor, a nerve growth factor receptor, a transforming growth factor receptor, a bone morphogenic protein growth factor receptor, a hepatocyte growth factor receptor, a vascular endothelial growth factor receptor, a stem cell factor receptor, an insulin growth factor receptor, a somatomedin receptor, an erythropoietin receptor and homologs and fragments thereof. The receptor may be a hormone receptor. The receptor may be an insulin receptor. By way of non-limiting example, the receptor may be selected from an eicosanoid receptor, a prostaglandin receptor, an oestrogen receptor, a follicle stimulating hormone receptor, a progesterone receptor, a growth hormone receptor, a gonadotropin-releasing hormone receptor, homologs thereof and fragments thereof. The receptor may be an adrenergic receptor. The receptor may be an integrin. The receptor may be an Eph receptor. The receptor may be a luteinising hormone receptor. The cell surface molecule may be at least about 50% homologous to a luteinising hormone receptor. The receptor may be an immune receptor. By way of non-limiting example, the immune receptor may be selected from a pattern recognition receptor, a toll-like receptor, a NOD-like receptor, a killer-activated receptor, a killer inhibitor receptor, an Fc receptor, a B cell receptor, a complement receptor, a chemokine receptor and a cytokine receptor. By way of non-limiting example, the cytokine receptor may be selected from an interleukin receptor, an interferon receptor, a transforming growth factor receptor, a tumour necrosis factor receptor, a colony stimulating factor receptor, homologs thereof and fragments thereof. The receptor may be a receptor kinase. The receptor kinase may be a tyrosine kinase receptor. The receptor kinase may be a serine kinase receptor. The receptor kinase may be a threonine kinase receptor. By way of non-limiting example, the receptor kinase may activate a signalling protein selected from a Ras, a Raf, a PI3K, a protein kinase A, a protein kinase B, a protein kinase C, an AKT, an AMPK, a phospholipase, homo logs thereof and fragments thereof. The receptor kinase may activate a MAPK/ERK signalling pathway. The receptor kinase may activate Jak, Stat or Smad.

The cell surface molecule may be a non-receptor cell surface protein. The cell surface molecule may be a cluster of differentiation proteins. By way of non-limiting example, the cell surface molecule may be selected from CD3, CD4, CD8, CD11a, CD11b, CD13, CD14, CD15, CD16, CD22, CD24, CD25, CD30, CD31, CD33, CD34, CD38, CD45, CD56, CD61, CD91, CD114, CD117, CD182, CD200, fragments thereof, and homologs thereof.

The cell surface molecule of the target cell may be a molecule that does not comprise a peptide. The cell surface molecule may comprise a lipid. The cell surface molecule may comprise a lipid moiety or a lipid group. The lipid moiety may comprise a sterol. The lipid moiety may comprise a fatty acid. The antigen may comprise a glycolipid. The cell surface molecule may comprise a carbohydrate.

The cell surface molecule of the target cell may be an antigen. The antigen may be at least a portion of a surface antigen or a cell surface marker on a cell. The antigen may be a receptor or a co-receptor on a cell. The antigen may refer to a molecule or molecular fragment that may be bound by a major histocompatibility complex (MHC) and presented to a T-cell receptor. The term “antigen” may also refer to an immunogen. The immunogen may provoke an adaptive immune response if injected on its own into a subject. The immunogen may induce an immune response by itself. The antigen may be a superantigen, T-dependent antigen or a T-independent antigen. The antigen may be an exogenous antigen. Exogenous antigens are typically antigens that have entered the body from the outside, for example by inhalation, ingestion, or injection. Some antigens may start out as exogenous antigens, and later become endogenous (for example, intracellular viruses). The antigen may be an endogenous antigen. The endogenous antigen may be an antigen that has been generated within cells as a result of normal cell metabolism, or because of pathogenic infections (e.g., viral, bacterial, fungal, parasitic). The antigen may be an autoantigen. The autoantigen may be a normal protein or complex of proteins (and sometimes DNA or RNA) that is recognised by the immune system of patients suffering from a specific autoimmune disease. These antigens should, under normal conditions, not be the target of the immune system, but, due to genetic and/or environmental factors, the normal immunological tolerance for such an antigen is not present in these patients. The antigen may be present or over-expressed due to a condition or disease. The condition or disease may be a cancer or a leukaemia. The condition may be an inflammatory disease or condition. The condition or disease may be a metabolic disease. The condition may be a genetic disorder.

The present invention also may find application for the treatment of specific B- or T-lineage associated autoimmune diseases, for example using anti-idiotypic antibodies or fragments thereof or ligands thereof for targeting the B cell receptor and/or the T cell receptor. Such diseases include myasthenia gravis, systemic lupus erythematosus (SLE), rheumatoid arthritis (RA), multiple sclerosis (MS), solid organ transplant hyperacute, acute, chronic or mix-type rejection, bone marrow or stem cell transplant rejection, and graft versus host disease. The present invention may also find application in immunomodulation more broadly, for example, the targeting of one or more of the following proteins/receptors PD-1, CTLA-4, LAG-3, TIM-3, TIGIT, and KIR on an immune cell to promote immunosuppression.

The cell surface molecule of the target cell may be an antigen that has been designated as a tumour antigen. Tumour antigens or neo-antigens may be antigens that are presented by MHC I or MHC II molecules on the surface of tumour cells. These antigens may sometimes be presented by tumour cells and never by the normal ones. In this case, they are called tumour-specific antigens (TSAs) and, in general, result from a tumour-specific mutation. More common are antigens that are presented by tumour cells and normal cells, and they are called tumour-associated antigens (TAAs). Cytotoxic T lymphocytes that recognise these antigens may be able to destroy the tumour cells before they proliferate or metastasise. Tumour antigens may also be on the surface of the tumour in the form of, for example, a mutated receptor, in which case they may be recognised by B cells.

The cell surface molecule of the target cell may be an antigen selected from the group consisting of any surface expressed antigens. Exemplary target antigens may comprise but are not limited to: CD33 (Siglec-3), CD123 (IL3RA), CD135 (FLT-3), CD44 (HCAM), CD44V6, CD47, CD184 (CXCR4), CLEC12A (CLL1), LeY, FRP, MICA/B, CD305 (LAIR-1), CD366 (TIM-3), CD96 (TACTILE), CD133, CD56, CD29 (ITGB1), CD44 (HCAM), CD47 (IAP), CD66 (CEA), CD112 (Nectin2), CD117 (c-Kit), CD133, CD146 (MCAM), CD155 (PVR), CD171 (L1CAM), CD200 (OX-2), CD221 (IGF1), CD227 (MUC1), CD243 (MRD1), CD246 (ALK), CD271 (LNGFR), CD19, CD20, GD2, and EGFR. The cell surface molecule of the target cell may include chains of the TCR, MHC I or II presented peptides, sugars, lipids, carbohydrates or any accessible epitope that may be recognised by a binding domain. The antigen may be any referred to in Table 1 in the context of a bridging (BRiDGE) molecule.

Suitable cancer antigens which may be bound by the targeting moiety of the bridging molecule include, but are not limited to, mesothelin (MSLN), prostate specific membrane antigen (PSMA), prostate stem cell antigen (PCSA), carbonic anhydrase IX (CAIX), carcinoembryonic antigen (CEA), CD5, CD7, CD10, CD19, CD20, CD22, CD30, CD33, CD34, CD38, CD41, CD44, CD49f, CD56, CD74, CD123, CD133, CD138, epithelial glycoprotein (EGP 2), epithelial glycoprotein-40 (EGP-40), epithelial cell adhesion molecule (EpCAM), folate-binding protein (FBP), foetal acetylcholine receptor (AChR), folate receptor-α and β (FRα and β), Ganglioside G2 (GD2), Ganglioside G3 (GD3), human Epidermal Growth Factor Receptor 2 (HER-2/ERB2), Epidermal Growth Factor Receptor vIII (EGFRvIII), ERB3, ERB4, human telomerase reverse transcriptase (hTERT), Interleukin-13 receptor subunit alpha-2 (IL-13Rα2), κ-light chain, kinase insert domain receptor (KDR), Lewis A (CA19.9), Lewis Y (LeY), L1 cell adhesion molecule (L1CAM), melanoma-associated antigen 1 (melanoma antigen family A1, MAGE-A1), Mucin 16 (Muc-16), Mucin 1 (Muc-1), NKG2D ligands, cancer-testis antigen NY-ESO-1, oncofoetal antigen (h5T4), tumour-associated glycoprotein 72 (TAG-72), vascular endothelial growth factor R2 (VEGF-R2), Wilms' tumour protein (WT-1), type 1 tyrosine-protein kinase transmembrane receptor (ROR1), B7-H3 (CD276), B7-H6 (Nkp30), Chondroitin sulfate proteoglycan-4 (CSPG4), DNAX Accessory Molecule (DNAM-1), Ephrin type A Receptor 2 (EpHA2), Fibroblast Associated Protein (FAP), Gp100/HLA-A2, Glypican 3 (GPC3), HA-1H, HERK-V, IL-1 1Ra, Latent Membrane Protein 1 (LMP1), Neural cell-adhesion molecule (N-CAM/CD56), and Trail Receptor (TRAIL R). It is understood that these or other cancer antigens can be utilised for targeting by a bridging molecule in the present invention.

The targeting moiety of the bridging molecule may be any binding molecule, for example, a full-size antibody, or fragment thereof, or any antibody or fragment thereof described herein, an immunocytokine (antibody linked to a cytokine, or fragments thereof), a ligand (protein related, peptides, sugar molecules, processed molecules, lipids, cytokines, hormones), a soluble T cell receptor (TcR), a single chain (sc) TcR, single chain T cell receptor binding motifs and a T cell receptor like mAb, an aptamer (such as DNA or RNA), a peptide (e.g. aptamers or bicyclic peptides), a toxin, a lipid or a carbohydrate.

The targeting moiety of the bridging molecule may be a polypeptide and may be a targeting antibody or antibody fragment. The targeting antibody or antibody fragment may be an immunoglobulin (Ig). The immunoglobulin may be selected from an IgG, an IgA, an IgD, an IgE, an IgM, a fragment thereof or a modification thereof. The immunoglobulin may be IgG. The IgG may be IgG1. The IgG may be IgG2. The IgG may be IgG3. The IgG may be IgG4. The IgG may have one or more Fc mutations for modulating endogenous T cell FcR binding to the bridging molecule. The IgG may have one or more Fc mutations for removing the Fc binding capacity to the FcR of FcR-positive cells. The one or more Fc mutations may remove a glycosylation site. The one or more Fc mutations may be selected from E233P, L234V, L235A, delG236, A327G, A330S, P331S, N297Q and any combination thereof. The one or more Fc mutations may be in IgG1. The one or more Fc mutations in the IgG1 may be L234A, L235A, or both. Alternatively, or additionally, the one or more Fc mutations in the IgG1 may be L234A, L235E, or both. Alternatively, or additionally, the one or more Fc mutations in the IgG1 may be N297A. Alternatively, or additionally, the one or more mutations may be in IgG2. The one or more Fc mutations in the IgG2 may be V234A, V237A, or both.

The targeting antibody or antibody fragment may be an Fc null immunoglobulin or a fragment thereof.

As used herein, the term “antibody fragment” refers to any form of an antibody other than the full-length form. Antibody fragments herein include antibodies that are smaller components that exist within full-length antibodies, and antibodies that have been engineered. Antibody fragments include, but are not limited to, Fv, Fc, Fab, and (Fab′)2, single chain Fv (scFv), diabodies, triabodies, tetrabodies, bifunctional hybrid antibodies, CDR1, CDR2, CDR3, combinations of CDRs, variable regions, framework regions, constant regions, heavy chains, light chains, alternative scaffold non-antibody molecules, and bispecific antibodies. Unless specifically noted otherwise, statements and claims that use the term “antibody” or “antibodies” may specifically include “antibody fragment” and “antibody fragments.”

The targeting antibody fragment may be human, fully human, humanised, human engineered, non-human, and/or chimeric antibody. The non-human antibody may be humanised to reduce immunogenicity to humans, while retaining the specificity and affinity of the parental non-human antibody. Chimeric antibodies may refer to antibodies created through the joining of two or more antibody genes that originally encoded for separate antibodies. A chimeric antibody may comprise at least one amino acid from a first antibody and at least one amino acid from a second antibody, wherein the first and second antibodies are different. At least a portion of the antibody or antibody fragment may be from a bovine species, a human species, or a murine species. At least a portion of the antibody or antibody fragment may be from a rat, a goat, a guinea pig or a rabbit. At least a portion of the antibody or antibody fragment may be from a human. At least a portion of the antibody or antibody fragment antibody may be from cynomolgus monkey.

The targeting antibody or antibody fragment may be based on or derived from an antibody or antibody fragment from a mammal, bird, fish, amphibian or reptile. Mammals include, but are not limited to, carnivores, rodents, elephants, marsupials, rabbits, bats, primates, seals, anteaters, cetaceans, odd-toed ungulates and even-toed ungulates. The mammal may be a human, non-human primate, mouse, sheep, cat, dog, cow, horse, goat, or pig.

The targeting antibody or an antibody fragment may recognise or bind an antigen selected from, by non-limiting example, CD19, Her2, CLL-1, CD33, EGFRvIII, CD20, CD22, BCMA or a fragment thereof. The antigen may comprise a wild-type antigen. The antigen may comprise one or more mutations.

The targeting antibody or antibody fragment may be an anti-CD19 antibody or a fragment thereof. The targeting polypeptide may be an anti-CD22 antibody. The targeting polypeptide may be an anti-BCMA antibody or a fragment thereof. The targeting polypeptide may be an anti-EGFRvIII antibody or a fragment thereof. The targeting polypeptide may be an anti-Her2 antibody or a fragment thereof. The targeting polypeptide may comprise an anti-CD20 antibody or antibody fragment. The targeting polypeptide may comprise rituximab. The targeting polypeptide may comprise an anti-EGFR antibody or antibody fragment. The targeting polypeptide may comprise an anti-CEA antibody or antibody fragment. The targeting polypeptide may comprise an anti-CLL-1 antibody or antibody fragment. The targeting polypeptide may comprise an anti-CD33 antibody or antibody fragment. The targeting polypeptide may comprise an anti-EpCAM antibody or fragment thereof.

The targeting antibody or antibody fragment may be selected from any commercially available antibody. The targeting antibody or antibody fragment may be selected from ado-trastuzumab emtansine, alemtuzumab, bevacizumab, brentuximab, vedotin, gemtuzumab, ozogamicin, ipilimumab, ibritumomab, tiuxetan, panitumumab, cetuximab, erbitux, rituximab, trastuzumab and fragments thereof. The targeting antibody or antibody fragment may be any referred to in Table 1.

The targeting moiety of the bridging molecule may target peptide MHC complexes and in such embodiments, the target moiety may be a soluble TcR molecule or single chain TcR molecule.

Non-limiting examples of the sequences of various targeting antibodies, or antigen binding fragments thereof, are provided herein in Table 1.

### Dysfunctional P2X₇ Receptor Epitope Moiety

A dysfunctional P2X₇ receptor epitope moiety may be provided in the form of a dysfunctional P2X₇ receptor, or a fragment of a dysfunctional P2X₇ receptor, that has at least one of the three ATP binding sites that are formed at the interface between adjacent correctly packed monomers that are unable to bind ATP. Such receptors are unable to extend the opening of the non-selective calcium channels to apoptotic pores.

A range of peptide fragments of a dysfunctional P2X₇ receptor are known and discussed in PCT/AU2002/000061 (and in corresponding publications WO 2002/057306 and U.S. Pat. Nos. 7,326,415, 7,888,473, 7,531,171, 8,080,635, 8,399,617, 8,709,425, 9,663,584, or U.S. Pat. No. 10,450,380), PCT/AU2008/001364 (and in corresponding publications WO 2009/033233 and U.S. Pat. Nos. 8,440,186, 9,181,320, 9,944,701 or U.S. Pat. No. 10,597,45) and PCT/AU2009/000869 (and in corresponding publications WO 2010/000041 and U.S. Pat. No. 8,597,643, 9,328,155 or 10,238,716) the contents of all of which are incorporated in entirety. Exemplary peptides within these specifications that include epitopes contemplated for use in this invention are described below.

| PCT | |
|---|---|
| publication | Peptide sequence |
| WO 2002/ | GHNYTTRNILPGLNITC (SEQ ID NO: 2) |
| 057306 | (also referred to herein as |
| | the “E200” epitope) |
| WO 2009/ | KYYKENNVEKRTLIKVF (SEQ ID NO: 12) |
| 033233 | (also referred to herein as |
| | the “E300” epitope) |
| WO 2010/ | GHNYTTRNILPGAGAKYYKENNVEK |
| 000041 | (SEQ ID NO: 14) (also referred to |
| | herein as the “E200/E300” or |
| | “composite” epitope) |

In any embodiment, the amino acid sequence of the dysfunctional P2X₇ receptor epitope moiety of any bridging molecule described herein, is a sequence as set forth in any of SEQ ID Nos: 2 to 30,168,361-396, 437 and 438 or sequences at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, identical thereto. Preferably, the dysfunctional P2X₇ receptor epitope moiety comprises at least the sequence of SEQ ID NO: 11.

The dysfunctional P2X₇ receptor epitope moiety may have any functional chemical group such as a carboxyl group, an active ester, an acetamide or maleimide capable of coupling to a targeting moiety as disclosed herein, for example an antibody or fragment thereof using NH2 or SH groups for coupling thereto.

### EGFRvIII Epitope Moiety

In any embodiment, the amino acid sequence of the EGFRvIII epitope moiety of any bridging molecule described herein, is a sequence as set forth in any of SEQ ID Nos: 267, or sequences at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, identical thereto. Preferably, the EGFRvIII epitope moiety comprises at least the sequence of SEQ ID NO: 267.

### CLDN6 Epitope Moiety

In any embodiment, the amino acid sequence of the CLDN6 epitope moiety of any bridging molecule described herein, is a sequence as set forth in any of SEQ ID Nos: 273, 274 or 275, or sequences at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, identical thereto. Preferably, the CLDN6 epitope moiety comprises at least the sequence of SEQ ID NO: 273, 274 or 275.

### Exemplary Bridging Molecules

The present specification provides various non-limiting examples of tumour-specific antigen epitope moiety (e.g. dysfunctional P2X₇ receptor epitope moiety)/targeting moiety pairs.

Exemplary bridging molecules are described in Table 1. For those bridging molecules that are described in Table 1 that include a nfP2X₇ epitope moiety, the specification includes those BRiDGEs but with the nfP2X₇ epitope moiety substituted for a EGFRvIII or CLDN6 epitope moiety.

In examples where the bridging molecules comprise a targeting moiety for binding to CD19, the targeting moiety may comprise a heavy and paired light variable chain combination as set forth in SEQ ID NOs: 31 and 32; or 143 and 144 (heavy and light chain, respectively; or sequences at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, identical thereto.

In the above examples, the bridging molecules may comprise the tumour-specific antigen epitope moiety (e.g. dysfunctional P2X₇ receptor epitope moiety) conjugated to the heavy chain, or the tumour-specific antigen epitope moiety (e.g. dysfunctional P2X₇ receptor epitope moiety) conjugated to the light chain. Preferably, the tumour-specific antigen epitope moiety (e.g. dysfunctional P2X₇ receptor epitope moiety) is conjugated to the light chain of the target binding moiety.

In any embodiment wherein the bridging molecule comprises CD19-binding heavy/light chain pairs where the heavy chain comprises the dysfunctional P2X₇ receptor epitope moiety, the sequences of the variable sequences of the heavy and light chain pairs are preferably selected from: SEQ ID NOs: 33 and 32; 34 and 32, 37 and 32; 37 and 38; (heavy and light chain sequences recited, respectively) or sequences at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, identical thereto.

In any embodiment wherein the bridging molecule comprises or consists of CD19-binding heavy/light chain pairs where the light chain comprises the dysfunctional P2X₇ receptor epitope moiety, the sequences of the variable sequences of the heavy and light chain pairs are preferably selected from: SEQ ID NOs: 31 and 35; 31 and 36; 39 and 31; 52 and 51; 143 and 145; 143 and 146; 143 and 147; 143 and 148; 143 and 149; 143 and 150; 143 and 151; 143 and 152; 143 and 153; 143 and 154; 143 and 155; 143 and 1561; 143 and 157; 143 and 158; 143 and 159; 143 and 160; 143 and 161; 143 and 162; 143 and 163; or 143 and 164 (heavy and light chain sequences recited, respectively) or sequences at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, identical thereto. In another embodiment wherein the bridging molecule comprises CD19-binding heavy/light chain pairs where the light chain is any one of the light chains above and a heavy chain selected from SEQ ID NO: 141 or 142.

The targeting moiety may be in the form of an scFv comprising a heavy and a light chain.

In any embodiment, a CD19-binding scFv for use in the bridging molecules of the invention may be one having a sequence as set forth in SEQ ID NOs: 40 or 41 or sequences at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, identical thereto. It will be appreciated that in the context of an scFv, the dysfunctional P2X₇ receptor epitope moiety may be conjugated to the light chain of the scFv, such as in any of SEQ ID NOs: 42, 43, 46, 48, or to the heavy chain of the scFv, such as in any of SEQ ID NOs: 44, 45, 360, 47, 49, 50 or sequences at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, identical thereto.

In any embodiment, a bridging molecule for binding to CD20 may comprise or consist of the sequences set forth in SEQ ID NOs: 53 and 54, or in 55 and 56 (light and heavy chain sequences recited, respectively) or a sequence at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, identical thereto.

In any embodiment, a bridging molecule for binding to CD22 may comprise or consist of the sequences set forth in SEQ ID NOs: 57 and 58; or in 59 and 60 (light and heavy chain sequences recited, respectively) or a sequence at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, identical thereto.

In any embodiment, a bridging molecule for binding to CD79B may comprise or consist of the sequences set forth in SEQ ID NOs: 61 and 62, (light and heavy chain sequences recited, respectively) or a sequence at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, identical thereto.

In any embodiment, a bridging molecule for binding to CD37 may comprise or consist of the sequences set forth in SEQ ID NOs: 63 and 64, (light and heavy chain sequences recited, respectively) or a sequence at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, identical thereto.

In any embodiment, a bridging molecule for binding to CD38 may comprise or consist of the sequences set forth in SEQ ID NOs: 65 and 66, (light and heavy chain sequences recited, respectively) or a sequence at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, identical thereto.

In any embodiment, a bridging molecule for binding to CD70 may comprise or consist of the sequences set forth in SEQ ID NOs: 67 and 68, (light and heavy chain sequences recited, respectively) or a sequence at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, identical thereto.

In any embodiment, a bridging molecule for binding to CD30 may comprise or consist of the sequences set forth in SEQ ID NOs: 39 and 70, (light and heavy chain sequences recited, respectively) or a sequence at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, identical thereto.

In any embodiment, a bridging molecule for binding to CD33 may comprise or consist of the sequences set forth in SEQ ID NOs: 71 and 72 or 73 and 74, (light and heavy chain sequences recited, respectively) or a sequence at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, identical thereto.

In any embodiment, a bridging molecule for binding to Her2 may comprise or consist of the sequences set forth in SEQ ID NOs: 75 and 75; or 77 and 78, (light and heavy chain sequences recited, respectively) or a sequence at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, identical thereto.

In any embodiment, a bridging molecule for binding to EGFR may comprise or consist of the sequences set forth in SEQ ID NOs: 79 and 80 or 81 and 82 or 83 and 84, (light and heavy chain sequences recited, respectively) or a sequence at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, identical thereto.

In any embodiment, a bridging molecule for binding to CD276 may comprise or consist of the sequences set forth in SEQ ID NOs: 85 and 86, (light and heavy chain sequences recited, respectively) or a sequence at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, identical thereto.

In any embodiment, a bridging molecule for binding to GD2 may comprise or consist of the sequences set forth in SEQ ID NOs: 87 and 88, (light and heavy chain sequences recited, respectively) or a sequence at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, identical thereto.

In any embodiment, a bridging molecule for binding to BCMA may comprise or consist of the sequences set forth in SEQ ID NOs: 89 and 90, (light and heavy chain sequences recited, respectively) or a sequence at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, identical thereto.

In any embodiment, a bridging molecule for binding to CD371 may comprise or consist of the sequences set forth in SEQ ID NOs: 91 and 92, (light and heavy chain sequences recited, respectively) or a sequence at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, identical thereto.

In any embodiment, a bridging molecule for binding to CD135 may comprise or consist of the sequences set forth in SEQ ID NOs: 93 and 94, (light and heavy chain sequences recited, respectively) or a sequence at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, identical thereto.

In any embodiment, a bridging molecule for binding to CD123 may comprise or consist of the sequences set forth in SEQ ID NOs: 95 and 95, (light and heavy chain sequences recited, respectively) or a sequence at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, identical thereto.

In any embodiment, a bridging molecule for binding to CD105 may comprise or consist of the sequences set forth in SEQ ID NOs: 97 and 98, (light and heavy chain sequences recited, respectively) or a sequence at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, identical thereto.

In any embodiment, a bridging molecule for binding to ROR-1 may comprise or consist of the sequences set forth in SEQ ID NOs: 99 and 100, (light and heavy chain sequences recited, respectively) or a sequence at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, identical thereto.

In any embodiment, a bridging molecule for binding to PD-L1 may comprise or consist of the sequences set forth in SEQ ID NOs: 101 and 102, (light and heavy chain sequences recited, respectively) or a sequence at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, identical thereto.

In any embodiment, a bridging molecule for binding to MET-R may comprise or consist of the sequences set forth in SEQ ID NOs: 103 ad 104, (light and heavy chain sequences recited, respectively) or a sequence at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, identical thereto.

In any embodiment, a bridging molecule for binding to PDGFRalpha may comprise or consist of the sequences set forth in SEQ ID NOs: 105 and 106 or 107 and 108 (light and heavy chain sequences recited, respectively) or a sequence at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, identical thereto.

In any embodiment, a bridging molecule for binding to Her3 may comprise or consist of the sequences set forth in SEQ ID NOs: 109 and 110, (light and heavy chain sequences recited, respectively) or a sequence at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, identical thereto.

In any embodiment, a bridging molecule for binding to FRalpha may comprise or consist of the sequences set forth in SEQ ID NOs: 111 and 112, (light and heavy chain sequences recited, respectively) or a sequence at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, identical thereto.

In any embodiment, a bridging molecule for binding to CGPC3 may comprise or consist of the sequences set forth in SEQ ID NOs: 113 and 114, (light and heavy chain sequences recited, respectively) or a sequence at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, identical thereto.

In any embodiment, a bridging molecule for binding to SLAMF7 may comprise or consist of the sequences set forth in SEQ ID NOs: 115 and 116, (light and heavy chain sequences recited, respectively) or a sequence at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, identical thereto.

In any embodiment, a bridging molecule for binding to TNFRSF10B may comprise or consist of the sequences set forth in SEQ ID NOs: 117 and 118, (light and heavy chain sequences recited, respectively) or a sequence at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, identical thereto.

In any embodiment, a bridging molecule for binding to GPNMB may comprise or consist of the sequences set forth in SEQ ID NOs: 119 and 120, (light and heavy chain sequences recited, respectively) or a sequence at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, identical thereto.

In any embodiment, a bridging molecule for binding to VEGFR2 may comprise or consist of the sequences set forth in SEQ ID NOs: 121 and 122, (light and heavy chain sequences recited, respectively) or a sequence at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, identical thereto.

In any embodiment, a bridging molecule for binding to α4β7 and/or αEβ7 may comprise or consist of the sequences set forth in SEQ ID NOs: 123 and 124; or 125 and 126, (light and heavy chain sequences recited, respectively) or a sequence at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, identical thereto.

In any embodiment, a bridging molecule for binding to CSPG4 may comprise or consist of the sequences set forth in SEQ ID NOs: 127 and 128, (light and heavy chain sequences recited, respectively) or a sequence at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, identical thereto.

In any embodiment, a bridging molecule for binding to CD80 may comprise or consist of the sequences set forth in SEQ ID NOs: 129 and 130, (light and heavy chain sequences recited, respectively) or a sequence at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, identical thereto.

In any embodiment, a bridging molecule for binding to CCR4 may comprise or consist of the sequences set forth in SEQ ID NOs: 131 and 132, (light and heavy chain sequences recited, respectively) or a sequence at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, identical thereto.

In any embodiment, a bridging molecule for binding to CD115 may comprise or consist of the sequences set forth in SEQ ID NOs: 133 and 134, (light and heavy chain sequences recited, respectively) or a sequence at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, identical thereto.

In any embodiment, a bridging molecule for binding to ENOX-2 may comprise or consist of the sequences set forth in SEQ ID NOs: 135 and 136, (light and heavy chain sequences recited, respectively) or a sequence at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, identical thereto.

In any embodiment, a bridging molecule for binding to CD56 may comprise or consist of the sequences set forth in SEQ ID NOs: 137 and 138, (light and heavy chain sequences recited, respectively) or a sequence at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, identical thereto.

In any embodiment, a bridging molecule for binding to huVH1-69 may comprise or consist of the sequences set forth in SEQ ID NOs: 139 and 140, (light and heavy chain sequences recited, respectively) or a sequence at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, identical thereto.

In any embodiment, a bridging molecule for binding to CD117 may comprise or consist of the sequences set forth in SEQ ID NOs: 169 and 170, (light and heavy chain sequences recited, respectively) or a sequence at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, identical thereto.

In any embodiment, a bridging molecule for binding to CD133 may comprise or consist of the sequences set forth in SEQ ID NOs: 171 and 172, (light and heavy chain sequences recited, respectively) or a sequence at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, identical thereto.

In any embodiment, a bridging molecule for binding to MUC1 may comprise or consist of the sequences set forth in SEQ ID NOs: 173 and 174, (light and heavy chain sequences recited, respectively) or a sequence at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, identical thereto.

In any embodiment, a bridging molecule for binding to mesothelin may comprise or consist of the sequences set forth in SEQ ID NOs: 175 and 176, (light and heavy chain sequences recited, respectively) or a sequence at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, identical thereto.

In any embodiment, a bridging molecule for binding to ROR2 may comprise or consist of the sequences set forth in SEQ ID NOs: 177 and 178, (light and heavy chain sequences recited, respectively) or a sequence at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, identical thereto.

In any embodiment, a bridging molecule for binding to IL13Ra2 may comprise or consist of the sequences set forth in SEQ ID NOs: 179 and 180, (light and heavy chain sequences recited, respectively) or a sequence at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, identical thereto.

In any embodiment, a bridging molecule for binding to IL13Ra2 may comprise or consist of the sequences set forth in SEQ ID NOs: 181, or a sequence at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, identical thereto.

In any embodiment, a bridging molecule for binding to EPHA2 may comprise or consist of the sequences set forth in SEQ ID NOs: 182 and 183, (light and heavy chain sequences recited, respectively) or a sequence at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, identical thereto.

In any embodiment, a bridging molecule for binding to EGFRvIII may comprise or consist of the sequences set forth in SEQ ID NOs: 184 and 185, (light and heavy chain sequences recited, respectively) or a sequence at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, identical thereto.

In any embodiment, a bridging molecule for binding to PSMA may comprise or consist of the sequences set forth in SEQ ID NOs: 186 and 187, (light and heavy chain sequences recited, respectively) or a sequence at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, identical thereto.

In any embodiment, a bridging molecule for binding to CEA may comprise or consist of the sequences set forth in SEQ ID NOs: 188 and 189, (light and heavy chain sequences recited, respectively) or a sequence at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, identical thereto.

In any embodiment, a bridging molecule for binding to PSCA may comprise or consist of the sequences set forth in SEQ ID NOs: 190 and 191, (light and heavy chain sequences recited, respectively) or a sequence at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, identical thereto.

In any embodiment, a bridging molecule for binding to Lewis Y may comprise or consist of the sequences set forth in SEQ ID NOs: 192 and 193, (light and heavy chain sequences recited, respectively) or a sequence at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, identical thereto.

In any embodiment, a bridging molecule for binding to CD171 L1CAM may comprise or consist of the sequences set forth in SEQ ID NOs: 194 and 195, (light and heavy chain sequences recited, respectively) or a sequence at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, identical thereto.

In any embodiment, a bridging molecule for binding to EpCAM may comprise or consist of the sequences set forth in SEQ ID NOs: 196 and 197, (light and heavy chain sequences recited, respectively) or a sequence at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, identical thereto.

In any embodiment, a bridging molecule for binding to ALK may comprise or consist of the sequences set forth in SEQ ID NOs: 198 and 199, (light and heavy chain sequences recited, respectively) or a sequence at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, identical thereto.

In any embodiment, a bridging molecule for binding to IGF-1R CD221 may comprise or consist of the sequences set forth in SEQ ID NOs: 200 and 201, (light and heavy chain sequences recited, respectively) or a sequence at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, identical thereto.

In any embodiment, a bridging molecule for binding to Nectin 4 may comprise or consist of the sequences set forth in SEQ ID NOs: 202 and 203, (light and heavy chain sequences recited, respectively) or a sequence at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, identical thereto.

In any embodiment, a bridging molecule for binding to FAP may comprise or consist of the sequences set forth in SEQ ID NOs: 204 and 205, (light and heavy chain sequences recited, respectively) or a sequence at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, identical thereto.

In any embodiment, a bridging molecule for binding to AXL may comprise or consist of the sequences set forth in SEQ ID NOs: 206 and 207, (light and heavy chain sequences recited, respectively) or a sequence at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, identical thereto.

In any embodiment, a bridging molecule for binding to CD138 may comprise or consist of the sequences set forth in SEQ ID NOs: 208 and 209, (light and heavy chain sequences recited, respectively) or a sequence at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, identical thereto.

In any embodiment, a bridging molecule for binding to CLDN6 may comprise or consist of the sequences set forth in SEQ ID NOs: 210 and 211, (light and heavy chain sequences recited, respectively) or a sequence at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, identical thereto.

In any embodiment, a bridging molecule for binding to Her4 may comprise or consist of the sequences set forth in SEQ ID NOs: 212 and 213, (light and heavy chain sequences recited, respectively) or a sequence at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, identical thereto.

In any embodiment, a bridging molecule for binding to Claudin 18.2 may comprise or consist of the sequences set forth in SEQ ID NOs: 214 and 215, (light and heavy chain sequences recited, respectively) or a sequence at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, identical thereto.

In any embodiment, a bridging molecule for binding to O-acetylated GD2 may comprise or consist of the sequences set forth in SEQ ID NOs: 216 and 217, (light and heavy chain sequences recited, respectively) or a sequence at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, identical thereto.

In any embodiment, a bridging molecule for binding to GD3 may comprise or consist of the sequences set forth in SEQ ID NOs: 218 and 219, (light and heavy chain sequences recited, respectively) or a sequence at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, identical thereto.

In any embodiment, a bridging molecule for binding to GM2 may comprise or consist of the sequences set forth in SEQ ID NOs: 220 and 221, (light and heavy chain sequences recited, respectively) or a sequence at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, identical thereto.

In any embodiment, a bridging molecule for binding to TM4SF1 may comprise or consist of the sequences set forth in SEQ ID NOs: 222 and 223, (light and heavy chain sequences recited, respectively) or a sequence at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, identical thereto.

In any embodiment, a bridging molecule for binding to CD147 may comprise or consist of the sequences set forth in SEQ ID NOs: 224 and 225, (light and heavy chain sequences recited, respectively) or a sequence at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, identical thereto.

In any embodiment, a bridging molecule for binding to CEACAM5 may comprise or consist of the sequences set forth in SEQ ID NOs: 226 and 227, (light and heavy chain sequences recited, respectively) or a sequence at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, identical thereto.

In any embodiment, a bridging molecule for binding to VEGFR-1 may comprise or consist of the sequences set forth in SEQ ID NOs: 228 and 229, (light and heavy chain sequences recited, respectively) or a sequence at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, identical thereto.

In any embodiment, a bridging molecule for binding to Podoplanin (PDPN) may comprise or consist of the sequences set forth in SEQ ID NOs: 230 and 231, (light and heavy chain sequences recited, respectively) or a sequence at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, identical thereto.

In any embodiment, a bridging molecule for binding to WT1 may comprise or consist of the sequences set forth in SEQ ID NOs: 232 and 233, (light and heavy chain sequences recited, respectively) or a sequence at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, identical thereto.

In any embodiment, a bridging molecule for binding to GPC2 may comprise or consist of the sequences set forth in SEQ ID NOs: 234 and 235, (light and heavy chain sequences recited, respectively) or a sequence at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, identical thereto.

In any embodiment, a bridging molecule for binding to FGFR4 may comprise or consist of the sequences set forth in SEQ ID NOs: 236 and 237, (light and heavy chain sequences recited, respectively) or a sequence at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, identical thereto.

In any embodiment, a bridging molecule for binding to EphB4 may comprise or consist of the sequences set forth in SEQ ID NOs: 238 and 239, (light and heavy chain sequences recited, respectively) or a sequence at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, identical thereto.

In any embodiment, a bridging molecule for binding to STEAP-1 may comprise or consist of the sequences set forth in SEQ ID NOs: 240 and 241, (light and heavy chain sequences recited, respectively) or a sequence at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, identical thereto.

In any embodiment, a bridging molecule for binding to STEAP-2 may comprise or consist of the sequences set forth in SEQ ID NOs: 242 and 243, (light and heavy chain sequences recited, respectively) or a sequence at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, identical thereto.

In any embodiment, a bridging molecule for binding to IL11 Ra may comprise or consist of the sequences set forth in SEQ ID NOs: 244 and 245, (light and heavy chain sequences recited, respectively) or a sequence at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, identical thereto.

In any embodiment, a bridging molecule for binding to CD163 may comprise or consist of the sequences set forth in SEQ ID NOs: 246 and 247, (light and heavy chain sequences recited, respectively) or a sequence at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, identical thereto.

In any embodiment, a bridging molecule for binding to Chlorotoxin may comprise or consist of the sequences set forth in SEQ ID NOs: 248 and 249, (light and heavy chain sequences recited, respectively) or a sequence at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, identical thereto.

In any embodiment, a bridging molecule for binding to CD206 may comprise or consist of the sequences set forth in SEQ ID NOs: 250, (heavy chain sequence recited) or a sequence at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, identical thereto.

In any embodiment, a bridging molecule for binding to IL1RAP may comprise or consist of the sequences set forth in SEQ ID NOs: 251 and 252, (light and heavy chain sequences recited, respectively) or a sequence at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, identical thereto.

In any embodiment, a bridging molecule for binding to MICA may comprise or consist of the sequences set forth in SEQ ID NOs: 253 and 254, (light and heavy chain sequences recited, respectively) or a sequence at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, identical thereto.

In any embodiment, a bridging molecule for binding to MAGE-A1 may comprise or consist of the sequences set forth in SEQ ID NOs: 255, or a sequence at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, identical thereto.

In any embodiment, a bridging molecule for binding to MAGE-A1 may comprise or consist of the sequences set forth in SEQ ID NOs: 256 and 257, or a sequence at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, identical thereto.

In any embodiment, a bridging molecule for binding to MAGE-A1 may comprise or consist of the sequences set forth in SEQ ID NOs: 258 and 259, or a sequence at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, identical thereto.

In any embodiment, a bridging molecule for binding to TRBC1 may comprise or consist of the sequences set forth in SEQ ID NOs: 260 and 261, (light and heavy chain sequences recited, respectively) or a sequence at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, identical thereto.

In any embodiment, a bridging molecule for binding to TRBC2 may comprise or consist of the sequences set forth in SEQ ID NOs: 262 and 263, (light and heavy chain sequences recited, respectively) or a sequence at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, identical thereto.

In any embodiment, a bridging molecule for binding to urokinase-type plasminogen activator receptor (uPAR) may comprise or consist of the sequences set forth in SEQ ID NOs: 264 and 265, (light and heavy chain sequences recited, respectively) or a sequence at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, identical thereto.

In any embodiment, a bridging molecule for binding to CD33 may comprise or consist of the sequences set forth in SEQ ID NOs: 268 and 269, (light and heavy chain sequences recited, respectively) or a sequence at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, identical thereto.

In any embodiment, a bridging molecule for binding to Her2 may comprise or consist of the sequences set forth in SEQ ID NOs: 276 and 277, (light and heavy chain sequences recited, respectively) or a sequence at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, identical thereto.

In any embodiment, a bridging molecule for binding to CD33 may comprise or consist of the sequences set forth in SEQ ID NOs: 278 and 279, (light and heavy chain sequences recited, respectively) or a sequence at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, identical thereto.

In any embodiment, a bridging molecule for binding to Her2 may comprise or consist of the sequences set forth in SEQ ID NOs: 270 and 271, (light and heavy chain sequences recited, respectively) or a sequence at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, identical thereto.

In any embodiment, a bridging molecule for binding to B7-H₇ (HHLA2) may comprise or consist of the sequences set forth in SEQ ID NOs: 280 and 281, (light and heavy chain sequences recited, respectively) or a sequence at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, identical thereto.

In any embodiment, a bridging molecule for binding to CD34 may comprise or consist of the sequences set forth in SEQ ID NOs: 282 and 283, (light and heavy chain sequences recited, respectively) or a sequence at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, identical thereto.

In any embodiment, a bridging molecule for binding to CD7 may comprise or consist of the sequences set forth in SEQ ID NOs: 284 and 285, (light and heavy chain sequences recited, respectively) or a sequence at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, identical thereto.

In any embodiment, a bridging molecule for binding to CD7 may comprise or consist of the sequences set forth in SEQ ID NOs: 286, (heavy chain sequence) or a sequence at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, identical thereto.

In any embodiment, a bridging molecule for binding to GPRC5D may comprise or consist of the sequences set forth in SEQ ID NOs: 287 and 288, (light and heavy chain sequences recited, respectively) or a sequence at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, identical thereto.

In any embodiment, a bridging molecule for binding to TIM-3 may comprise or consist of the sequences set forth in SEQ ID NOs: 289 and 290, (light and heavy chain sequences recited, respectively) or a sequence at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, identical thereto.

In any embodiment, a bridging molecule for binding to CD191 (CCR1) may comprise or consist of the sequences set forth in SEQ ID NOs: 291 and 292, (light and heavy chain sequences recited, respectively) or a sequence at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, identical thereto.

In any embodiment, a bridging molecule for binding to CD66b (CEACAM8) may comprise or consist of the sequences set forth in SEQ ID NOs: 293 and 294, (light and heavy chain sequences recited, respectively) or a sequence at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, identical thereto.

In any embodiment, a bridging molecule for binding to CD11 b (MAC-1) may comprise or consist of the sequences set forth in SEQ ID NOs: 295 and 296, (light and heavy chain sequences recited, respectively) or a sequence at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, identical thereto.

In any embodiment, a bridging molecule for binding to EMR2 (ADGRE2) may comprise or consist of the sequences set forth in SEQ ID NOs: 297 and 298, (light and heavy chain sequences recited, respectively) or a sequence at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, identical thereto.

In any embodiment, a bridging molecule for binding to MUC16 may comprise or consist of the sequences set forth in SEQ ID NOs: 299 and 300, (light and heavy chain sequences recited, respectively) or a sequence at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, identical thereto.

In any embodiment, a bridging molecule for binding to NYESO-1 HLA-A2 may comprise or consist of the sequences set forth in SEQ ID NOs: 301 and 302, or a sequence at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, identical thereto.

In any embodiment, a bridging molecule for binding to Survivin HLA-A2 may comprise or consist of the sequences set forth in SEQ ID NOs: 303 and 304, or a sequence at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, identical thereto.

In any embodiment, a bridging molecule for binding to BCMA may comprise or consist of the sequences set forth in SEQ ID NOs: 305, or a sequence at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, identical thereto.

In any embodiment, a bridging molecule for binding to BCMA may comprise or consist of the sequences set forth in SEQ ID NOs: 306, or a sequence at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, identical thereto.

In any embodiment, a bridging molecule for binding to CD200 may comprise or consist of the sequences set forth in SEQ ID NOs: 349 and 348, (light and heavy chain sequences recited, respectively) or a sequence at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, identical thereto.

In any aspect, the bridging molecule described herein does not have a HIS tag. Also contemplated, is a bridging molecule that comprises an amino acid sequence specified in the Sequence information table above, but without a HIS tag specified in the sequence, or a sequence at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, identical thereto. Further, in one embodiment, the bridging molecule may comprise a tag other than a HIS tag, or may comprise an amino acid sequence specified in the Sequence information table above but with a different tag in the position of the HIS tag specified in the sequence.

### Nucleic Acids

In a second aspect, the present invention provides a nucleic acid molecule encoding an antigen binding protein of the invention, or part thereof. The nucleic acid may further encode a bridging molecule described herein.

The nucleic acid molecule may comprise any polyribonucleotide or polydeoxyribonucleotide, which may be unmodified, or modified, RNA or DNA. For example, the nucleic acid molecule may include single- and/or double-stranded DNA, DNA that is a mixture of single- and double-stranded regions, single- and double-stranded RNA, and RNA that is mixture of single- and double-stranded regions, hybrid molecules comprising DNA and RNA that may be single-stranded or, more typically, double-stranded or a mixture of single- and double-stranded regions. In addition, the nucleic acid molecule may comprise triple-stranded regions comprising RNA or DNA or both RNA and DNA. The nucleic acid molecule may also comprise one or more modified bases or DNA or RNA backbones modified for stability or for other reasons. A variety of modifications can be made to DNA and RNA; thus the term “nucleic acid molecule” embraces chemically, enzymatically, or metabolically modified forms.

In some embodiments of the second aspect of the invention, the nucleic acid molecule comprises a nucleotide sequence encoding the amino acid sequence of any one of SEQ ID NOs: 307 to 347. Preferably, the nucleic acid comprises a nucleotide sequence encoding an antigen binding protein described above. Preferably, the nucleic acid further comprises a nucleotide sequence encoding the amino acid sequence of any one of SEQ ID NOs: 2 to 30, 168 361-396, 437 or 438. Preferably, the nucleic acid comprises a nucleotide sequence encoding the heavy chain and light chain pairs of the bridging molecules described above.

Further, the present invention provides a nucleic acid construct including a nucleic acid molecule encoding an antigen binding protein molecule of the invention, or part thereof. The nucleic acid construct may further comprise one or more of: an origin of replication for one or more hosts; a selectable marker gene that is active in one or more hosts; and/or one or more transcriptional control sequences.

As used herein, the term “selectable marker gene” includes any gene that confers a phenotype on a cell in which it is expressed, to facilitate the identification and/or selection of cells that are transfected or transformed with the construct.

“Selectable marker genes” include any nucleotide sequences which, when expressed by a cell transformed with the construct, confer a phenotype on the cell that facilitates the identification and/or selection of these transformed cells. A range of nucleotide sequences encoding suitable selectable markers are known in the art (for example Mortesen, R M. and Kingston R E. Curr Protoc Mol Biol, 2009; Unit 9.5). Exemplary nucleotide sequences that encode selectable markers include: Adenosine deaminase (ADA) gene; Cytosine deaminase (CDA) gene; Dihydrofolate reductase (DHFR) gene; Histidinol dehydrogenase (hisD) gene; Puromycin-N-acetyl transferase (PAC) gene; Thymidine kinase (TK) gene; Xanthine-guanine phosphoribosyltransferase (XGPRT) gene or antibiotic resistance genes such as ampicillin-resistance genes, puromycin-resistance genes, Bleomycin-resistance genes, hygromycin-resistance genes, kanamycin-resistance genes and ampicillin-resistance genes; fluorescent reporter genes such as the green, red, yellow or blue fluorescent protein-encoding genes; and luminescence-based reporter genes such as the luciferase gene, amongst others which permit optical selection of cells using techniques such as Fluorescence-Activated Cell Sorting (FACS).

Furthermore, it should be noted that the selectable marker gene may be a distinct open reading frame in the construct or may be expressed as a fusion protein with another polypeptide (e.g. the CAR).

As set out above, the nucleic acid construct may also comprise one or more transcriptional control sequences. The term “transcriptional control sequence” should be understood to include any nucleic acid sequence that effects the transcription of an operably connected nucleic acid. A transcriptional control sequence may include, for example, a leader, polyadenylation sequence, promoter, enhancer or upstream activating sequence, and transcription terminator. Typically, a transcriptional control sequence at least includes a promoter. The term “promoter” as used herein, describes any nucleic acid that confers, activates or enhances expression of a nucleic acid in a cell.

In some embodiments, at least one transcriptional control sequence is operably connected to the nucleic acid molecule of the second aspect of the invention. For the purposes of the present specification, a transcriptional control sequence is regarded as “operably connected” to a given nucleic acid molecule when the transcriptional control sequence is able to promote, inhibit or otherwise modulate the transcription of the nucleic acid molecule. Therefore, in some embodiments, the nucleic acid molecule is under the control of a transcription control sequence, such as a constitutive promoter or an inducible promoter.

The “nucleic acid construct” may be in any suitable form, such as in the form of a plasmid, phage, transposon, cosmid, chromosome, vector, etc., which is capable of replication when associated with the proper control elements and which can transfer gene sequences, contained within the construct, between cells. Thus, the term includes cloning and expression vehicles, as well as viral vectors. In some embodiments, the nucleic acid construct is a vector. In some embodiments the vector is a viral vector.

A promoter may regulate the expression of an operably connected nucleic acid molecule constitutively, or differentially, with respect to the cell, tissue, or organ at which expression occurs. As such, the promoter may include, for example, a constitutive promoter, or an inducible promoter. A “constitutive promoter” is a promoter that is active under most environmental and physiological conditions. An “inducible promoter” is a promoter that is active under specific environmental or physiological conditions. The present invention contemplates the use of any promoter that is active in a cell of interest. As such, a wide array of promoters would be readily ascertained by one of ordinary skill in the art.

Mammalian constitutive promoters may include, but are not limited to, Simian virus 40 (SV40), cytomegalovirus (CMV), P-actin, Ubiquitin C (UBC), elongation factor-1 alpha (EF1A), phosphoglycerate kinase (PGK) and CMV early enhancer/chicken p actin (CAGG).

Inducible promoters may include, but are not limited to, chemically inducible promoters and physically inducible promoters. Chemically inducible promoters include promoters that have activity that is regulated by chemical compounds such as alcohols, antibiotics, steroids, metal ions or other compounds. Examples of chemically inducible promoters include: tetracycline regulated promoters (e.g. see U.S. Pat. Nos. 5,851,796 and 5,464,758); steroid responsive promoters such as glucocorticoid receptor promoters (e.g. see U.S. Pat. No. 5,512,483), ecdysone receptor promoters (e.g. see U.S. Pat. No. 6,379,945) and the like; and metal-responsive promoters such as metallothionein promoters (e.g. see U.S. Pat. Nos. 4,940,661, 4,579,821 and 4,601,978) amongst others.

In the context of the present invention, it will be appreciated that it may be desirable in certain circumstances for the expression of the bridging molecule to be under the control of an inducible promoter. This enables a switching on and switching off of the expression of the nucleic acid encoding the bridging molecule.

In certain embodiment, and in the case of an inducible expression construct, an immune cell expressing a CAR can be genetically modified with a) a nucleic acid encoding an antigen binding receptor and b) an inducible expression construct encoding the antigen binding protein and/or bridging molecule. Upon binding of dysfunctional P2X₇ receptor, the immune cell induces expression of the gene encoding the antigen binding protein and/or bridging molecule. In certain embodiments, expression of such gene facilitates and/or improves treatment of cancer.

As mentioned above, the control sequences may also include a terminator. The term “terminator” refers to a DNA sequence at the end of a transcriptional unit that signals termination of transcription. Terminators are 3′-non-translated DNA sequences generally containing a polyadenylation signal, which facilitate the addition of polyadenylate sequences to the 3′-end of a primary transcript. As with promoter sequences, the terminator may be any terminator sequence that is operable in the cells, tissues or organs in which it is intended to be used. Suitable terminators would be known to a person skilled in the art.

As will be understood, the nucleic acid constructs of the invention can further include additional sequences, for example sequences that permit enhanced expression, cytoplasmic or membrane transportation, and location signals. Specific non-limiting examples include an Internal Ribosome Entry Site (IRES) or cleavage site (e.g. P2A, T2A).

The present invention extends to all genetic constructs essentially as described herein. These constructs may further include nucleotide sequences intended for the maintenance and/or replication of the genetic construct in eukaryotes and/or the integration of the genetic construct or a part thereof into the genome of a eukaryotic cell.

Methods are known in the art for the deliberate introduction (transfection/transduction) of exogenous genetic material, such as the nucleic acid construct of the third aspect of the present invention, into eukaryotic cells. As will be understood, the method best suited for introducing the nucleic acid construct into the desired host cell is dependent on many factors, such as the size of the nucleic acid construct, the type of host cell, the desired rate of efficiency of the transfection/transduction and the final desired, or required, viability of the transfected/transduced cells. Non-limiting examples of such methods include; chemical transfection with chemicals such as cationic polymers, calcium phosphate, or structures such as liposomes and dendrimers; non-chemical methods such as electroporation, sonoporation, heat-shock or optical transfection; particle-based methods such as ‘gene gun’ delivery, magnetofection, or impalefection or viral transduction.

The nucleic acid construct will be selected depending on the desired method of transfection/transduction. In some embodiments of the third aspect of the invention, the nucleic acid construct is a viral vector, and the method for introducing the nucleic acid construct into a host cell is viral transduction. Methods are known in the art for utilising viral transduction to elicit expression of a CAR in a PBMC (Parker, L L. et al. Hum Gene Ther. 2000; 11: 2377-87) and more generally utilising retroviral systems for transduction of mammalian cells (Cepko, C. and Pear, W. Curr Protoc Mol Biol. 2001, unit 9.9). In other embodiments, the nucleic acid construct is a plasmid, a cosmid, an artificial chromosome or the like, and can be transfected into the cell by any suitable method known in the art.

### Modified Cells

As described herein, in certain embodiments the invention includes methods of treatment involving the use of a cell expressing a chimeric antigen receptor (CAR) comprising an antigen-recognition domain, for example, wherein the antigen-recognition domain recognises a dysfunctional P2X₇ receptor expressed on a cell surface. For example, the orchestration molecules of the present invention may be used to facilitate the killing of cancer (target) cells by recruiting cells expressing a chimeric antigen receptor. Such orchestration molecules will typically comprise a first antigen binding domain for binding to a tumour specific antigen (e.g. dysfunctional P2X₇ receptor) and a second antigen binding domain for binding to any immune effector cell. Optionally the immune effector cell may be a cell expressing a CAR (eg: where the cell is a CAR T cell, the second binding domain may bind to CD3 or other antigen expressed by a CAR T cell).

The cell may be an “engineered cell”, “genetically modified cell”, “immune cell” or “immune effector cell” as described herein. Further, the cell may be capable of differentiating into an immune cell. A cell that is capable of differentiating into an immune cell (e.g. T cell that will express the dysfunctional P2X₇ CAR) may be a stem cell, multi-lineage progenitor cell or induced pluripotent stem.

In any embodiment, the cell may be a T cell, wherein optionally said T cell does not express TcRαβ, PD1, CD3 or CD96 (e.g. by way of knocking down or knocking out one of these genes on a genetic level or functional level).

In any embodiment, the cell may be an immune cell, wherein optionally said cell does not express accessory molecules that can be checkpoint, exhaustion or apoptosis-associated signalling receptors as well as ligands such as PD-1, LAG-3, TIGIT, CTLA-4, FAS-L and FAS-R, (e.g. by way of knocking out one of these genes on a genetic level or functional level).

In some embodiments, the genetically modified cell includes two or more different CARs.

In some embodiments of the invention, the genetically modified cell includes a nucleic acid molecule, or a nucleic acid construct, that encodes for two or more different CARs. In some embodiments of the invention, the genetically modified cell includes two or more nucleic acid molecules, or two or more nucleic acid constructs, each of which encodes for a different CAR.

As referred to herein, a “genetically modified cell” includes any cell comprising a non-naturally occurring and/or introduced nucleic acid molecule or nucleic acid construct encompassed by the present invention. The introduced nucleic acid molecule or nucleic acid construct may be maintained in the cell as a discreet DNA molecule, or it may be integrated into the genomic DNA of the cell.

Genomic DNA of a cell should be understood in its broadest context to include any and all endogenous DNA that makes up the genetic complement of a cell. As such, the genomic DNA of a cell should be understood to include chromosomes, mitochondrial DNA and the like. As such, the term “genomically integrated” contemplates chromosomal integration, mitochondrial DNA integration, and the like. The “genomically integrated form” of the construct may be all or part of the construct. However, in some embodiments the genomically integrated form of the construct at least includes the nucleic acid molecule of the second aspect of the invention.

As used herein, the term “different CARs” or “different chimeric antigen receptors” refers to any two or more CARs that have either non-identical antigen-recognition and/or non-identical signalling domains. In one example, “different CARs” includes two CARs with the same antigen-recognition domains (e.g. both CARs may recognise a dysfunctional P2X₇ receptor), but have different signalling domains, such as one CAR having a signalling domain with a portion of an activation receptor and the other CAR having a signalling domain with a portion of an co-stimulatory receptor. As will be understood, at least one of the two or more CARs within this embodiment will have an antigen-recognition domain that recognises the dysfunctional P2X₇ receptor and the other CAR(s) may take any suitable form and may be directed against any suitable antigen.

Accordingly, in some embodiments of the invention the two or more different CARs have different signalling domains, and may have identical, or different, antigen-recognition domains. Specifically, the genetically modified cell of the invention may include a first chimeric antigen receptor with a signalling domain that includes a portion derived from an activation receptor and a second chimeric antigen receptor with a signalling domain including a portion derived from a co-stimulatory receptor.

In some embodiments, the activation receptor (from which a portion of signalling domain is derived) is the CD3 co-receptor complex or is an Fc receptor.

In some embodiments, the co-stimulatory receptor (from which a portion of signalling domain is derived) is selected from the group consisting of CD27, CD28, CD-30, CD40, DAP10, OX40, 4-1 BB (CD137) and ICOS.

In some embodiments, the co-stimulatory receptor (from which a portion of signalling domain is derived) is selected from the group consisting of CD28, OX40 or 4-1BB.

In some embodiments, the genetically modified cell is further modified to constitutively express co-stimulatory receptors.

As described above, a cellular immune response is typically only induced when an activation signal (typically in response to an antigen) and a co-stimulation signal are simultaneously experienced. Therefore, by having a genetically modified cell in accordance with some of the above embodiments, which includes two or more CARs that in combination provide both an intracellular activation signal and an intracellular co-stimulation signal, ensures that a sufficient immune response can be induce in response to the recognition by the CAR(s) of their cognate antigen. Alternatively, the genetically modified cell may include only one CAR, which has an antigen-recognition domain that recognises a dysfunctional P2X₇ receptor, and may constitutively express co-stimulatory receptors, thereby increasing the likelihood of co-stimulation being provided simultaneously when the CAR is activated. Alternatively, the genetically modified cell may be further modified to constitutively express both co-stimulatory receptor(s) and its/their ligand(s). In this way the cell is continuously experiencing co-stimulation and only needs the activation of a CAR, with a signalling domain including a portion from an activation receptor, for immune activation of the cell.

Therefore, in some embodiments, the genetically modified cell expressing the CAR is further modified so as to constitutively express co-stimulatory receptors. In further embodiments, the genetically modified cell is further modified so as to express ligands for the co-stimulatory receptors, thereby facilitating auto-stimulation of the cell. Examples of CAR-expressing T cells that also express both co-stimulatory receptors and their cognate ligands (so as to induce auto-stimulation) are known in the art and include, inter alia, those disclosed in Stephen M T. et al. Nat Med, 2007; 13: 1440-9.

The potency of a genetically modified cell including a CAR can be enhanced by further modifying the cell so as to secrete cytokines, preferably pro-inflammatory or pro-proliferative cytokines. This secretion of cytokines provides both autocrine support for the cell expressing the CAR, and alters the local environment surrounding the CAR-expressing cell such that other cells of the immune system are recruited and activated. Consequently, in some embodiments of the fourth or fifth aspects of the invention the genetically modified cell is further modified to secret cytokines. This secretion may be constitutive, or may be inducible upon recognition of a CAR of its cognate antigen of ligand.

Whilst any one or more cytokines can be selected depending on the desired immune response, preferable cytokines and/or chemokines include IL-2, IL-7, IL-12, IL-15, IL-17, IL-18 and IL-21, CCL9, CCL21 or a combination thereof.

The immune cell can be any suitable immune cell, or progenitor cell thereof, or can be a homogeneous or a heterogeneous cell population. In some embodiments, the cell is a leukocyte, a Peripheral Blood Mononuclear Cell (PBMC), a lymphocyte, a T cell, a CD4+ T cell, a CD8+ T cell, a natural killer cell, a natural killer T cell, or a γδ T cell.

The immune cell may be a T cell, wherein optionally said T cell does not express TcRαβ, PD1, CD3 or CD96 (e.g. by way of knocking down or knocking out one of these genes on a genetic level or functional level).

The immune cell may not express accessory molecules that can be checkpoint, exhaustion or apoptosis-associated signalling receptors as well as ligands such as PD-1, LAG-3, TIGIT, CTLA-4, FAS-L and FAS-R, (e.g. by way of knocking out, or knocking down, one of these genes on a genetic level or functional level).

### Methods of Treatment and Administration

As discussed further in this document, the present invention finds application in the treatment of a variety of conditions, although preferably in the treatment of cancers.

The present invention also contemplates various scenarios for the use of the antigen binding protein described herein, preferably in conjunction with a bridging molecule. Optionally, a modified or engineered immune cell is also used.

In one scenario, the individual requiring treatment is administered a single composition comprising both the CAR T cells and antigen binding protein, optionally with a bridging molecule.

In further scenarios, the individual requiring treatment is administered a population of CAR T cells, which cells comprise an expression vector encoding the antigen binding protein, preferably also a bridging molecule. The expression vector may facilitate constitutive or inducible expression of the nucleic acid sequence encoding the antigen binding protein and/or bridging molecule.

Further still, the individual requiring treatment may be administered the CAR T cells, and at a later date, be administered a composition comprising the antigen binding protein (optionally also a bridging molecule) (e.g., via infusion), or a nucleic acid sequence encoding the antigen binding protein, optionally also a bridging molecule. Such a scenario may be appropriate in circumstances where the individual is first treated with the CAR T cells for targeted treatment of cancers that are positive for dysfunctional P2X₇ receptor and wherein the subsequent administration of the antigen binding protein is for the purposes of increasing the efficacy of the CAT T cell or additional recruitment of endogenous immune cells. A bridging molecule is typically for the purposes of redirecting the CARs or endogenous immune cells to alternative cancer antigens, or to peptides derived from an infectious agent and which are presented on MHC I or II molecules of cells.

Thus the antigen binding protein (referred to herein as an orchestration molecule), preferably also bridging molecule, may be administered prior to, at the same time as, or after the subject receives treatment with the CAR T cell.

Where the antigen binding protein, preferably also a bridging molecule, and CAR T cells are administered to the subject at the same time, they can be administered via the same route of administration (including in a single composition), or alternatively via different routes of administration. For example, the CAR T cells may be administered by injection into the blood stream of the subject, while the antigen binding protein (preferably also bridging molecule) may be administered via another route of administration such as intramuscularly, intradermally, subcutaneously or intraperitoneally.

An antigen binding protein and/or bridging molecule may be produced or expressed inside the body by genetically engineered cells secreting antigen binding proteins and/or bridging molecules spontaneously or upon stimulation via a stimulating agent e.g. a small molecule. Alternatively, cells may continuously secrete antigen binding proteins and/or bridging molecules and will stop secreting them upon application of a stimulating agent, e.g. a small molecule.

It will be clearly understood that, although this specification refers specifically to applications in humans, the invention is also useful for veterinary purposes. Thus in all aspects the invention is useful for domestic animals such as cattle, sheep, horses and poultry; for companion animals such as cats and dogs; and for zoo animals. Therefore, the general term “subject” or “subject to be/being treated” is understood to include all animals (such as humans, apes, dogs, cats, horses, and cows).

The term “administered” means administration of a therapeutically effective dose of the aforementioned composition including the respective cells to an individual. By “therapeutically effective amount” is meant a dose that produces the effects for which it is administered. The exact dose will depend on the purpose of the treatment, and will be ascertainable by one skilled in the art using known techniques. As is known in the art and described above, adjustments for systemic versus localised delivery, age, body weight, general health, sex, diet, time of administration, drug interaction and the severity of the condition may be necessary, and will be ascertainable with routine experimentation by those skilled in the art.

Subjects requiring treatment include those already having a benign, pre-cancerous, or non-metastatic tumour as well as those in which the occurrence or recurrence of cancer is to be prevented. Subjects may have metastatic cells, including metastatic cells present in the ascites fluid and/or lymph node.

The objective or outcome of treatment may be to reduce the number of cancer cells; reduce the primary tumour size; inhibit (i.e., slow to some extent and preferably stop) cancer cell infiltration into peripheral organs; inhibit (i.e., slow to some extent and preferably stop) tumour metastasis; inhibit, to some extent, tumour growth; and/or relieve to some extent one or more of the symptoms associated with the disorder.

Efficacy of treatment can be measured by assessing the duration of survival, time to disease progression, the response rates (RR), duration of response, and/or quality of life.

The method is particularly useful for extending time to disease progression.

The method is particularly useful for extending survival of the human, including overall survival as well as progression free survival.

The method is particularly useful for providing a complete response to therapy whereby all signs of cancer in response to treatment have disappeared. This does not always mean the cancer has been cured.

The method is particularly useful for providing a partial response to therapy whereby there has been a decrease in the size of one or more tumours or lesions, or in the extent of cancer in the body, in response to treatment.

The objective or outcome of treatment may be any one or more of the following:
to reduce the number of cancer cells;
reduce the primary tumour size;
inhibit (i.e., slow to some extent and preferably stop) cancer cell infiltration into peripheral organs;
inhibit (i.e., slow to some extent and preferably stop) tumour metastasis;
inhibit, to some extent, tumour growth;
relieve to some extent one or more of the symptoms associated with the disorder.

In one embodiment, subjects requiring treatment include those having a benign, pre-cancerous, non-metastatic tumour.

In one embodiment, the cancer is pre-cancerous or pre-neoplastic.

In one embodiment, the cancer is a secondary cancer or metastasis. The secondary cancer may be located in any organ or tissue, and particularly those organs or tissues having relatively higher haemodynamic pressures, such as lung, liver, kidney, pancreas, bowel and brain. The secondary cancer may be detected in the ascites fluid and/or lymph nodes.

In one embodiment, the cancer may be substantially undetectable.

“Pre-cancerous” or “preneoplasia” generally refers to a condition or a growth that typically precedes or develops into a cancer. A “pre-cancerous” growth may have cells that are characterised by abnormal cell cycle regulation, proliferation, or differentiation, which can be determined by markers of cell cycle.

The cancer may be a solid or a “liquid” tumour. In other words, the cancer may be growth in a tissue (carcinoma, sarcoma, adenomas etc) or it may be a cancer present in bodily fluid such as in blood or bone marrow (e.g., lymphomas and leukaemias).

In certain preferred embodiments, the cancer requiring treatment may be a cancer characterised by low levels of expression of dysfunctional P2X₇ receptor. Examples of such cancers include Burkitt's lymphoma. However, immunohistochemical analyses of surface expression of the dysfunctional P2X₇ (nfP2X₇) receptor on patient tumour biopsies reveals a range from 1+ to 3+ in IHC score. Samples with low expression may therefore be found in a wide range of tumour types. Examples are found in solid tumours of various types, including but not limited to neuroblastoma, colorectal cancers, lung cancers, kidney cancers, skin cancers, breast cancers, brain cancers and prostate cancer. Such differences in expression level in different tissues may be due to the formation of tumours from cells that are at an earlier state of transformation (the tissues with the highest receptor expression may be those undergoing the highest rate of proliferation).

Other examples of cancers that can be treated in accordance with the methods of the present invention include blastoma (including medulloblastoma and retinoblastoma), sarcoma (including liposarcoma and synovial cell sarcoma), neuroendocrine tumours (including carcinoid tumours, gastrinoma, and islet cell cancer), mesothelioma, schwannoma (including acoustic neuroma), meningioma, adenocarcinoma, melanoma, leukaemia or lymphoid malignancies, lung cancer including small-cell lung cancer (SCKC), non-small cell lung cancer (NSCLC), adenocarcinoma of the lung and squamous carcinoma of the lung, cancer of the peritoneum, hepatocellular cancer, gastric or stomach cancer including gastrointestinal cancer, pancreatic cancer, glioblastoma, cervical cancer, ovarian cancer, liver cancer, bladder cancer, hepatoma, breast cancer (including metastatic breast cancer), colon cancer, rectal cancer, colorectal cancer, endometrial or uterine carcinoma, salivary gland carcinoma, kidney or renal cancer, prostate cancer, vulval cancer, thyroid cancer, hepatic carcinoma, anal carcinoma, penile carcinoma, testicular cancer, oesophageal cancer, tumours of the biliary tract, as well as head and neck cancer.

In further examples, the methods of treatment contemplated within the scope of the present invention, include methods for treating or preventing an infectious disease. Thus, the bridging molecules of the invention can be utilised to redirect the CAR T cells towards an additional surface accessible antigen, for example wherein the antigen is a non-cancer associated pathogenic antigen presented on an MHC I or MHC II molecule as further described herein.

The subject requiring treatment for an infectious disease may be at risk or have been diagnosed with the disease. Subjects at risk include those who are immunocompromised. Thus, the methods of the present invention also allow for the prevention of onset of infectious disease in individuals receiving therapy (such as for treating cancer) that renders them immunocompromised and therefore susceptible to infection.

Examples of intracellular pathogens from which peptides are presented on MHC I or MHC II molecules include: viral infections, intracellular bacterial infections, protozoan infections, and intracellular fungal infections.

Examples of viral infections that may be treated using the methods of the present invention include: HIV, hepatitis (e.g., Hepatitis A, B or C), a coronavirus (e.g. SARS-CoV-2), an influenza virus, varicella zoster virus, mumps virus.

Examples of intracellular bacterial infections which may be treated using the methods of the present invention include: mycobacterial infections (e.g., Mycobacterium tuberculosis), Bartonella henselae, Francisella tularensis, Listeria monocytogenes, Salmonella Typhi, Brucella, Legionella, Nocardia, Neisseria, Rhodococcus, Yersinia, Staphylococcus aureus, Chlamydia, Rickettsia, Coxiella, and Chlamydophila pneumoniae.

Examples of intracellular infections caused by fungal pathogens: Histoplasma capsulatum, Cryptococcus neoformans, and Pneumocystitis jirovecii.

Examples of obligate intracellular protozoan pathogens include: Apicomplexans (Plasmodium spp., Toxoplasma gondii and Cryptosporidium parvum), and Trypanosomatids (Leishmania spp. and Trypanosoma cruzi).

Immune cells that may be targeted to modulate the immune system in the context of cancer and/or autoimmune disease may be B cells (CD19, CD20, CD22), plasma cells (BCMA, CD38, CD138), T cell subsets via (TRBC1 or TRBC2, α4β7 & αEβ7, CD7), macrophages and TAMs (CD163 and CD206). In the context of allogeneic stem cell transplantation, immune-based conditioning may be undertaken by targeting (CD34, CD117, CD133, CD33 and CD38) especially in case of non-malignant diseases e.g. thalassaemia major or sickle cell anaemia and/or in case of DNA-repair defects like Fanconi anaemia.

Targeting senescent tumour cells via the marker (uPAR) will help to eliminate tumour cells in a resting state and which are likely to expand at later time points and promote even faster proliferation of cancer cells in the latter by secreting tumour promoting cytokines and shaping a tumour-suppressive environment protecting new cancerous subclones.

CAR T cells may be constructed in a way that they are able to immunosuppress other immune cells, e.g. T_{REG} CAR T cells or by secreting immunosuppressive cytokines (TGFbeta, IL10) and chemokines by introducing the corresponding inducible expression cassette [NFAT-dependent cytokine secretion] and the signalling in the construct.

The antigen binding proteins and bridging molecules of the invention may be formulated for administration to a subject using techniques known to the skilled artisan. Formulations of the bridging molecules may include pharmaceutically acceptable excipient(s) (carriers or diluents). Examples of generally used excipients include, without limitation: saline, buffered saline, dextrose, water-for-injection, glycerol, ethanol, and combinations thereof, stabilising agents, solubilising agents and surfactants, buffers and preservatives, tonicity agents, bulking agents, and lubricating agents.

A formulation of antigen binding proteins and bridging molecules may include one type of antigen binding protein and/or bridging molecule, or more than one type of antigen binding proteins and/or bridging molecule (i.e., wherein the bridging molecules may have the same or different targeting and/or dysfunctional P2X₇ receptor epitope moieties).

The bridging molecules may be administered to a subject using modes and techniques known to the skilled artisan. Exemplary modes include, but are not limited to, intravenous, intraperitoneal, and intratumoural injection. Other modes include, without limitation, intradermal, subcutaneous (s.c, s.q., sub-Q, Hypo), intramuscular (i.m.), intra-arterial, intramedullary, intracardiac, intra-articular (joint), intrasynovial (joint fluid area), intracranial, intraspinal, and intrathecal (spinal fluids).

Formulations comprising the antigen binding protein(s) or bridging molecule(s) are administered to a subject in an amount that is effective for treating the specific indication or disorder. In general, formulations comprising at least about 0.01 μg/kg to about 100 mg/kg body weight of the antigen binding protein or bridging molecule may be administered to a subject in need of treatment. In most cases, the dosage may be from about 100 μg/kg to about 10 mg/kg body weight of the antigen binding proteins or bridging molecules daily, taking into account the routes of administration, symptoms, etc. However, the amount of antigen binding proteins or bridging molecules in formulations administered to a subject may vary between wide limits, depending upon the location, source, identity, extent and severity of the disorder, the age and condition of the individual to be treated, etc. A physician may ultimately determine appropriate dosages to be used. The antigen binding proteins or bridging molecules may be administered as a continuous infusion or a bolus application.

The timing between the administration of the CAR T cell and the antigen binding protein and/or bridging molecule formulation may range widely depending on factors that include the type of (immune) cells being used, the binding specificity of the CAR, the identity of the targeting moiety and the identity of the target cell, e.g. cancer cell to be treated, the location of the target cell in the subject, the means used to administer the formulations to the subject, and the health, age and weight of the subject being treated. Indeed, the formulation may be administered prior to, simultaneous with, or after the genetically engineered (immune) cell formulation.

It will be understood that the invention disclosed and defined in this specification extends to all alternative combinations of two or more of the individual features mentioned or evident from the text or drawings. All of these different combinations constitute various alternative aspects of the invention.

It will be understood that the invention disclosed and defined in this specification extends to all alternative combinations of two or more of the individual features mentioned or evident from the text or drawings. All of these different combinations constitute various alternative aspects of the invention.

### EXAMPLES

### Example 1—Materials and Methods Including Generation of Antigen Binding Proteins (OR Molecules) and Bridging Molecules

Cultivation, transfection and protein production were performed as per ExpiCHO Expression System User Guide (Thermo—ExpiCHO™ Expression System USER GUIDE. For transfection of ExpiCHO-S™ Cells in a defined, serum-free medium Catalogue Number A29133, Publication Number MAN0014337). In summary, ExpiCHO were routinely passaged and maintained at less than 4-6×10⁶ cell/mL in ExpiCHO medium. Cells in the mid-log growth phase were transfected when cell number was in the range of 5-7×10⁶ cell/mL. For transfection, liposome complex was prepared with 1 μg DNA for each mL of culture. For co-transfection of vectors (for either OR or bridging molecule production) coded with heavy and light chains separately, the vector ratio was set at 1:1 unless specified otherwise. “High Titer” or “Max Titer” expression protocols were followed after transfections, and cultures were harvested when cell viabilities dropped below 70%. Harvest was done by centrifugation at 300×g for 5 min at 20° C. Cells were discarded and the supernatant was centrifuged again at 4000×g for 30 mins at 4° C. The harvested supernatants were clarified by 0.2 μm filtration using PES membrane before freezing for storage.

Harvested samples could be enriched and buffer-exchanged by spin-columns or TFF cassette with nominal molecular size cut off of 5, 10 or 30 kDa.

For HIS-tagged column purification, the harvested supernatants were dialysed via SnakeSkin dialysis tube with nominal molecular size cut off 5, 10 or 30 kDa depending on the protein of interest. For large scale production, the supernatants were washed through a TFF cassette with a certain molecular size cut off membrane. Buffer-exchange to the desired column loading buffer also was achieved through the above-mentioned procedures to prepare the sample for the His-tagged column purification. The purification was performed on either a HisTrap excel column (Cytiva) or PureCube 100 Compact Cartridge Ni-INDOGO affinity Column (Cat #75302, Cube Biotech) or other equivalent column. Purification was performed on a AKTA Pure system (Cytiva) equipped with UV detector at 280 nm wavelength, conductivity detector and pH probe. Loading and washing buffer consisted of 50 mM Sodium Phosphate Monobasic and 0.3 M Sodium Chloride, pH 8.0. The elution buffer contained 500 mM Imidazole. The eluted protein was buffer exchanged to PBS using Vivaspin (Sartorius) and stored under 4° C.

Protein was quantified via Nanodrop at 280 nm wavelength and standard Bicinchoninic acid (BCA) protein assay. The protein purity was confirmed by SDS PAGE gel electrophoresis.

The detailed experimental data generated by the inventor(s) and described herein includes the generation of a wide variety of antigen binding proteins or OR molecules that include:
1. Antigen binding proteins generated in multiple antibody formats, e.g. Fab, scFv and IgG;
2. Antigen binding domains of the antigen binding proteins that bind to a wide range of cell surface antigens that are present on immune cells;
3. Antigen binding domains of the antigen binding proteins that bind to tumour-specific antigens.

The detailed experimental data generated by the inventor(s) and described herein includes the generation of a wide variety of bridging molecules that include:
1. Targeting moieties generated in multiple antibody formats, e.g. Fab and scFv;
2. Various positioning of the dysfunctional P2X₇ receptor epitope moiety on the targeting moiety including, for example, on the V_{L} and V_{H};
3. Inclusion of linkers between the targeting moiety and the dysfunctional P2X₇ receptor epitope moiety;
4. Targeting moieties that bind to a wide range of cell surface antigens that are present on tumour cells from different tissue origins.

### Antibody/Fab Conjugation

Conjugation of BIL03s 2-2-1-Fc (an anti-nfP2X₇ receptor antibody) with fluorochrome Alexa Fluor® 647 (AF647) was performed according to manufacturer's instruction (Cat #A20186, ThermoFisher). The AF647 labelled BIL03s 2-2-1-Fc antibody was reconstituted in PBS, pH 7.2, with 2 mM sodium azide.

### Testing Binding of Bridging Molecules to Cells by Flow Cytometry

### Reagents

Abs and Fabs:
BIL03 pure antibody: prediluted to 100 ug/mL by PBS.
BIL03-AF647 antibody: prediluted to 100 ug/mL by PBS
Anti-His antibody-FITC (1 mg/mL) (Abcam Cat #ab1206, Cat #GR3361939-1)
Rabbit IgG-FITC isotype control (Abcam Cat #ab3706, Cat #GR3356160-1)
Bridging molecules, generated in-house and harvested from supernatant see below

Cell lines used for binding assays: JeKo-1 (CD19, CD20, CD79B, CD37, CD22, ROR1, Her2), MOLM-13 (CD33, CD38, CD37, CD135, CD123), PC3 (Her2), MDA-MB-231 (EGFR, PD-L1), Raji (CD22, CD70, CD79B), Karpas299 (CD30), U937 (CD105), HL60, RPM18226 (BCMA, CD38, CD33).

Cells were resuspended at a density of in 5×10⁶ cells/ml and 100 μL aliquots used per well for staining (0.5×10⁶/sample for testing).

| | | | Growth | | Culture |
|---|---|---|---|---|---|
| Cell Line | Disease Type | Source | Properties | Subculture Ratio | Medium |
| MOLM-13 | Acute Myeloid | ATCC | Suspension | 4 × 10⁵-2 × 10⁶ | RPMI |
| | Leukaemia, AML | | | cells/mL | 1640 + |
| | | | | | 10% FBS |
| JeKo-1 | Mantle Cell | ATCC | Suspension | 2 × 10⁵-2 × 10⁶ | RPMI |
| | Lymphoma, MCL | | | cells/mL | 1640 + |
| | | | | | 20% FBS |
| MDA-MB | Mammary | NCI-60 | Adherent | 1:4 | Leibovitz's |
| 231 | | panel | | | L-15 |
| | | | | | medium + |
| | | | | | 10% FBS; |
| | | | | | RPMI1640 + |
| | | | | | 10% |
| | | | | | FBS |
| PC-3 | Prostate | CellBank | Adherent | 1:6 | F12K or |
| | | Australia | | | RPMI |
| | | | | | 1640 + |
| | | | | | 10% FBS |
| U937 | Histiocytic | CellBank | Suspension | 2 × 10⁵-9 × 10⁵ | RPMI |
| | Lymphoma | Australia | | cells/mL | 1640 + |
| | | | | | 2 mM |
| | | | | | Glutamine + |
| | | | | | 10% |
| | | | | | FBS |
| Raji | Burkitt's lymphoma | CellBank | Suspension | 3 × 10⁵-9 × 10⁵ | RPMI |
| | | Australia | | cells/mL | 1640 + |
| | | | | | 2 mM |
| | | | | | Glutamine + |
| | | | | | 10% FBS |
| HL-60 | Accute | NCI-60 | Suspension | 1 × 10⁵-1 × 10⁶ | IMDM + |
| | promyelocytic | panel | | cells/mL | 10% FBS |
| | leukaemia | | | | |
| RPMI- | Multiple Myleoma | NCI-60 | Semi | 5 × 10⁵-2 × 10⁶ | RPMI |
| 8226 | | panel | adhension | cells/mL | 1640 + |
| | | | | | 10% |
| | | | | | FBS |
| Karpas299 | anaplastic large | CellBank | Suspension | 0.5-2 × 106 | RPMI |
| | cell lymphoma | Australia | | cells/mL | 1640 + |
| | | | | | 2 mM |
| | | | | | Glutamine + |
| | | | | | 10-20% |
| | | | | | FBS |

### Procedure

### 1. Staining Condition List:

Bridging molecule binding to BIL03s Antibody Staining and His Tag Antibody detection

### Control Conditions:

Control bridging molecule binding to BIL03s Antibody Staining and His Tag Antibody detection
No bridging molecule binding to BIL03s Antibody Staining and His Tag Antibody detection
Unstained cells

### 2. Staining Procedure

Block the cells with human FcR blocker (20% v/v, Miltenyi) on ice for 10 min and wash off the unbounded FCR blocker.
Incubate the cells in 50 μL of crude prep supernatant and stain on ice for 15 min.
Wash the cell suspension by FACS buffer×2
Stain the cells with 1 μg/mL of BIL03s-AF647 and 1 μL of His Tag antibody per 100 μL cell suspension.
Wash the cells by FACS buffer ×2.
Cells ready to be analysed on MacsQuant16.

### Lentiviral Vector Production Using Adherent Lenti-X HEK293T (Takara) Cells and PEI

### Lenti-X 293T Culture Media

For cell culture pre transduction:

90% Dulbecco's Modified Eagle's Medium (DMEM) with high glucose (4.5 g/L), 4 mM L-glutamine, and sodium bicarbonate (Sigma-Aldrich, D5796); 10% Foetal Bovine Serum (FBS); 1 mM sodium pyruvate (Sigma-Aldrich, S8636).

For cell culture post transduction:

90% Dulbecco's Modified Eagle's Medium (DMEM) with high glucose (4.5 g/L), 4 mM L-glutamine, and sodium bicarbonate (Sigma-Aldrich, D5796); 10% Foetal Bovine Serum (FBS); 1 mM sodium pyruvate (Sigma-Aldrich, S8636), and 10 mM sodium butyrate.

| Plasmid DNA ID# | Construct Name |
|---|---|
| Transfer | Various |
| A | pRSV/REV (expresses HIV-1 REV) |
| B | pMDL/RRE (expresses HIV GAG/POL) |
| C | pMD2.G (expresses VSV glycoprotein) |

### Protocol, Part I Lenti-X 293T Cells Lentivirus Transfection

### Day 0:

1. Seed 1.7×10⁶ cells per 15 cm dish so that they will be ˜80% confluent on the day of transfection—the following Monday (˜16×10⁶ cells).

### Day 1:

1. Check cells under microscope. The cells should be about 75-90% confluent.
2. Gently aspirate media, add 20 mL fresh DMEM supplemented with 10% FCS to each 15 cm dish, and incubate for at least two hours before transfection.
3. Perform the transfection procedure in the afternoon (˜2.30-4.30 μm). Warm an aliquot of serum-free DMEM to 37° C.
4. Prepare the Mixture A (Plasmid DNA solution) and Mixture B (PEIpro solution).
5. Determine the required volumes of DMEM and plasmid DNA in Mixture A according to the table below.

| | | | | |
|---|---|---|---|---|
| Mixture A | T175 or 15cm | T75 | 10cm dish | 6 well |
| (DNA) | dish (175 cm²) | (75 cm²) | (60 cm²) | (9.6 cm²) |

| | |
|---|---|
| Plasmid DNA* | VSV-G 0.06 μg/cm² |
| | Rev 0.06 μg/cm² |
| | Gag/pol 0.12 μg/cm² |
| | Transfer plasmid 0.08 μg/cm² |
| DMEM (w/o | 5% final culture volume |
| additives) | |

6. Determine the volumes required for each plasmid DNA component for the number of plates required. 15 cm dish has an area of ˜175 cm².

| | μg/plate | | ug/plate | |
|---|---|---|---|---|
| Mixture A | CCT | #___plates | VGEF | #___plates |
| Transfer plasmid | 14 | | 2 | |
| pMDL/RRE | 21 | | 7.5 | |
| pRSV/REV | 10.5 | | 7.5 | |
| pMD2.G | 10.5 | | 5 | |
| DMEM high glucose | 0.75 mL | | 0.75 mL | |
| (w/o additives) | | | | |

7. Determine the volumes required for each component of Mixture B (PEIpro solution).

For each 15 cm dish,

| Mixture B | | μL/plate | | μL/plate | |
|---|---|---|---|---|---|
| (PElpro) | | CCT | #___plates | VGEF | #___plates |
| PElpro | PElpro:DNA = | 56 uL | | 22 uL | |
| 1 mg/mL | 1:1 ratio | | | | |
| DMEM high | | 0.75 mL | | 0.75 mL | |
| glucose (w/o | | | | | |
| additives) | | | | | |

8. Vortex PEIpro for 5 seconds and then spin down if needed to collect liquid in the bottom of the tube.
9. Prepare Mixture B (PEIpro in media) in a 15 mL tube by adding PEIpro into the DMEM high glucose without any additives. Add PEI into DMEM Invert up and down a few times and spin down quickly.
10. Prepare Mixture A (plasmid DNA dilution in media) in a 50 mL conical tube by adding DNA into the media. Mix gently by inverting up and down and spin down quickly.
11. Mix and prepare transfection mixture (PEIpro/DNA solution) by adding Mixture B (PEIpro solution) to Mixture A (plasmid DNA dilution). Using a p1000 micropipette, add the PEIpro solution dropwise to DNA solution and immediately invert 3-4 times to mix. Do not vortex.
12. Incubate at room temperature for 15 min, no longer than 30 min. Do not agitate the tube during this time.
13. After the 15-min incubation, add the transfection mixture to the flasks/dishes containing cells and fresh media (dropwise if possible). Mix by gently rocking the flask/dish horizontally with back and forth and left and right motions.
14. Incubate the flasks/dishes at 37° C. with 5% CO2 overnight.

### Day 2:

15. 16-18 hours after transfection, replace media. Working with 2 plates at a time, aspirate old media. Using a 25 mL pipette, carefully add 15 mL fresh DMEM supplemented with 10% FCS, and 10 mM sodium butyrate.

### Day 3 First Harvest 24 Hour Post Media Change:

16. Collect the supernatants from the culture flasks/dishes in 50 mL Falcon tubes. Carefully replace each plate with 15 mL fresh DMEM supplemented with 10% FCS and 10 mMm sodium butyrate and return plates to incubator.
17. Centrifuge the supernatant at 500×g for 10 min at 4° C. (for concentration by Lenti-X concentrator). For ultracentrifugation, centrifuge at 3800 rpm for 30 min at RT.
18. Draw up the virus-containing supernatant with a 20 mL syringe and filter through a 0.45 μm PES filter (Millipore) into a new 50 mL Falcon tube. This can be used as (a) Crude preparation for transduction, or proceed to (b) concentration by ultracentrifugation, or (c) concentration by Lenti-X concentrator. Alternatively, store crude virus 4° C. overnight and pool with 48 hour harvest. Store concentrated virus in aliquots at −80° C. for extended periods.
19. Discard plates, tubes and filters in a biohazard bag in the tissue culture hood. Seal the biohazard bag before taking it out of the hood for disposal.

### Day 4 Second Harvest 48 Hour Post Media Change:

20. Collect the supernatants from the culture flasks/dishes in 50 mL Falcon tubes.
21. Centrifuge the supernatant at 2000×g for 30 min at room temp.
22. Draw up the virus-containing supernatant with a 20 mL syringe and filter through a 0.45 μm filter (Millipore) into a new 50 mL Falcon tube. This can be pooled with the 24 hr harvest or process separately and proceed to (b) concentration by ultracentrifugation, or (c) concentration by Lenti-X concentrator.
23. Discard plates, tubes and filters in a biohazard bag in the tissue culture hood. Seal the biohazard bag before taking it out of the hood for disposal.

### Part II Concentrating Lentiviruses by Lenti-X Concentrator

24. Harvest the lentivirus-containing supernatants. Caution: supernatants contain live lentivirus. Pool similar stocks together, if desired. Centrifuge briefly at 500×g for 10 min or filter through a 0.45 μm filter.
25. Transfer clarified supernatants to a sterile container and combine 1 volume of Lenti-X Concentrator with 3 volumes of clarified supernatant. Mix by gentle inversion. Larger volumes may be accommodated through the use of larger (i.e., 250 mL or 500 mL) centrifuge tubes.
26. The incubation with the Lenti-X concentrator is done once. Either at harvest (after 1 day) or after 2 days (pooled harvest). Incubation at least for 30 min or overnight and then centrifugation of the pre-incubated fluid.
27. Centrifuge samples at 1,500×g for 45 minutes at 4° C. After centrifugation, an off-white pellet will be visible.
28. Carefully remove supernatant, taking care not to disturb the pellet. Residual supernatant can be removed with either a pipette tip or by brief centrifugation at 1,500×g.
29. Gently resuspend the pellet in 1/10 to 1/100th of the original volume using complete DMEM, PBS, or TNE. The pellet can be somewhat sticky at first, but will go into suspension quickly.
30. Immediately titrate sample or store at −80° C. in single-use aliquots.

### CAR T Cell Generation Protocol

nfP2X₇ BRiDGE CAR T cells were generated by lentiviral transduction of CD4/CD8 positive selected T cells (1:1 ratio) via magnetic activated cell sorting (MACS) stimulated with TransAct (all according to manufacturer's instructions) cultivated in IL7/IL15 supplemented TexMACS media (both 10 ng/mL). The donor source was a buffy coat.

CAR T cells were treated in the very same way but underwent lentiviral transduction to express the nfP2X₇ BRiDGE CAR. Activated untransduced T cells (aUT) do not express any receptor that can either engage with the EGFR nor the CD33 bridging molecules.

### Hypothesis:

nfP2X₇ BRiDGE CAR T cells have a superior effector function over aUT as they are redirected towards cancer cells directly via nfP2X₇ recognition on the cell surface of MOLM-13 leukaemic cells.

nfP2X₇ BRiDGE CAR T cells have a superior effector function over aUT as they are redirected towards cancer cells indirectly via nfP2X₇ E200 derived epitope on the CD33 Fab-bridging molecules on the surface of MOLM-13 leukaemic cells.

### Reagent and Equipment Preparation

CAR T culture medium: TexMACS with human IL-7 and IL-15. IL-7 stock concentration was 100 μg/mL, each vial has 55 μL. IL-15 stock concentration is 50 μg/mL, each vial had 55 μL.

For preparation of TexMACS with final concentration of 10 ng/mL of IL-7, 5 ng/mL of IL-15 and 3% FBS, add 50 μL of IL-7, 50 μL of IL-15 stock, and 15 mL FBS into each bottle (500 mL) of TexMACS medium. Label the date of adding of cytokines on the medium bottle.

Freezing medium preparation on the day of harvest: 10% of DMSO, 90% of FBS. Note: Add the reagent into 50 mL falcon tube according to the following order: DMSO to FBS.

### Part I: T Cell Activation and T Cell Transduction

### Day 1: T Cell Activation

1. CD4+ and CD8+ CAR T cells separation from whole blood or buffy coat. Refer to protocol of PBMC separation and CD4 and CD8 cell separation.
2. Wash the CD4+ and CD8+ cells twice with pre-warmed TexMACS Medium (without supplement cytokines) by filling the falcon tube to the maximum volume and centrifuge at 300×g for 5 minutes. Aspirate supernatant completely.
3. Resuspend the CD4+ and CD8+ cells in pre-warmed TexMACS medium supplemented with 10 ng/mL IL-7, 5 ng/mL IL-15 and 3% FBS to a final concentration of 10\hat{ }6 cells/mL.
4. Plate the CD4+ cells and the CD8+ cells with a ratio of 1:1 by adding 0.5 ml of CD4+ cells and 0.5 mL of CD8+ cells into each well in a 24 well plate.
5. Add 10 μL of T cell TransAct to a final dilution of 1:100 in the cell culture and carefully resuspend.
6. Incubate for ˜36 hours at 37° C. with 5% CO2 before transduction.

### Day 3: T Cell Transduction

7. Use fresh viral vectors for transduction if possible. Otherwise, thaw slowly frozen viral vectors on ice.
8. Remove 800 μL of the media slowly using a P1000 pipette from the side of the wells, taking care not to disrupt the bottom cell layer.
9. Add in dropwise 150 μL (one plate's worth of viral vectors) or 300 μL (2 plates' worth of viral vectors) to the T cells. Top up the wells with fresh supplemented TexMACS media up to 600 μL and add polybrene to each well at a final concentration of 4 μg/mL. From this time onwards, the T cells should be kept in an incubator for lentiviral work only.
10. After transduction, appropriate clean up procedures should be performed. Decontaminate the biosafety cabinet and the aspirator line by cleaning surfaces and running the line with 2% Virkon solution following by 70% ethanol. Dispose of contaminated waste, such as tips and serological pipettes etc. in a biohazard bag inside of the biosafety cabinet, seal the bag before taking the waste outside for disposal.

Day 4 onwards—T cell maintenance
11. Transduced T cells were maintained at in IL-7, IL-15 and 3% FBS containing TexMACS media.
12. Observe the T cell growth in the 24 well plate, transfer the cells to T75 flasks on day 2 post transduction.
13. Monitor the lactate levels in the media using CCS Analyzer daily. Replenish fresh media if the lactate level is >10 mmol/L; Ideally, keep the lactate level <4 mmol/L.

### Day 9-Day 5 Post T Cell Transduction—Expression Analysis

Transduced T cells are counted on day 5 using a flow cytometer. A sample is taken for flow cytometry analysis to determine expression efficiency based on a standard flow cytometry protocol.

### Luciferase Expressing Cancer Cell Line Generation

Firefly luciferase lentiviral transfer plasmids used for viral vector production:
pRRLsin18.cPPT.EF1a_firefly_luciferase_T2A_EGFP.WPRE
pRRLsin18.cPPT.hPGK_firefly_luciferase_T2A_EGFP.WPRE
1. Plate cells at a density of 300,000 cells/well in a 24-well plate in a total volume of 850 μL.
2. Viral vectors containing the firefly luciferase gene are produced using a 4-plasmid transfection protocol as described in the lentiviral production section. Use fresh concentrated viral vectors for transduction if possible. Otherwise, thaw slowly frozen viral vectors on ice.
3. Add in dropwise 150 μL (one plate's worth of viral vectors) to the cells. From this time onwards, the cell should be kept in an incubator for lentiviral work only.
4. Incubate cells at 37° C. with 5% CO₂.
5. After transduction, appropriate clean up procedures should be performed. Decontaminate the biosafety cabinet and the aspirator line by cleaning surfaces and running the line with 2% Virkon solution following by 70% ethanol. Dispose of contaminated waste, such as tips and serological pipettes etc. in a biohazard bag inside of the biosafety cabinet, seal the bag before taking the waste outside for disposal.
6. On day 2 post transduction, transfer cells to T25 flasks.
7. On day 5 post transduction, count cells and take a sample for expression analysis by flow cytometry.
8. On day 7 post transduction, transduced cells are bulk sorted based on eGFP expression using a live cell sorter.
9. After expansion, bulk sorted cells are further sorted by single cell clones. Single cell clones are grown, expanded and frozen down to make stocks.

### Functional Assays

Target cells constitutively expressing firefly luciferase and eGFP (sorted and grown as single cell clones defined as high performance cell lines) were used in the functional assays to measure viability via bioluminescence and/or fluorescence. The amount of light emitted correlates to the total number of cells in bioluminescence and the fluorescent target cells identified via flow cytometry correlate with the total number of cells alive.

Effector and target cells were seeded according to indicated effector to target ratio (ET). The indicated ET ratio, e.g. 10:1 is always referred to the total number of T cells and the total number of target cells. As the CAR expressing fraction is different from the total number of T cells the ET ratio referred to the CAR expressing cells is indicated separately. Target cells were seeded with 25,000 or 50,000 cells per 96 well plate.

### Luciferase Killing Assay

Effector and target cells were seeded according to indicated effector to target ratios (ET). The BRiDGE molecules were added in the indicated format (Fab, IgG1) at the indicated concentrations. D-luciferin was added and bioluminescence was measured at the indicated time points after incubation was started under standard conditions in incubators at 37° C. and 5% CO₂ on a SpectraMaxi3.

Viability of cells was calculated according to a serial dilution derived bioluminescence activity curve of cells (100%, 75%, 50%, 25%, 10% and 0% target cells) and depicted in percent viable cells. In general, the lysis was calculated by (bioluminescence of testing condition−0% bioluminescence)/(100% bioluminescence−0% bioluminescence).

### Flow Based Kill Assay

Effector and target cells were seeded according to indicated effector to target ratios (ET), the BRiDGE molecules were added in the indicated format (Fab, IgG1) at the indicated concentrations. Cell number was measured at the indicated time points (24 h or 48 h) after incubation was started under standard conditions in incubators at 37° C. and 5% CO₂ on a MACSQuant16 flow cytometer according to standard protocols. The staining of cells included a viability dye to exclude all dead cells from the analysis. T cells were clearly differentiated from eGFP positive cancer cells via CD3. Further T cells were characterised by CD25 and CD69 as a measure for specific T cell activation according to standard protocols after 24 h or 48 h. The final data analysis was performed by FlowJo10.

### Antibody Cocktail

| Channel | Antibody | Source | Cat# |
|---|---|---|---|
| R1 | CD3 APC | Miltenyi | 130-113-135 |
| R3 | CD25 APCVio770 | Miltenyi | 130-123-469 |
| V2 | CD69 VioGreen | Miltenyi | 130-112-611 |
| B1 | eGFP | Constitutive stable | Target cell marker |
| | | expression | |
| V1 | Viobility405/452 | Miltenyi | 130-109-816 |

### Flow Based Acquisition of Cytokine Secretion

Effector and target cells were seeded according to indicated effector to target ratios (ET), the BRiDGE molecules were added in the indicated format (Fab, IgG1) at the indicated concentrations. Supernatant was collected after 24 h or 48 h and measured at the indicated time points after incubation was started under standard conditions in incubators at 37° C. and 5% CO₂ on a MACSQuant16 flow cytometer according to standard protocols using the Miltenyi cytokine beads. The final data analysis was performed by FlowJo10.

### Example 2—Characterisation of Orchestration (OR) Molecules—Dysfunctional P2X₇ Receptor Binding

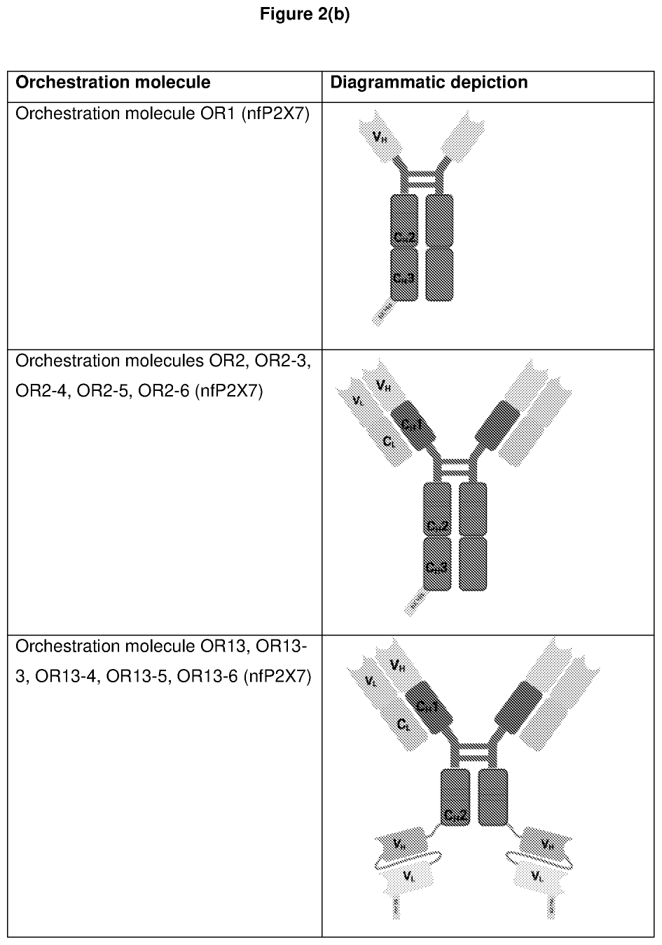

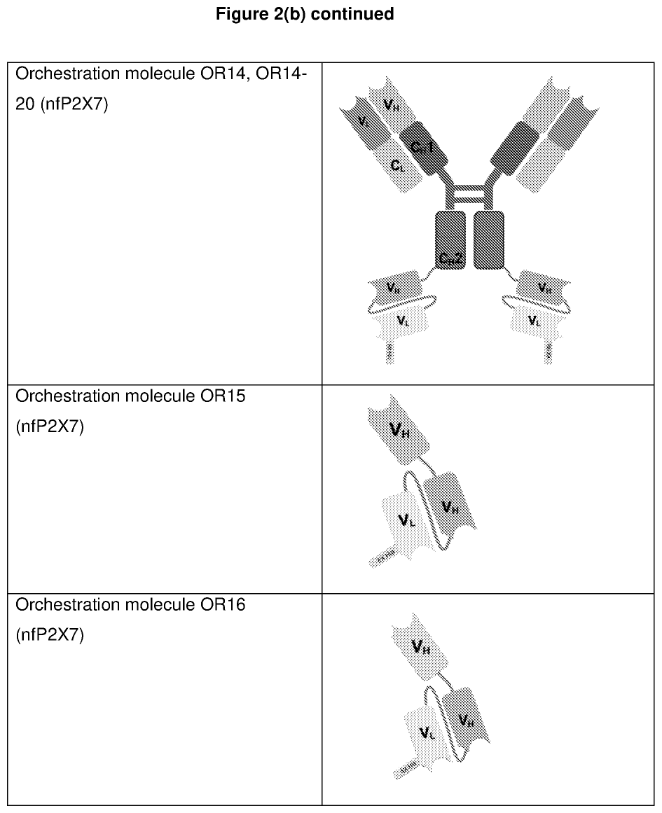

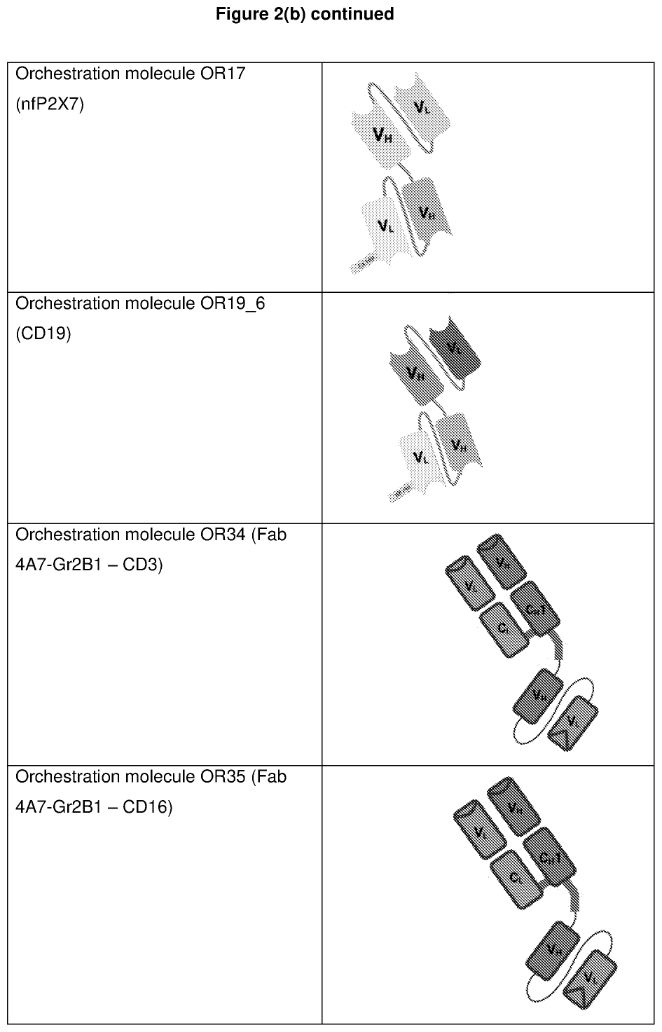

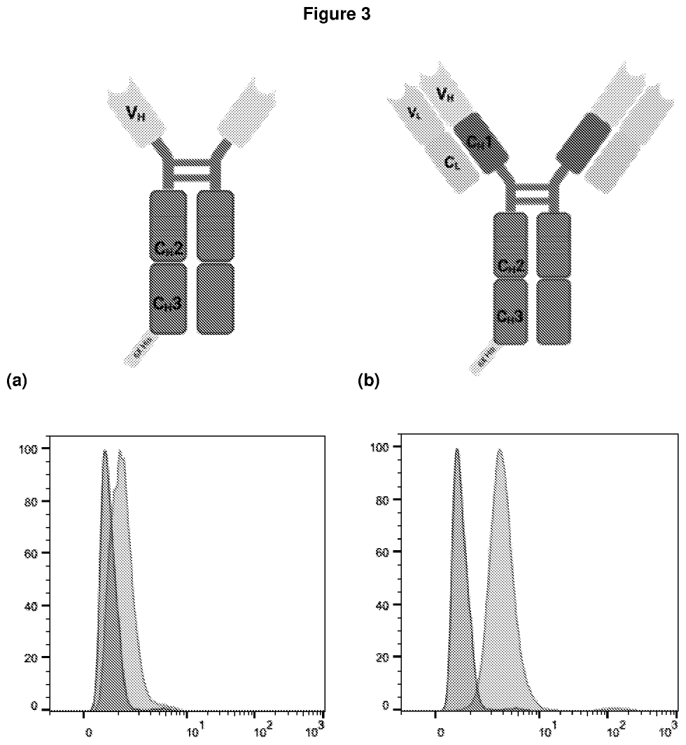

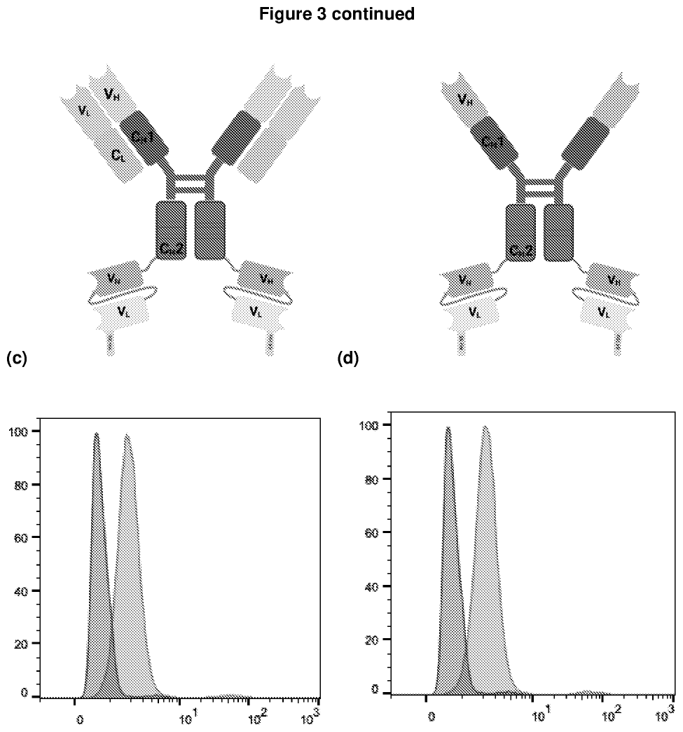

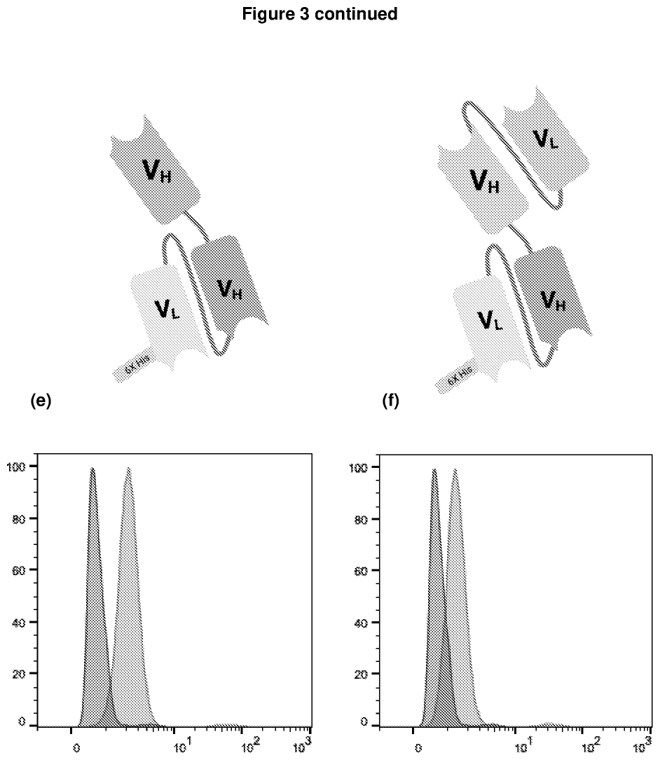

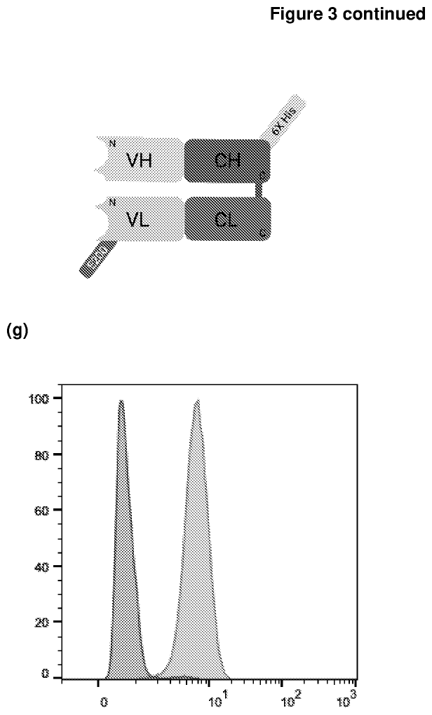

Antigen binding proteins (or orchestration (OR) molecules) as shown in FIG. 2 were generated using the protocols in Example 1. Flow cytometric detection of binding capacity of OR molecules on MOLM-13 (AML wildtype cell line) is shown in FIG. 3. As MOLM-13 are a cancer cell line they express dysfunctional P2X₇ receptors on their cell surface and therefore were used to confirm the ability of the OR molecules to bind dysfunctional P2X₇ receptors on live cells.

FIG. 3 shows indirect staining of artificial proteins on MOLM-13 cells via anti-HIS staining. As a control fusion protein the CD33 BRiDGE (B031) was used. There is a clear binding to MOLM-13 cells compared to the isotype control which was anti-HIS antibody only. Histogram overlay of HIS-FITC positivity on MOLM-13. Grey: Isotype control (left), Red: binding of HIS ab (right-shifted). HIS-ab was used according to manufacturer with 1 μl per test, target cells were incubated with 50 μl of supernatant from Lenti-X™ 293T cells for 10 min at room temperature. Supernatant harvested 48 h after transfection. Transfection with CD33 Fab as positive control.

The results in FIG. 3 show that OR molecules with a first antigen binding domain in a wide variety of formats—single domains, V_{H}-V_{L} or V_{L}-V_{H}—can bind to cell surface presented dysfunctional P2X₇ receptors. FIG. 3 confirms dysfunctional P2X₇ receptor binding for (a) OR1, (b) OR2-3, (c) OR13-3, (d) OR14, (e) OR15, (f) OR17, and (g) CD33 bridging molecules.

| | | | Targeting |
|---|---|---|---|
| Plasmid | Structure | Paired to | antigen |
| OR1 | BIL03_lgG1_HC_SDIE_ | | nfP2X₇ |
| | hinge_CH2_CH3 | | |
| OR2 | BIL03_IgG1_HC_SDIE_ | OR3 | nfP2X₇ |
| | CH1_hinge_CH2_CH2 | | |
| OR3 | WTB1 LC | | nfP2X₇ |
| OR13 | Bispecific mAb BIL03_IgG1_ | OR3 | CD3 x nfP2X₇ |
| | HC_CH1_hinge_CH2- | | |
| | SDIE_CD3_OKT3 | | |
| OR14 | Bispecific mAb | OR20 | CD3 x nfP2X₇ |
| | VHH_4B11_lgG1_HC_CH1_ | | |
| | hinge_CH2-SDIE_CD3_OKT3 | | |
| OR20 | Group 2 B1 | | CD3 x nfP2X₇ |
| OR15 | BiTE OKT3-VHH 4B11 | | CD3 x nfP2X₇ |
| OR16 | BiTE OKT3-VHH BILO3 | | CD3 x nfP2X₇ |
| OR17 | BiTE OKT3-BIL03 WTB1 | | CD3 x nfP2X₇ |
| OR19_6 | BiTE OKT3-hu4G7 | | CD3 x CD19 |

### Example 3—Characterisation of Orchestration (OR) Molecules—T Cell Molecule Binding

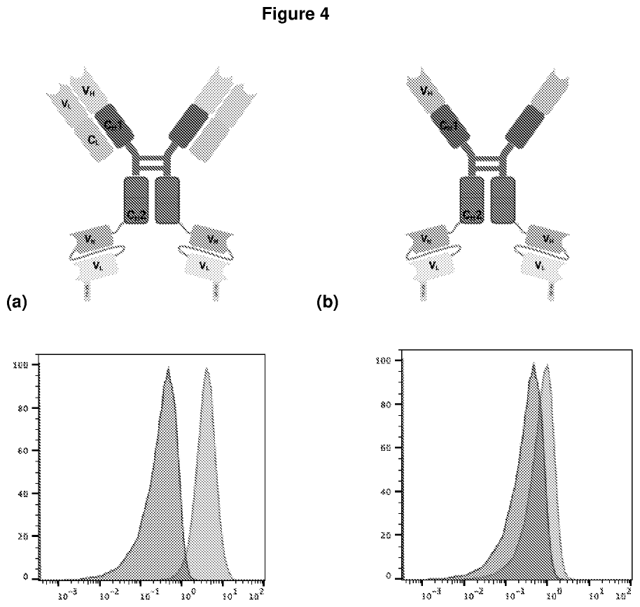

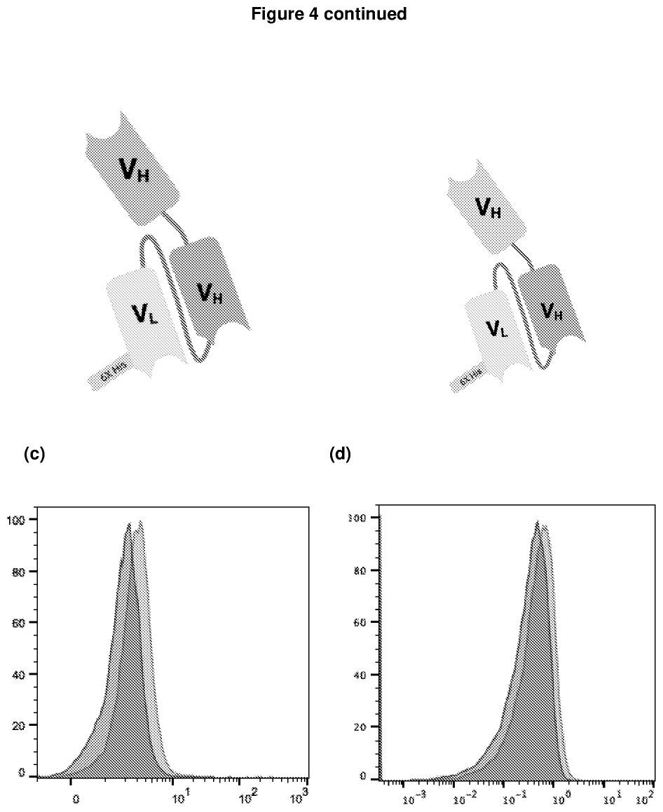

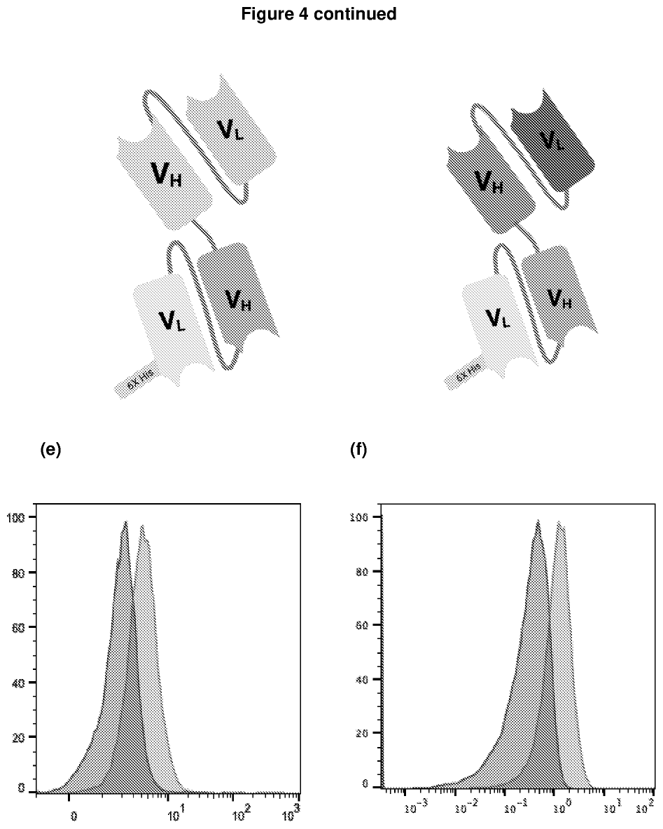

Those OR molecules characterised in Example 2 that are bispecific and that contain a second antigen binding domain intended to bind to a cell surface molecule on a T cell were tested by flow cytometric detection of their capacity to bind to T cells. Flow cytometric detection of binding capacity of OR molecules on T cells is shown in FIG. 4.

T cells express CD3 on their cell surface and therefore were used to confirm the ability of the OR molecules to bind CD3 on live cells.

FIG. 4 shows binding of bispecific orchestration molecules on T cells as determined by flow cytometric detection. Grey: Isotype control (left), Red: binding of HIS antibody (right shifted). HIS-antibody was used according to manufacturer with 1 μl per test, target cells were incubated with 50 μl of supernatant from Lenti-X™ 293T cells for 10 min at room temperature. Supernatant harvested 48 h after transfection. T-Cells were isolated by and cultured in TexMacs medium with IL-7/IL-15 for 7 days. (a) OR13-3, (b) OR14, (c) OR16, (d) OR17, and (e) OR19_6.

The results in FIG. 4 show that OR molecules with a second antigen binding domain in a wide variety of formats can bind to cell surfaces molecules present on immune cells. FIG. 4 confirms CD3 binding for (a) OR13-3, (b) OR14, (c) OR16, (d) OR17, and (e) OR19_6.

In summary, the functional characterisation in Examples 2 and 3 confirm that the OR molecules have the capacity to bind both a tumour-specific antigen (eg dysfunctional P2X₇ receptor) and a cell surface molecule on an immune cell (eg CD3 on T cells).

### Example 4—Characterisation of Various Bridging Molecules

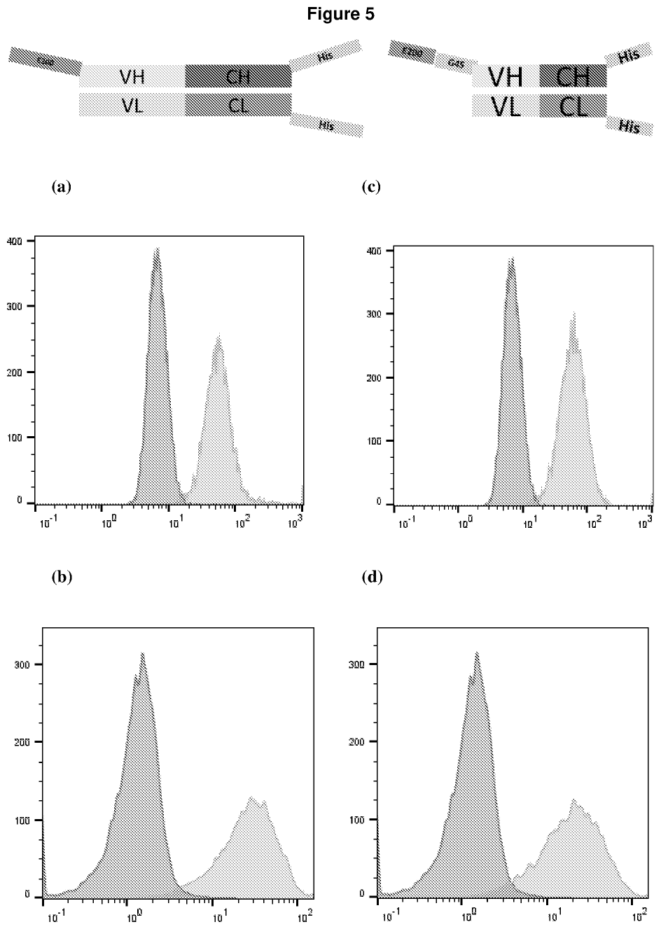

The results in FIGS. 5 to 8 show that bridging molecules comprising a targeting moiety in the form of a Fab or scFv (FMC63 clone; amino acid sequences described herein) that can bind to CD19, binds to CD19 on the surface of live cells and can present the dysfunctional P2X₇ receptor epitope moiety (e.g. E200 moiety) such that it is accessible by an anti-P2X₇ receptor antibody (BIL03s 2-2-1-Fc). The location of the dysfunctional P2X₇ receptor epitope moiety can vary and the targeting moiety can still bind to its target cell surface antigen and the dysfunctional P2X₇ receptor epitope moiety is still available for binding to an antibody. Note that BIL03s 2-2-1-Fc—AF647/HIS—FITC did not bind to control bridging molecules that did not contain the dysfunctional P2X₇ receptor epitope moiety, nor did the anti-HIS antibody bind to control bridging molecules that did contain a HIS tag (data not shown).

### Fab Format+/−Linker—Epitope on V_{H}

FIG. 5 shows that bridging molecules in Fab format with a single E200 epitope either directly linked to the V_{H} or via a linker binds to CD19 on JeKo-1 (mantle cell lymphoma) cell line and the E200 epitope is available for binding to an antibody.

scFV Format+/−Linker—Epitope on V_{H}

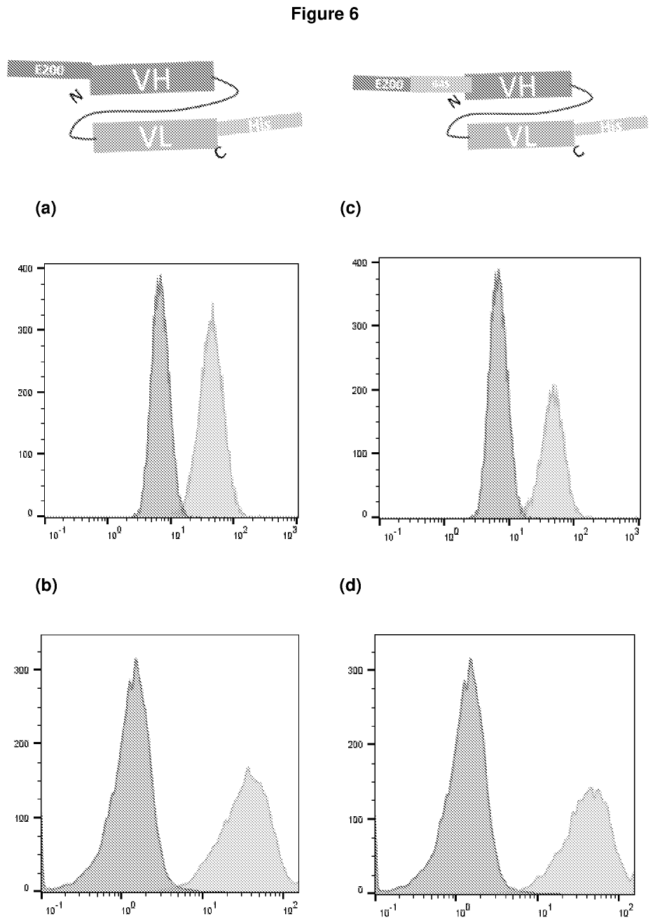

FIG. 6 shows that bridging molecules in scFv format with a single E200 epitope either directly linked to the V_{H} or via a linker binds to CD19 on JeKo-1 (mantle cell lymphoma) cell line and the E200 epitope is available for binding to an antibody.

### Fab Format+/−Linker—Epitope on V_{L}

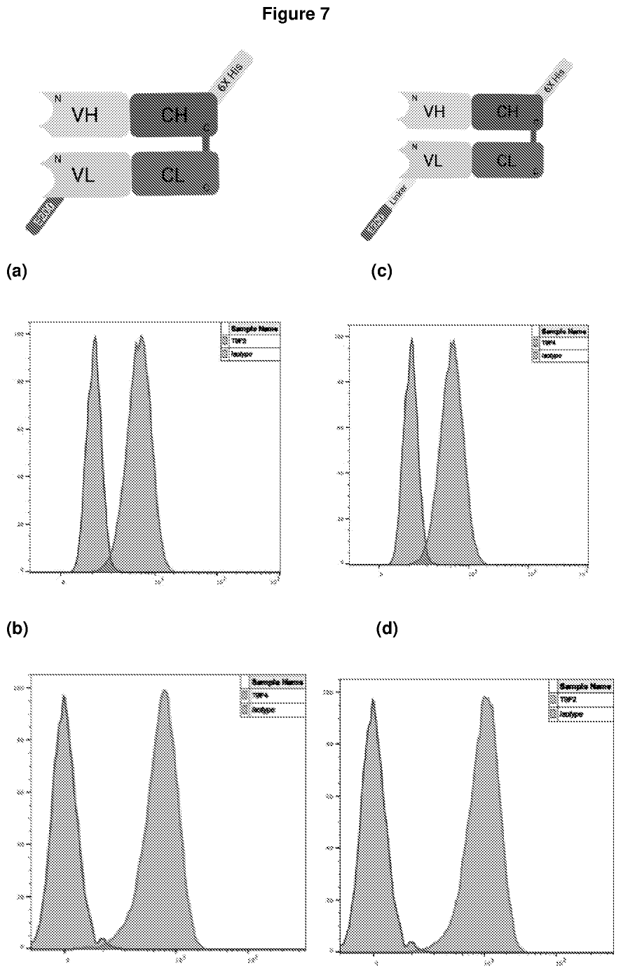

FIG. 7 shows that bridging molecules in Fab format with a single E200 epitope either directly linked to the V_{L} or via a linker binds to CD19 on JeKo-1 (mantle cell lymphoma) cell line and the E200 epitope is available for binding to an antibody.

scFv Format+/−Linker—Epitope on V_{L}

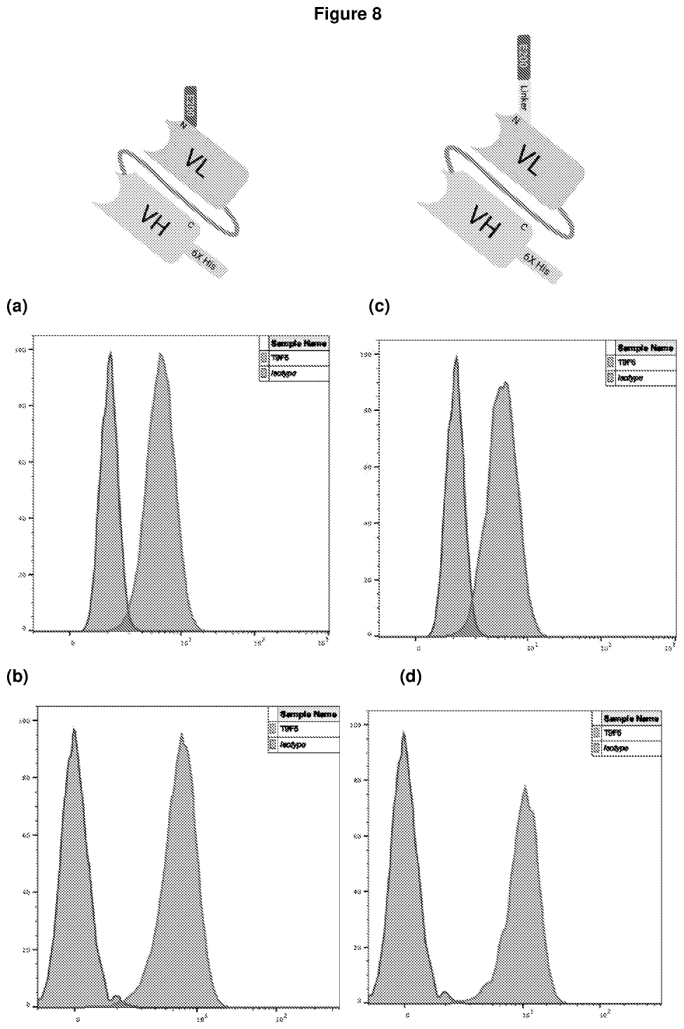

FIG. 8 shows that bridging molecules in scFv format with a single E200 epitope either directly linked to the V_{L} or via a linker binds to CD19 on JeKo-1 (mantle cell lymphoma) cell line and the E200 epitope is available for binding to an antibody.

### Bridging Molecules Targeting Various Antigens

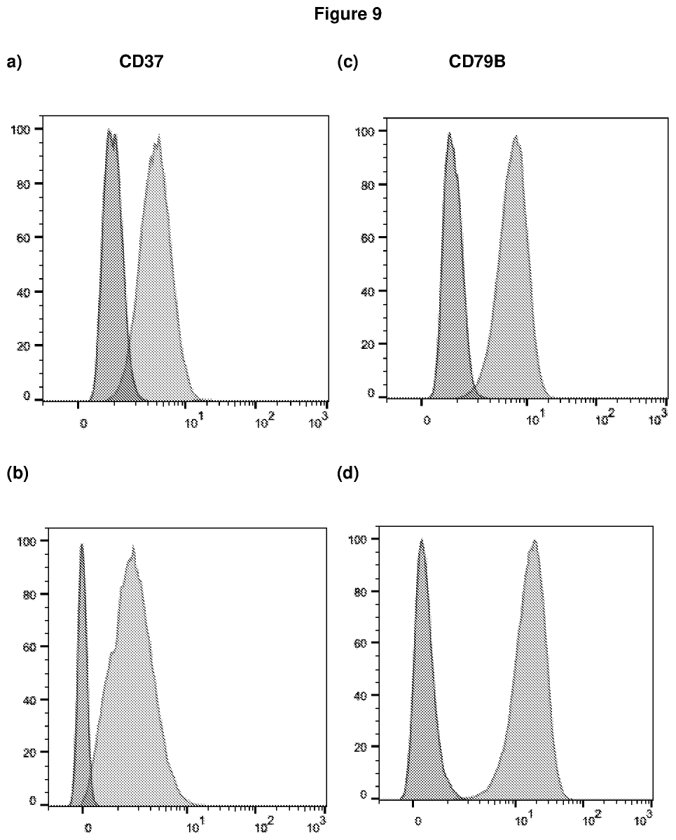

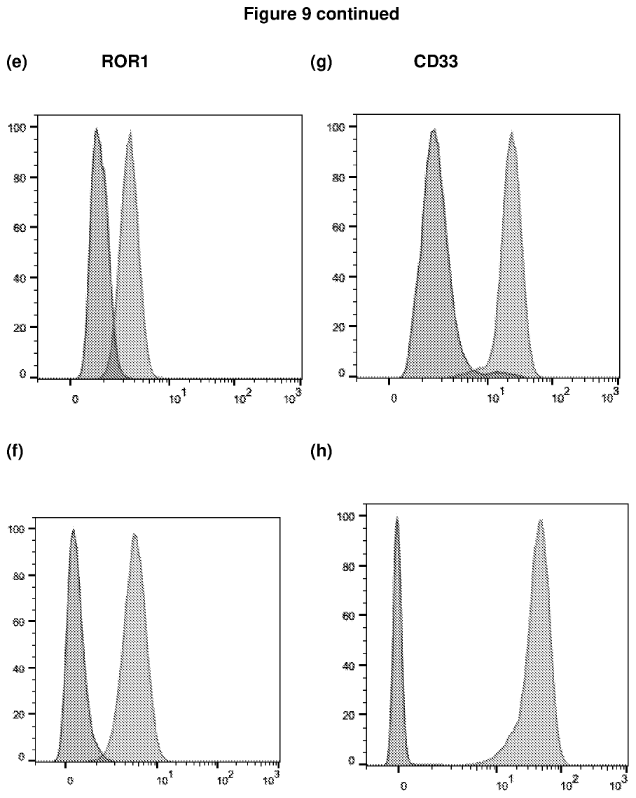

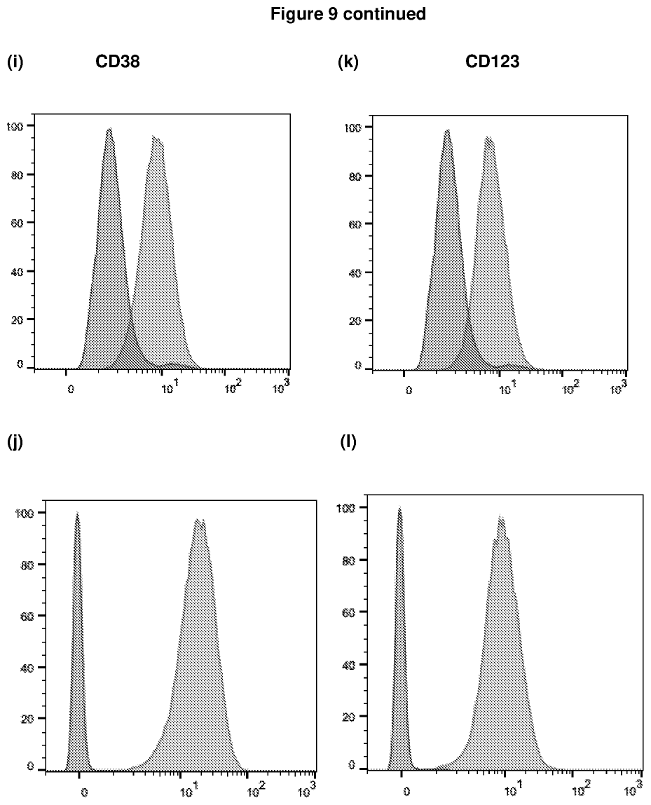

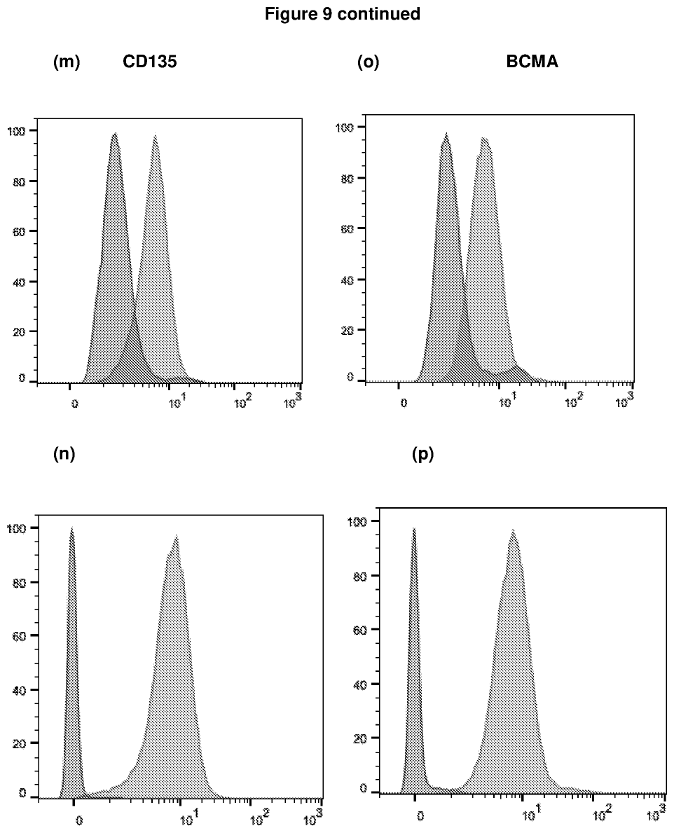

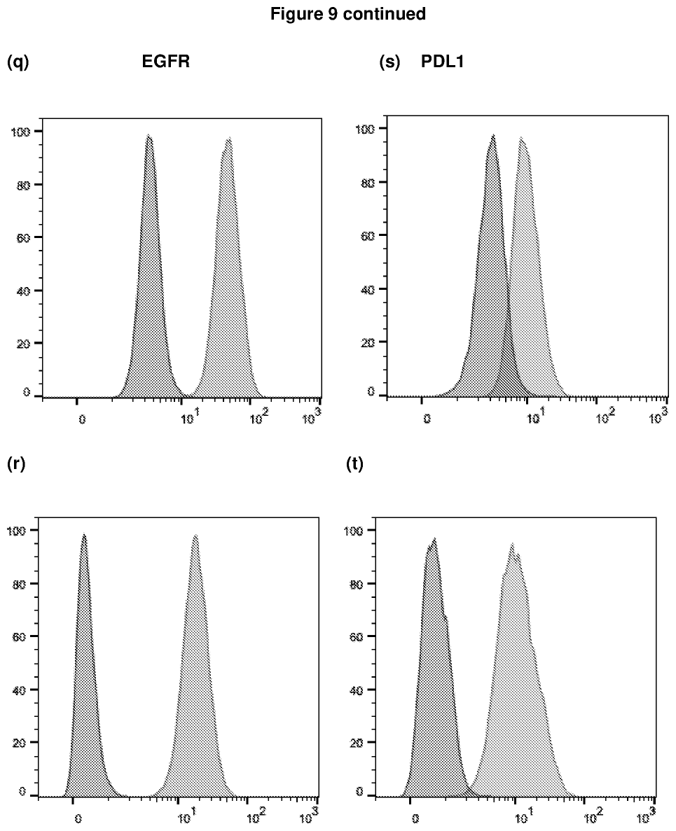

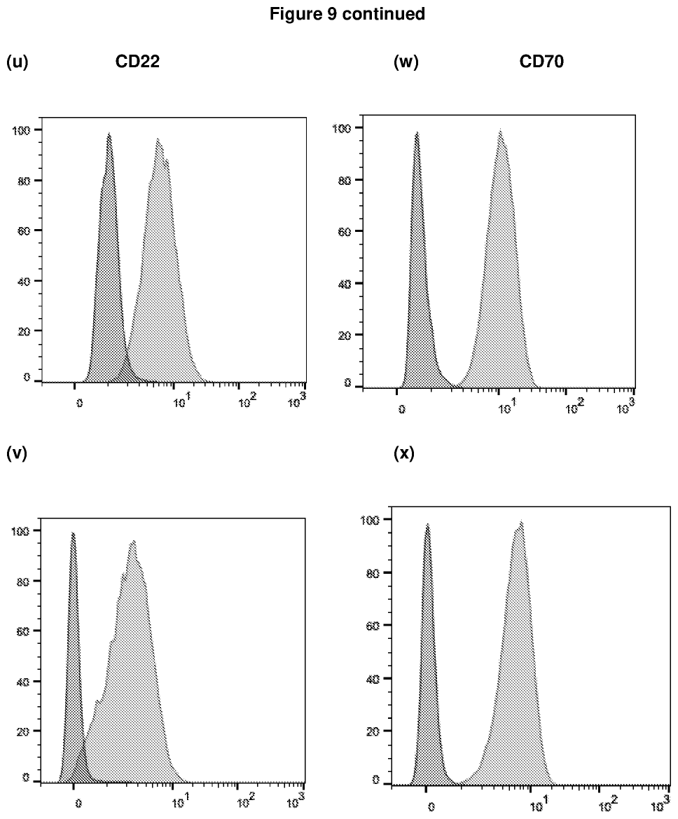

FIG. 9: Binding of bridging molecules to various antigens CD37, CD79B, ROR1, CD33, CD38, CD123, CD135, BCMA, EGFR, PDL1, CD22, CD70 and CD20. (a), (c), (e), (g), (i), (k), (m), (o), (q), (s), (u), (w) and (y) show anti-HIS antibody binding, (b), (d), (f), (h), (j), (1), (n), (p), (r), (t), (v), (x) and (z) show binding of antibody to dysfunctional P2X₇ receptor epitope.

CD37 targeting moieties derived from otlertuzumab, CD79B targeting moieties derived from polatuzumab, ROR1 targeting moieties derived from ROR1 APC (WO2016016344A1_D10v3), CD33 targeting moieties derived from lintuzumab, CD38 targeting moieties derived from daratumumab, CD123 targeting moieties derived from clone 32716, CD135 targeting moieties derived from 4G8, BCMA targeting moieties derived from clone CA8 J9MO, EGFR targeting moieties derived from necitumumab or matuzumab, PDL1 targeting moieties derived from atezolizumab, CD22 targeting moieties derived from m971-L7 (or inotuzumab (data not shown)), CD70 targeting moieties derived from cusatuzumab, CD19 targeting moieties derived from tafasitamab, CD20 targeting moieties derived from ofatumumab (or ocrelizumab (data not shown)). Exemplary amino acid sequences of bridging molecules targeting these antigens and derived from the antibodies mentioned immediately above are described in the sequence information table herein.

FIG. 9 shows binding of various bridging molecules to JeKo-1 (MCL) wild type cell line (CD37, CD79B, ROR1) Raji (Burkitt's lymphoma) wild type cell line (CD22, CD70, CD19, CD20, CD22), MOLM-13 (AML) wild type cell line (CD33, CD38, CD123 and CD135), RPMI 8226 (multiple myeloma) wild type cell line (CD33, BCMA and CD38), MDA-MB 231 (breast cancer) wildtype cell line (EGFR and PDL1) and PC-3 (prostate cancer) wild type cell line (EGFR).

### Example 5—Dose Dependent Increasing in Cell Binding by CD19 Targeting Bridging Molecule

JeKo-1 (MCL) CRL-3006™ wild type cell line purchased from ATCC as part of the NC160 panel. The cells were cultured according to general recommendations and standards for this particular cell line.

FIG. 10 shows “painting” of JeKo-1 with CD19 targeted Fab bridging molecules in the illustrated format.

Cells were incubated at indicated concentrations with Fab bridging molecules. CD33-targeted Fab-bridging molecules served as negative control in JeKo-1 at 10 ng/mL and 1000 ng/mL. CD19 targeted Fab-bridging molecule were used at 1 ng/mL, 10 ng/mL, 100 ng/mL and 1000 ng/mL.

The flow cytometric staining was undertaken in two steps according to standards in flow cytometric staining using the Fc block reagent (Miltenyi). First the target cells were incubated with Fab-bridging molecules at indicated concentrations for 15 min, washed three times and then the secondary antibodies anti-HIS FITC and the single domain antibody BIL03 2-2-1 AF647 was used at saturating concentrations (1 ug/mL) to indirectly stain the target cells via the 6×HIS and the nfP2X₇ E200 derived epitope on the bound Fab-bridging molecules. After 15 min of incubation the sample was washed and then analysed on a MACSQuant16 (Miltenyi). The flow data was analysed via FlowJo v10.7 (BD).

There is no expression of CD33 in JeKo-1 cells. CD19 staining showed increasing expression with increasing concentrations of CD19-targeted bridging molecules.

### Example 6—Dose Dependent Increase in Cell Binding by CD33 Targeting Bridging Molecule

MOLM-13 (AML) wild type cell line purchased from ATCC as part of the NC160 panel. The cells were cultured according to general recommendations and standards for this particular cell line.

FIG. 11 “painting” of MOLM-13 with CD33 targeted Fab bridging molecules in the illustrated format.

Cells were incubated at indicated concentrations with Fab bridging molecules. CD19 targeted Fab bridging molecule served as negative control in MOLM-13 at 10 ng/mL and 1000 ng/mL, while CD33 targeted Fab-bridging molecule was used at 1 ng/mL, 10 ng/mL, 100 ng/mL and 1000 ng/mL.

The flow cytometric staining was undertaken in two steps according to standards in flow cytometric staining using the Fc block reagent (Miltenyi). First the target cells were incubated with Fab-bridging molecules at indicated concentrations for 15 min, washed three times and then the secondary antibodies anti-HIS FITC and the single domain antibody BIL03 2-2-1 AF647 was used at saturating concentrations (1 ug/mL) to indirectly stain the target cells via the 6×HIS and the nfP2X₇ E200 derived epitope on the bound Fab-bridging molecules. After 15 min of incubation the sample was washed and then analysed on a MACSQuand16 (Miltenyi). The flow data was analysed via FlowJo v10.7 (BD).

There is no expression of CD19 in MOLM-13 cells. CD33 staining showed increasing expression with increasing concentrations of CD33-targeted bridging molecules.

### Example 7—Immune Cell Activation by Induced by Different OR Molecules with and without CD33 Targeted BRiDGEs at Different Fab-BRiDGE Molecule Concentration

The gating strategy is illustrated in FIG. 12 starting with a cell gate, a singlet gate to exclude doublets, a live-dead discrimination gate using only cells alive. T cells were discriminated from MOLM-13 via CD3 APC and MOLM-13 were generated to constitutively express eGFP. Only the T cell population was analysed for CD25 APCVio770 and CD69 VioGreen expression.

FIG. 13 show the flow cytometric analysis of T cells that were incubated with MOLM-13 cells under different conditions. In the left panel neither OR molecules nor BRiDGE molecules were added and the basic T cell activation referred to as CD25 and CD69 double positive cells was 3.65%. In the middle panel OR17 was added and the specific activation is increased to 8.05% whereas the combination of OR17 and B031 increased the specific activation to 31%.

### Example 8—Cytotoxicity of T Cells Versus MOLM-13 Cells by OR Molecules with and without Bridging Molecules

Viability was measured by the bioluminescence activity of MOLM-13 wildtype cell line transduced to constitutively express firefly luciferase and eGFP. Protocol is described in Example 1.

CD33 targeted BRiDGE molecules do not exert any toxicities on MOLM-13 cells, there is no toxicity of Fab monomers on their own when MOLM-13 cells are cultured in the presence of the BRiDGE molecules for 4 or 20 hours at concentrations up to 1000 ng/mL (data not shown).

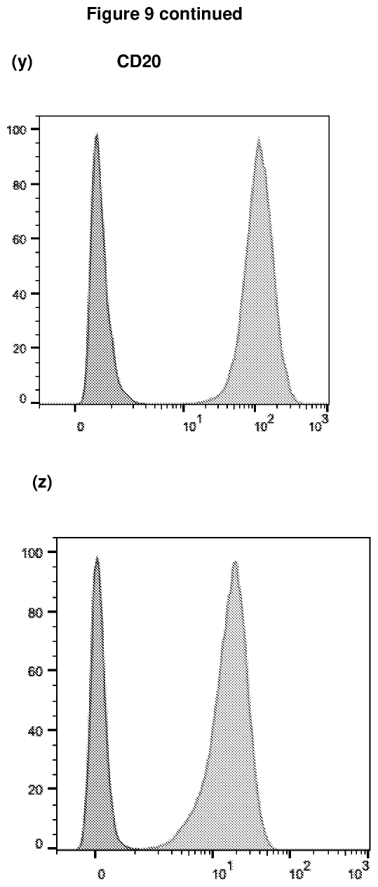

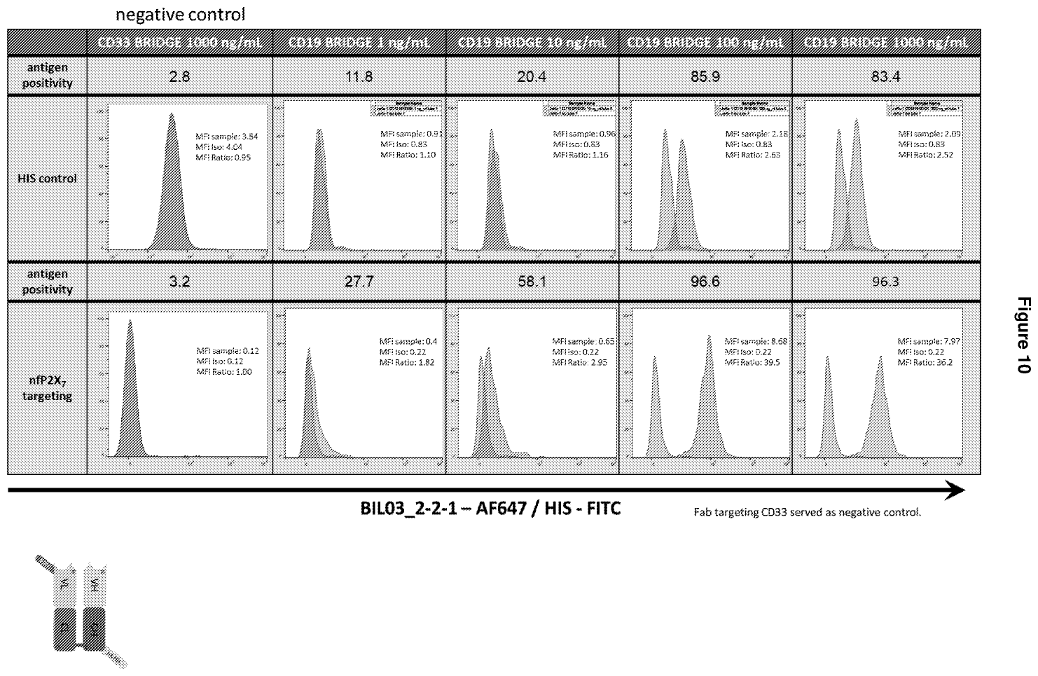

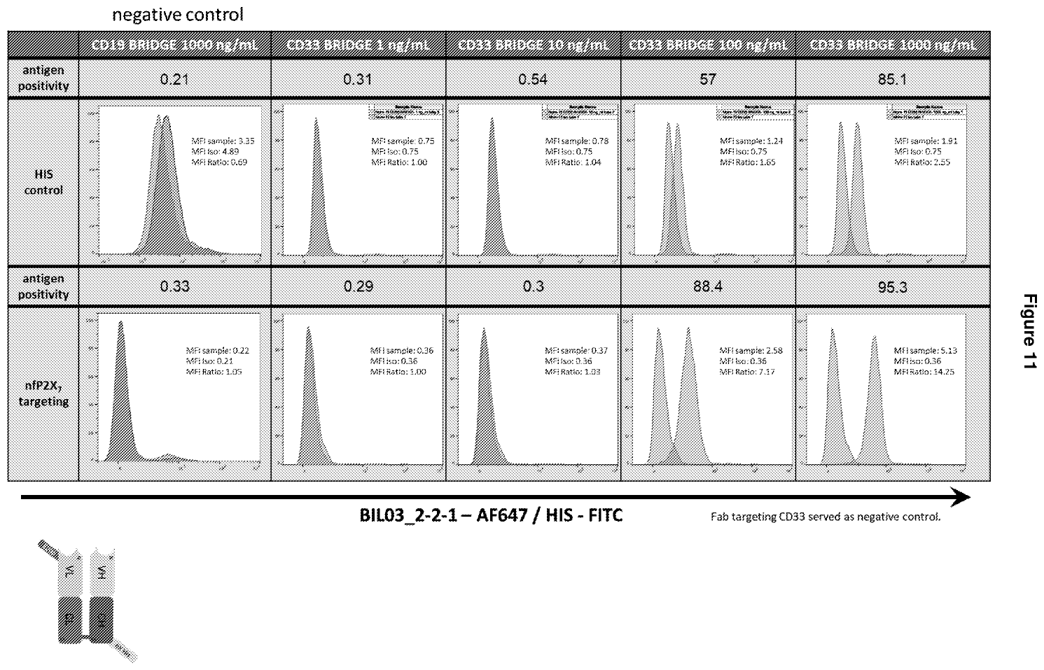

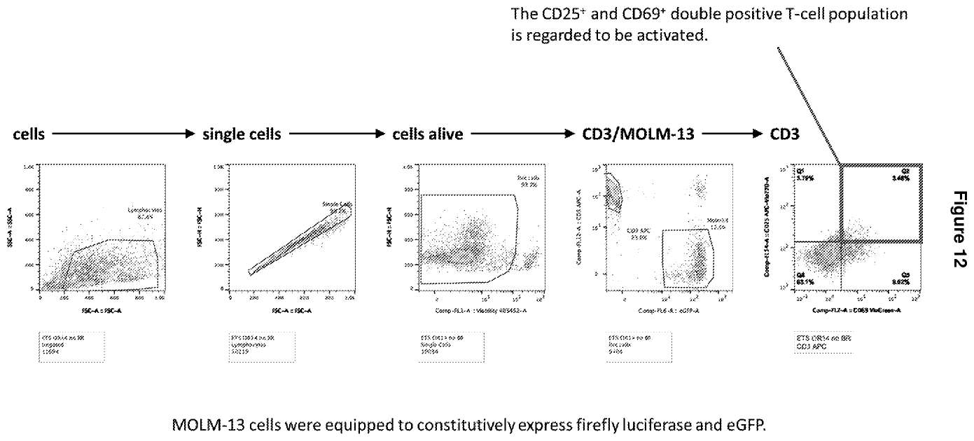

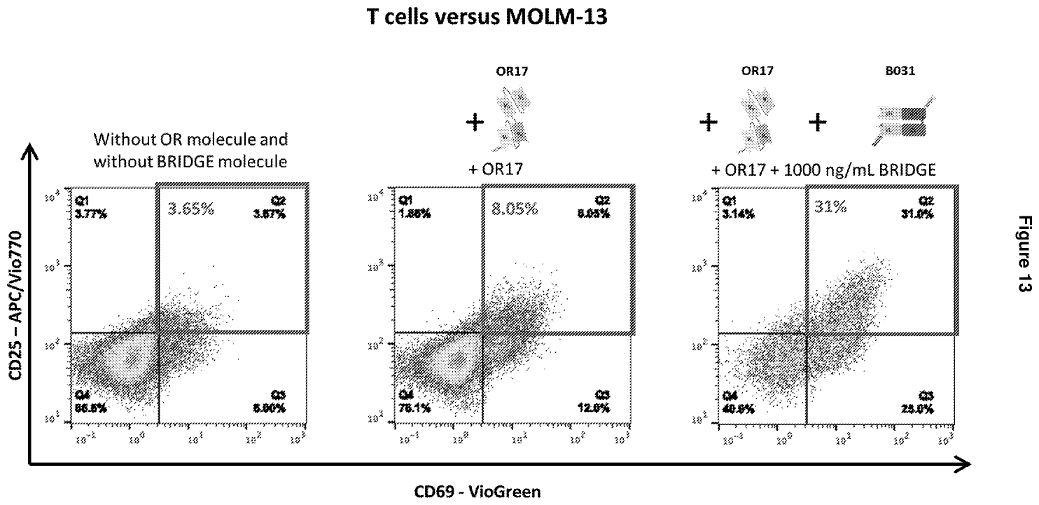

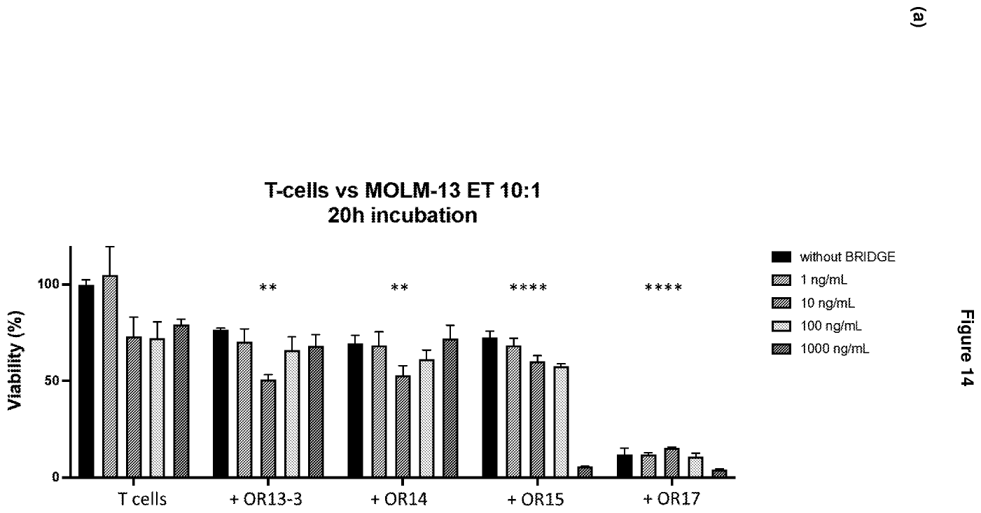

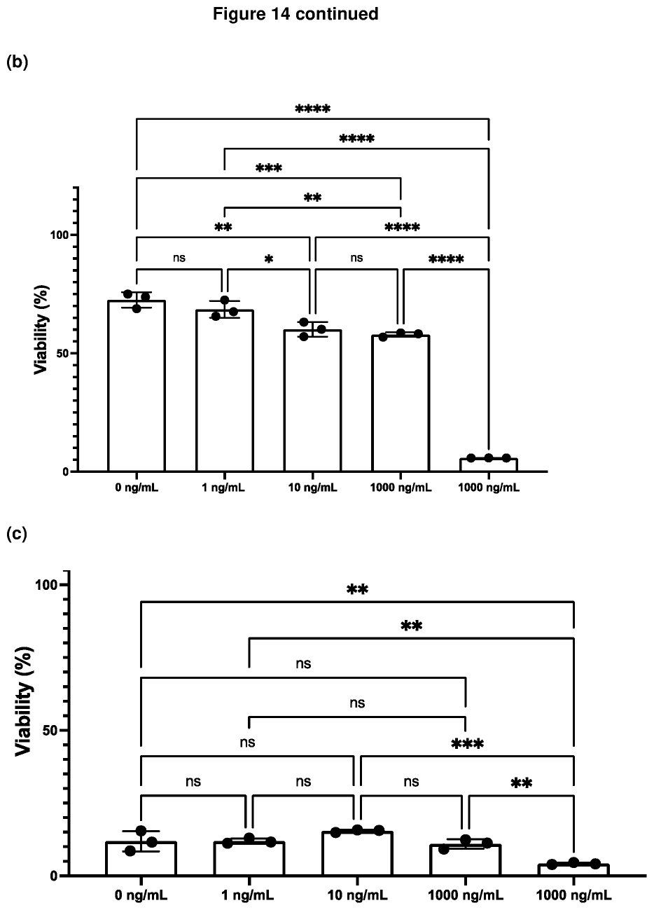

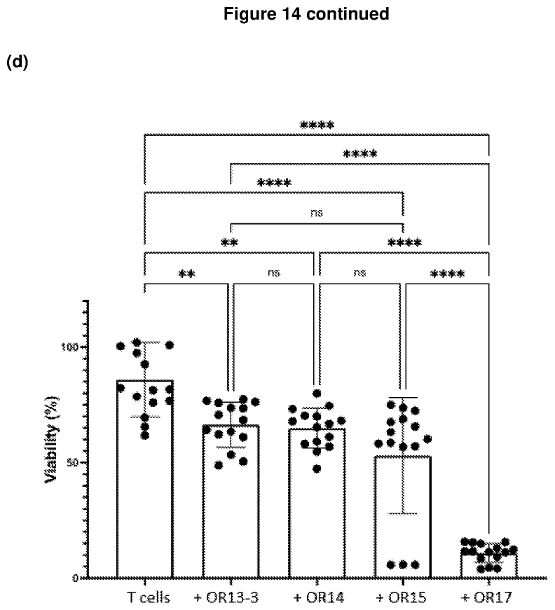

FIG. 14 shows luciferase-based cytotoxicity assay T cells versus MOLM-13. Viability was measured by the bioluminescence activity of MOLM-13 wildtype cell line transduced to constitutively express firefly luciferase and eGFP. OR molecules alone lead to a significant reduction of cancer cells, especially after 24 h, however this was further improved by the presence of a bridging molecule. (a) % viability of MOLM-13 cells after 20 hour incubation with T-cells in the presence of OR molecules, without, or with bridging molecules are various concentrations. (b) Summary of OR15, with and without CD33 bridging molecule (B031). (c) Summary of OR17, with and without CD33 bridging molecule (B031). (d) Summary of various OR molecules, with and without CD33 bridging molecule (B031).

### Example 8—Cytotoxicity of T Cells Versus JeKo-1 Cells by OR Molecules with and without Bridging Molecules

Viability was measured by the bioluminescence activity of JeKo-1 wildtype cell line transduced to constitutively express firefly luciferase and eGFP. Protocol is described in Example 1.

CD19 targeted BRiDGE molecules do not exert any toxicities on Jeko-1 cells, there is no toxicity of Fab monomers on their own when MOLM-13 cells are cultured in the presence of the BRiDGE molecules for 4 or 20 hours at concentrations up to 1000 ng/mL (data not shown).

FIG. 15 shows kinetics of luciferase-based cytotoxicity assay T cells from healthy donor versus JeKo-1 cells without bridging molecules. Cytolysis was measured by the bioluminescence activity of Jeko-1 cell line transduced to constitutively express firefly luciferase and eGFP. OR molecules alone lead to a significant reduction of cancer cells, especially after 23 h. Relevantly, OR17 was as potent as OR19_6 (which corresponds to a very similar protein like Blinatumomab the CD3×CD19 bispecific T cell engager [BiTE]) and OR13+3 that was significantly more potent than any other OR molecule tested.

Bridging molecule B19_8_Fab significantly increased the potency of man OR molecules as shown in FIG. 16 and in IgG1 format (B19_8_IgG1) in FIG. 17. B19_8_Fab has a light chain as shown in SEQ ID NO: 144, a Fab heavy chain (CD19, tafasitamab, B020-2_HC, SEQ ID NO: 52/143) and a dysfunctional P2X₇ receptor epitope moiety (SEQ ID NO: 4).

Neither B19_7_Fab nor B19_7_IgG1 which do not contain E200 epitope moiety increased the potency of the OR molecules (data not shown).

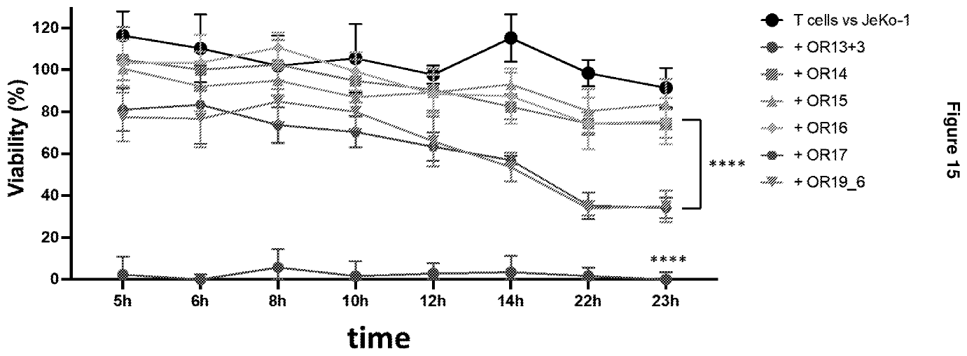

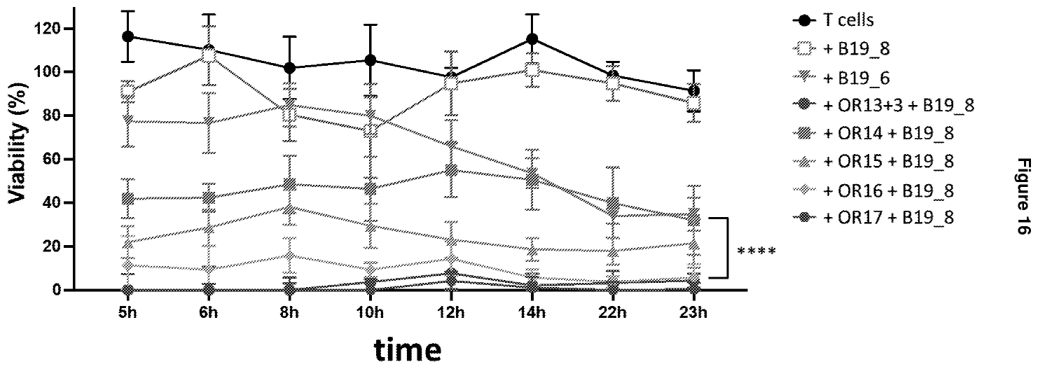

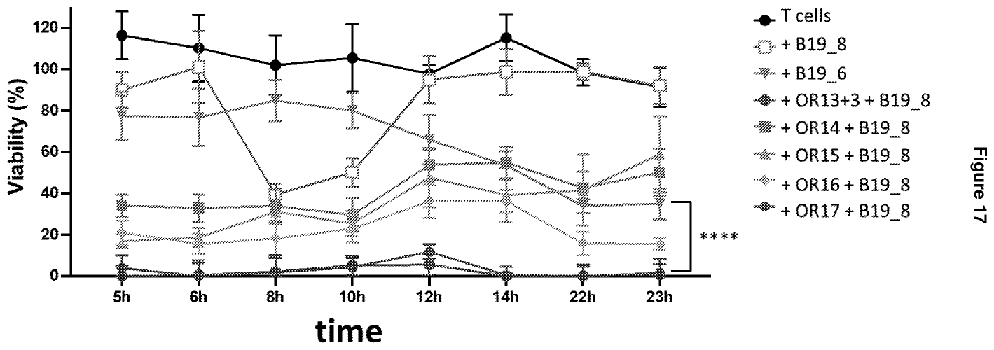

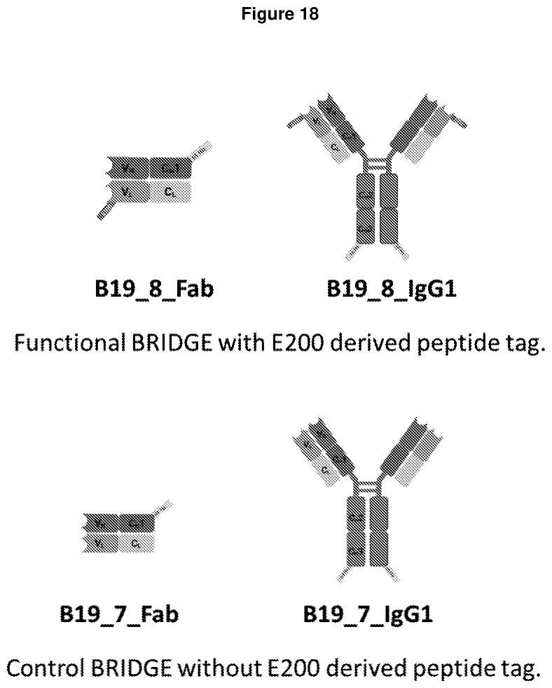

FIG. 18 shows evaluated bridging molecule variants.

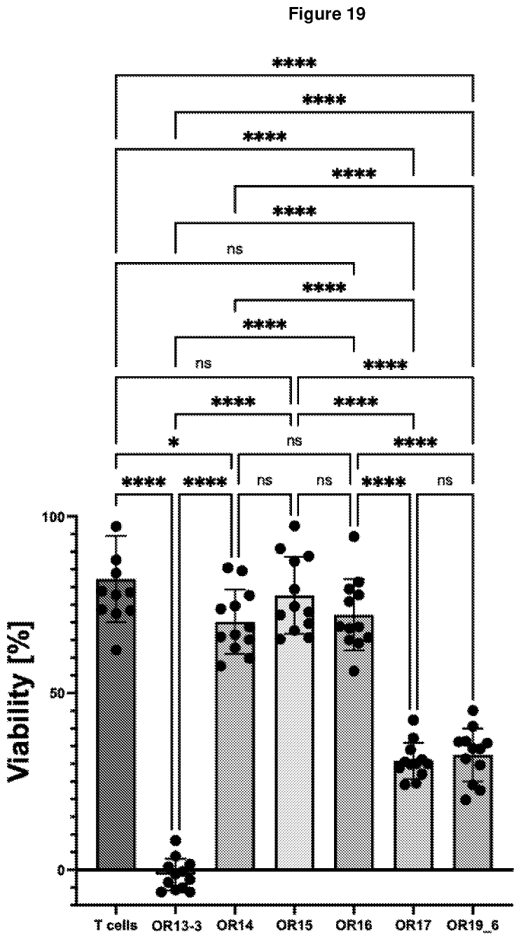

FIG. 19 shows viability of T cells without and with OR molecules versus JeKo-1 cells after 24 hour incubation and an effector cells to target ratio of 10:1. No bridging molecules present. T cells from 2 healthy volunteer donors and 6 technical replicates.

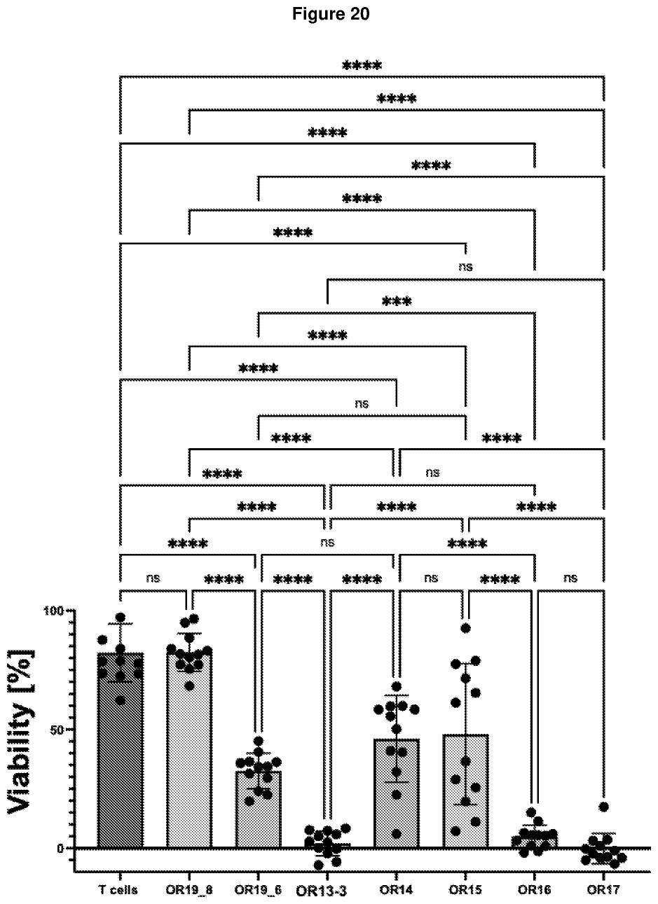

FIG. 20 shows viability of T cells without and with OR molecules versus JeKo-1 cells after 24 hour incubation and an effector cells to target ratio of 10:1. B19_8_Fab bridging molecules present at 100 ng/mL. CD19 targeted BRiDGE derived from tafasitamab. T cells from 2 healthy volunteer donors and 6 technical replicates.

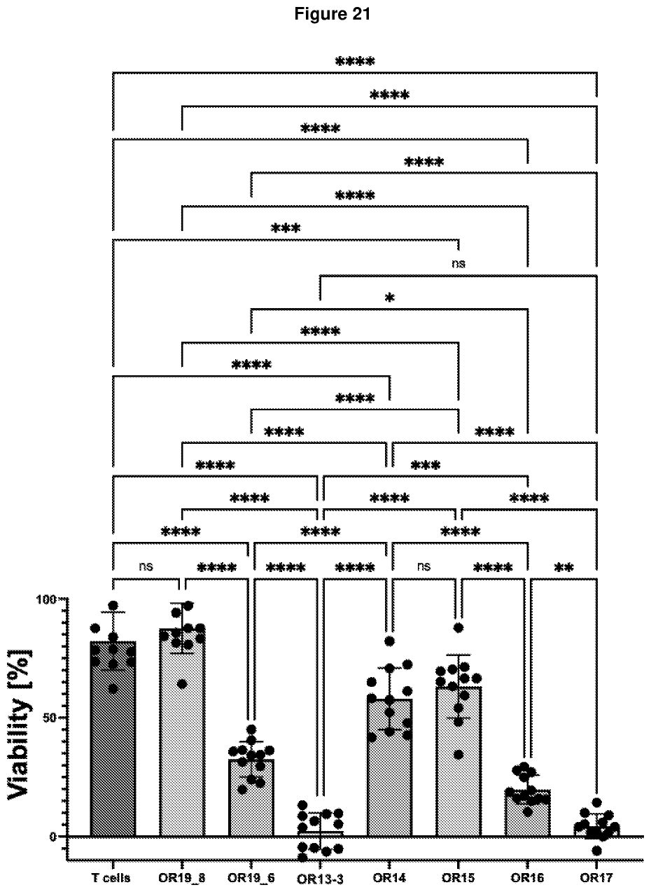

FIG. 21 shows viability of T cells without and with OR molecules versus JeKo-1 cells after 24 hour incubation and an effector cells to target ratio of 10:1. B19_8_IgG1 bridging molecules present at 100 ng/mL. CD19 targeted BRiDGE derived from tafasitamab.T cells from 2 healthy volunteer donors and 6 technical replicates.

Experimental data in this Example shows that different OR molecules increase the recruitment of T cells to cancer cells that leads to a significant reduction of JeKo-1 viability.

The recruitment of T cells may be increased by the combination of OR molecules and BRiDGE molecules that carry the nfP2X₇ E200 derived peptide tag variations represented by B19_8.

The increase in potency of T cells recruited by OR molecules depends on the specific mechanism that the BRiDGE molecules co-express the nfP2X₇ E200 derived peptide tag. As the BRiDGE variant B19_7 does not co-express the nfP2X₇ E200 derived peptide tag, it does not increase the recruitment of T cells in combination with OR molecules.

Utilising the ORCHESTRATION technology is not restricted to a specific BRiDGE format but Fab as well as IgG1 BRiDGEs may be used to engage with the OR molecules and T cells.

### Example 9—Evaluation of Bridging Molecules with Different Tumour-Specific Epitope Moiety Arrangements

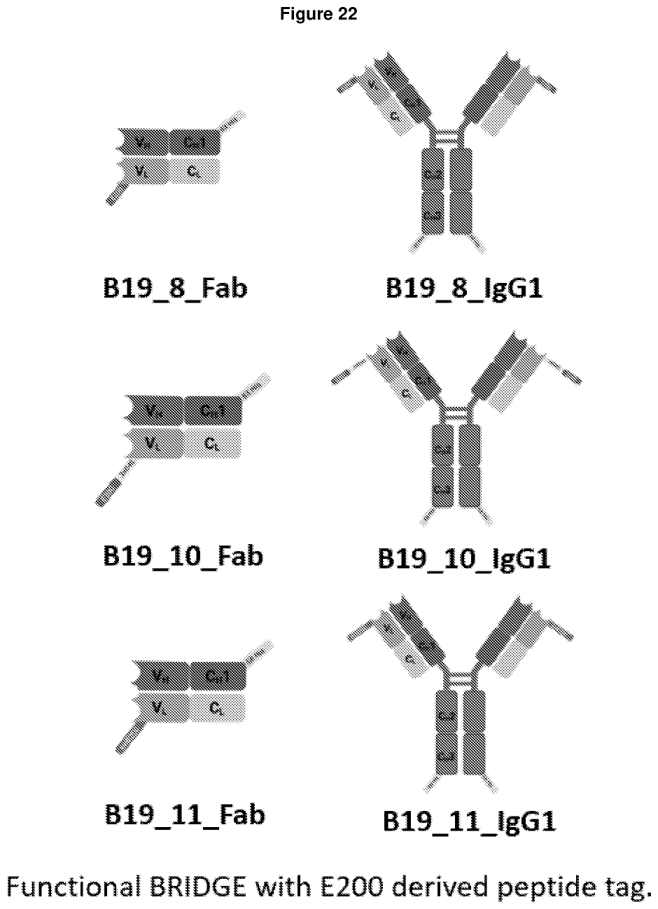

The experimental data described herein shows different E200 tag variants may recruit T cells to target cells, e.g. B19_8, B19_10, B19_11, in both bridging molecule formats Fab and IgG1 (see schematic diagrams of molecules in FIG. 22).

FIG. 23 shows viability of T cells without and with OR17 versus JeKo-1 cells after 24 hour incubation and an effector cells to target ratio of 10:1. B19_10_Fab bridging molecules present various concentrations. CD19 targeted BRiDGE derived from tafasitamab. T cells from 1 healthy volunteer donors and 6 technical replicates.

FIG. 24 shows viability of T cells without and with OR17 versus JeKo-1 cells after 24 hour incubation and an effector cells to target ratio of 10:1. B19_10_IgG1 bridging molecules present various concentrations. CD19 targeted BRiDGE derived from tafasitamab. T cells from 1 healthy volunteer donors and 6 technical replicates.

FIG. 25 shows viability of T cells without and with OR17 versus JeKo-1 cells after 24 hour incubation and an effector cells to target ratio of 10:1. B19_11_Fab bridging molecules present various concentrations. CD19 targeted BRiDGE derived from tafasitamab. T cells from 1 healthy volunteer donors and 6 technical replicates.

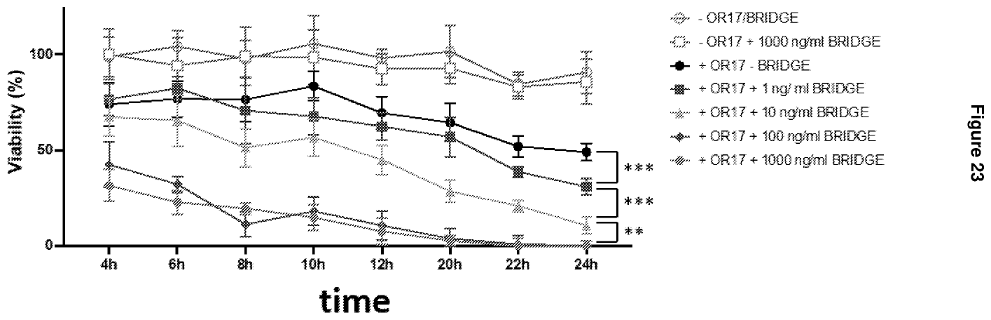

FIG. 26 shows viability of T cells without and with OR17 versus JeKo-1 cells after 24 hour incubation and an effector cells to target ratio of 10:1. B19_11_IgG1 bridging molecules present various concentrations. CD19 targeted BRiDGE derived from tafasitamab. T cells from 1 healthy volunteer donors and 6 technical replicates.

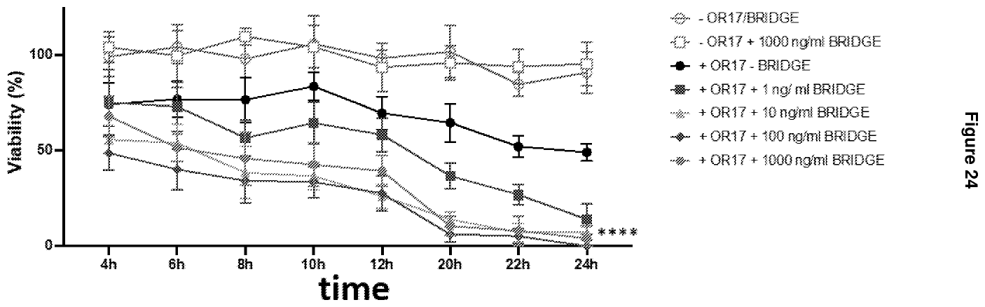

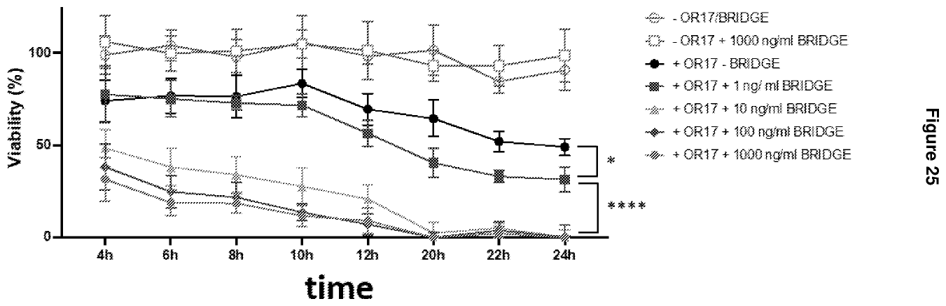

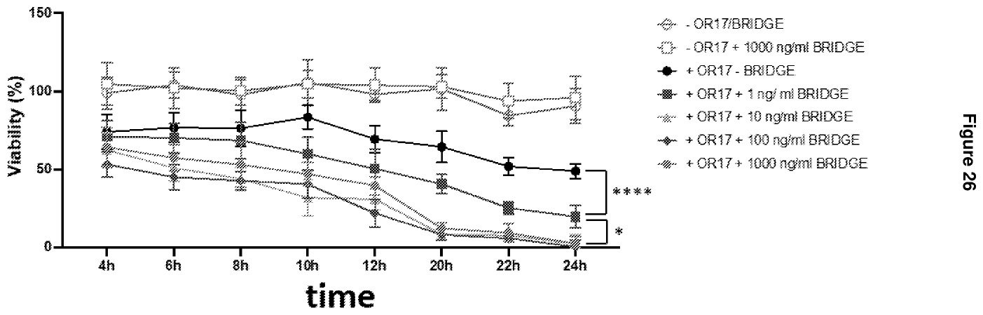

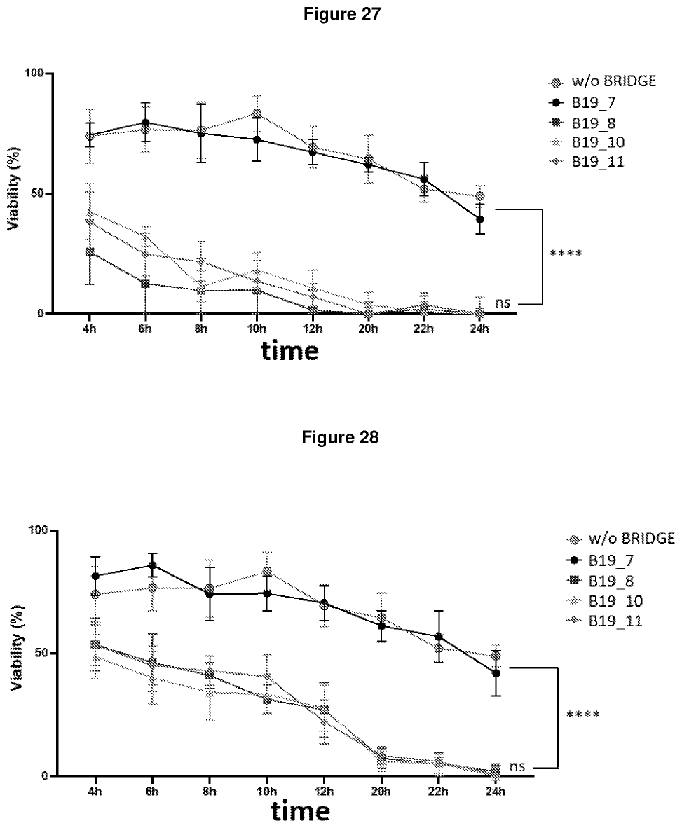

FIG. 27 shows a comparison of different BRiDGE variants at the same concentration 100 ng/mL in Fab format.

FIG. 28 shows a comparison of different BRiDGE variants at the same concentration 100 ng/mL in IgG1.

### Example 10—Alternative Antigen Targeting on AML MOLM-13 Cancer Cells—CD38

Viability was measured by the bioluminescence activity of MOLM-13 wildtype cell line transduced to constitutively express firefly luciferase and eGFP. Protocol is described in Example 1.

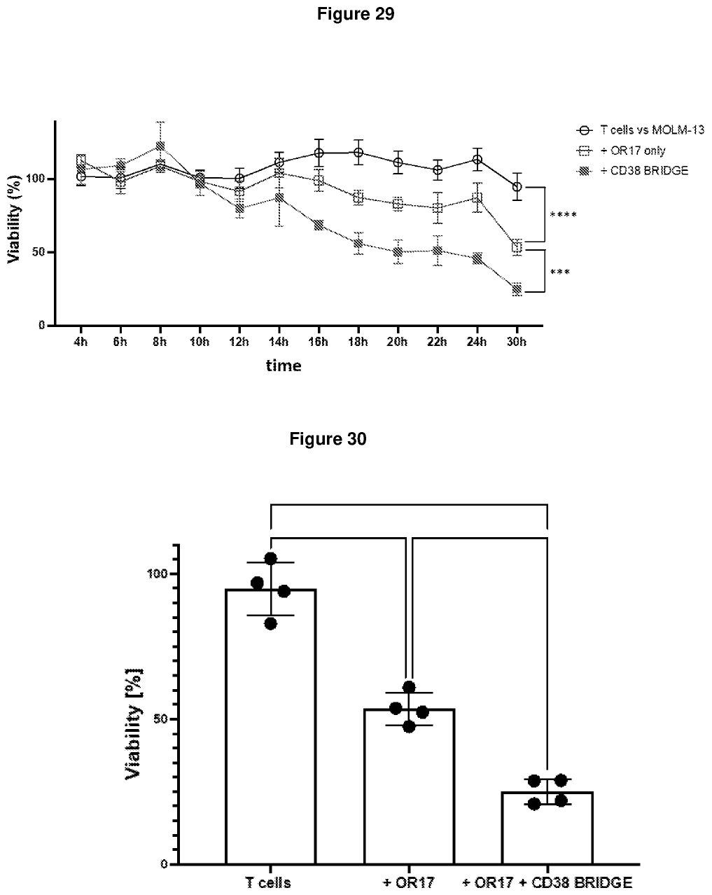

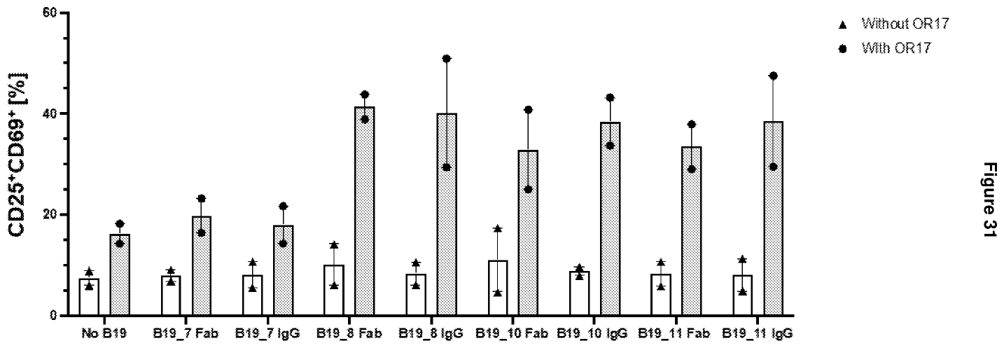

FIGS. 29 and 30 shows luciferase-based cytotoxicity assay T cells versus MOLM-13. Viability was measured by the bioluminescence activity of MOLM-13 wildtype cell line transduced to constitutively express firefly luciferase and eGFP. OR molecules alone lead to a significant reduction of cancer cells, especially after 24 h, however this was further improved by the presence of a bridging molecule B027 targeting CD38.

### Example 11—Activation of T Cells by OR Molecules and Different Bridging Molecules

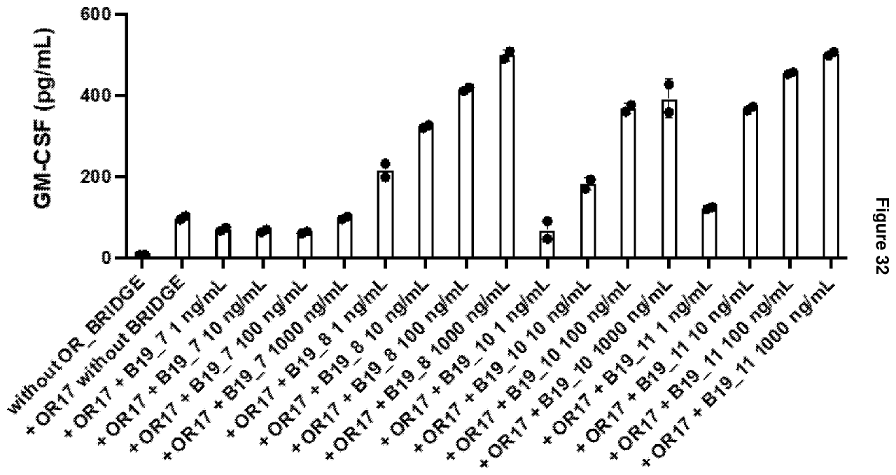

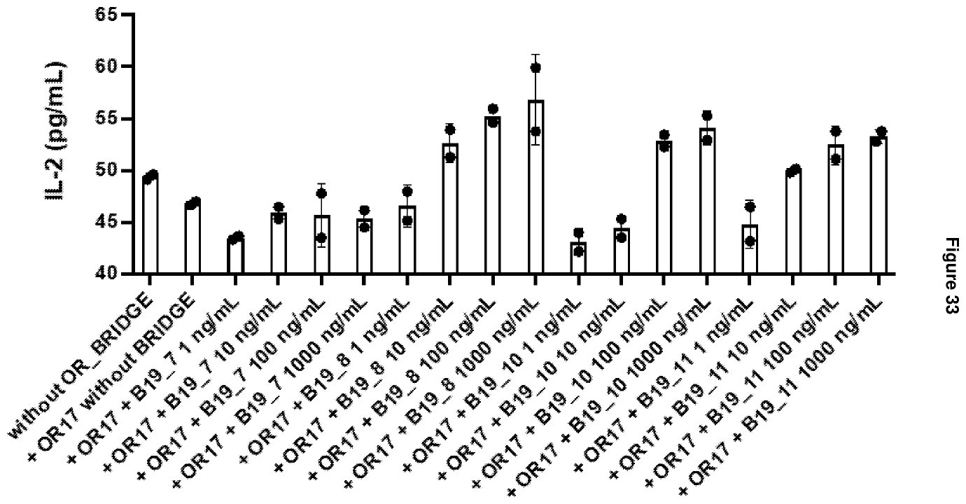

FIG. 31 shows the activation of T cells measured by CD25+CD69+ cells with the variables+/−OR17 and the different bridging molecules in Fab and IgG1 format and tag variants OR19_7 (control without E200), B19_8, B19_10, B19_11. 2 healthy donors. Effector/target ratio 10:1. Bridging molecule in Fab/IgG1 format, final concentration 100 ng/mL, incubation time 24 h.

### Example 12—Cytokine Induction from T Cells

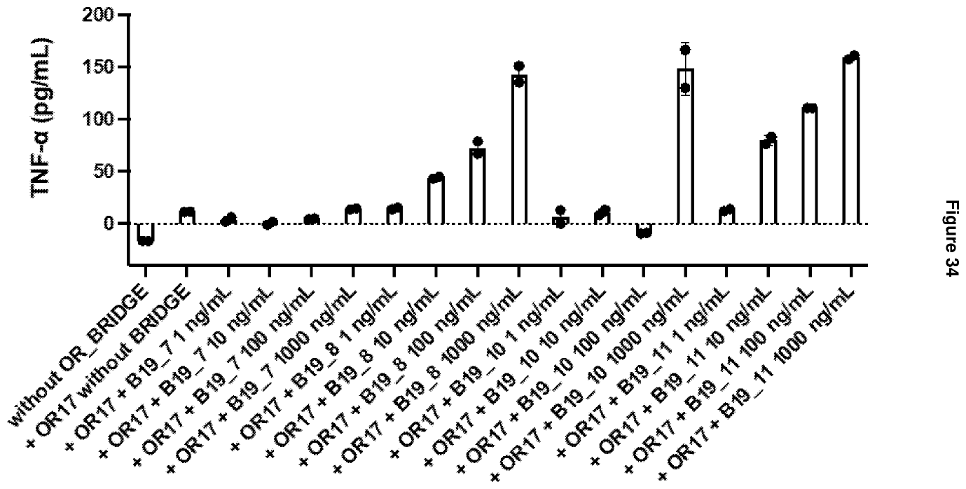

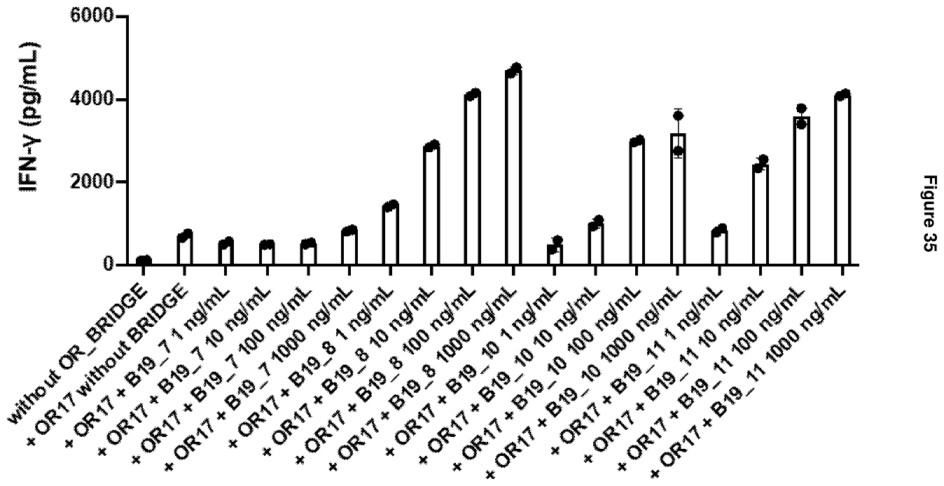

Cytokine secretion of GMCSF, IL2, TNFa and IFNgamma under different conditions T cells targeting JeKo-1 with the variables+/−OR17 and different BRiDGE molecule variants in the Fab format with OR19_7 as the control BRiDGE and the OR19_8, OR19_10 and OR19_11 as the functional BRiDGEs is shown in FIGS. 32, 33, 34 and 35 respectively.

### Example 13—Recruitment of Innate Immune Cells

The OR molecules and methods of the present application can also be used to recruit innate immune cells such as natural killer cells. In other words, an orchestration molecule (OR) can be used to bind to both a cancer cell antigen (e.g. nfP2X₇ receptor) and an antigen on an innate immune effector cell (such as an NK cell). The potency of OR molecules can be significantly enhanced by adding bridging molecules.

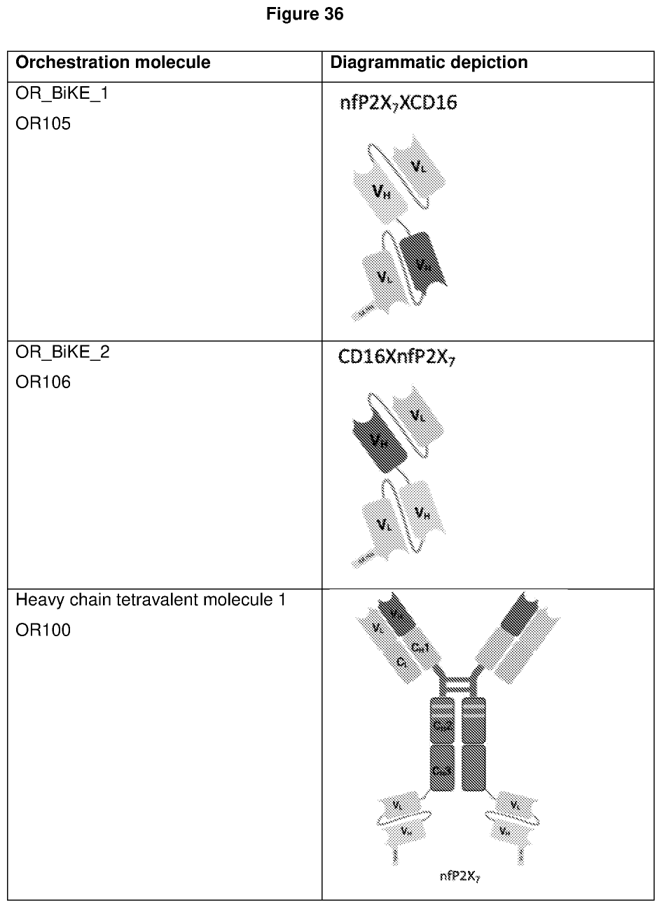

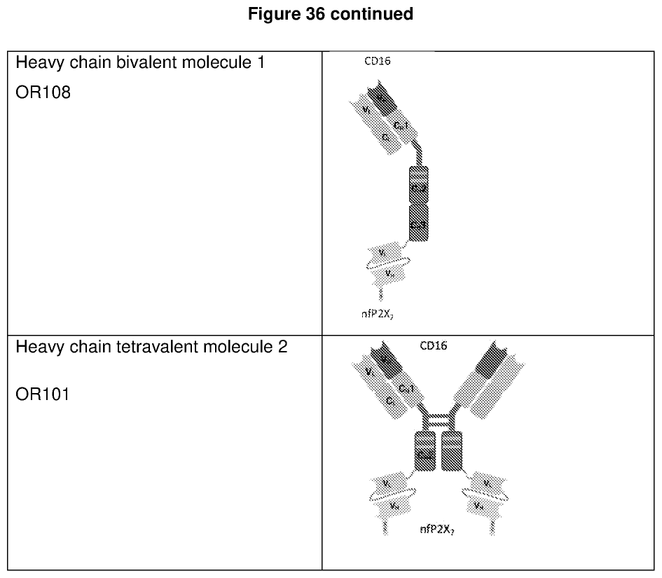

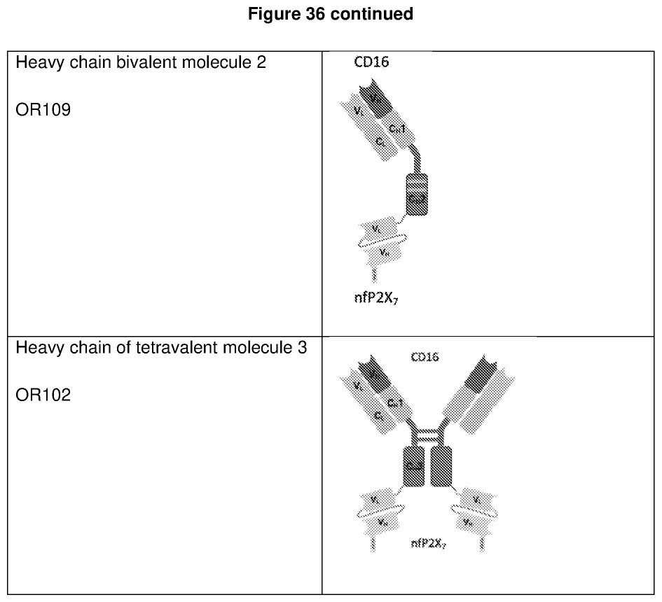

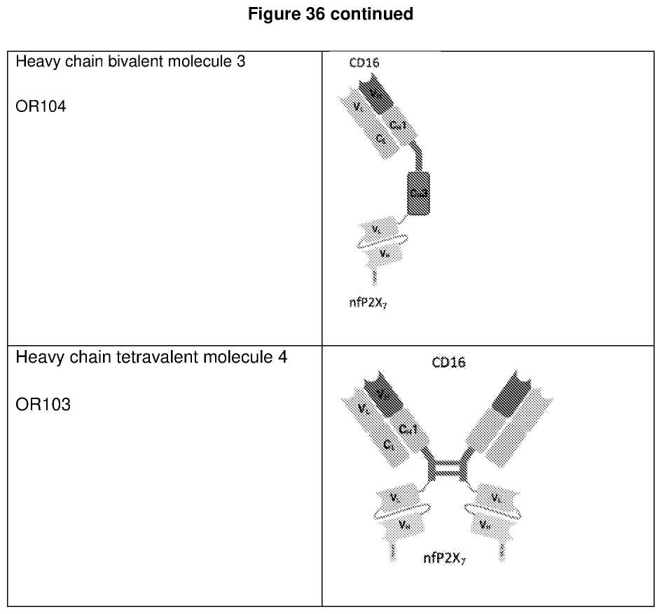

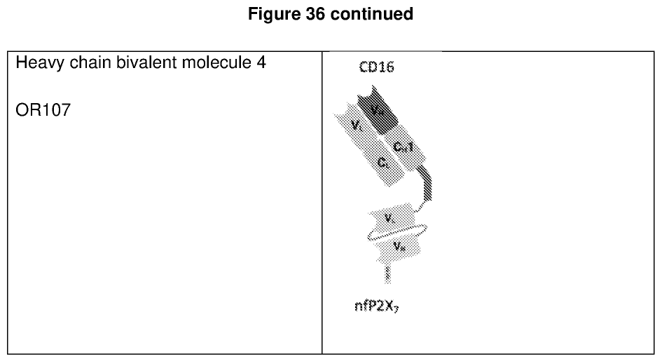

In one example, OR molecules are designed having an antigen binding domain for binding CD16 (also known as FcγRIII; on innate immune effector cells) and an antigen binding domain for binding to nfP2X₇ receptor. Examples of various ORs having different arrangements of antigen binding domains are listed below and also shown in FIG. 36. Exemplary amino acid sequences are provided in SEQ ID NOs: 350 to 359 (these sequences include a signal sequence that is interchangeable and could also be deleted from the sequence.

Molecules can be in the format of a “BiKE” (bispecific killer cell engager), tetravalent or bivalent molecule, as further defined herein. In the present example, a representative CD16-binder (3G8 clone) and binding protein comprising VL (WTB1) and VH (BIL03)—as elsewhere herein described, are utilised although it will be appreciated that any CD16 or nfP2X₇ binders could be used.

| | | Targeting |
|---|---|---|
| Plasmid | Structure | antigens |
| OR_BIKE1 | VL(WTB1)4GS*3_VH(BIL03)_ | nfP2X₇ x CD16 |
| (LHHL | linker_VH_VL(3G8clone)_(SEQ ID | |
| config) | NO: 350) | |
| OR105 | | |
| OR_BIKE2 | [3G8 clone]_linker_VL(WTB1)_ | CD16 x nfP2X₇ |
| (LHHL | 4GS*3_VH(BIL03) (SEQ ID NO: | |
| config) | 351) | |
| OR106 | | |
| Heavy | [3G8 clone]_CH1_lgG1hinge_CH2_CH3 | CD16 x nfP2X₇ |
| chain | VL(WTB1)_4GS*3_VH(BIL03)_ | |
| tetravalent | HISx6 (SEQ ID NO: 352) | |
| molecule 1 | | |
| OR100 | | |
| (Fc | | |
| attenuated) | | |
| Heavy | [3G8 | CD16 x nfP2X₇ |
| chain | clone]_CH1_lgG1hinge_CH2_ | |
| bivalent | CH3_VL(WTB1)_4GS*3_VH(BIL03)- | |
| molecule 1 | _HISx6_ (SEQ ID NO: 353) | |
| Heavy | [3G8 clone]_CH1_IgG1hinge_ | CD16 x nfP2X₇ |
| chain | CH2_VL(WTB1)_4GS*3_VH- | |
| tetravalent | (BIL03_HISx6 (SEQ ID NO: 354) | |
| molecule 2 | | |
| OR101 | | |
| (Fc | | |
| attenuated- | | |
| no CH3) | | |
| Heavy | [3G8 clone]_CH1_IgG1hinge_ | CD16 x nfP2X₇ |
| chain | CH2_VL(WTB1)_4GS*3_VH- | |
| bivalent | (BIL03)_HISx6_1-2 (SEQ ID NO: 355) | |
| molecule 2 | | |
| Heavy | [3G8 clone]_CH1_IgG1hinge_ | CD16 x nfP2X₇ |
| chain | CH3_VL-WTB1_4GS*3_VH- | |
| tetravalent | BIL03_HISx6 (SEQ ID NO: 356) | |
| molecule 3 | | |
| OR102 | | |
| (no CH2) | | |
| Heavy | [3G8 clone]_CH1_IgG1hinge_ | CD16 x nfP2X₇ |
| chain | CH3_VL-WTB1_4GS*3_VH- | |
| bivalent | BIL03_HISx6_1-2 (SEQ ID NO: 357) | |
| molecule 3 | | |
| OR104 | | |
| (no CH2) | | |
| Heavy | [3G8 clone]_CH1_IgG1hinge_VL- | CD16 x nfP2X₇ |
| chain | WTB1_4GS*3_VH-BIL03_HISx6 | |
| tetravalent | (SEQ ID NO: 359) | |
| molecule 4 | | |
| OR103 | | |
| (no CH2 or | | |
| CH3) | | |
| Heavy | [3G8 clone]_CH1_lgG1hinge_VL- | CD16 x nfP2X₇ |
| chain | WTB1_4GS*3_VH- | |
| bivalent | BIL03_HISx6_1-2 (SEQ ID NO: 359) | |
| molecule 4 | | |

These bispecific fusion proteins closely connect the effector cell to the cancer cell. It will be appreciated that any other number of binding proteins for binding to alternative innate immune cell antigens can be used, for instance antibodies for binding NKp46, NKG21D, NKp44 and DNAM-1 and others.

The molecules may be designed as tetravalent or bivalent molecules, as exemplified above.

The CD16 antibody clone 3G8 is targeted to an epitope on human CD16, also known as low-affinity IgG receptor III (FcγRIII). There are two distinct forms of CD16, CD16a (FcγRIIIa) and CD16b (FcγRIIIb). CD16a is a 50-65 kDa heterooligomeric polypeptide-anchored transmembrane protein expressed by NK cells, macrophages, and subsets of monocytes. CD16b is a 48 kDa monomeric glycosylphosphatidylinositol (GPI)-anchored protein expressed on neutrophils. CD16a shows a 10-fold higher affinity to IgG-based antibodies compared to CD16b. Upon binding to the Fc portion of IgG or IgG-antigen complex, both CD16 isoforms induce signalling cascades resulting in multiple functions, including antibody-dependent phagocytosis (ADPC), cytokine release, proliferation, degranulation, and antibody dependent cell-mediated cytotoxicity (ADCC).

CD16 is expressed on cytotoxic NK cells, activated monocytes, macrophages, polymorphonuclear neutrophils (during maturation), subsets of T cells and subsets of T cells, placental trophoblasts.

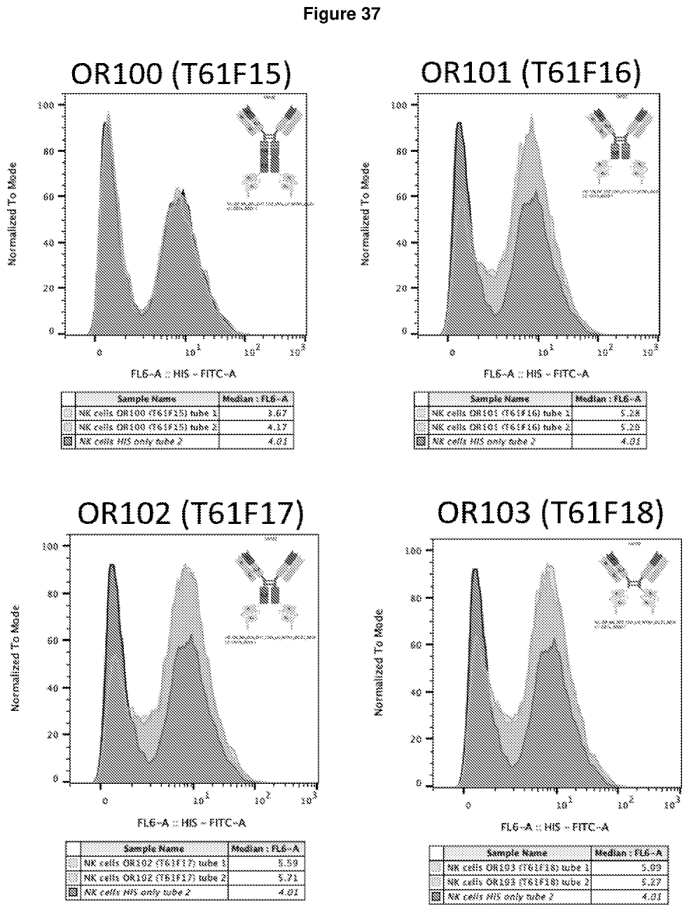

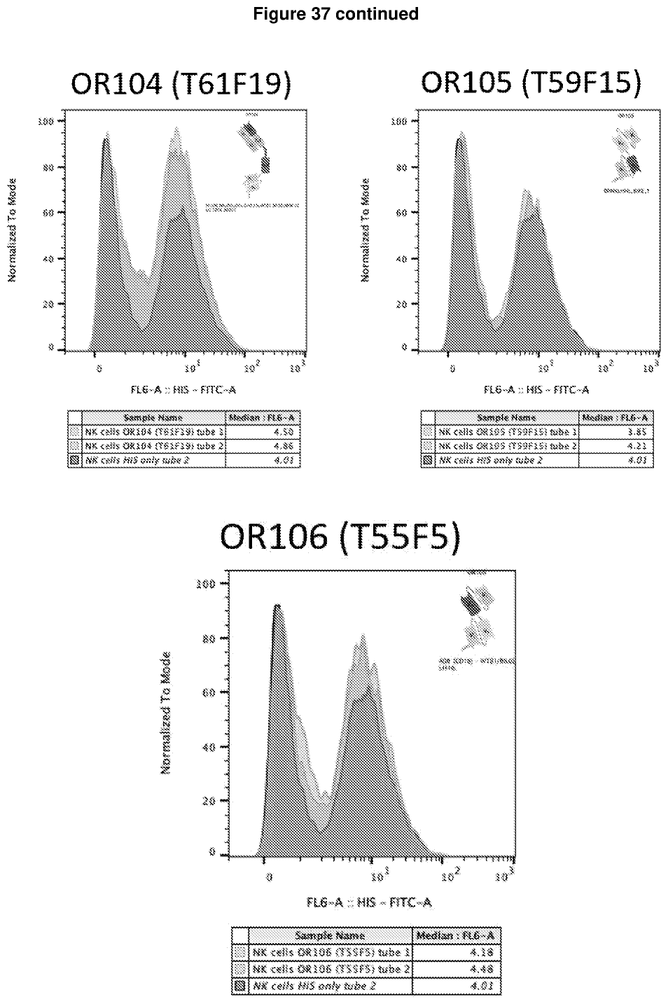

The OR molecules designed and referred to above were demonstrated to bind to NK cells (FIG. 37).

A Luciferase-based cell killing assay was performed to demonstrate the ability of OR molecules to recruit expanded NK cells to kill cancer cells. Briefly: freshly isolated PBMCs (comprising NK cells) or expanded NK cells were co-cultured with target JeKo-1 MG4 cancer cells at various effector:target ratios+/−orchestration molecules. In one example OR106 (SEQ ID NO: 351) was utilised. The same experiments were conducted, further including use of a BRiDGEmolecule comprising 1) an antigen for binding by the nfP2X₇ receptor antigen binding domain of OR106 and 2) an anti-CD 19 binding domain for binding the cancer cells.

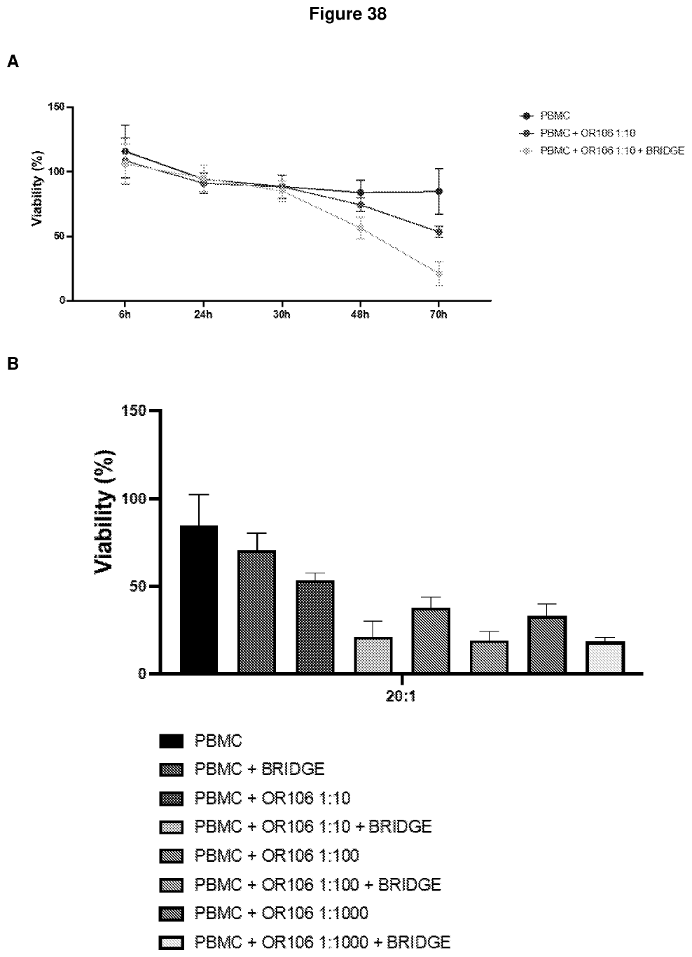

The results, shown in FIG. 38A demonstrate that when using an effector:target ratio of 1:10, OR106 molecules in combination with PBMCs (comprising NK cells), significantly reduced the viability of JeKo-1 cancer cells. Thus the Orchestration molecules are capable of reducing cancer cell numbers by bringing innate immune cells (such as NK cells) into closer proximity to nfP2X₇ receptor expressing cancer cells. Addition of a bridging (BRiDGE) molecule comprising an epitope of nfP2X₇ receptor (capable of being bound by the OR) and an antigen binding domain for binding CD19, further reduces the viability of the cancer cells, demonstrating that the inclusion of a bridging molecule ostensibly enhances the efficacy of the OR molecules by creating more epitopes on cancer cells via which the NK cells can be engaged.

Similar experiments were conducted using OR molecules 100, 103 and 104 as defined herein, including in the presence of either a CD19-binding bridging molecule or a CD20-binding bridging molecule. The results demonstrate that the OR molecule can bind to both NK cells and the CD19- or CD20-binding bridging molecules to enable interaction and cell killing of the JeKo-1 cells by NK cells. Enhanced binding of NK cells to JeKo-1 cells was observed in the presence of bridging molecules.

FIG. 38B further demonstrates this effect across increasing effector:target ratios.

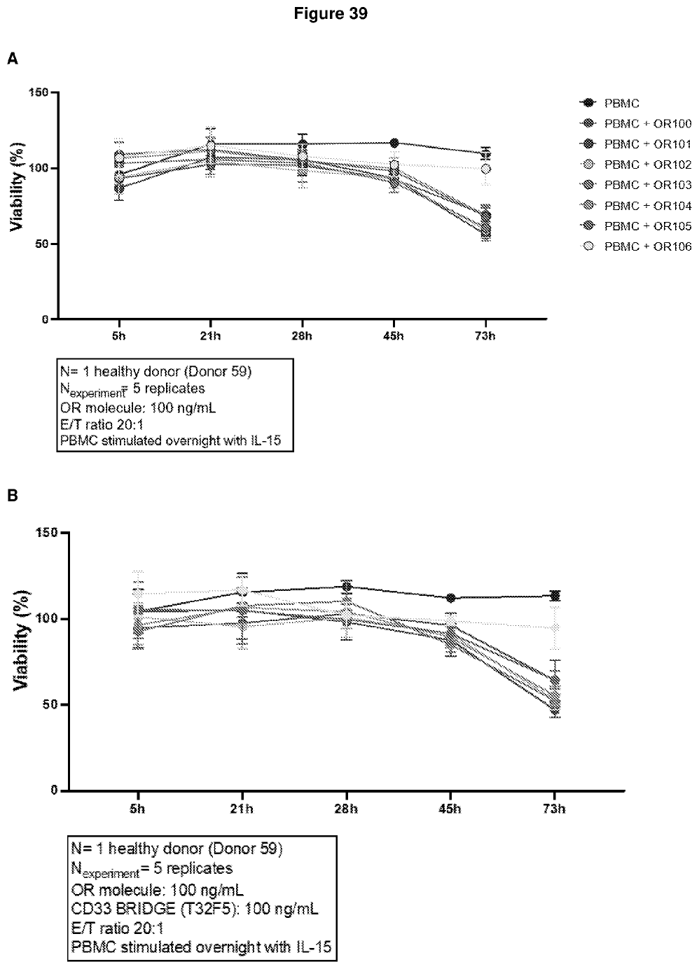

FIG. 39A shows the results of similar experiments, in which various OR molecules were combined at a concentration of 100 ng/ml with PBMCs (E/T ratio: 20:1) to target MOLM-13 cancer cells. The results show the efficacy of a range of different OR molecule architectures for reducing MOLM-13 cell viability.

FIG. 39B shows that the efficacy of the OR molecules is enhanced through the addition of a CD33-binding BRiDGE molecule (100 ng/ml).

1. A method for treating a condition comprising administering to a subject:
an antigen binding protein comprising:
(i) a first antigen binding domain that binds to a tumour-specific antigen; and
(ii) a second antigen binding domain that binds to a cell surface molecule on an immune cell, thereby treating the condition.

2. A composition comprising:
an antigen binding protein comprising:
(i) a first antigen binding domain that binds to a tumour-specific antigen; and
(ii) a second antigen binding domain that binds to a cell surface molecule on an immune cell,
preferably the composition further comprises a pharmaceutically acceptable carrier, diluent or excipient.

3. A kit comprising:
an antigen binding protein comprising:
(i) a first antigen binding domain that binds to a tumour-specific antigen; and
(ii) a second antigen binding domain that binds to a cell surface molecule on an immune cell,
preferably the kit further comprises written instructions to use the antigen binding protein in a method according to claim 1.

4. A method, composition or kit according to any one of claims 1 to 3, wherein the tumour-specific antigen is an antigen expressed on a solid tumour.

5. A method, composition or kit according to any one of claims 1 to 4, wherein the tumour-specific antigen is any one of: dysfunctional P2X₇ receptor, EGFRvIII or CLDN6.

6. A method, composition or kit according to any one of claims 1 to 5, wherein the tumour-specific antigen is dysfunctional P2X₇ receptor.

7. A method, composition or kit according to any one of claims 1 to 6, wherein first antigen binding domain binds to an epitope associated with an adenosine triphosphate (ATP)-binding site of the dysfunctional P2X₇ receptor.

8. A method, composition or kit according to any one of claims 5 to 7, wherein the dysfunctional P2X₇ receptor has a conformational change that renders the receptor dysfunctional.

9. A method, composition or kit according to claim 8, wherein the conformational change is a change of an amino acid from the trans-conformation to the cis-conformation.

10. A method, composition or kit according to claim 9, wherein the amino acid that has changed from a trans-conformation to a cis-conformation is proline at amino acid position 210 of the dysfunctional P2X₇ receptor.

11. A method, composition or kit according to any one of claims 1 to 10, wherein the first antigen binding site binds to an epitope that includes one or more amino acid residues spanning from glycine at amino acid position 200 to cysteine at amino acid position 216, inclusive, of the dysfunctional P2X₇ receptor.

12. A method, composition or kit according to any one of claims 1 to 11, wherein the cell surface molecule on an immune cell is present on the surface of a lymphoid or myeloid lineage cell.

13. A method, composition or kit according to claim 12, wherein the lymphoid lineage cell is a natural killer cell or lymphocyte.

14. A method, composition or kit according to claim 13, wherein the lymphocyte is a T lymphocyte (eg cytotoxic T cell, γδ T cell, or NKT cell) or a B lymphocyte.

15. A method, composition or kit according to any one of claims 12 to 14, wherein the myeloid lineage cell is a monocyte, preferably a macrophage.

16. A method, composition or kit according to any one of claims 1 to 15, wherein the cell surface molecule on an immune cell is only present on an immune cell and not present on a non-immune cell.

17. A method, composition or kit according to any one of claims 1 to 16, wherein the cell surface molecule on an immune cell is a receptor that directly or indirectly causes activation of the immune cell.

18. A method, composition or kit according to any one of claims 1 to 17, wherein the second antigen binding domain binds to a T cell receptor or a molecule associated with a T cell receptor.

19. A method, composition or kit according to any one of claims 1 to 18, wherein the second antigen binding domain binds to CD3.

20. A method, composition or kit according to any one of claims 1 to 18, wherein the second antigen binding domain binds to a costimulatory receptor, preferably CD27, CD28, CD30, CD40, DAP10, OX40, 4-1 BB (CD137) and ICOS.

21. A method, composition or kit according to any one of claims 1 to 17, the second antigen binding domain binds to an Fc receptor, or portion thereof, preferably as FcγRI (CD64), FcγRIIa (CD32), FcγRIIb (CD32), FcγRIIIa (CD16a), FcγRIIIb (CD16b).

22. A method, composition or kit according to claim 21, wherein the second binding domain is an Fc region of an antibody or a polypeptide comprising an Fc receptor binding domain.

23. A method according to any one of claims 1 to 22, further comprising administering:
a bridging molecule comprising:
(i) a targeting moiety that binds to a cell surface molecule on a target cell; and
(ii) a tumour-specific antigen epitope moiety that is bound by the first antigen binding domain of the antigen binding protein.

24. A composition or kit according to any one of claims 1 to 22, further comprising a bridging molecule comprising:
(i) a targeting moiety that binds to a cell surface molecule on a target cell; and
(ii) a tumour-specific antigen epitope moiety that is bound by the first antigen binding domain of the antigen binding protein.

25. A method, composition or kit according to claim 23 or 24, wherein the bridging molecule is a polypeptide.

26. A method, composition or kit according to claim 25, wherein the polypeptide, is a fusion or chimeric protein.

27. A method, composition or kit according to any one of claims 23 to 26, wherein targeting moiety of the bridging molecule comprises an antibody or antibody fragment.

28. A method, composition or kit according to claim 27, wherein the antibody or antibody fragment is an immunoglobulin (Ig), optionally selected from an IgG, an IgA, an IgD, an IgE, an IgM, a fragment thereof or a modification thereof.

29. A method, composition or kit according to any one of claims 23 to 28, wherein the antibody or fragment thereof binds to an antigen on a cancer cell.

30. A method, composition or kit according to claim 29, wherein the antigen on the cancer cell is a tumour associated antigen.

31. A method, composition or kit according to claim 30, wherein the tumour associated antigen is selected from the group consisting of: CD33 (Siglec-3), CD123 (IL3RA), CD135 (FLT-3), CD44 (HCAM), CD44V6, CD47, CD184 (CXCR4), CLEC12A (CLL1), LeY, FRp, MICA/B, CD305 (LAIR-1), CD366 (TIM-3), CD96 (TACTILE), CD133, CD56, CD29 (ITGB1), CD44 (HCAM), CD47 (IAP), CD66 (CEA), CD112 (Nectin2), CD117 (c-Kit), CD133, CD146 (MCAM), CD155 (PVR), CD171 (L1 CAM), CD221 (IGF1), CD227 (MUC1), CD243 (MRD1), CD246 (ALK), CD271 (LNGFR), CD19, CD20, CD22, CD37, CD38, CD79B, CD276, uPAR, GD2, IL13Ra, PSMA, PSCA, EpCAM, ROR1, ROR2, CD117, CD70, CD30, Her2, Her3, BCMA, PDL1, MET-R, PDGFRalpha, GPC3, SLAMF7, GPNMB, VEGFR2, a4β7, aEβ7, CSPG4, CD80, CCR4, ENOX-2, MSLN, EphA2, IGF1R, FAP, AXL, Her4, Claudin 18.2, o-acetylated GD-2, GD3, CD147, CD163, Podoplanin, WT1, GPC2, NY-ESO-1, FGFR4, EphB4, STEAP-1, STEAP-2, IL1RAP, MAGE-A1, TRBC1, TRBC2, CD105, CD138, CEACAM5, IL1Ra, Nectin-4, and EGFR.

32. A method, composition or kit according to any one of claims 1 to 31, wherein the condition is cancer.

33. A method, composition or kit according to any one of claims 23 to 32, wherein the cell surface molecule bound by the targeting moiety is selected from a protein, a lipid moiety, a glycoprotein, a glycolipid, a carbohydrate, a polysaccharide, a nucleic acid, an MHC-bound peptide, or a combination thereof.

34. A method, composition or kit according to any one of claims 23 to 32, wherein the cell surface molecule bound by the targeting moiety comprises parts (e.g., coats, capsules, cell walls, flagella, fimbrae, and toxins) of bacteria, viruses, and other microorganisms.

35. A method, composition or kit according to any one of claims 23 to 33, wherein the tumour-specific antigen epitope moiety comprises or consists of an epitope from a tumour specific antigen.

36. A method, composition or kit according to claim 35, wherein the tumour specific antigen is any one of dysfunctional P2X₇ receptor, EGFRvIII or CLDN6.

37. A method, composition or kit according to any one of claims 23 to 36, wherein the tumour-specific antigen epitope moiety is capable of being bound by the first antigen binding domain of the antigen binding protein.

38. A method, composition or kit according to any one of claims 23 to 37, wherein the tumour-specific antigen epitope moiety is a dysfunctional P2X₇ receptor epitope moiety in the form of a P2X₇ receptor, or a fragment of a P2X₇ receptor that has at least one of the three ATP binding sites that are formed at the interface between adjacent correctly packed monomers that are unable to bind ATP.

39. A method, composition or kit according to claim 38, wherein the dysfunctional P2X₇ receptor epitope moiety comprises or consists of a fragment of a dysfunctional P2X₇ receptor, preferably GHNYTTRNILPGLNITC (SEQ ID NO: 2), or a variant thereof (such as defined in any one of SEQ ID NOs: 3 to 10 and 15 to 30, 168, 361-396, 437, 438), KYYKENNVEKRTLIKVF (SEQ ID NO: 12 and 13); or GHNYTTRNILPGAGAKYYKENNVEK (SEQ ID NO: 14).

40. A method, composition or kit according to any one of claims 23 to 39, wherein the dysfunctional P2X₇ receptor epitope moiety is bound by an antibody that binds to dysfunctional P2X₇ receptors, but is not bound by antibodies that bind to functional P2X₇ receptors.

41. A method according to any one of claims 1 to 40, further comprising administering an immune cell or progenitor thereof, expressing a receptor comprising an antigen-recognition domain and a signalling domain.

42. A method according to claim 41, wherein the immune cell is a T cell expressing a chimeric antigen receptor (CAR), i.e. a CAR-T cell.

43. An antigen binding protein comprising:
(i) a first antigen binding domain that binds to a tumour-specific antigen; and
(ii) a second antigen binding domain that binds to a cell surface molecule on an immune cell.

44. The antigen binding protein according to claim 43, wherein the first antigen binding domain binds to dysfunctional P2X₇ receptor.

45. The antigen binding protein according to claims 43 or 44, wherein the first antigen binding domain binds to an epitope associated with an adenosine triphosphate (ATP)-binding site of the dysfunctional P2X₇ receptor.

46. The antigen binding protein according to any one of claims 43 to 45, wherein the dysfunctional P2X₇ receptor has a conformational change that renders the receptor dysfunctional.

47. The antigen binding protein according to claim 46, wherein the conformational change is a change of an amino acid from the trans-conformation to the cis-conformation.

48. The antigen binding protein according to claim 47, wherein the amino acid that has changed from a trans-conformation to a cis-conformation is proline at amino acid position 210 of the dysfunctional P2X₇ receptor.

49. The antigen binding protein according to any one of claims 43 to 48, wherein the first antigen binding site binds to an epitope that includes one or more amino acid residues spanning from glycine at amino acid position 200 to cysteine at amino acid position 216, inclusive, of the dysfunctional P2X₇ receptor.

50. The antigen binding protein according to any one of claims 43 to 49, wherein the first antigen binding domain binds to an epitope of dysfunctional P2X₇ receptor as defined in any of SEQ ID NO:s 2 to 10, 15 to 30, 16, 361 to 396, 437 and 438.

51. The antigen binding protein according to any one of claims 43 to 50, wherein the second antigen binding domain binds to a cell surface molecule on an immune cell that is present on the surface of a lymphoid or myeloid lineage cell.

52. The antigen binding protein according to claim 51, wherein the lymphoid lineage cell is a natural killer cell or lymphocyte.

53. The antigen binding protein according to claim 52, wherein the lymphocyte is a T lymphocyte (eg cytotoxic T cell, γδ T cell, or NKT cell) or a B lymphocyte.

54. The antigen binding protein according to any one of claims 51 to 53, wherein the myeloid lineage cell is a monocyte, preferably a macrophage.

55. The antigen binding protein according to any one of claims 43 to 54, wherein the second antigen binding domain binds to a cell surface antigen that is present on a T lymphocyte (eg cytotoxic T cell, γδ T cell, or NKT cell), a B lymphocyte or on a natural killer cell.

56. The antigen binding protein according to any one of claims 43 to 54, wherein the second antigen binding domain binds to a cell surface antigen that is present on a T cell and is not present on a non-immune cell.

57. The antigen binding protein according to any one of claims 43 to 54, wherein the second antigen binding domain binds to a cell surface antigen that is present on a natural killer cell.

58. The antigen binding protein according to any one of claims 43 to 57, wherein the cell surface molecule on an immune cell is only present on an immune cell and not present on a non-immune cell.

59. The antigen binding protein according to any one of claims 43 to 58, wherein the cell surface molecule on an immune cell is a receptor that directly or indirectly causes activation of the immune cell.

60. The antigen binding protein according to any one of claims 43 to 59, wherein the second antigen binding domain binds to a T cell receptor or a molecule associated with a T cell receptor.

61. The antigen binding protein according to any one of claims 43 to 60, wherein the second antigen binding domain binds to CD3.

62. The antigen binding protein according to any one of claims 43 to 61, wherein the second antigen binding domain binds to a costimulatory receptor, preferably CD27, CD28, CD30, CD40, DAP10, OX40, 4-1 BB (CD137) and ICOS.

63. The antigen binding protein according to any one of claims 43 to 59, wherein the second antigen binding domain binds to an Fc receptor, or portion thereof, preferably as FcγRI (CD64), FcγRIIa (CD32), FcγRIIb (CD32), FcγRIIIa (CD16a), FcγRIIIb (CD16b).

64. The antigen binding protein according to any one of claims 43 to 59, wherein the second antigen binding domain binds to an antigen on a natural killer cell selected from: CD16A, NKp44, NKp46, NKG2D, and DNAM-1.

65. The antigen binding protein according to any one of claims 43 to 54, wherein the second binding domain is an Fc region of an antibody or a polypeptide comprising an Fc receptor binding domain.

66. The antigen binding protein according to any one of claims 43 to 65 wherein the protein is a bivalent or tetravalent molecule.

67. The antigen binding protein according to any one of claims 43 to 65 wherein the protein is a fusion protein.

68. The antigen binding protein according to any one of claims 43 to 65, wherein the protein comprises a combination of different antibody fragment formats.

69. The antigen binding protein according to any one of claims 43 to 65, wherein the first and/or second antigen binding domains are immunoglobulins, or comprise immunoglobulin domains.

70. The antigen binding protein according to any one of claims 43 to 65, wherein the first and/or second antigen binding domains comprise antigen binding fragments of an antibody.

71. An antigen binding protein defined in any one of claims 1 to 70.

72. An antigen binding protein comprising an antigen binding domain that binds to a dysfunctional P2X₇ receptor, wherein the antigen binding protein comprises an antigen binding domain comprising:
a variable heavy (VH) chain comprising FR1-CDR1-FR2-CDR2-FR3-CDR3-FR4,
wherein:
FR1, FR2, FR3 and FR4 are each framework regions;
CDR1, CDR2 and CDR3 are each complementarity determining regions;
and wherein the antigen binding domain comprises the CDRs1-3 of any of the VH chains as defined in any one of SEQ ID NOs: 400, 402 or 411.

73. The antigen binding protein according to claim 72, wherein the protein comprises, consists or consists essentially of an amino acid sequence as set forth in any one of SEQ ID NOs: 400 or 402.

74. The antigen binding protein according to claim 72, wherein the protein comprises an antigen binding domain of an antibody, wherein the antigen binding domain binds to or specifically binds to a dysfunctional P2X₇ receptor, wherein the antigen binding domain comprises at least one of:
(i) a VH comprising a complementarity determining region (CDR) 1 comprising a sequence at least about 80%, at least 85%, at least 90%, at least 92%, at least 95%, at least 97%, at least 99% identical to a sequence set forth in SEQ ID NO: 397, a CDR2 comprising a sequence at least about 80%, at least 85%, at least 90%, at least 92%, at least 95%, at least 97%, at least 99% identical to a sequence set forth in SEQ ID NO: 398 and a CDR3 comprising a sequence at least about 80%, at least 85%, at least 90%, at least 92%, at least 95%, at least 97%, at least 99% identical to a sequence set forth in any one of SEQ ID NOs: 399 or 401;
(ii) a VH comprising a sequence at least about 95% or 96% or 97% or 98% or 99% identical to a sequence set forth in any one of SEQ ID NOs: 400 or 402;
(iii) a VH comprising a CDR1 comprising a sequence set forth SEQ ID NO: 397, a CDR2 comprising a sequence set forth in any one of SEQ ID NO: 398, and a CDR3 comprising a sequence set forth in any one of SEQ ID NOs: 399 or 401;
(iv) a VH comprising a sequence set forth in any one of SEQ ID NOs: 400 or 402.

75. The antigen binding protein of claim 74, wherein the antigen binding domain further comprises at least one of:
(iii) a VH comprising a framework region (FR) 1 comprising a sequence at least about 80%, at least 85%, at least 90%, at least 92%, at least 95%, at least 97%, at least 99% identical to a sequence set forth in any one of SEQ ID NOs: 403 or 404, a FR2 comprising a sequence at least about 80%, at least 85%, at least 90%, at least 92%, at least 95%, at least 97%, at least 99% identical to a sequence set forth in SEQ ID NO: 405, a FR3 comprising a sequence at least about 80%, at least 85%, at least 90%, at least 92%, at least 95%, at least 97%, at least 99% identical to a sequence set forth in SEQ ID NO: 406, and a FR4 comprising a sequence at least about 80%, at least 85%, at least 90%, at least 92%, at least 95%, at least 97%, at least 99% identical to a sequence set forth in SEQ ID NO: 407; or
(iv) a VH comprising a FR1 comprising a sequence set forth in any one of SEQ ID NOs: 403 or 404, a FR2 comprising a sequence set forth in SEQ ID NO: 405, a FR3 comprising a sequence set forth in SEQ ID NO: 406, and a FR4 comprising a sequence set forth in SEQ ID NO: 407.

76. The antigen binding protein according to claim 72, wherein the antigen binding protein competitively inhibits binding of an antigen binding protein comprising, consisting or consisting essentially of an amino acid sequence as set forth in any one of SEQ ID NOs: 400 or 402.

77. An antigen binding protein according to claim 72 wherein the protein comprises an antigen binding domain that comprises at least one of:
(i) a VH comprising a complementarity determining region (CDR) 1 comprising a sequence at least about 80%, at least 85%, at least 90%, at least 92%, at least 95%, at least 97%, at least 99% identical to a sequence set forth in SEQ ID NO: 408, 416, 423, or 430, a CDR2 comprising a sequence at least about 80%, at least 85%, at least 90%, at least 92%, at least 95%, at least 97%, at least 99% identical to a sequence set forth in SEQ ID NO: 409, 417, 424 or 431 and a CDR3 comprising a sequence at least about 80%, at least 85%, at least 90%, at least 92%, at least 95%, at least 97%, at least 99% identical to a sequence set forth in any one of SEQ ID NOs: 410, 418, 424 or 432;
(ii) a VH comprising a sequence at least about 95% or 96% or 97% or 98% or 99% identical to a sequence set forth in SEQ ID NO: 411;
(iii) a VH comprising a CDR1 comprising a sequence set forth SEQ ID NO: 408, 416, 423, or 430, a CDR2 comprising a sequence set forth in any one of SEQ ID NO: 409, 417, 424 or 431, and a CDR3 comprising a sequence set forth in any one of SEQ ID NOs: 410, 418, 424 or 432;
(iv) a VH comprising a sequence set forth in SEQ ID NO: 411.

78. The antigen binding protein according to claim 77, wherein the antigen binding domain further comprises at least one of:
(i) a VH comprising a framework region (FR) 1 comprising a sequence at least about 80%, at least 85%, at least 90%, at least 92%, at least 95%, at least 97%, at least 99% identical to a sequence set forth in any one of SEQ ID NOs: 412, 419, 426 or 433, a FR2 comprising a sequence at least about 80%, at least 85%, at least 90%, at least 92%, at least 95%, at least 97%, at least 99% identical to a sequence set forth in SEQ ID NO: 413, 420, 427 or 434, a FR3 comprising a sequence at least about 80%, at least 85%, at least 90%, at least 92%, at least 95%, at least 97%, at least 99% identical to a sequence set forth in SEQ ID NO: 414, 421, 428 or 435, and a FR4 comprising a sequence at least about 80%, at least 85%, at least 90%, at least 92%, at least 95%, at least 97%, at least 99% identical to a sequence set forth in SEQ ID NO: 415, 422, 429 or 436; or
(ii) a VH comprising a FR1 comprising a sequence set forth in any one of SEQ ID NOs: 412, 419, 426 or 433, a FR2 comprising a sequence set forth in SEQ ID NO: 413, 420, 427 or 434, a FR3 comprising a sequence set forth in SEQ ID NO: 414, 421, 428 or 435, and a FR4 comprising a sequence set forth in SEQ ID NO: 415, 422, 429 or 436.

79. The antigen binding protein according to any one of claims 72 to 78 wherein the protein additionally comprises a FR1a-CDR1a-FR2a-CDR2a-FR3a-CDR3a-FR4a, wherein FR1a, FR2a, FR3a and FR4a are each framework regions; and CDR1a, CDR2a and CDR3a are each complementarity determining regions; optionally wherein FR1a-CDR1a-FR2a-CDR2a-FR3a-CDR3a-FR4a corresponds to the sequence of a variable light (VL) chain.

80. The antigen binding protein according to claim 79, wherein the protein comprises FR1-CDR1-FR2-CDR2-FR3-CDR3-FR4-linker-FR1a-CDR1a-FR2a-CDR2a-FR3a-CDR3a-FR4a; or FR1a-CDR1a-FR2a-CDR2a-FR3a-CDR3a-FR4a-FR1-CDR1-FR2-CDR2-FR3-CDR3-FR4.

81. The antigen binding protein of claim 80, wherein the linker is a chemical, one or more amino acids, or a disulphide bond formed between two cysteine residues.

82. The antigen binding protein according to any one of claims 72 to 81, wherein the protein comprises a variable light chain (VL) comprising a CDR of any of the sequences defined in any one of SEQ ID NOs: 309, 310, 311, 312, 319, 330, 331, 332 or 333.

83. The antigen binding protein according to any one of claims 72 to 82, wherein the antigen binding protein comprises a variable light chain (VL) comprising a the sequence as defined in any one of SEQ ID NOs: 309, 310, 311, 312, 319, 330, 331, 332 or 333, or sequences at least about 80%, 81%, 82%, 83%, 84%, 85%, 86%, 87%, 88%, 89%, 90%, 91%, 92%, 93%, 94%, 95%, 96%, 97%, 98% or at least 99% identical thereto.

84. The antigen binding protein according to any one of claims 72 to 83, wherein the antigen binding protein comprises, consists essentially of or consists of the amino acid sequence of (in order of N to C terminus or C to N terminus) i) any one of SEQ ID NOs: 400, 402 or 411; and ii) any of SEQ ID NOs: 309, 310, 311, 312, 319, 330, 331, 332 or 333.

85. The antigen binding protein according to any one of claims 72 to 84, wherein the antigen binding protein is in the form of:
(i) a single chain Fv fragment (scFv);
(ii) a dimeric scFv (di-scFv); or
(iii) one of (i) or (ii) linked to a constant region of an antibody, Fc or a heavy chain constant domain (CH) 2 and/or CH3.

86. The antigen binding protein according to any one of claims 72 to 84, wherein the antigen binding protein is in the form of:
(i) a diabody;
(ii) a triabody;
(iii) a tetrabody;
(iv) a Fab;
(v) a F(ab′)2;
(vi) a Fv;
(vii) a bispecific antibody or other form of multispecific antibody (including a BiTE); or
(viii) one of (i) to (vii) linked to a constant region of an antibody, Fc or a heavy chain constant domain (CH) 2 and/or CH3.

87. The antigen binding protein according to any one of claims 72 to 86, wherein, the antigen binding protein does not comprise a constant region from an immunoglobulin.

88. A composition comprising an antigen binding protein according to any one of claims 72 to 87.

89. A fusion protein comprising an antigen binding protein according to any one of claims 72 to 87.

90. A pharmaceutical composition comprising an antigen binding protein according to any one of claims 43 to 87, or a fusion protein of claim 89, and a pharmaceutically acceptable carrier or excipient.

91. A method for the prevention or treatment a condition or disease associated with expression of nfP2X₇ in an individual comprising the step of providing an antigen binding protein according to any one of claims 43 to 87, a fusion protein according to claim 89, or pharmaceutical composition according to claim 90.

92. The method according to claim 91 wherein the condition or disease is cancer.

93. Use of an antigen binding protein according to any one of claims 43 to 87, or the fusion protein of claim 89, in the manufacture of a medicament for the treatment or prevention of cancer in a subject.

94. An antigen binding protein according to any one of claims 43 to 87, fusion protein according to claim 89, or the pharmaceutical composition according to claim 90, for use in the treatment or prevention of cancer in a subject.


SEQUENCE LISTING (396 sequences)

SEQ ID NO: 1 - protein, 595 aa, Homo sapiens
MPACCSCSDVFQYETNKVTRIQSMNYGTIKWFFHVIIFSYVCFALVSDKLYQRKEPVISSVHTKVKGIAEVKEEIVENGVKKLVHSVFDTADYTFPLQGNSFFVMTNFLKTEGQEQRLCPEYPTRRTLCSSDRGCKKGWMDPQSKGIQTGRCVVYEGNQKTCEVSAWCPIEAVEEAPRPALLNSAENFTVLIKNNIDFPGHNYTTRNILPGLNITCTFHKTQNPQCPIFRLGDIFRETGDNFSDVAIQGGIMGIEIYWDCNLDRWFHHCRPKYSFRRLDDKTTNVSLYPGYNFRYAKYYKENNVEKRTLIKVFGIRFDILVFGTGGKFDIIQLVVYIGSTLSYFGLAAVFIDFLIDTYSSNCCRSHIYPWCKCCQPCVVNEYYYRKKCESIVEPKPTLKYVSFVDESHIRMVNQQLLGRSLQDVKGQEVPRPAMDFTDLSRLPLALHDTPPIPGQPEEIQLLRKEATPRSRDSPVWCQCGSCLPSQLPESHRCLEELCCRKKPGACITTSELFRKLVLSRHVLQFLLLYQEPLLALDVDSTNSRLRHCAYRCYATWRFGSQDMADFAILPSCCRWRIRKEFPKSEGQYSGFKSPY

SEQ ID NO: 2 - protein, 17 aa, synthetic construct
GHNYTTRNILPGLNITC

SEQ ID NO: 3 - protein, 16 aa, synthetic construct
GHNYTTRNILPGLNIT

SEQ ID NO: 4 - protein, 17 aa, synthetic construct
GHNYTTRNILPGLNITS

SEQ ID NO: 5 - protein, 21 aa, synthetic construct
GHNYTTRNILPGLNITSTFHK

SEQ ID NO: 6 - protein, 22 aa, synthetic construct
GHNYTTRNILPGLNITSTFHKT

SEQ ID NO: 7 - protein, 23 aa, synthetic construct
GHNYTTRNILPGLNITSTFHKTC

SEQ ID NO: 8 - protein, 16 aa, synthetic construct
DFPGHNYTTRNILPGC

SEQ ID NO: 9 - protein, 23 aa, synthetic construct
GHNYTTRNILPGLNITSTFHKTS

SEQ ID NO: 10 - protein, 27 aa, synthetic construct
GHNYTTRNILPGLNITSTFHKTSGSGK

SEQ ID NO: 11 - protein, 11 aa, synthetic construct
NYTTRNILPGL

SEQ ID NO: 12 - protein, 15 aa, synthetic construct
KYYKENNVEKRTLIK

SEQ ID NO: 13 - protein, 17 aa, synthetic construct
KYYKENNVEKRTLIKVF

SEQ ID NO: 14 - protein, 25 aa, synthetic construct
GHNYTTRNILPGAGAKYYKENNVEK

SEQ ID NO: 15 - protein, 22 aa, synthetic construct
GHNYTTRNILPGLNITSGGGGS

SEQ ID NO: 16 - protein, 27 aa, synthetic construct
GHNYTTRNILPGLNITSGGGGSGGGGS

SEQ ID NO: 17 - protein, 24 aa, synthetic construct
GHNYTTRNILPGLNITSTFHKTGS

SEQ ID NO: 18 - protein, 22 aa, synthetic construct
GHNYTTRNILPGLNITSTFHGS

SEQ ID NO: 19 - protein, 19 aa, synthetic construct
GHNYTTRNILPGLNITSGS

SEQ ID NO: 20 - protein, 22 aa, synthetic construct
DFPGHNYTTRNILPGLNITSGS

SEQ ID NO: 21 - protein, 25 aa, synthetic construct
DFPGHNYTTRNILPGLNITSGGGGS

SEQ ID NO: 22 - protein, 30 aa, synthetic construct
DFPGHNYTTRNILPGLNITSGGGGSGGGGS

SEQ ID NO: 23 - protein, 35 aa, synthetic construct
DFPGHNYTTRNILPGLNITSGGGGSGGGGSGGGGS

SEQ ID NO: 24 - protein, 30 aa, synthetic construct
DFPGHNYTTRNILPGLNITSTFHKTSGSGK

SEQ ID NO: 25 - protein, 32 aa, synthetic construct
DFPGHNYTTRNILPGLNITSTFHKTSGSGKGS

SEQ ID NO: 26 - protein, 35 aa, synthetic construct
DFPGHNYTTRNILPGLNITSTFHKTSGSGKGGGGS

SEQ ID NO: 27 - protein, 28 aa, synthetic construct
DFPGHNYTTRNILPGLNITSTFHGGGGS

SEQ ID NO: 28 - protein, 25 aa, synthetic construct
GHNYTTRNILPGLNITSTFHGGGGS

SEQ ID NO: 29 - protein, 30 aa, synthetic construct
DFPGHNYTTRNILPGLNITSTFHKTGGGGS

SEQ ID NO: 30 - protein, 27 aa, synthetic construct
GHNYTTRNILPGLNITSTFHKTGGGGS

SEQ ID NO: 31 - protein, 229 aa, synthetic construct
EVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCHHHHHH

SEQ ID NO: 32 - protein, 216 aa, synthetic construct
DIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITKARTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 33 - protein, 246 aa, synthetic construct
GHNYTTRNILPGLNITSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCHHHHHH

SEQ ID NO: 34 - protein, 251 aa, synthetic construct
GHNYTTRNILPGLNITSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCHHHHHH

SEQ ID NO: 35 - protein, 233 aa, synthetic construct
GHNYTTRNILPGLNITSDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITKARTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 36 - protein, 238 aa, synthetic construct
GHNYTTRNILPGLNITSGGGGSDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITKARTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 37 - protein, 246 aa, synthetic construct
EVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCGHNYTTRNILPGLNITSHHHHHH

SEQ ID NO: 38 - protein, 222 aa, synthetic construct
DIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITKARTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECHHHHHH

SEQ ID NO: 39 - protein, 233 aa, synthetic construct
DIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITKARTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGHNYTTRNILPGLNITS

SEQ ID NO: 40 - protein, 248 aa, synthetic construct
DIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSHHHHHH

SEQ ID NO: 41 - protein, 248 aa, synthetic construct
EVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSGGGGSGGGGSGGGGSDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITHHHHHH

SEQ ID NO: 42 - protein, 265 aa, synthetic construct
GHNYTTRNILPGLNITSDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSHHHHHH

SEQ ID NO: 43 - protein, 270 aa, synthetic construct
GHNYTTRNILPGLNITSGGGGSDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSHHHHHH

SEQ ID NO: 44 - protein, 265 aa, synthetic construct
GHNYTTRNILPGLNITSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSGGGGSGGGGSGGGGSDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITHHHHHH

SEQ ID NO: 45 - protein, 270 aa, synthetic construct
GHNYTTRNILPGLNITSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSGGGGSGGGGSGGGGSDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITHHHHHH

SEQ ID NO: 46 - protein, 265 aa, synthetic construct
EVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSGGGGSGGGGSGGGGSDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGHNYTTRNILPGLNITSHHHHHH

SEQ ID NO: 47 - protein, 282 aa, synthetic construct
GHNYTTRNILPGLNITSDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSGHNYTTRNILPGLNITSHHHHHH

SEQ ID NO: 48 - protein, 287 aa, synthetic construct
GHNYTTRNILPGLNITSGGGGSDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSGHNYTTRNILPGLNITSHHHHHH

SEQ ID NO: 49 - protein, 282 aa, synthetic construct
GHNYTTRNILPGLNITSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSGGGGSGGGGSGGGGSDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGHNYTTRNILPGLNITSHHHHHH

SEQ ID NO: 50 - protein, 287 aa, synthetic construct
GHNYTTRNILPGLNITSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSGGGGSGGGGSGGGGSDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGHNYTTRNILPGLNITSHHHHHH

SEQ ID NO: 51 - protein, 242 aa, synthetic construct
GHNYTTRNILPGLNITSDIVMTQSPATLSLSPGERATLSCRSSKSLQNVNGNTYLYWFQQKPGQSPQLLIYRMSNLNSGVPDRFSGSGSGTEFTLTISSLEPEDFAVYYCMQHLEYPITFGAGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECHHHHHH

SEQ ID NO: 52 - protein, 230 aa, synthetic construct
EVQLVESGGGLVKPGGSLKLSCAASGYTFTSYVMHWVRQAPGKGLEWIGYINPYNDGTKYNEKFQGRVTISSDKSISTAYMELSSLRSEDTAMYYCARGTYYYGTRVFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCHHHHHH

SEQ ID NO: 53 - protein, 236 aa, synthetic construct
GHNYTTRNILPGLNITSDIQMTQSPSSLSASVGDRVTITCRASSSVSYMHWYQQKPGKAPKPLIYAPSNLASGVPSRFSGSGSGTDFTLTISSLQPEDFATYYCQQWSFNPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECHHHHHH

SEQ ID NO: 54 - protein, 231 aa, synthetic construct
EVQLVESGGGLVQPGGSLRLSCAASGYTFTSYNMHWVRQAPGKGLEWVGAIYPGNGDTSYNQKFKGRFTISVDKSKNTLYLQMNSLRAEDTAVYYCARVVYYSNSYWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCHHHHHH

SEQ ID NO: 55 - protein, 237 aa, synthetic construct
GHNYTTRNILPGLNITSEIVLTQSPATLSLSPGERATLSCRASQSVSSYLAWYQQKPGQAPRLLIYDASNRATGIPARFSGSGSGTDFTLTISSLEPEDFAVYYCQQRSNWPITFGQGTRLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECHHHHHH

SEQ ID NO: 56 - protein, 231 aa, synthetic construct
EVQLVESGGGLVQPGRSLRLSCAASGFTFNDYAMHWVRQAPGKGLEWVSTISWNSGSIGYADSVKGRFTISRDNAKKSLYLQMNSLRAEDTALYYCAKDIQYGNYYYGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCHHHHHH

SEQ ID NO: 57 - protein, 237 aa, synthetic construct
GHNYTTRNILPGLNITSDIQMIQSPSSLSASVGDRVTITCRASQTIWSYLNWYRQRPGEAPNLLIYAASSLQSGVPSRFSGRGSGTDFTLTISSLQAEDFATYYCQQSYSIPQTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECHHHHHH

SEQ ID NO: 58 - protein, 233 aa, synthetic construct
QVQLQQSGPGMVKPSQTLSLTCAISGDSVSSNSVAWNWIRQSPSRGLEWLGRTYYRSTWYNDYAVSMKSRITINPDTNKNQFSLQLNSVTPEDTAVYYCAREVTGDLEDAFDIWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCHHHHHH

SEQ ID NO: 59 - protein, 242 aa, synthetic construct
GHNYTTRNILPGLNITSDVQVTQSPSSLSASVGDRVTITCRSSQSLANSYGNTFLSWYLHKPGKAPQLLIYGISNRFSGVPDRFSGSGSGTDFTLTISSLQPEDFATYYCLQGTHQPYTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECHHHHHH

SEQ ID NO: 60 - protein, 230 aa, synthetic construct
EVQLVQSGAEVKKPGASVKVSCKASGYRFTNYWIHWVRQAPGQGLEWIGGINPGNNYATYRRKFQGRVTMTADTSTSTVYMELSSLRSEDTAVYYCTREGYGNYGAWFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCHHHHHH

SEQ ID NO: 61 - protein, 241 aa, synthetic construct
GHNYTTRNILPGLNITSDIQLTQSPSSLSASVGDRVTITCKASQSVDYEGDSFLNWYQQKPGKAPKLLIYAASNLESGVPSRFSGSGSGTDFTLTISSLQPEDFATYYCQQSNEDPLTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECHHHHHH

SEQ ID NO: 62 - protein, 226 aa, synthetic construct
EVQLVESGGGLVQPGGSLRLSCAASGYTFSSYWIEWVRQAPGKGLEWIGEILPGGGDTNYNEIFKGRATFSADTSKNTAYLQMNSLRAEDTAVYYCTRRVPIRLDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCHHHHHH

SEQ ID NO: 63 - protein, 237 aa, synthetic construct
GHNYTTRNILPGLNITSEIVLTQSPATLSLSPGERATLSCRASENVYSYLAWYQQKPGQAPRLLIYFAKTLAEGIPARFSGSGSGTDFTLTISSLEPEDFAVYYCQHHSDNPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECHHHHHH

SEQ ID NO: 64 - protein, 225 aa, synthetic construct
EVQLVQSGAEVKKPGESLKISCKGSGYSFTGYNMNWVRQMPGKGLEWMGNIDPYYGGTTYNRKFKGQVTISADKSISTAYLQWSSLKASDTAMYYCARSVGPFDSWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCHHHHHH

SEQ ID NO: 65 - protein, 237 aa, synthetic construct
GHNYTTRNILPGLNITSEIVLTQSPATLSLSPGERATLSCRASQSVSSYLAWYQQKPGQAPRLLIYDASNRATGIPARFSGSGSGTDFTLTISSLEPEDFAVYYCQQRSNWPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECHHHHHH

SEQ ID NO: 66 - protein, 231 aa, synthetic construct
EVQLLESGGGLVQPGGSLRLSCAVSGFTFNSFAMSWVRQAPGKGLEWVSAISGSGGGTYYADSVKGRFTISRDNSKNTLYLQMNSLRAEDTAVYFCAKDKILWFGEPVFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCHHHHHH

SEQ ID NO: 67 - protein, 241 aa, synthetic construct
GHNYTTRNILPGLNITSQAVVTQEPSLTVSPGGTVTLTCGLKSGSVTSDNFPTWYQQTPGQAPRLLIYNTNTRHSGVPDRFSGSILGNKAALTITGAQADDEAEYFCALFISNPSVEFGGGTQLTVLKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECHHHHHH

SEQ ID NO: 68 - protein, 231 aa, synthetic construct
EVQLVESGGGLVQPGGSLRLSCAASGFTFSVYYMNWVRQAPGKGLEWVSDINNEGGTTYYADSVKGRFTISRDNSKNSLYLQMNSLRAEDTAVYYCARDAGYSNHVPIFDSWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCHHHHHH

SEQ ID NO: 69 - protein, 241 aa, synthetic construct
GHNYTTRNILPGLNITSDIVLTQSPASLAVSLGQRATISCKASQSVDFDGDSYMNWYQQKPGQPPKVLIYAASNLESGIPARFSGSGSGTDFTLNIHPVEEEDAATYYCQQSNEDPWTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECHHHHHH

SEQ ID NO: 70 - protein, 226 aa, synthetic construct
QIQLQQSGPEVVKPGASVKISCKASGYTFTDYYITWVKQKPGQGLEWIGWIYPGSGNTKYNEKFKGKATLTVDTSSSTAFMQLSSLTSEDTAVYFCANYGNYWFAYWGQGTQVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCHHHHHH

SEQ ID NO: 71 - protein, 241 aa, synthetic construct
GHNYTTRNILPGLNITSDIQLTQSPSTLSASVGDRVTITCRASESLDNYGIRFLTWFQQKPGKAPKLLMYAASNQGSGVPSRFSGSGSGTEFTLTISSLQPDDFATYYCQQTKEVPWSFGQGTKVEVKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECHHHHHH

SEQ ID NO: 72 - protein, 225 aa, synthetic construct
EVQLVQSGAEVKKPGSSVKVSCKASGYTITDSNIHWVRQAPGQSLEWIGYIYPYNGGTDYNQKFKNRATLTVDNPTNTAYMELSSLRSEDTAFYYCVNGNPWLAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCHHHHHH

SEQ ID NO: 73 - protein, 241 aa, synthetic construct
GHNYTTRNILPGLNITSDIQMTQSPSSLSASVGDRVTITCRASESVDNYGISFMNWFQQKPGKAPKLLIYAASNQGSGVPSRFSGSGSGTDFTLTISSLQPDDFATYYCQQSKEVPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECHHHHHH

SEQ ID NO: 74 - protein, 225 aa, synthetic construct
QVQLVQSGAEVKKPGSSVKVSCKASGYTFTDYNMHWVRQAPGQGLEWIGYIYPYNGGTGYNQKFKSKATITADESTNTAYMELSSLRSEDTAVYYCARGRPAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCHHHHHH

SEQ ID NO: 75 - protein, 237 aa, synthetic construct
GHNYTTRNILPGLNITSDIQMTQSPSSLSASVGDRVTITCKASQDVSIGVAWYQQKPGKAPKLLIYSASYRYTGVPSRFSGSGSGTDFTLTISSLQPEDFATYYCQQYYIYPYTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECHHHHHH

SEQ ID NO: 76 - protein, 228 aa, synthetic construct
EVQLVESGGGLVQPGGSLRLSCAASGFTFTDYTMDWVRQAPGKGLEWVADVNPNSGGSIYNQRFKGRFTLSVDRSKNTLYLQMNSLRAEDTAVYYCARNLGPSFYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCHHHHHH

SEQ ID NO: 77 - protein, 237 aa, synthetic construct
GHNYTTRNILPGLNITSDIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECHHHHHH

SEQ ID NO: 78 - protein, 229 aa, synthetic construct
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCHHHHHH

SEQ ID NO: 79 - protein, 237 aa, synthetic construct
GHNYTTRNILPGLNITSEIVMTQSPATLSLSPGERATLSCRASQSVSSYLAWYQQKPGQAPRLLIYDASNRATGIPARFSGSGSGTDFTLTISSLEPEDFAVYYCHQYGSTPLTFGGGTKAEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECHHHHHH

SEQ ID NO: 80 - protein, 230 aa, synthetic construct
QVQLQESGPGLVKPSQTLSLTCTVSGGSISSGDYYWSWIRQPPGKGLEWIGYIYYSGSTDYNPSLKSRVTMSVDTSKNQFSLKVNSVTAADTAVYYCARVSIFGVGTFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCHHHHHH

SEQ ID NO: 81 - protein, 236 aa, synthetic construct
GHNYTTRNILPGLNITSDIQMTQSPSSLSASVGDRVTITCSASSSVTYMYWYQQKPGKAPKLLIYDTSNLASGVPSRFSGSGSGTDYTFTISSLQPEDIATYYCQQWSSHIFTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECHHHHHH

SEQ ID NO: 82 - protein, 230 aa, synthetic construct
QVQLVQSGAEVKKPGASVKVSCKASGYTFTSHWMHWVRQAPGQGLEWIGEFNPSNGRTNYNEKFKSKATMTVDTSTNTAYMELSSLRSEDTAVYYCASRDYDYDGRYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCHHHHHH

SEQ ID NO: 83 - protein, 237 aa, synthetic construct
GHNYTTRNILPGLNITSDIQMTQSPSSLSASVGDRVTITCQASQDISNYLNWYQQKPGKAPKLLIYDASNLETGVPSRFSGSGSGTDFTFTISSLQPEDIATYFCQHFDHLPLAFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECHHHHHH

SEQ ID NO: 84 - protein, 228 aa, synthetic construct
QVQLQESGPGLVKPSETLSLTCTVSGGSVSSGDYYWTWIRQSPGKGLEWIGHIYYSGNTNYNPSLKSRLTISIDTSKTQFSLKLSSVTAADTAIYYCVRDRVTGAFDIWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCHHHHHH

SEQ ID NO: 85 - protein, 237 aa, Synthetic construct
GHNYTTRNILPGLNITSEIVMTQSPATLSVSPGERVTLSCRASQSISDYLYWYQQKSHESPRLLIKYASQSISGIPARFSGSGSGSEFTLTINSVEPEDVGVYYCQNGHSFPLTFGQGTKLELKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECHHHHHH

SEQ ID NO: 86 - protein, 227 aa, synthetic construct
QVQLVQSGAEVVKPGASVKLSCKTSGYTFTNYDINWVRQRPGQGLEWIGWIFPGDDSTQYNEKFKGKATLTTDTSTSTAYMELSSLRSEDTAVYFCARQTTGTWFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCHHHHHH

SEQ ID NO: 87 - protein, 234 aa, synthetic construct
GHNYTTRNILPGLNITSKIVMTQTPATLSVSAGERVTITCKASQSVSNHVTWYQQKPGQAPRLLIYSASNRYSGVPARFSGSGYGTEFTFTISSVQSEDFAVYFCQQDYSSFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECHHHHHH

SEQ ID NO: 88 - protein, 228 aa, synthetic construct
QVQLVESGPGVVQPGRSLRLSCAVSGFSVTNYGVHWVRQPPGKGLEWLGVIWAGGITNYNSSVKGRLTISKDNSKNTVYLQMNSLRAEDTAVYYCASRGGHYGYALDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCHHHHHH

SEQ ID NO: 89 - protein, 237 aa, synthetic construct
GHNYTTRNILPGLNITSDIQMTQSPSSLSASVGDRVTITCSASQDISNYLNWYQQKPGKAPKLLIYYTSNLHSGVPSRFSGSGSGTDFTLTISSLQPEDFATYYCQQYRKLPWTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECHHHHHH

SEQ ID NO: 90 - protein, 230 aa, synthetic construct
QVQLVQSGAEVKKPGSSVKVSCKGSGYTFTNYWMHWVRQAPGQGLEWIGATYRGHSDTYYNQKFKGRATLTADTSTSTAYMELSSLRSEDTAVYYCTRGAIYDGYDVLDNWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCHHHHHH

SEQ ID NO: 91 - protein, 237 aa, synthetic construct
GHNYTTRNILPGLNITSDIVMTQSPSSVSASVGDRVTITCRASQDISSWLAWYQQKPGKAPKLLIYAASSLQSGVPSRFNGSGSGTDFTLTISSLQPEDFATYYCQQSYSTPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECHHHHHH

SEQ ID NO: 92 - protein, 228 aa, synthetic construct
QVQLVQSGAEVKEPGASVKVSCKAPANTFSDHVMHWVRQAPGQRFEWMGYIHAANGGTHYSQKFQDRVTITRDTSANTVYMDLSSLRSEDTAVYYCARGGYNSDAFDIWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCHHHHHH

SEQ ID NO: 93 - protein, 237 aa, synthetic construct
GHNYTTRNILPGLNITSDIVLTQSPATLSVTPGDSVSLSCRASQSISNNLHWYQQKSHESPRLLIKYASQSISGIPSRFSGSGSGTDFTLSINSVETEDFGVYFCQQSNTWPYTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECHHHHHH

SEQ ID NO: 94 - protein, 227 aa, synthetic construct
QVQLQQPGAELVKPGASLKLSCKSSGYTFTSYWMHWVRQRPGHGLEWIGEIDPSDSYKDYNQKFKDKATLTVDRSSNTAYMHLSSLTSDDSAVYYCARAITTTPFDFWGQGTTLTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCHHHHHH

SEQ ID NO: 95 - protein, 241 aa, synthetic construct
GHNYTTRNILPGLNITSDIVLTQSPASLAVSLGQRATISCRASESVDNYGNTFMHWYQQKPGQPPKLLIYRASNLESGIPARFSGSGSRTDFTLTINPVEADDVATYYCQQSNEDPPTFGAGTKLELKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECHHHHHH

SEQ ID NO: 96 - protein, 227 aa, synthetic construct
QIQLVQSGPELKKPGETVKISCKASGYIFTNYGMNWVKQAPGKSFKWMGWINTYTGESTYSADFKGRFAFSLETSASTAYLHINDLKNEDTATYFCARSGGYDPMDYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCHHHHHH

SEQ ID NO: 97 - protein, 236 aa, synthetic construct
GHNYTTRNILPGLNITSQIVLSQSPAILSASPGEKVTMTCRASSSVSYMHWYQQKPGSSPKPWIYATSNLASGVPVRFSGSGSGTSYSLTISRVEAEDAATYYCQQWSSNPLTFGAGTKLELKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECHHHHHH

SEQ ID NO: 98 - protein, 227 aa, synthetic construct
EVKLEESGGGLVQPGGSMKLSCAASGFTFSDAWMDWVRQSPEKGLEWVAEIRSKASNHATYYAESVKGRFTISRDDSKSSVYLQMNSLRAEDTGIYYCTRWRRFFDSWGQGTTLTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCHHHHHH

SEQ ID NO: 99 - protein, 236 aa, synthetic construct
GHNYTTRNILPGLNITSEIVLSQSPAITAASLGQKVTITCSASSNVSYIHWYQQRSGTSPRPWIYEISKLASGVPVRFSGSGSGTSYSLTISSMEAEDAAIYYCQQWNYPLITFGSGTKLEIQRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECHHHHHH

SEQ ID NO: 100 - protein, 226 aa, synthetic construct
QVQLKESGPGLVAPSQTLSITCTVSGFSLTSYGVHWVRQPPGKGLEWLGVIWAGGFTNYNSALKSRLSISKDNSKSQVLLKMTSLQTDDTAMYYCARRGSSYSMDYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCHHHHHH

SEQ ID NO: 101 - protein, 237 aa, synthetic construct
GHNYTTRNILPGLNITSDIQMTQSPSSLSASVGDRVTITCRASQDVSTAVAWYQQKPGKAPKLLIYSASFLYSGVPSRFSGSGSGTDFTLTISSLQPEDFATYYCQQYLYHPATFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECHHHHHH

SEQ ID NO: 102 - protein, 227 aa, synthetic construct
EVQLVESGGGLVQPGGSLRLSCAASGFTFSDSWIHWVRQAPGKGLEWVAWISPYGGSTYYADSVKGRFTISADTSKNTAYLQMNSLRAEDTAVYYCARRHWPGGFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCHHHHHH

SEQ ID NO: 103 - protein, 243 aa, synthetic construct
GHNYTTRNILPGLNITSDIQMTQSPSSLSASVGDRVTITCKSSQSLLYTSSQKNYLAWYQQKPGKAPKLLIYWASTRESGVPSRFSGSGSGTDFTLTISSLQPEDFATYYCQQYYAYPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECHHHHHH

SEQ ID NO: 104 - protein, 228 aa, synthetic construct
EVQLVESGGGLVQPGGSLRLSCAASGYTFTSYWLHWVRQAPGKGLEWVGMIDPSNSDTRFNPNFKDRFTISADTSKNTAYLQMNSLRAEDTAVYYCATYRSYVTPLDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCHHHHHH

SEQ ID NO: 105 - protein, 238 aa, synthetic construct
GHNYTTRNILPGLNITSDIQMTQSPSSLSASVGDRVSITCRPSQSFSRYINWYQQKPGKAPKLLIHAASSLVGGVPSRFSGSGSGTDFTLTISSLQPEDFATYYCQQTYSNPPITFGQGTRLEMKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECHHHHHH

SEQ ID NO: 106 - protein, 229 aa, synthetic construct
QVQLVESGGGLVKPGGSLRLSCAASGFTFSDYYMNWIRQAPGKGLEWVSYISSSGSIIYYADSVKGRFTISRDNAKNSLYLQMNSLRAEDTAVYYCAREGRIAARGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCHHHHHH

SEQ ID NO: 107 - protein, 237 aa, synthetic construct
GHNYTTRNILPGLNITSEIVLTQSPATLSLSPGERATLSCRASQSVSSYLAWYQQKPGQAPRLLIYDASNRATGIPARFSGSGSGTDFTLTISSLEPEDFAVYYCQQRSNWPPAFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECHHHHHH

SEQ ID NO: 108 - protein, 236 aa, synthetic construct
QLQLQESGPGLVKPSETLSLTCTVSGGSINSSSYYWGWLRQSPGKGLEWIGSFFYTGSTYYNPSLRSRLTISVDTSKNQFSLMLSSVTAADTAVYYCARQSTYYYGSGNYYGWFDRWDQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCHHHHHH

SEQ ID NO: 109 - protein, 241 aa, synthetic construct
GHNYTTRNILPGLNITSQSALTQPASVSGSPGQSITISCTGTSSDVGSYNVVSWYQQHPGKAPKLIIYEVSQRPSGVSNRFSGSKSGNTASLTISGLQTEDEADYYCCSYAGSSIFVIFGGGTKVTVLRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECHHHHHH

SEQ ID NO: 110 - protein, 228 aa, synthetic construct
EVQLLESGGGLVQPGGSLRLSCAASGFTFSHYVMAWVRQAPGKGLEWVSSISSSGGWTLYADSVKGRFTISRDNSKNTLYLQMNSLRAEDTAVYYCTRGLKMATIFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCHHHHHH

SEQ ID NO: 111 - protein, 240 aa, synthetic construct
GHNYTTRNILPGLNITSDIQLTQSPSSLSASVGDRVTITCSVSSSISSNNLHWYQQKPGKAPKPWIYGTSNLASGVPSRFSGSGSGTDYTFTISSLQPEDIATYYCQQWSSYPYMYTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECHHHHHH

SEQ ID NO: 112 - protein, 228 aa, synthetic construct
EVQLVESGGGVVQPGRSLRLSCSASGFTFSGYGLSWVRQAPGKGLEWVAMISSGGSYTYYADSVKGRFAISRDNAKNTLFLQMDSLRPEDTGVYFCARHGDDPAWFAYWGQGTPVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCHHHHHH

SEQ ID NO: 113 - protein, 242 aa, synthetic construct
GHNYTTRNILPGLNITSDVVMTQSPLSLPVTPGEPASISCRSSQSLVHSNRNTYLHWYLQKPGQSPQLLIYKVSNRFSGVPDRFSGSGSGTDFTLKISRVEAEDVGVYYCSQNTHVPPTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECHHHHHH

SEQ ID NO: 114 - protein, 224 aa, synthetic construct
QVQLVQSGAEVKKPGASVKVSCKASGYTFTDYEMHWVRQAPGQGLEWMGALDPKTGDTAYSQKFKGRVTLTADKSTSTAYMELSSLTSEDTAVYYCTRFYSYTYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCHHHHHH

SEQ ID NO: 115 - protein, 237 aa, synthetic construct
GHNYTTRNILPGLNITSDIQMTQSPSSLSASVGDRVTITCKASQDVGIAVAWYQQKPGKVPKLLIYWASTRHTGVPDRFSGSGSGTDFTLTISSLQPEDVATYYCQQYSSYPYTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECHHHHHH

SEQ ID NO: 116 - protein, 228 aa, synthetic construct
EVQLVESGGGLVQPGGSLRLSCAASGFDFSRYWMSWVRQAPGKGLEWIGEINPDSSTINYAPSLKDKFIISRDNAKNSLYLQMNSLRAEDTAVYYCARPDGNYWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCHHHHHH

SEQ ID NO: 117 - protein, 236 aa, synthetic construct
GHNYTTRNILPGLNITSDIQMTQSPSSLSASVGDRVTITCKASQDVGTAVAWYQQKPGKAPKLLIYWASTRHTGVPSRFSGSGSGTDFTLTISSLQPEDFATYYCQQYSSYRTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECHHHHHH

SEQ ID NO: 118 - protein, 228 aa, synthetic construct
EVQLVESGGGLVQPGGSLRLSCAASGFTFSSYVMSWVRQAPGKGLEWVATISSGGSYTYYPDSVKGRFTISRDNAKNTLYLQMNSLRAEDTAVYYCARRGDSMITTDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCHHHHHH

SEQ ID NO: 119 - protein, 238 aa, synthetic construct
GHNYTTRNILPGLNITSEIVMTQSPATLSVSPGERATLSCRASQSVDNNLVWYQQKPGQAPRLLIYGASTRATGIPARFSGSGSGTEFTLTISSLQSEDFAVYYCQQYNNWPPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECHHHHHH

SEQ ID NO: 120 - protein, 228 aa, synthetic construct
QVQLQESGPGLVKPSQTLSLTCTVSGGSISSFNYYWSWIRHHPGKGLEWIGYIYYSGSTYSNPSLKSRVTISVDTSKNQFSLTLSSVTAADTAVYYCARGYNWNYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCHHHHHH

SEQ ID NO: 121 - protein, 237 aa, synthetic construct
GHNYTTRNILPGLNITSDIQMTQSPSSVSASIGDRVTITCRASQGIDNWLGWYQQKPGKAPKLLIYDASNLDTGVPSRFSGSGSGTYFTLTISSLQAEDFAVYFCQQAKAFPPTFGGGTKVDIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECHHHHHH

SEQ ID NO: 122 - protein, 225 aa, synthetic construct
EVQLVQSGGGLVKPGGSLRLSCAASGFTFSSYSMNWVRQAPGKGLEWVSSISSSSSYIYYADSVKGRFTISRDNAKNSLYLQMNSLRAEDTAVYYCARVTDAFDIWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCHHHHHH

SEQ ID NO: 123 - protein, 237 aa, synthetic construct
GHNYTTRNILPGLNITSDIQMTQSPSSLSASVGDRVTITCRASESVDDLLHWYQQKPGKAPKLLIKYASQSISGVPSRFSGSGSGTDFTLTISSLQPEDFATYYCQQGNSLPNTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECHHHHHH

SEQ ID NO: 124 - protein, 226 aa, synthetic construct
EVQLVESGGGLVQPGGSLRLSCAASGFFITNNYWGWVRQAPGKGLEWVGYISYSGSTSYNPSLKSRFTISRDTSKNTFYLQMNSLRAEDTAVYYCARTGSSGYFDFWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCHHHHHH

SEQ ID NO: 125 - protein, 237 aa, synthetic construct
GHNYTTRNILPGLNITSDIQMTQSPSSVSASVGDRVTITCRASQGISSWLAWYQQKPGKAPKLLIYGASNLESGVPSRFSGSGSGTDFTLTISSLQPEDFANYYCQQANSFPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECHHHHHH

SEQ ID NO: 126 - protein, 227 aa, synthetic construct
QVQLVQSGAEVKKPGASVKVSCKVSGYTLSDLSIHWVRQAPGKGLEWMGGFDPQDGETIYAQKFQGRVTMTEDTSTDTAYMELSSLKSEDTAVYYCATGSSSSWFDPWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCHHHHHH

SEQ ID NO: 127 - protein, 245 aa, synthetic construct
GHNYTTRNILPGLNITSRSTQSALTQPASVSGSPGQSITISCTGTSSDVGGYNYVSWYQQHPGKAPKLMIYDVSNRPSGVSKRFSGSKSGNTASLTISGLQAEDEADYYCSSYTSSSTRHVFGTGTQLTVLGRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECHHHHHH

SEQ ID NO: 128 - protein, 227 aa, synthetic construct
EVQLVESGAEVKKPGDSLKISCKGSGYSFTSYWIGWVRQMPGKGLEWMGIIYPGDSVTTYSPAFQGDVTISVDKSISTAYLQWNSLKASDTGIYYCARRRGNYYMDVWGNGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCHHHHHH

SEQ ID NO: 129 - protein, 240 aa, synthetic construct
GHNYTTRNILPGLNITSESALTQPPSVSGAPGQKVTISCTGSTSNIGGYDLHWYQQLPGTAPKLLIYDINKRPSGISDRFSGSKSGTAASLAITGLQTEDEADYYCQSYDSSLNAQVFGGGTRLTVLRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECHHHHHH

SEQ ID NO: 130 - protein, 236 aa, synthetic construct
QVQLQESGPGLVKPSETLSLTCAVSGGSISGGYGWGWIRQPPGKGLEWIGSFYSSSGNTYYNPSLKSQVTISTDTSKNQFSLKLNSMTAADTAVYYCVRDRLFSVVGMVYNNWFDVWGPGVLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCHHHHHH

SEQ ID NO: 131 - protein, 242 aa, synthetic construct
GHNYTTRNILPGLNITSDVLMTQSPLSLPVTPGEPASISCRSSRNIVHINGDTYLEWYLQKPGQSPQLLIYKVSNRFSGVPDRFSGSGSGTDFTLKISRVEAEDVGVYYCFQGSLLPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECHHHHHH

SEQ ID NO: 132 - protein, 228 aa, synthetic construct
EVQLVESGGDLVQPGRSLRLSCAASGFIFSNYGMSWVRQAPGKGLEWVATISSASTYSYYPDSVKGRFTISRDNAKNSLYLQMNSLRVEDTALYYCGRHSDGNFAFGYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCHHHHHH

SEQ ID NO: 133 - protein, 236 aa, synthetic construct
GHNYTTRNILPGLNITSDIQMTQSPSSLSASVGDRVTITCRASEDVNTYVSWYQQKPGKAPKLLIYAASNRYTGVPSRFSGSGSGTDFTLTISSLQPEDFATYYCQQSFSYPTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECHHHHHH

SEQ ID NO: 134 - protein, 225 aa, synthetic construct
QVQLVQSGAEVKKPGASVKVSCKASGYTFTSYDISWVRQAPGQGLEWMGVIWTDGGTNYAQKLQGRVTMTTDTSTSTAYMELRSLRSDDTAVYYCARDQRLYFDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCHHHHHH

SEQ ID NO: 135 - protein, 236 aa, synthetic construct
GHNYTTRNILPGLNITSENVLTQSPAIMSASPGERVTMTCSASSSIRYIYWYQQKPGSSPRLLIYDTSNVAPGVPFRFSGSGSGTSYSLTINRMEAEDAATYYCQEWSGYPYTFGGGTKLELKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECHHHHHH

SEQ ID NO: 136 - protein, 228 aa, synthetic construct
EVKLQESGTEVVKPGASVKLSCKASGYIFTSYDIDWVRQTPEQGLEWIGWIFPGEGSTEYNEKFKGRATLSVDKSSSTAYMELTRLTSEDSAVYFCARGDYYRRYFDLWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCHHHHHH

SEQ ID NO: 137 - protein, 242 aa, synthetic construct
GHNYTTRNILPGLNITSDVVMTQSPLSLPVTLGQPASISCRSSQIIIHSDGNTYLEWFQQRPGQSPRRLIYKVSNRFSGVPDRFSGSGSGTDFTLKISRVEAEDVGVYYCFQGSHVPHTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECHHHHHH

SEQ ID NO: 138 - protein, 227 aa, synthetic construct
QVQLVESGGGVVQPGRSLRLSCAASGFTFSSFGMHWVRQAPGKGLEWVAYISSGSFTIYYADSVKGRFTISRDNSKNTLYLQMNSLRAEDTAVYYCARMRKGYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCHHHHHH

SEQ ID NO: 139 - protein, 237 aa, synthetic construct
GHNYTTRNILPGLNITSDIQLTQSPSSLSASVGDRVTITCRASQGISSNIVWLQQKPGKAPKGLIYHGTNLESGVPSRFSGSGSGTDYTLTISSLEPEDFATYYCVQYSQFPPTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECHHHHHH

SEQ ID NO: 140 - protein, 227 aa, synthetic construct
QVQLVQSGAEVVKPGASVKVSCKASGYTFTSYWMHWVKQAPGQGLEWIGAVSPGNSDTSYNEKFKGKATLTVDTSASTAYMELSSLRSEDTAVYYCTRSRYGNNALDYWGQGTLVTVSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCHHHHHH

SEQ ID NO: 141 - protein, 457 aa, synthetic construct
EVQLVESGGGLVKPGGSLKLSCAASGYTFTSYVMHWVRQAPGKGLEWIGYINPYNDGTKYNEKFQGRVTISSDKSISTAYMELSSLRSEDTAMYYCARGTYYYGTRVFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKHHHHHH

SEQ ID NO: 142 - protein, 457 aa, synthetic construct
EVQLVESGGGLVKPGGSLKLSCAASGYTFTSYVMHWVRQAPGKGLEWIGYINPYNDGTKYNEKFQGRVTISSDKSISTAYMELSSLRSEDTAMYYCARGTYYYGTRVFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPDVFLFPPKPKDTLMISRTPEVTCVVVDVSHEDPEVKFNWYVDGVEVHNAKTKPREEQYNSTYRVVSVLTVLHQDWLNGKEYKCKVSNKALPAPEEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKHHHHHH

SEQ ID NO: 143 - protein, 230 aa, synthetic construct; identical to SEQ ID NO: 52

SEQ ID NO: 144 - protein, 219 aa, synthetic construct
DIVMTQSPATLSLSPGERATLSCRSSKSLQNVNGNTYLYWFQQKPGQSPQLLIYRMSNLNSGVPDRFSGSGSGTEFTLTISSLEPEDFAVYYCMQHLEYPITFGAGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 145 - protein, 236 aa, synthetic construct
GHNYTTRNILPGLNITSDIVMTQSPATLSLSPGERATLSCRSSKSLQNVNGNTYLYWFQQKPGQSPQLLIYRMSNLNSGVPDRFSGSGSGTEFTLTISSLEPEDFAVYYCMQHLEYPITFGAGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 146 - protein, 241 aa, synthetic construct
GHNYTTRNILPGLNITSGGGGSDIVMTQSPATLSLSPGERATLSCRSSKSLQNVNGNTYLYWFQQKPGQSPQLLIYRMSNLNSGVPDRFSGSGSGTEFTLTISSLEPEDFAVYYCMQHLEYPITFGAGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 147 - protein, 251 aa, synthetic construct
GHNYTTRNILPGLNITSGGGGSGGGGSGGGGSDIVMTQSPATLSLSPGERATLSCRSSKSLQNVNGNTYLYWFQQKPGQSPQLLIYRMSNLNSGVPDRFSGSGSGTEFTLTISSLEPEDFAVYYCMQHLEYPITFGAGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 148 - protein, 246 aa, synthetic construct
GHNYTTRNILPGLNITSTFHKTSGSGKDIVMTQSPATLSLSPGERATLSCRSSKSLQNVNGNTYLYWFQQKPGQSPQLLIYRMSNLNSGVPDRFSGSGSGTEFTLTISSLEPEDFAVYYCMQHLEYPITFGAGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 149 - protein, 241 aa, synthetic construct
GHNYTTRNILPGLNITSTFHKTDIVMTQSPATLSLSPGERATLSCRSSKSLQNVNGNTYLYWFQQKPGQSPQLLIYRMSNLNSGVPDRFSGSGSGTEFTLTISSLEPEDFAVYYCMQHLEYPITFGAGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 150 - protein, 243 aa, synthetic construct
GHNYTTRNILPGLNITSTFHKTGSDIVMTQSPATLSLSPGERATLSCRSSKSLQNVNGNTYLYWFQQKPGQSPQLLIYRMSNLNSGVPDRFSGSGSGTEFTLTISSLEPEDFAVYYCMQHLEYPITFGAGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 151 - protein, 241 aa, synthetic construct
GHNYTTRNILPGLNITSTFHGSDIVMTQSPATLSLSPGERATLSCRSSKSLQNVNGNTYLYWFQQKPGQSPQLLIYRMSNLNSGVPDRFSGSGSGTEFTLTISSLEPEDFAVYYCMQHLEYPITFGAGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 152 - protein, 238 aa, synthetic construct
GHNYTTRNILPGLNITSGSDIVMTQSPATLSLSPGERATLSCRSSKSLQNVNGNTYLYWFQQKPGQSPQLLIYRMSNLNSGVPDRFSGSGSGTEFTLTISSLEPEDFAVYYCMQHLEYPITFGAGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 153 - protein, 241 aa, synthetic construct
DFPGHNYTTRNILPGLNITSGSDIVMTQSPATLSLSPGERATLSCRSSKSLQNVNGNTYLYWFQQKPGQSPQLLIYRMSNLNSGVPDRFSGSGSGTEFTLTISSLEPEDFAVYYCMQHLEYPITFGAGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 154 - protein, 244 aa, synthetic construct
DFPGHNYTTRNILPGLNITSGGGGSDIVMTQSPATLSLSPGERATLSCRSSKSLQNVNGNTYLYWFQQKPGQSPQLLIYRMSNLNSGVPDRFSGSGSGTEFTLTISSLEPEDFAVYYCMQHLEYPITFGAGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 155 - protein, 249 aa, synthetic construct
DFPGHNYTTRNILPGLNITSGGGGSGGGGSDIVMTQSPATLSLSPGERATLSCRSSKSLQNVNGNTYLYWFQQKPGQSPQLLIYRMSNLNSGVPDRFSGSGSGTEFTLTISSLEPEDFAVYYCMQHLEYPITFGAGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 156 - protein, 254 aa, synthetic construct
DFPGHNYTTRNILPGLNITSGGGGSGGGGSGGGGSDIVMTQSPATLSLSPGERATLSCRSSKSLQNVNGNTYLYWFQQKPGQSPQLLIYRMSNLNSGVPDRFSGSGSGTEFTLTISSLEPEDFAVYYCMQHLEYPITFGAGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 157 - protein, 249 aa, synthetic construct
DFPGHNYTTRNILPGLNITSTFHKTSGSGKDIVMTQSPATLSLSPGERATLSCRSSKSLQNVNGNTYLYWFQQKPGQSPQLLIYRMSNLNSGVPDRFSGSGSGTEFTLTISSLEPEDFAVYYCMQHLEYPITFGAGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 158 - protein, 251 aa, synthetic construct
DFPGHNYTTRNILPGLNITSTFHKTSGSGKGSDIVMTQSPATLSLSPGERATLSCRSSKSLQNVNGNTYLYWFQQKPGQSPQLLIYRMSNLNSGVPDRFSGSGSGTEFTLTISSLEPEDFAVYYCMQHLEYPITFGAGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 159 - protein, 254 aa, synthetic construct
DFPGHNYTTRNILPGLNITSTFHKTSGSGKGGGGSDIVMTQSPATLSLSPGERATLSCRSSKSLQNVNGNTYLYWFQQKPGQSPQLLIYRMSNLNSGVPDRFSGSGSGTEFTLTISSLEPEDFAVYYCMQHLEYPITFGAGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 160 - protein, 247 aa, synthetic construct
DFPGHNYTTRNILPGLNITSTFHGGGGSDIVMTQSPATLSLSPGERATLSCRSSKSLQNVNGNTYLYWFQQKPGQSPQLLIYRMSNLNSGVPDRFSGSGSGTEFTLTISSLEPEDFAVYYCMQHLEYPITFGAGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 161 - protein, 244 aa, synthetic construct
GHNYTTRNILPGLNITSTFHGGGGSDIVMTQSPATLSLSPGERATLSCRSSKSLQNVNGNTYLYWFQQKPGQSPQLLIYRMSNLNSGVPDRFSGSGSGTEFTLTISSLEPEDFAVYYCMQHLEYPITFGAGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 162 - protein, 249 aa, synthetic construct
DFPGHNYTTRNILPGLNITSTFHKTGGGGSDIVMTQSPATLSLSPGERATLSCRSSKSLQNVNGNTYLYWFQQKPGQSPQLLIYRMSNLNSGVPDRFSGSGSGTEFTLTISSLEPEDFAVYYCMQHLEYPITFGAGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 163 - protein, 246 aa, synthetic construct
GHNYTTRNILPGLNITSTFHKTGGGGSDIVMTQSPATLSLSPGERATLSCRSSKSLQNVNGNTYLYWFQQKPGQSPQLLIYRMSNLNSGVPDRFSGSGSGTEFTLTISSLEPEDFAVYYCMQHLEYPITFGAGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 164 - protein, 246 aa, synthetic construct
GHNYTTRNILPGLNITSGGGGSGGGGSDIVMTQSPATLSLSPGERATLSCRSSKSLQNVNGNTYLYWFQQKPGQSPQLLIYRMSNLNSGVPDRFSGSGSGTEFTLTISSLEPEDFAVYYCMQHLEYPITFGAGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 165 - protein, 786 aa, synthetic construct
MALPVTALLLPLALLLHAARPEVQLLESGGGLVQPGGSLRLSCAASGFTFRNHDMGWVRQAPGKGLEWVSAISGSGGSTYYANSVKGRFTISRDNSKNTLYLQMNSLRAEDTAVYYCAEPKPMDTEFDYRSPGTLVTVSSRAAAIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRLEGGGEGRGSLLTCGDVEENPGPRMLLLVTSLLLCELPHPAFLLIPRKVCNGIGIGEFKDSLSINATNIKHFKNCTSISGDLHILPVAFRGDSFTHTPPLDPQELDILKTVKEITGFLLIQAWPENRTDLHAFENLEIIRGRTKQHGQFSLAVVSLNITSLGLRSLKEISDGDVIISGNKNLCYANTINWKKLFGTSGQKTKIISNRGENSCKATGQVCHALCSPEGCWGPEPRDCVSCRNVSRGRECVDKCNLLEGEPREFVENSECIQCHPECLPQAMNITCTGRGPDNCIQCAHYIDGPHCVKTCPAGVMGENNTLVWKYADAGHVCHLCHPNCTYGCTGPGLEGCPTNGPKIPSIATGMVGALLLLLVVALGIGLFM

SEQ ID NO: 166 - protein, 788 aa, synthetic construct
MALPVTALLLPLALLLHAARPEVQLLESGGGLVQPGGSLRLSCAASGFTFRNHDMGWVRQAPGKGLEWVSAISGSGGSTYYANSVKGRFTISRDNSKNTLYLQMNSLRAEDTAVYYCAEPKPMDTEFDYRSPGTLVTVSSTTTPAPRPPTPAPTIASQPLSLRPEACRPAAGGAVHTRGLDFACDFWVLVVVGGVLACYSLLVTVAFIIFWVRSKRSRLLHSDYMNMTPRRPGPTRKHYQPYAPPRDFAAYRSKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRLEGGGEGRGSLLTCGDVEENPGPRMLLLVTSLLLCELPHPAFLLIPRKVCNGIGIGEFKDSLSINATNIKHFKNCTSISGDLHILPVAFRGDSFTHTPPLDPQELDILKTVKEITGFLLIQAWPENRTDLHAFENLEIIRGRTKQHGQFSLAVVSLNITSLGLRSLKEISDGDVIISGNKNLCYANTINWKKLFGTSGQKTKIISNRGENSCKATGQVCHALCSPEGCWGPEPRDCVSCRNVSRGRECVDKCNLLEGEPREFVENSECIQCHPECLPQAMNITCTGRGPDNCIQCAHYIDGPHCVKTCPAGVMGENNTLVWKYADAGHVCHLCHPNCTYGCTGPGLEGCPTNGPKIPSIATGMVGALLLLLVVALGIGLFM

SEQ ID NO: 167 - protein, 755 aa, synthetic construct
MALPVTALLLPLALLLHAARPEVQLLESGGGLVQPGGSLRLSCAASGFTFRNHDMGWVRQAPGKGLEWVSAISGSGGSTYYANSVKGRFTISRDNSKNTLYLQMNSLRAEDTAVYYCAEPKPMDTEFDYRSPGTLVTVSSESKYGPPCPPCPFWVLVVVGGVLACYSLLVTVAFIIFWVRSKRSRLLHSDYMNMTPRRPGPTRKHYQPYAPPRDFAAYRSKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRLEGGGEGRGSLLTCGDVEENPGPRMLLLVTSLLLCELPHPAFLLIPRKVCNGIGIGEFKDSLSINATNIKHFKNCTSISGDLHILPVAFRGDSFTHTPPLDPQELDILKTVKEITGFLLIQAWPENRTDLHAFENLEIIRGRTKQHGQFSLAVVSLNITSLGLRSLKEISDGDVIISGNKNLCYANTINWKKLFGTSGQKTKIISNRGENSCKATGQVCHALCSPEGCWGPEPRDCVSCRNVSRGRECVDKCNLLEGEPREFVENSECIQCHPECLPQAMNITCTGRGPDNCIQCAHYIDGPHCVKTCPAGVMGENNTLVWKYADAGHVCHLCHPNCTYGCTGPGLEGCPTNGPKIPSIATGMVGALLLLLVVALGIGLFM

SEQ ID NO: 168 - protein, 32 aa, synthetic construct
GHNYTTRNILPGLNITSGGGGSGGGGSGGGGS

SEQ ID NO: 169 - protein, 236 aa, synthetic construct
GHNYTTRNILPGLNITSDIQMTQSPSSLSASVGDRVTITCRASQSINSYLNWYQQKPGKAPKLLIYAASSLQSGVPSRFSGSGSGTDFTLTISSLQPEDFATYYCQQGVSDITFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECHHHHHH

SEQ ID NO: 170 - protein, 228 aa, synthetic construct
QVQLVQSGAEVKKPGSSVKVSCKASGGTFRIYAISWVRQAPGQGLEWMGGIIPDFGVANYAQKFQGRVTITADESTSTAYMELSSLRSEDTAVYYCARGGLDTDEFDLWGRGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCHHHHHH

SEQ ID NO: 171 - protein, 236 aa, synthetic construct
GHNYTTRNILPGLNITSDIQMTQSPSSLSASVGDRVTITCRASQGSSYVAWYQQKPGKAPKLLIYSASYLYSGVPSRFSGSRSGTDFTLTISSLQPEDFATYYCQQGVWSLITFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECHHHHHH

SEQ ID NO: 172 - protein, 227 aa, synthetic construct
EVQLVESGGGLVQPGGSLRLSCAASGFNLSSSSIHWVRQAPGKGLEWVAYIYPYYSYTYYADSVKGRFTISADTSKNTAYLQMNSLRAEDTAVYYCAREGSVAGEDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCHHHHHH

SEQ ID NO: 173 - protein, 243 aa, synthetic construct
GHNYTTRNILPGLNITSDIVMTQSPDSLAVSLGERATINCKSSQSLLNSGDQKNYLTWYQQKPGQPPKLLIYWASTRESGVPDRFSGSGSGTDFTLTISSLQAEDVAVYYCQNDYSYPLTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECHHHHHH

SEQ ID NO: 174 - protein, 225 aa, synthetic construct
QVQLVQSGAEVKKTGSSVKVSCKASGYTFTDHAIHWVRQAPGQALEWMGHFSPGNTDIKYNDKFKGRVTLTVDRSMSTAYMELSSLRSEDTAMYYCKTSTFFFDYWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCHHHHHH

SEQ ID NO: 175 - protein, 237 aa, synthetic construct
GHNYTTRNILPGLNITSDIQMTQSPSSLSASVGDRVTITCRASQSISSYLNWYQQKPGKAPKLLIYAASSLQSGVPSGFSGSGSGTDFTLTISSLQPEDFATYYCQQSYSTPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECHHHHHH

SEQ ID NO: 176 - protein, 228 aa, synthetic construct
QVQLQESGPGLVKPSETLSLTCTVSGGSVSSGSYYWSWIRQPPGKGLEWIGYIYYSGSTNYNPSLKSRVTISVDTSKNQFSLKLSSVTAADTAVYYCAREGKNGAFDIWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCHHHHHH

SEQ ID NO: 177 - protein, 241 aa, synthetic construct
GHNYTTRNILPGLNITSQSALTQPASVSGSPGQSITISCTGTSGDVGGYNYVSWYQHHPGKAPKLIIYDVNKRPSGFSDRFSGSKSGNTASLTISGLQAEDEADYYCSSYTSTSTVFGGGTKLTVLGKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECHHHHHH

SEQ ID NO: 178 - protein, 230 aa, synthetic construct
QITLKESGPELVKPTQTLTLTCTFSGFSLSTSGMSVSWIRQPPGKALEWLARIDWDDDKYYSTSLKTRLTISKDTSKNQVVLTMTNTDPVDTATYYCARGFYLAYGSYDSWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCHHHHHH

SEQ ID NO: 179 - protein, 238 aa, synthetic construct
GHNYTTRNILPGLNITSDIQMTQSPSSLSASVGDRVTITCTASLSVSSTYLHWYQQKPGSSPKLWIYSTSNLASGVPSRFSGSGSGTSFTLTISSLQPEDFATYYCHQYHRSPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECHHHHHH

SEQ ID NO: 180 - protein, 225 aa, synthetic construct
EVQLVESGGGLVQPGGSLRLSCAASGFSLTKYGVHWVRQAPGKGLEWVGVKWAGGSTDYNSALMSRFTISKDNAKNSLYLQMNSLRAEDTAVYYCARDHRDAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCHHHHHH

SEQ ID NO: 181 - protein, 140 aa, synthetic construct
HHHHHHGPVPPSTALRYLIEELVNITQNQKAPLCNGSMVWSINLTAGMYCAALESLINVSGCSAIEKTQRMLSGFCPHKVSAGQFSSLHVRDTKIEVAQFVKDLLLHLKKLFREGRFNGGGGSGHNYTTRNILPGLNITS

SEQ ID NO: 182 - protein, 237 aa, synthetic construct
GHNYTTRNILPGLNITSDIQLTQSPSSLSASVGDRVTITCKASQDINNYLSWYQQKPGQAPRLLIYRANRLVDGVPDRFSGSGYGTDFTLTINNIESEDAAYYFCLKYDVFPYTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECHHHHHH

SEQ ID NO: 183 - protein, 223 aa, synthetic construct
QVQLLESGGGLVQPGGSLRLSCAASGFTFSSYTMSWVRQAPGQALEWMGTISSGGTYTYYPDSVKGRFTISRDNAKNSLYLQMNSLRAEDTAVYYCAREAIFTYWGRGTLVTSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCHHHHHH

SEQ ID NO: 184 - protein, 237 aa, synthetic construct
GHNYTTRNILPGLNITSDIQMTQSPSSLSASVGDRVTITCRASQGIRNNLAWYQQKPGKAPKRLIYAASNLQSGVPSRFTGSGSGTEFTLIVSSLQPEDFATYYCLQHHSYPLTSGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECHHHHHH

SEQ ID NO: 185 - protein, 225 aa, synthetic construct
EVQVLESGGGLVQPGGSLRLSCAASGFTFSSYAMSWVRQAPGKGLEWVSAISGSGGSTNYADSVKGRFTISRDNSKNTLYLQMNSLRAEDTAVYYCAGSSGWSEYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCHHHHHH

SEQ ID NO: 186 - protein, 237 aa, synthetic construct
GHNYTTRNILPGLNITSDIVMTQSPSSLSASVGDRVTITCKASQDVGTAVDWYQQKPGKAPKLLIYWASTRHTGVPDRFTGSGSGTDFTLTISSLQPEDFADYFCQQYNSYPLTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECHHHHHH

SEQ ID NO: 187 - protein, 224 aa, synthetic construct
EVQLVQSGAEVKKPGASVKISCKTSGYTFTEYTIHWVKQASGKGLEWIGNINPNNGGTTYNQKFEDRATLTVDKSTSTAYMELSSLRSEDTAVYYCAAGWNFDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCHHHHHH

SEQ ID NO: 188 - protein, 240 aa, synthetic construct
GHNYTTRNILPGLNITSDIVLTQSPASLTVSLGLRATISCRASKSVSASGYSYMHWYQQRPGQPPKLLIYLASNLQSGVPARFSGSGSGTDFTLNIHPVEEEDAATYYCQHSRELPTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECHHHHHH

SEQ ID NO: 189 - protein, 233 aa, synthetic construct
EVQLQQSGAELVRSGASVKMSCTASGFNIKDYYMHWVKQRPEQGLEWIGWIDPENGDTEYAPKFQGKATMTTDYSSNTAYLQLSSLTSEDTAVYYCNTRGLSTMITTRWFFDVWGAGTTVAVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCHHHHHH

SEQ ID NO: 190 - protein, 236 aa, Synthetic construct
GHNYTTRNILPGLNITSDIQLTQSPSSLSASVGDRVTITCSASSSVRFIHWYQQKPGKAPKRLIYDTSKLASGVPSRFSGSGSGTDFTLTISSLQPEDFATYYCQQWSSSPFTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECHHHHHH

SEQ ID NO: 191 - protein, 221 aa, synthetic construct
EVQLVESGGGLVQPGGSLRLSCAASGFNIKDYYIHWVRQAPGKGLEWVAWIDPENGDTEFADSVKGRFTISADTSKNTAYLQMNSLRAEDTAVYYCKTGGFWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCHHHHHH

SEQ ID NO: 192 - protein, 243 aa, synthetic construct
GHNYTTRNILPGLNITSDIQMTQSPSSLSASVGDRVTITCRSSQRIVHSNGNTYLEWYQQTPGKAPKLLIYKVSNRFSGVPSRFSGSGSGTDFTFTISSLQPEDIATYYCFQGSHVPFTFGQGTKLQITKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECHHHHHH

SEQ ID NO: 193 - protein, 228 aa, unidentified
EVQLVESGGGVVQPGRSLRLSCSSSGFTFSDYYMYWVRQAPGKGLEWVAYMSNVGAITDYPDTVKGRFTISRDNSKNTLFLQMDSLRPEDTGVYFCARGTRDGSWFAYWGQGTPVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCHHHHHH

SEQ ID NO: 194 - protein, 237 aa, synthetic construct
GHNYTTRNILPGLNITSDIQMTQSPSSLSASVGDRVTITCRASQDISNYLNWYQQKPGKAPKLLIYYTSRLHSGVPSRFSGSGSGTDYTLTISSLQPEDFATYFCQQGNTLPWTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECHHHHHH

SEQ ID NO: 195 - protein, 226 aa, synthetic construct
EVQLVQSGGGLVQSGGSLRLSCRASGYTFTRYWMLWVRQRPGHGLEWVGEINPRNDRTNYNEKFKTRFTISVDRSKSTAYLQMDSLRAEDTAVYFCALGGGYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCHHHHHH

SEQ ID NO: 196 - protein, 239 aa, synthetic construct
GHNYTTRNILPGLNITSEIVMTQSPATLSVSPGERATLSCRASQSVSSNLAWYQQKPGQAPRLIIYGASTTASGIPARFSASGSGTDFTLTISSLQSEDFAVYYCQQYNNWPPAYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECHHHHHH

SEQ ID NO: 197 - protein, 224 aa, synthetic construct
QVQLVQSGAEVKKPGSSVKVSCKASGGTFSSYAISWVRQAPGQGLEWMGGIIPIFGTANYAQKFQGRVTITADESTSTAYMELSSLRSEDTAVYYCARGLLWNYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCHHHHHH

SEQ ID NO: 198 - protein, 240 aa, synthetic construct
GHNYTTRNILPGLNITSEIVLTQSPATLSLSPGERATLSCRASESVDNYGISFAWYQQKPGQAPRLLIYRASRATGIPARFSGSGSGTDFTLTISSLEPEDFAVYYCQQNNKDPPTFFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECHHHHHH

SEQ ID NO: 199 - protein, 230 aa, synthetic construct
QVQLQQSGAEVKKPGSSVKVSCKASGYAFSSYISWVRQAPGQGLEWMGGQIYPGDGDTNYAQKFQGRVTITADESTSTAYMELSSLRSEDTAVYYCVRYYYGSSGYFDYWWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCHHHHHH

SEQ ID NO: 200 - protein, 242 aa, synthetic construct
GHNYTTRNILPGLNITSDVVMTQTPLSLPVSLGDPASISCRSSQSIVHSNVNTYLEWYLQKPGQSPRLLIYKVSNRFSGVPDRFSGSGAGTDFTLRISRVEAEDLGIYYCFQGSHVPPTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECHHHHHH

SEQ ID NO: 201 - protein, 233 aa, synthetic construct
QVQLVQSGAEVVKPGASVKLSCKASGYTFTSYWMHWVKQRPGQGLEWIGEINPSNGRTNYNQKFQGKATLTVDKSSSTAYMQLSSLTSEDSAVYYFARGRPDYYGSSKWYFDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCHHHHHH

SEQ ID NO: 202 - protein, 237 aa, synthetic construct
GHNYTTRNILPGLNITSSIVMTQTPKFLLVSAGDRVTITCKASQSVSNDVAWYQQKPGQSPKLLIYYASNRYTGVPDRFTGSGYGTDFTFTISAVQAEDLAVYFCQQDYSSPYTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECHHHHHH

SEQ ID NO: 203 - protein, 226 aa, synthetic construct
QVQLQQSGPELVKPGASVRISCKASGYTFTTYYIHWVKQRPGQGLEWIGWIYPGNVNTKNNEKFKVKATLTADKSSSTAYMQLSSLTSEDSAVYFCARSNPYVMDYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCHHHHHH

SEQ ID NO: 204 - protein, 238 aa, unidentified
GHNYTTRNILPGLNITSEIVLTQSPGTLSLSPGERATLSCRASQSVTSSYLAWYQQKPGQAPRLLINVGSRRATGIPDRFSGSGSGTDFTLTISRLEPEDFAVYYCQQGIMLPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECHHHHHH

SEQ ID NO: 205 - protein, 226 aa, synthetic construct
EVQLLESGGGLVQPGGSLRLSCAASGFTFSSYAMSWVRQAPGKGLEWVSAIIGSGSITYYADSVKGRFTISRDNSKNTLYLQMNSLRAEDTAVYYCAKGWFGGFNYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCHHHHHH

SEQ ID NO: 206 - protein, 244 aa, synthetic construct
GHNYTTRNILPGLNITSDVVMTQSPLSLPVTLGQPASISCRSSQNIVHTNGNTYLEWYQQKPGKAPELLIYKVSNRFSGVPDRFSGSGSGTDFTLKISRVEAEDVGVYYCFQGSHLLEPFTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECHHHHHH

SEQ ID NO: 207 - protein, 228 aa, synthetic construct
QVTLKESGPVLVKPTETLTLTCTVSGFSLSSFGVDWVRQAPGKGLEWMGVIWGGGSTNYNSALKSRLTISKDNSKSQVVLTMTNMDPVDTATYYCAGEGSKYGAWFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCHHHHHH

SEQ ID NO: 208 - protein, 237 aa, synthetic construct
GHNYTTRNILPGLNITSDIQMTQSTSSLSASLGDRVTISCSASQGINNYLNWYQQKPDGTVELLIYYTSTLQSGVPSRFSGSGSGTDYSLTISNLEPEDIGTYYCQQYSKLPRTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECHHHHHH

SEQ ID NO: 209 - protein, 231 aa, synthetic construct
QVQLQQSGSELMMPGASVKISCKATGYTFSNYWIEWVKQRPGHGLEWIGEILPGTGRTIYNEKFKGKATFTADISSNTVQMQLSSLTSEDSAVYYCARRDYYGNFYYAMDYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCHHHHHH

SEQ ID NO: 210 - protein, 237 aa, synthetic construct
GHNYTTRNILPGLNITSDIQMTQSPSSLSASVGDRVTITCRISENIYSYLAWYQQKPGKAPKLLIYNAKILVEGVPSRFSGSGSGTDFTLTISSLQPEDFATYYCQHHYTVPWTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECHHHHHH

SEQ ID NO: 211 - protein, 226 aa, synthetic construct
EVQLLESGGGLVQPGGSMRLSCAASGFTFSNYWMNWVRQAPGKGLEWVAQIRLKSDNYATHYADSVKGRFTISRDDSKNTVYLQMNSLRAEDTGVYYCNDGPPSGYWGQGTLLTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCHHHHHH

SEQ ID NO: 212 - protein, 242 aa, synthetic construct
GHNYTTRNILPGLNITSQSVLTQPASVSGSPGQSITISCAGTSSDVGGSSYVSWYQQHPGKAPKLMIYYDSYRPSGVSNRFSGSKSGNTASLTISGLQAEDEADYYCSSNTYYSTRVFGGGTKLAVLGKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECHHHHHH

SEQ ID NO: 213 - protein, 230 aa, synthetic construct
EVQLVESGGSLVKPGGSLRLSCAASGFTFSNYYMNWVRQAPGKGLEWISSIDGSSRYIDYADFVKGRFTISRDNATNSLYLQMNSLRAEDTAVYYCVRSSSDYFGGGMDVWGRGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCHHHHHH

SEQ ID NO: 214 - protein, 243 aa, synthetic construct
GHNYTTRNILPGLNITSDIVMTQSPSSLTVTAGEKVTMSCKSSQSLLNSGNQKNYLTWYQQKPGQPPKLLIYWASTRESGVPDRFTGSGSGTDFTLTISSVQAEDLAVYYCQNDYSYPFTFGSGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECHHHHHH

SEQ ID NO: 215 - protein, 227 aa, synthetic construct
QVQLQQPGAELVRPGASVKLSCKASGYTFTSYWINWVKQRPGQGLEWIGNIYPSDSYTNYNQKFKDKATLTVDKSSSTAYMQLSSPTSEDSAVYYCTRSWRGNSFDYWGQGTTLTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCHHHHHH

SEQ ID NO: 216 - protein, 242 aa, synthetic construct
GHNYTTRNILPGLNITSDVVMTQSPLSLPVTLGQPASISCRSSQSLLKNNGNTFLHWYQQRPGQSPRLLIYKVSNRLSGVPDRFSGSGSGTDFTLKISRVEAEDVGVYFCSQSTHIPYTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECHHHHHH

SEQ ID NO: 217 - protein, 228 aa, synthetic construct
EVQLVESGGGLVQPGRSLRLSCTTSEFTFTDYYMTWVRQAPGKGLEWLGFIRNRANGYTTEYNPSVKGRFTISRDNSKSILYLQMNSLKTEDTAVYYCARVSNWAFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCHHHHHH

SEQ ID NO: 218 - protein, 237 aa, synthetic construct
GHNYTTRNILPGLNITSDIQMTQSPSSLSASVGDRVTITCSASQDISNYLNWYQQKPDKAVKLLIFYSSNLHSGVPSRFSGGGSGTDYTLTISSLQPEDIATYFCHQYSKLPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECHHHHHH

SEQ ID NO: 219 - protein, 228 aa, synthetic construct
EVQLVESGGDFVQPGGSLRVSCAASGFAFSHYAMSWVRQAPGKGLEWVAYISSGGSGTYYSDSVKGRFTISRDNSKNTLYLQMRSLRAEDSAVYFCTRVKLGTYYFDSWGQGTLLTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCHHHHHH

SEQ ID NO: 220 - protein, 236 aa, synthetic construct
GHNYTTRNILPGLNITSDIQLTQSPSSLSASPGDRVTITCSASSSVSYMHWFQQKPGKAPKLWIYSTSNLASGVPSRFSGSGSGTSYSLTISRLQPEDIATYYCQQRSSYPYTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECHHHHHH

SEQ ID NO: 221 - protein, 229 aa, synthetic construct
EVQLVQSGAEVKKPGASVKVSCKASGYTFTDYNMDWVRQAPGQGLEWMGYIYPNNGGTGYNQKFKSKVTITVDTSTSTAYMELHSLRSEDTAVYYCATYGHYYGYMFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCHHHHHH

SEQ ID NO: 222 - protein, 242 aa, synthetic construct
GHNYTTRNILPGLNITSAVVMTQTPLSLPVSLGDQASISCRSSQSLVHSNGNTYLHWYMQKPGQSPKVLIYKVSNRFSGVPDRFSGSGSGTDFTLKISRVEADDLGIYFCSQSTHIPLAFGAGTKLELKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECHHHHHH

SEQ ID NO: 223 - protein, 232 aa, synthetic construct
EVILVESGGGLVKPGGSLKLSCAASGFTFSSFAMSWVRQTPEKRLEWVATISSGSIYIYYTDGVKGRFTISRDNAKNTVHLQMSSLRSEDTAMYYCARRGIYYGYDGYAMDYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCHHHHHH

SEQ ID NO: 224 - protein, 237 aa, synthetic construct
GHNYTTRNILPGLNITSDIQMTQSPSTLSASVGDRVTLSCKASENVGTYVSWYQQKPGKAPKLLIYGASNRYTGVPSRFTGSGSGTDFTLTISSLQPEDFATYYCGQSYSYPFTFGSGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECHHHHHH

SEQ ID NO: 225 - protein, 225 aa, synthetic construct
EVQLVESGGGLVQPGGSLRLSCAASGFTFSNFWMNWVRQAPGKGLEWVSEIRLKSNNYATHYAESVKGRFTISRDDSKNTLYLQMNSLKTEDTAVYYCTSYDYEYWGQGTLVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCHHHHHH

SEQ ID NO: 226 - protein, 236 aa, synthetic construct
GHNYTTRNILPGLNITSDIQLTQSPSSLSASVGDRVTITCKASQDVGTSVAWYQQKPGKAPKLLIYWTSTRHTGVPSRFSGSGSGTDFTFTISSLQPEDIATYYCQQYSLYRSFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECHHHHHH

SEQ ID NO: 227 - protein, 228 aa, synthetic construct
EVQLVESGGGVVQPGRSLRLSCSASGFDFTTYWMSWVRQAPGKGLEWIGEIHPDSSTINYAPSLKDRFTISRDNAKNTLFLQMDSLRPEDTGVYFCASLYFGFPWFAYWGQGTPVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCHHHHHH

SEQ ID NO: 228 - protein, 234 aa, synthetic construct
GHNYTTRNILPGLNITSDIVMTQSPDSLAVSLGERATINCSASSSVSYMHWYQQKPGQPPKLLIYRTSNLASGVPDRFSGSGSGTDFTLTISSLQAEDVAVYYCHQWSMYTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECHHHHHH

SEQ ID NO: 229 - protein, 226 aa, unidentified
QVQLVQSGAEVKKPGASVKVSCKASGYTFINYNMHWVRQAPGQGLEWMGAIFPGNGFTSYNQKFKGRVTITVDKSTSTAYMELSSLRSEDTAVYYCARDGDYYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCHHHHHH

SEQ ID NO: 230 - protein, 243 aa, synthetic construct
GHNYTTRNILPGLNITSDVLMTQTPLSLPVSLGDQASISCRSSRNIVQSTGNTYLEWYLQKPGQSPKLLIFKVSNRFSGVPDRFSGSGSGTDFTLKISRVEAEDLGVYYCFQGSHVPPWTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECHHHHHH

SEQ ID NO: 231 - protein, 228 aa, synthetic construct
DVQLVESGGGLVQPGGSRKLSCAASGFTFSGFGMHWVRQAPEKGLEWVAYISSVSSRIYYADTVKGRFTISRDNPKNTLFLQMTSLRSEDTAMYYCAREQTGPAWFAYWGQGTLVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCHHHHHH

SEQ ID NO: 232 - protein, 243 aa, synthetic construct
GHNYTTRNILPGLNITSQTWTQPPSASGTPGQRVTISCSGSSSNIGSNYVYWYQQLPGTAPKLVLLIYRSNQRPSGVPDRFSGSKSGTSASLAISGPRSVDEADYYCAAWDDSLNGVVFGGGTKLTVLGKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECHHHHHH

SEQ ID NO: 233 - protein, 230 aa, synthetic construct
QVQLVQSGAEVKKPGSSVKVSCKASGGTFSSYAISWVRQAPGQGLEVHWMGGIIPIFGTANYAQKFQGRVTITADESTSTAYMELSSLRSEDTAVYYCARRIPPYYGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCHHHHHH

SEQ ID NO: 234 - protein, 236 aa, synthetic construct
GHNYTTRNILPGLNITSENVLTQSPAIMSASLGEKVTMSCRASSSVNYIYWYQQKSDASPKLWIYYTSNLAPGVPARFSGSGSGNSYSLTISSMEGEDAATYYCQQFSSSPSTFGTGTKLELKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECHHHHHH

SEQ ID NO: 235 - protein, 232 aa, unidentified
EVQLQQSGPELVKPGASVKMSCKASRFTFTDYNIHWVKQSPGKTLEWIGYINPNNGDIFYKQKFNGKATLTINKSSNTAYMELRSLTSEDSAVYYCVRSSNIRYTFDRFFDVWGTGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCHHHHHH

SEQ ID NO: 236 - protein, 237 aa, synthetic construct
GHNYTTRNILPGLNITSDIQMTQSPSSLSASVGDRVTITCRASESVSTLMHWYQQKPGKAPKLLIYGTSNLESGVPSRFSGSGSGTDFTLTISSLQPEDFATYYCQQSWNDPPTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECHHHHHH

SEQ ID NO: 237 - protein, 227 aa, unidentified
EVQLVESGGGLVQPGGSLRLSCAASGFTFSNYYMSWVRQAPGKGLEWVATINPSGTRTYYPDSVKGRFTISRDNAKNSLYLQMNSLRAEDTAVYYCARLYNNYAFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCHHHHHH

SEQ ID NO: 238 - protein, 237 aa, synthetic construct
GHNYTTRNILPGLNITSDIQMTQSPSSLSASVGDRVTITCRASQDVSTAVAWYQQKPGKAPKLLIYSASFLYSGVPSRFSGSGSGTDFTLTISSLQPEDFATYYCQQTAQTPETFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECHHHHHH

SEQ ID NO: 239 - protein, 237 aa, synthetic construct
EVQLVESGGGLVQPGGSLRLSCAASGFTISGYYIHWVRQAPGKGLEWVGGIYLYSGSTDYADSVKGYADSVKGRFTISADTSKNTAYLQMNSLRAEDTAVYYCARGSGLRLGGLDYAMDYWGQGTLVTASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCHHHHHH

SEQ ID NO: 240 - protein, 243 aa, unidentified
GHNYTTRNILPGLNITSDIQMTQSPSSLSASVGDRVTITCKSSQSLLYRSNQKNYLAWYQQKPGKAPKLLIYWASTRESGVPSRFSGSGSGTDFTLTISSLQPEDFATYYCQQYYNYPRTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECHHHHHH

SEQ ID NO: 241 - protein, 233 aa, synthetic construct
EVQLVESGGGLVQPGGSLRLSCAVSGYSITSDYAWNWVRQAPGKGLEWVGYISNSGSTSYNPSLKSRFTISRDTSKNTLYLQMNSLRAEDTAVYYCARERNYDYDDYYYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCHHHHHH

SEQ ID NO: 242 - protein, 237 aa, synthetic construct
GHNYTTRNILPGLNITSDIQMTQSPSTLSASVGDRVTITCRASQSISSWLAWYQQKPGRAPNLLISKASSLKSGVPSRFSGSGSGTEFTLTVSSLQPDDFATYYCQQYYSYSYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECHHHHHH

SEQ ID NO: 243 - protein, 224 aa, synthetic construct
QVQLVESGGGVVQPGRSLRLSCVASGFTISSYGMNWVRQAPGKGLEWVAVISYDGGNKYSVDSVKGRFTISRDNSKNTLYLQMNSLRAEDSAVYYCARGRYFDLWGRGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCHHHHHH

SEQ ID NO: 244 - protein, 237 aa, synthetic construct
GHNYTTRNILPGLNITSDIVLTQSPATLSMTPGDSVSLSCRASQSISNNLHWYQQKSHESPRLLIKYASQSISGIPSRFSGSGSGTDFTLSFNSVETEDFGVYFCQQRYSWPLTFGAGTKLEMKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECHHHHHH

SEQ ID NO: 245 - protein, 226 aa, synthetic construct
QVQLQQPGAELVRPGSSVKLSCKASGYTFTNYWMHWLKQRPVQGLEWIGNIGPSDSKTHYNQKFKDKATLTVDKSSSTAYMQLNSLTSEDSAVYYCARGDYVLFTYWGQGTLVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCHHHHHH

SEQ ID NO: 246 - protein, 237 aa, synthetic construct
GHNYTTRNILPGLNITSDIVMTQSPSSLSASVGDRVTITCRASQSVSSDVAWFQQKPGKSPKPLIYYASNRYSGVPSRFSGSGSGTDFTLTISSLQAEDFAVYFCGQDYTSPRTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECHHHHHH

SEQ ID NO: 247 - protein, 225 aa, synthetic construct
QVQLQESGPGLVKPSETLSLTCTVSGYSITSDYAWNWIRQFPGNKLEWMGYITYSGSTYYNPSLKSRVTISVDTSKNQFSLKLSSVTAADTATYYCVSGTYYFDYWGQGTTLTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCHHHHHH

SEQ ID NO: 248 - protein, 167 aa, synthetic construct
GHNYTTRNILPGLNITSMCMPCFTTDHQMARKCDDCCGGKGRGKCYGPQCLCRKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECHHHHHH

SEQ ID NO: 249 - protein, 145 aa, synthetic construct
MCMPCFTTDHQMARKCDDCCGGKGRGKCYGPQCLCRASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCHHHHHH

SEQ ID NO: 250 - protein, 244 aa, synthetic construct
GHNYTTRNILPGLNITSQVQLQESGGGLVQPGGSLRLSCAASGFSLDYYAIGWFRQAPGKEREGISCISYKGGSTTYADSVKGRFTISKDNAKNTAYLQMNSLKPEDTGIYSCAAGFVCYNYDYWGQGTQVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCHHHHHH

SEQ ID NO: 251 - protein, 238 aa, synthetic construct
GHNYTTRNILPGLNITSDVQMTQSPSSLSASVGDRVTITCQASQSIYSFLSWYQQKPGQAPKLLIYAASDLESGVPSRFSGSGSGTDFTLTISSLQPEDFATYYCQCNYIIDYGAFGQGTKVVIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECHHHHHH

SEQ ID NO: 252 - protein, 229 aa, synthetic construct
EVQLEESGGRLVQPGTSLRLSCAVSGFSLSSYDMSWVRQAPGKGLEWVSTIYIGGTTAYASWPKGRFTISKTNSKNTLYLQMNSLRAEDTAVYFCARLQGANYYNSLALWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCHHHHHH

SEQ ID NO: 253 - protein, 237 aa, synthetic construct
GHNYTTRNILPGLNITSAIQLTQSPSSLSASVGDRVTITCRASQGISSALAWYQQKPGKVPKSLIYDASSLESGVPSRFSGSGSGTDFTLTISSLQPEDFATYYCQQFNSYPITFGQGTRLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECHHHHHH

SEQ ID NO: 254 - protein, 224 aa, synthetic construct
QVQLVESGGGVVQPGRSLRLSCAASGFTFSNYAMHWVRQAPGEGLEWVALIWYDGSNKFYGDSVKGRFTISRDNSKNTLYLQMNSLSAEDTAVYYCAREGSGHYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCHHHHHH

SEQ ID NO: 255 - protein, 299 aa, synthetic construct
GHNYTTRNILPGLNITSMKPTLISVLVIIFILRGTRAQRVTQPEKLLSVFKGAPVELKCNYSYSGSPELFWYVQYSRQRLQLLLRHISRESIKGFTADLNKGETSFHLKKPFAQEEDSAMYYCALRSGGYQKVTFGTGTKLQVIPGGGGSGGGGSGGGGSMGIRLLCRVAFCFLAVGLVDVKVTQSSRYLVKRTGEKVFLECVQDMDHENMFWYRQDPGLGLRLIYFSYDVKMKEKGDIPEGYSVSREKKERFSLILESASTNQTSMYLCASNNRDSYNSPLHFGNGTRLTVTHHHHHH

SEQ ID NO: 256 - protein, 238 aa, synthetic construct
GHNYTTRNILPGLNITSMKPTLISVLVIIFILRGTRAQRVTQPEKLLSVFKGAPVELKCNYSYSGSPELFWYVQYSRQRLQLLLRHISRESIKGFTADLNKGETSFHLKKPFAQEEDSAMYYCALRSGGYQKVTFGTGTKLQVIPIQNPDPAVYQLRDSKSSDKSVCLFTDFDSQTNVSQSKDSDVYITDKCVLDMRSMDFKSNSAVAWSNKSDFACANAFNNSIIPEDTFFPSPESS

SEQ ID NO: 257 - protein, 268 aa, synthetic construct
MGIRLLCRVAFCFLAVGLVDVKVTQSSRYLVKRTGEKVFLECVQDMDHENMFWYRQDPGLGLRLIYFSYDVKMKEKGDIPEGYSVSREKKERFSLILESASTNQTSMYLCASNNRDSYNSPLHFGNGTRLTVTDLKNVFPPEVAVFEPSEAEISHTQKATLVCLATGFYPDHVELSWWVNGKEVHSGVCTDPQPLKEQPALNDSRYALSSRLRVSATFWQDPRNHFRCQVQFYGLSENDEWTQDRAKPVTQIVSAEAWGRADHHHHHH

SEQ ID NO: 258 - protein, 243 aa, synthetic construct
MKPTLISVLVIIFILRGTRAQRVTQPEKLLSVFKGAPVELKCNYSYSGSPELFWYVQYSRQRLQLLLRHISRESIKGFTADLNKGETSFHLKKPFAQEEDSAMYYCALRSGGYQKVTFGTGTKLQVIPIQNPDPAVYQLRDSKSSDKSVCLFTDFDSQTNVSQSKDSDVYITDKCVLDMRSMDFKSNSAVAWSNKSDFACANAFNNSIIPEDTFFPSPESSGGGGSGHNYTTRNILPGLNITS

SEQ ID NO: 259 - protein, 268 aa, synthetic construct; identical to SEQ ID NO: 257

SEQ ID NO: 260 - protein, 241 aa, synthetic construct
GHNYTTRNILPGLNITSDWMTQSPLSLPVSLGDQASISCRSSQRLVHSNGNTYLHWYLQKPGQSPKLLIYRVSNRFPGVPDRFSGSGSGTDFTLKISRVEAEDLGIYFCSQSTHVPYTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECHHHHHH

SEQ ID NO: 261 - protein, 233 aa, synthetic construct
EVRLQQSGPDLIKPGASVKMSCKASGYTFTGYVMHVWKQRPGQGLEWIGFINPYNDDIQSNERFRGKATLTSDKSSTTAYMELSSLTSEDSAVYYCARGAGYNFDGAYRFFDFWGQGTTLTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCHHHHHH

SEQ ID NO: 262 - protein, 240 aa, synthetic construct
GHNYTTRNILPGLNITSQPQSVSESPGKTVTISCTRSSGNFASKYVQWYQQRPGSSPTTVIYENYQRPSGVPDRFSGSIDSSSNSATLTISGLKTEDEADYYCQSYDEVSWFGGGTQLTVLGQPAAKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECHHHHHH

SEQ ID NO: 263 - protein, 229 aa, synthetic construct
QMQLVQSGAEVKKPGASVKVSCKASGYTFASYYHWVRQAPGQGLEWGIINPSGGSTSYAQKFQGRVTMTRDTSTSTVYMELSRLRSDDTAVYYCASNRGGSYKSVGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCHHHHHH

SEQ ID NO: 264 - protein, 241 aa, synthetic construct
GHNYTTRNILPGLNITSDIVLTQSPASLAVSLGQRATISCRASKSVSTSGYSYMHWYQQKPGQPPKLLIYLASNLESGVPARFSGSGSGTDFTLDIHPVEEEDAATYYCQHSRELPYTFGGGTKLELKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECHHHHHH

SEQ ID NO: 265 - protein, 226 aa, synthetic construct
VQLQESGPELKKPGETVKISCKASGYTFTDYSMHWVKQAPGKGLKCMGWINTETTKSTYADDFKGRFALSLETSASTVYLQISNLKNEDTATYFCAREASYGEFDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCHHHHHH

SEQ ID NO: 266 - protein, 502 aa, synthetic construct
EVQVLESGGGLVQPGGSLRLSCAASGFTFSSYAMSWVRQAPGKGLEWVSAISGSGGSTNYADSVKGRFTISRDNSKNTLYLQMNSLRAEDTAVYYCAGSSGWSEYWGQGTLVTVSSGGGGSGGGGSGGGGSDIQMTQSPSSLSASVGDRVTITCRASQGIRNNLAWYQQKPGKAPKRLIYAASNLQSGVPSRFTGSGSGTEFTLIVSSLQPEDFATYYCLQHHSYPLTSGGGTKVEIKTTTPAPRPPTPAPTIASQPLSLRPEACRPAAGGAVHTRGLDFACDIYIWAPLAGTCGVLLLSLVITLYCRSKRSRLLHSDYMNMTPRRPGPTRKHYQPYAPPRDFAAYRSKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 267 - protein, 13 aa, synthetic construct
LEEKKGNYVVTDH

SEQ ID NO: 268 - protein, 237 aa, synthetic construct
LEEKKGNYVVTDHDIQLTQSPSTLSASVGDRVTITCRASESLDNYGIRFLTWFQQKPGKAPKLLMYAASNQGSGVPSRFSGSGSGTEFTLTISSLQPDDFATYYCQQTKEVPWSFGQGTKVEVKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECHHHHHH

SEQ ID NO: 269 - protein, 225 aa, synthetic construct; identical to SEQ ID NO: 72

SEQ ID NO: 270 - protein, 233 aa, synthetic construct
LEEKKGNYVVTDHDIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECHHHHHH

SEQ ID NO: 271 - protein, 229 aa, synthetic construct; identical to SEQ ID NO: 78

SEQ ID NO: 272 - protein, 503 aa, synthetic construct
EVQLLESGGGLVQPGGSMRLSCAASGFTFSNYWMNWVRQAPGKGLEWVAQIRLKSDNYATHYADSVKGRFTISRDDSKNTVYLQMNSLRAEDTGVYYCNDGPPSGYWGQGTLLTVSSGGGGSGGGGSGGGGSDIQMTQSPSSLSASVGDRVTITCRISENIYSYLAWYQQKPGKAPKLLIYNAKILVEGVPSRFSGSGSGTDFTLTISSLQPEDFATYYCQHHYTVPWTFGQGTKLEIKTTTPAPRPPTPAPTIASQPLSLRPEACRPAAGGAVHTRGLDFACDIYIWAPLAGTCGVLLLSLVITLYCRSKRSRLLHSDYMNMTPRRPGPTRKHYQPYAPPRDFAAYRSKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 273 - protein, 23 aa, synthetic construct
WTAHAIIRDFYNPLVAEAQKREL

SEQ ID NO: 274 - protein, 13 aa, synthetic construct
TAHAIIRDFYNPL

SEQ ID NO: 275 - protein, 10 aa, synthetic construct
LVAEAQKREL

SEQ ID NO: 276 - protein, 247 aa, synthetic construct
WTAHAIIRDFYNPLVAEAQKRELDIQLTQSPSTLSASVGDRVTITCRASESLDNYGIRFLTWFQQKPGKAPKLLMYAASNQGSGVPSRFSGSGSGTEFTLTISSLQPDDFATYYCQQTKEVPWSFGQGTKVEVKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECHHHHHH

SEQ ID NO: 277 - protein, 225 aa, synthetic construct; identical to SEQ ID NO: 72

SEQ ID NO: 278 - protein, 243 aa, synthetic construct
WTAHAIIRDFYNPLVAEAQKRELDIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECHHHHHH

SEQ ID NO: 279 - protein, 229 aa, synthetic construct; identical to SEQ ID NO: 78

SEQ ID NO: 280 - protein, 243 aa, synthetic construct
GHNYTTRNILPGLNITSDIVMTQSPSSLAVSAGEKVTISCLSSQSLFSSNTKRNYLNWYLQKPGQSPKLLIYHASTRLTGVPGRFIGSGSGTDFTLTVSTVQAEDLGDYFCQQHYETPLTFGDGTRLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECHHHHHH

SEQ ID NO: 281 - protein, 229 aa, synthetic construct
QIQLQESGPGLVKPSQSLSLTCSVTGFSITTGGYYWNWIRQFPGKKLEWMGYIYTSGRTSYNPSLKSRISITRDTSKNQFFLQLNSMTTEDTATYYCADMADKGGWFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCHHHHHH

SEQ ID NO: 282 - protein, 242 aa, synthetic construct
GHNYTTRNILPGLNITSDVLLTQSPLSLPVTLGQPASISCRSSQTIVHSNGNTYLEWFQQRPGQSPRLLIYQVSNRFSGVPDRFSGSGSGTDFTLKISRVEAEDVGVYYCFQGSHVPRTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECHHHHHH

SEQ ID NO: 283 - protein, 231 aa, synthetic construct
QIQLVQSGSELKKPGASVKVSCKASGYTFTNYGMNWVRQAPGQGLKWMGWINTNTGEPKYAEEFKGRFALSLDTSVSTAYLQINSLKAEDTAVYFCARGYGNYARGAWLAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCHHHHHH

SEQ ID NO: 284 - protein, 237 aa, synthetic construct
GHNYTTRNILPGLNITSDIQMTQTTSSLSASLGDRVTISCSASQGISNYLNWYQQKPDGTVKLLIYYTSSLHSGVPSRFSGSGSGTDYSLTISNLEPEDIATYYCQQYSKLPYTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECHHHHHH

SEQ ID NO: 285 - protein, 226 aa, synthetic construct
EVQLVESGGGLVKPGGSLKLSCAASGLTFSSYAMSWVRQTPEKRLEWVASISSGGFTYYPDSVKGRFTISRDNARNILYLQMSSLRSEDTAMYYCARDEVRGYLDVWGAGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCHHHHHH

SEQ ID NO: 286 - protein, 251 aa, synthetic construct
GHNYTTRNILPGLNITSMDVQLQESGGGSVQAGGSLRLSCPASGYTFSHYCMGWNRQAPGKEREEVATIDTDDTPTYADSVMGRFTISRDNANNALYLQMNDLKPEDTSMYYCAIWMKLRGSCHDRRLEVRGQGTQVTVSINASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCHHHHHH

SEQ ID NO: 287 - protein, 241 aa, synthetic construct
GHNYTTRNILPGLNITSQSVLTQPPSVSAAPGQKVTIPCSGSRSNVGNYYVSWYQQLPGTAPKLLIYDNNKRPSGIPDRFSGSKSGTSATLGITGLQTGDEADYFCGTWDGSLSAHVFGTGTKVTVLGRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECHHHHHH

SEQ ID NO: 288 - protein, 229 aa, synthetic construct
QVQLVQSGSELKKPGASVRVSCTASGYTFTSYYMHWVRQAPGQGLEWMGVINPNAGSTRYAQKFQGRVTMSTDTSTSTAYMDLSSLRSEDTAVYYCARGMYRSLLFYDPWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCHHHHHH

SEQ ID NO: 289 - protein, 237 aa, synthetic construct
GHNYTTRNILPGLNITSDIQMTQSPSSLSASVGDRVTITCLASQPIGIWLAWYQQKPGKAPKLLIYAATSLADGVPSRFSGSGSGTDFTFTISSLQPEDIATYYCQQLYSSPWTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECHHHHHH

SEQ ID NO: 290 - protein, 230 aa, synthetic construct
QVQLVQSGAEVKKPGASVKVSCKASGYTFTDYYMNWVRQAPGQGLEWMGDIIPNKGGSKYNQKFKDRVTMTTDTSTSTAYMELRSLRSDDTAVYYCATWGYGSSYRWFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCHHHHHH

SEQ ID NO: 291 - protein, 242 aa, synthetic construct
GHNYTTRNILPGLNITSDIVMTQSPLSLPVTLGEPASISCRSSQSLVHRNGITFFHWYLQKPGQSPKLLIYKISNRFSGVPDRFSGSGSGTDFTLKISRVEAEDVGVYFCSQGTHVPPTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECHHHHHH

SEQ ID NO: 292 - protein, 228 aa, synthetic construct
QVQLQQSGPGLVKPSQTLSITCTVSGFSLNNYGVHWVRQPPGKGLEWLGVIWSAGTTVYNAAAISRLTISKDTSKNQVSFKMSSLTAADTAVYYCAKDGSRYYTAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCHHHHHH

SEQ ID NO: 293 - protein, 236 aa, synthetic construct
GHNYTTRNILPGLNITSDIQMTQSPSSLSASVGDRVTITCRASSSVSYMHWYQQKPGKAPKPLIYATSNLASGVPSRFSGSGSGTDFTFTISSLQPEDIATYYCQQWSSNPLTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECHHHHHH

SEQ ID NO: 294 - protein, 228 aa, synthetic construct
QVQLQESGPGLVRPSQTLSLTCTVSGFSDYYMNWVRQPPGRGLEWIGFISNKPNGHTTEYSASVKGRVTMLRDTSKNQFSLRLSSVTAADTAVYYCARDKGIRWYFDVWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCHHHHHH

SEQ ID NO: 295 - protein, 237 aa, synthetic construct
GHNYTTRNILPGLNITSDIQMTQSPSSLSASLGERVSLTCRASQEISGYLSWHQQKPDGTIKRLLYSTSTLDSGVPKRFSGSRSGSDYSLTISSLESEDFADYYCLQYAISPPTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECHHHHHH

SEQ ID NO: 296 - protein, 230 aa, synthetic construct
QVTLKESGPGILQTSQTLSLTCSFSGFSLSTSGMGVSWIRQPSGKGLEWLAHIYWDDDKRYNPSLKSRLTISKDTSRNQVFLKITSVDTTDTATYYCALNYYNSTYNFDFWGQGTTLTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCHHHHHH

SEQ ID NO: 297 - protein, 237 aa, synthetic construct
GHNYTTRNILPGLNITSDIQMTQSPSSLSASVGDRVTITCKASQNVRTTVDWYQQKPGKAPKLLIYLASNRHTGVPSRFSGSGSGTDFTLTISSLQPEDFATYYCLQHRNYPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECHHHHHH

SEQ ID NO: 298 - protein, 229 aa, synthetic construct
EVQLVESGGGLVKPGGSLRLSCAASGFTFSSYDMSWVRQAPGKGLEWVSTISSGGNYNYYPDSVKGRFTISRDNAKNSLYLQMNSLRAEDTAVYYCARHYDYPDYAMDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCHHHHHH

SEQ ID NO: 299 - protein, 242 aa, synthetic construct
GHNYTTRNILPGLNITSDIVMTQAAPSVPVTPGESVSISCRSSKSLLHSNGNTYLYWFLQRPGQSPQRLIYYMSNLASGVPDRFSGRGSGTDFTLRISRVEAEDVGVYYCMQSLEYPLTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECHHHHHH

SEQ ID NO: 300 - protein, 232 aa, synthetic construct
QVTLKESGPGILQPSQTLSLTCSFSGFSLSTVGMGVGWSRQPSGKGLEWLAHIWWDDEDKYYNPALKSRLTISKDTSKNQVFLKIANVDTADTATYYCTRIGTAQATDALDYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCHHHHHH

SEQ ID NO: 301 - protein, 270 aa, synthetic construct
GHNYTTRNILPGLNITSAQSVAQPEDLVNVAEGNPLTVKCTYSVSGNPYLFWYVQYPNRGLQFLLKYLGDSALVKGSYGFEAEFNKSQTSFHLKKPSALVSDSALYFCAVRDINSGAGSYQLTFGKGTKLSVIPGGGGSGGGGSGGGGSSAVISQKPSRDIKQRGTSLTIQCQVDKRLALMFWYRQQPGQSPTLIATAWTGGEATYESGFVIDKFPISRPNLTFSTLTVSNMSPEDSSIYLCSVGGSGAADTQYFGPGTRLTVLHHHHHH

SEQ ID NO: 302 - protein, 275 aa, synthetic construct
HHHHHHAQSVAQPEDLVNVAEGNPLTVKCTYSVSGNPYLFWYVQYPNRGLQFLLKYLGDSALVKGSYGFEAEFNKSQTSFHLKKPSALVSDSALYFCAVRDINSGAGSYQLTFGKGTKLSVIPGGGGSGGGGSGGGGSSAVISQKPSRDIKQRGTSLTIQCQVDKRLALMFWYRQQPGQSPTLIATAWTGGEATYESGFVIDKFPISRPNLTFSTLTVSNMSPEDSSIYLCSVGGSGAADTQYFGPGTRLTVLGGGGSGHNYTTRNILPGLNITS

SEQ ID NO: 303 - protein, 280 aa, synthetic construct
GHNYTTRNILPGLNITSSQTIHQWPATLVQPVGSPLSLECTVEGTSNPNLYWYRQAAGRGLELLFYSVGIGQISSEVPQNLFASRPQDRQFILSSKKLLLSDSGFYLCAWSIGAEQFFGPGTRLTVLEDLKNGSADDAKKDAAKKDGKSGGGGSGGGGSGGGGSQKEVEQNSGPLSVPEGAIASLNCTYSDRGSQSFFWYRQYSGKSPELIMSIYSNGDKEDGRFTAQLNKASQYVSLLIRDSQPSDSATYLCAVSKGYKMFGDGTQLVVKPNIHHHHHH

SEQ ID NO: 304 - protein, 285 aa, synthetic construct
HHHHHHSQTIHQWPATLVQPVGSPLSLECTVEGTSNPNLYWYRQAAGRGLELLFYSVGIGQISSEVPQNLFASRPQDRQFILSSKKLLLSDSGFYLCAWSIGAEQFFGPGTRLTVLEDLKNGSADDAKKDAAKKDGKSGGGGSGGGGSGGGGSQKEVEQNSGPLSVPEGAIASLNCTYSDRGSQSFFWYRQYSGKSPELIMSIYSNGDKEDGRFTAQLNKASQYVSLLIRDSQPSDSATYLCAVSKGYKMFGDGTQLVVKPNIGGGGSGHNYTTRNILPGLNITS

SEQ ID NO: 305 - protein, 165 aa, synthetic construct
GHNYTTRNILPGLNITSSVLHLVPINATSKDDSDVTEVMWQPALRRGRGLQAQGYGVRIQDAGVYLLYSQVLFQDVTFTMGQVVSREGQGRQETLFRCIRSMPSHPDRAYNSCYSAGVFHLHQGDILSVIIPRARAKLNLSPHGTFLGFVKLSGGGSDPHHHHHH

SEQ ID NO: 306 - protein, 165 aa, synthetic construct
HHHHHHSVLHLVPINATSKDDSDVTEVMWQPALRRGRGLQAQGYGVRIQDAGVYLLYSQVLFQDVTFTMGQVVSREGQGRQETLFRCIRSMPSHPDRAYNSCYSAGVFHLHQGDILSVIIPRARAKLNLSPHGTFLGFVKLSGGGSDPGHNYTTRNILPGLNITS

SEQ ID NO: 307 - protein, 357 aa, synthetic construct
EVQLLESGGGLVQPGGSLRLSCAASGFTFRNHDMGWVRQAPGKGLEWVSAISGSGGSTYYANSVKGRFTISRDNSKNTLYLQMNSLRAEDTAVYYCAEPKPMDTEFDYRSPGTLVTVSSEPKSCDKTHTCPPCPAPELLGGPDVFLFPPKPKDTLMISRTPEVTCVVVDVSHEDPEVKFNWYVDGVEVHNAKTKPREEQYNSTYRVVSVLTVLHQDWLNGKEYKCKVSNKALPAPEEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKHHHHHH

SEQ ID NO: 308 - protein, 455 aa, synthetic construct
EVQLLESGGGLVQPGGSLRLSCAASGFTFRNHDMGWVRQAPGKGLEWVSAISGSGGSTYYANSVKGRFTISRDNSKNTLYLQMNSLRAEDTAVYYCAEPKPMDTEFDYRSPGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPDVFLFPPKPKDTLMISRTPEVTCVVVDVSHEDPEVKFNWYVDGVEVHNAKTKPREEQYNSTYRVVSVLTVLHQDWLNGKEYKCKVSNKALPAPEEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKHHHHHH

SEQ ID NO: 309 - protein, 213 aa, synthetic construct
DIQMTQSPSSLSASVGDRVTITCRASQYIYDYLNWYQQKPGKAPKLLIYAASYLQSGVPSRFSGSGSGTDFTLTISSLQPEDFATYYCQQYHHPSTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 310 - protein, 214 aa, synthetic construct
DIQMTQSPSSLSASVGDRVTITCRASQSISSYLNWYQQKPGKAPKLLIYSASALQSGVPSRFSGSGSGTDFTLTISSLQPEDFVTYYCQQLGQGPYTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 311 - protein, 214 aa, synthetic construct
DIQMTQSPSSLSASVGDRVTITCRASQSISSYLNWYQQKPGKAPKLLIYSASSLQSGVPSRFSGSGSGTDFTLTISSLQPEDFATYYCQQYGYSPHTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 312 - protein, 214 aa, synthetic construct
DIQMTQSPSSLSASVGDRVTITCRASQSISSYLNWYQQKPGKAPKLLIYDSSALQSGVPSRFSGSGSGTDFTLTISSLQPEDFATYYCQQSVALPYTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 313 - protein, 596 aa, synthetic construct
EVQLLESGGGLVQPGGSLRLSCAASGFTFRNHDMGWVRQAPGKGLEWVSAISGSGGSTYYANSVKGRFTISRDNSKNTLYLQMNSLRAEDTAVYYCAEPKPMDTEFDYRSPGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPDVFLFPPKPKDTLMISRTPEVTCVVVDVSHEDPEVKFNWYVDGVEVHNAKTKPREEQYNSTYRVVSVLTVLHQDWLNGKEYKCKVSNKALPAPEEKTISKAKGQPSGDIKLQQSGAELARPGASVKMSCKTSGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSSVEGGSGGSGGSGGSGGVDDIQLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGAGTKLELKHHHHHH

SEQ ID NO: 314 - protein, 213 aa, synthetic construct; identical to SEQ ID NO: 309

SEQ ID NO: 315 - protein, 214 aa, synthetic construct; identical to SEQ ID NO: 310

SEQ ID NO: 316 - protein, 214 aa, synthetic construct; identical to SEQ ID NO: 311

SEQ ID NO: 317 - protein, 214 aa, synthetic construct; identical to SEQ ID NO: 312

SEQ ID NO: 318 - protein, 596 aa, synthetic construct
EVQLLESGGGLVQPGGSLRLSCAASGVRISDEDMGWVRQAPGKGLEWVSAIDDAGDSTYYADSVKGRFTISRDNSKNTLYLQMNSLRAEDTAVYYCAKVYPYDTVFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPDVFLFPPKPKDTLMISRTPEVTCVVVDVSHEDPEVKFNWYVDGVEVHNAKTKPREEQYNSTYRVVSVLTVLHQDWLNGKEYKCKVSNKALPAPEEKTISKAKGQPSGDIKLQQSGAELARPGASVKMSCKTSGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSSVEGGSGGSGGSGGSGGVDDIQLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGAGTKLELKHHHHHH

SEQ ID NO: 319 - protein, 214 aa, synthetic construct
DIQMTQSPSSLSASVGDRVTITCRASQSISSYLNWYQQKPGKAPKLLIYAASDLQSGVPSRFSGSGSGTDFTLTISSLQPEDFATYYCQQSIWAPYTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 320 - protein, 373 aa, synthetic construct
EVQLLESGGGLVQPGGSLRLSCAASGVRISDEDMGWVRQAPGKGLEWVSAIDDAGDSTYYADSVKGRFTISRDNSKNTLYLQMNSLRAEDTAVYYCAKVYPYDTVFDYWGQGTLVTVSSGGGGSDIKLQQSGAELARPGASVKMSCKTSGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSSVEGGSGGSGGSGGSGGVDDIQLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGAGTKLELKHHHHHH

SEQ ID NO: 321 - protein, 373 aa, synthetic construct
EVQLLESGGGLVQPGGSLRLSCAASGFTFRNHDMGWVRQAPGKGLEWVSAISGSGGSTYYANSVKGRFTISRDNSKNTLYLQMNSLRAEDTAVYYCAEPKPMDTEFDYRSPGTLVTVSSGGGGSDIKLQQSGAELARPGASVKMSCKTSGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSSVEGGSGGSGGSGGSGGVDDIQLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGAGTKLELKHHHHHH

SEQ ID NO: 322 - protein, 494 aa, synthetic construct
DIQMTQSPSSLSASVGDRVTITCRASQYIYDYLNWYQQKPGKAPKLLIYAASYLQSGVPSRFSGSGSGTDFTLTISSLQPEDFATYYCQQYHHPSTFGQGTKVEIKGGGGSGGGGSGGGGSEVQLLESGGGLVQPGGSLRLSCAASGFTFRNHDMGWVRQAPGKGLEWVSAISGSGGSTYYANSVKGRFTISRDNSKNTLYLQMNSLRAEDTAVYYCAEPKPMDTEFDYRSPGTLVTVSSGGGGSDIKLQQSGAELARPGASVKMSCKTSGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSSVEGGSGGSGGSGGSGGVDDIQLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGAGTKLELKHHHHHH

SEQ ID NO: 323 - protein, 502 aa, synthetic construct
DIVMTQSPATLSLSPGERATLSCRSSKSLQNVNGNTYLYWFQQKPGQSPQLLIYRMSNLNSGVPDRFSGSGSGTEFTLTISSLEPEDFAVYYCMQHLEYPITFGAGTKLEIKGGGGSGGGGSGGGGSEVQLVESGGGLVKPGGSLKLSCAASGYTFTSYVMHWVRQAPGKGLEWIGYINPYNDGTKYNEKFQGRVTISSDKSISTAYMELSSLRSEDTAMYYCARGTYYYGTRVFDYWGQGTLVTVSSGGGGSDIKLQQSGAELARPGASVKMSCKTSGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSSVEGGSGGSGGSGGSGGVDDIQLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGAGTKLELKHHHHHH

SEQ ID NO: 324 - protein, 497 aa, synthetic construct
EVQLLESGGGLVQPGGSLRLSCAASGFRISDEDMGWVRQAPGKGLEWVSAIDDAGDSTYYADSVKGRFTISRDNSKNTLYLQMNSLRAEDTAVYYCAKVYPYDTVFDYWGQGTLVTVSSGGGGSGGGGSGGGGSTDIQMTQSPSSLSASVGDRVTITCRASQSISSYLNWYQQKPGKAPKLLIYAASDLQSGVPSRFSGSGSGTDFTLTISSLQPEDFATYYCQQSIWAPYTFGQGTKVEIKRGGGGSDIKLQQSGAELARPGASVKMSCKTSGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSSVEGGSGGSGGSGGSGGVDDIQLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGAGTKLELKHHHHHH

SEQ ID NO: 325 - protein, 496 aa, synthetic construct
DIQMTQSPSSLSASVGDRVTITCRASQSISSYLNWYQQKPGKAPKLLIYAASDLQSGVPSRFSGSGSGTDFTLTISSLQPEDFATYYCQQSIWAPYTFGQGTKVEIKRGGGGSGGGGSGGGGSEVQLLESGGGLVQPGGSLRLSCAASGFRISDEDMGWVRQAPGKGLEWVSAIDDAGDSTYYADSVKGRFTISRDNSKNTLYLQMNSLRAEDTAVYYCAKVYPYDTVFDYWGQGTLVTVSSGGGGSDIKLQQSGAELARPGASVKMSCKTSGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSSVEGGSGGSGGSGGSGGVDDIQLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGAGTKLELKHHHHHH

SEQ ID NO: 326 - protein, 498 aa, synthetic construct
EVQLLESGGGLVQPGGSLRLSCAASGFRISDEDMGWVRQAPGKGLEWVSAIDDAGDSTYYADSVKGRFTISRDNSKNTLYLQMNSLRAEDTAVYYCAKVYPYDTVFDYWGQGTLVTVSSGGGGSGGGGSGGGGSTDIQMTQSPSSLSASVGDRVTITCRASQSISSYLNWYQQKPGKAPKLLIYAASDLQSGVPSRFSGSGSGTDFTLTISSLQPEDFATYYCQQSIWAPYTFGQGTKVEIKRGGGGSQVTLKESGPGILQPSQTLSLTCSFSGFSLRTSGMGVGWIRQPSGKGLEWLAHIWWDDDKRYNPALKSRLTISKDTSSNQVFLKIASVDTADTATYYCAQINPAWFAYWGQGTLVTVSSGGGGSGGGGSGGGGSDIVLTQSPASLAVSLGQRATISCKASQSVDFDGDSFMNWYQQKPGQPPKLLIYTTSNLESGIPARFSASGSGTDFTLNIHPVEEEDTATYYCQQSNEDPYTFGGGTKLEIKHHHHHH

SEQ ID NO: 327 - protein, 497 aa, synthetic construct
DIQMTQSPSSLSASVGDRVTITCRASQSISSYLNWYQQKPGKAPKLLIYAASDLQSGVPSRFSGSGSGTDFTLTISSLQPEDFATYYCQQSIWAPYTFGQGTKVEIKRGGGGSGGGGSGGGGSEVQLLESGGGLVQPGGSLRLSCAASGFRISDEDMGWVRQAPGKGLEWVSAIDDAGDSTYYADSVKGRFTISRDNSKNTLYLQMNSLRAEDTAVYYCAKVYPYDTVFDYWGQGTLVTVSSGGGGSQVTLKESGPGILQPSQTLSLTCSFSGFSLRTSGMGVGWIRQPSGKGLEWLAHIWWDDDKRYNPALKSRLTISKDTSSNQVFLKIASVDTADTATYYCAQINPAWFAYWGQGTLVTVSSGGGGSGGGGSGGGGSDIVLTQSPASLAVSLGQRATISCKASQSVDFDGDSFMNWYQQKPGQPPKLLIYTTSNLESGIPARFSASGSGTDFTLNIHPVEEEDTATYYCQQSNEDPYTFGGGTKLEIKHHHHHH

SEQ ID NO: 328 - protein, 483 aa, synthetic construct
EVQLLESGGGLVQPGGSLRLSCAASGFRISDEDMGWVRQAPGKGLEWVSAIDDAGDSTYYADSVKGRFTISRDNSKNTLYLQMNSLRAEDTAVYYCAKVYPYDTVFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTSPGQPSGDIKLQQSGAELARPGASVKMSCKTSGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSSVEGGSGGSGGSGGSGGVDDIQLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGAGTKLELKHHHHHH

SEQ ID NO: 329 - protein, 214 aa, synthetic construct; identical to SEQ ID NO: 319

SEQ ID NO: 330 - protein, 214 aa, synthetic construct
DIQMTQSPSSLSASVGDRVTITCRASQSISSYLNWYQQKPGKAPKLLIYDASSLQSGVPSRFSGSGSGTDFTLTISSLQPEDFATYYCQQGYYLPGTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 331 - protein, 214 aa, synthetic construct
DIQMTQSPSSLSASVGDRVTITCRASQSISSYLNWYQQKPGKAPKLLIYAASSLQSGVPSRFSGSGSGTDFTLTISSLQPEDFATYYCQQSSSHPHTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 332 - protein, 214 aa, synthetic construct
DIQMTQSPSSLSASVGDRVTITCRASQSISSYLNWYQQKPGKAPKLLIYAASSLQSGVPSRFSGSGSGTDFTLTISSLQPEDFATYYCQQTYEHPYTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 333 - protein, 214 aa, synthetic construct
DIQMTQSPSSLSASVGDRVTITCRASQSISSYLNWYQQKPGKAPKLLIYSASNLQSGVPSRFSGSGSGTDFTLTISSLQPEDFATYYCQQVDGSPQTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 334 - protein, 484 aa, synthetic construct
EVQLLESGGGLVQPGGSLRLSCAASGFRISDEDMGWVRQAPGKGLEWVSAIDDAGDSTYYADSVKGRFTISRDNSKNTLYLQMNSLRAEDTAVYYCAKVYPYDTVFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTSPGQPSGQVTLKESGPGILQPSQTLSLTCSFSGFSLRTSGMGVGWIRQPSGKGLEWLAHIWWDDDKRYNPALKSRLTISKDTSSNQVFLKIASVDTADTATYYCAQINPAWFAYWGQGTLVTVSSGGGGSGGGGSGGGGSDIVLTQSPASLAVSLGQRATISCKASQSVDFDGDSFMNWYQQKPGQPPKLLIYTTSNLESGIPARFSASGSGTDFTLNIHPVEEEDTATYYCQQSNEDPYTFGGGTKLEIKHHHHHH

SEQ ID NO: 335 - protein, 214 aa, synthetic construct; identical to SEQ ID NO: 319

SEQ ID NO: 336 - protein, 214 aa, synthetic construct; identical to SEQ ID NO: 330

SEQ ID NO: 337 - protein, 214 aa, synthetic construct; identical to SEQ ID NO: 331

SEQ ID NO: 338 - protein, 214 aa, synthetic construct; identical to SEQ ID NO: 332

SEQ ID NO: 339 - protein, 214 aa, synthetic construct; identical to SEQ ID NO: 333

SEQ ID NO: 340 - protein, 453 aa, synthetic construct
EVQLLESGGGLVQPGGSMRLSCAASGFTFSNYWMNWVRQAPGKGLEWVAQIRLKSDNYATHYADSVKGRFTISRDDSKNTVYLQMNSLRAEDTGVYYCNDGPPSGYWGQGTLLTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPDVFLFPPKPKDTLMISRTPEVTCVVVDVSHEDPEVKFNWYVDGVEVHNAKTKPREEQYNSTYRVVSVLTVLHQDWLNGKEYKCKVSNKALPAPEEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKHHHHHH

SEQ ID NO: 341 - protein, 214 aa, synthetic construct
DIQMTQSPSSLSASVGDRVTITCRISENIYSYLAWYQQKPGKAPKLLIYNAKILVEGVPSRFSGSGSGTDFTLTISSLQPEDFATYYCQHHYTVPWTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 342 - protein, 493 aa, synthetic construct
DIQMTQSPSSLSASVGDRVTITCRISENIYSYLAWYQQKPGKAPKLLIYNAKILVEGVPSRFSGSGSGTDFTLTISSLQPEDFATYYCQHHYTVPWTFGQGTKLEIKGGGGSGGGGSGGGGSEVQLLESGGGLVQPGGSMRLSCAASGFTFSNYWMNWVRQAPGKGLEWVAQIRLKSDNYATHYADSVKGRFTISRDDSKNTVYLQMNSLRAEDTGVYYCNDGPPSGYWGQGTLLTVSSGGGGSDIKLQQSGAELARPGASVKMSCKTSGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSSVEGGSGGSGGSGGSGGVDDIQLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGAGTKLELKHHHHHH

SEQ ID NO: 343 - protein, 494 aa, synthetic construct
DIQMTQSPSSLSASVGDRVTITCRISENIYSYLAWYQQKPGKAPKLLIYNAKILVEGVPSRFSGSGSGTDFTLTISSLQPEDFATYYCQHHYTVPWTFGQGTKLEIKGGGGSGGGGSGGGGSEVQLLESGGGLVQPGGSMRLSCAASGFTFSNYWMNWVRQAPGKGLEWVAQIRLKSDNYATHYADSVKGRFTISRDDSKNTVYLQMNSLRAEDTGVYYCNDGPPSGYWGQGTLLTVSSGGGGSQVTLKESGPGILQPSQTLSLTCSFSGFSLRTSGMGVGWIRQPSGKGLEWLAHIWWDDDKRYNPALKSRLTISKDTSSNQVFLKIASVDTADTATYYCAQINPAWFAYWGQGTLVTVSSGGGGSGGGGSGGGGSDIVLTQSPASLAVSLGQRATISCKASQSVDFDGDSFMNWYQQKPGQPPKLLIYTTSNLESGIPARFSASGSGTDFTLNIHPVEEEDTATYYCQQSNEDPYTFGGGTKLEIKHHHHHH

SEQ ID NO: 344 - protein, 452 aa, synthetic construct
EVQVLESGGGLVQPGGSLRLSCAASGFTFSSYAMSWVRQAPGKGLEWVSAISGSGGSTNYADSVKGRFTISRDNSKNTLYLQMNSLRAEDTAVYYCAGSSGWSEYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPDVFLFPPKPKDTLMISRTPEVTCVVVDVSHEDPEVKFNWYVDGVEVHNAKTKPREEQYNSTYRVVSVLTVLHQDWLNGKEYKCKVSNKALPAPEEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKHHHHHH

SEQ ID NO: 345 - protein, 214 aa, synthetic construct
DIQMTQSPSSLSASVGDRVTITCRASQGIRNNLAWYQQKPGKAPKRLIYAASNLQSGVPSRFTGSGSGTEFTLIVSSLQPEDFATYYCLQHHSYPLTSGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 346 - protein, 493 aa, synthetic construct
DIQMTQSPSSLSASVGDRVTITCRASQGIRNNLAWYQQKPGKAPKRLIYAASNLQSGVPSRFTGSGSGTEFTLIVSSLQPEDFATYYCLQHHSYPLTSGGGTKVEIKRGGGGSGGGGSGGGGSEVQVLESGGGLVQPGGSLRLSCAASGFTFSSYAMSWVRQAPGKGLEWVSAISGSGGSTNYADSVKGRFTISRDNSKNTLYLQMNSLRAEDTAVYYCAGSSGWSEYWGQGTLVTVSSGGGGSDIKLQQSGAELARPGASVKMSCKTSGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSSVEGGSGGSGGSGGSGGVDDIQLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGAGTKLELKHHHHHH

SEQ ID NO: 347 - protein, 493 aa, synthetic construct
DIQMTQSPSSLSASVGDRVTITCRASQGIRNNLAWYQQKPGKAPKRLIYAASNLQSGVPSRFTGSGSGTEFTLIVSSLQPEDFATYYCLQHHSYPLTSGGGTKVEIKGGGGSGGGGSGGGGSEVQVLESGGGLVQPGGSLRLSCAASGFTFSSYAMSWVRQAPGKGLEWVSAISGSGGSTNYADSVKGRFTISRDNSKNTLYLQMNSLRAEDTAVYYCAGSSGWSEYWGQGTLVTVSSGGGGSQVTLKESGPGILQPSQTLSLTCSFSGFSLRTSGMGVGWIRQPSGKGLEWLAHIWWDDDKRYNPALKSRLTISKDTSSNQVFLKIASVDTADTATYYCAQINPAWFAYWGQGTLVTVSSGGGGSGGGGSGGGGSDIVLTQSPASLAVSLGQRATISCKASQSVDFDGDSFMNWYQQKPGQPPKLLIYTTSNLESGIPARFSASGSGTDFTLNIHPVEEEDTATYYCQQSNEDPYTFGGGTKLEIKHHHHHH

SEQ ID NO: 348 - protein, 226 aa, synthetic construct
QVQLQQSGSELKKPGASVKISCKASGYSFTDYIILWVRQNPGKGLEWIGHIDPYYGSSNYNLKFKGRVTITADQSTTTAYMELSSLRSEDTAVYYCGRSKRDYFDYWGQGTTLTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCHHHHHH

SEQ ID NO: 349 - protein, 220 aa, synthetic construct
DIQMTQSPSSLSASIGDRVTITCKASQDINSYLSWFQQKPGKAPKLLIYRANRLVDGVPSRFSGSGSGTDYTLTISSLQPEDFAVYYCLQYDEFPYTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECHHHHHH

SEQ ID NO: 350 - protein, 509 aa, synthetic construct
DIQMTQSPSSLSASVGDRVTITCRASQYIYDYLNWYQQKPGKAPKLLIYAASYLQSGVPSRFSGSGSGTDFTLTISSLQPEDFATYYCQQYHHPSTFGQGTKVEIKRGGGGSGGGGSGGGGSEVQLLESGGGLVQPGGSLRLSCAASGFTFRNHDMGWVRQAPGKGLEWVSAISGSGGSTYYANSVKGRFTISRDNSKNTLYLQMNSLRAEDTAVYYCAEPKPMDTEFDYRSPGTLVTVSSVEGGSGGSGGSGGSGGVDQVTLKESGPGILQPSQTLSLTCSFSGFSLRTSGMGVGWIRQPSGKGLEWLAHIWWDDDKRYNPALKSRLTISKDTSSNQVFLKIASVDTADTATYYCAQINPAWFAYWGQGTLVTVSSGGGGSGGGGSGGGGSDIVLTQSPASLAVSLGQRATISCKASQSVDFDGDSFMNWYQQKPGQPPKLLIYTTSNLESGIPARFSASGSGTDFTLNIHPVEEEDTATYYCQQSNEDPYTFGGGTKLEIKHHHHHH

SEQ ID NO: 351 - protein, 509 aa, synthetic construct
DIVLTQSPASLAVSLGQRATISCKASQSVDFDGDSFMNWYQQKPGQPPKLLIYTTSNLESGIPARFSASGSGTDFTLNIHPVEEEDTATYYCQQSNEDPYTFGGGTKLEIKGGGGSGGGGSGGGGSQVTLKESGPGILQPSQTLSLTCSFSGFSLRTSGMGVGWIRQPSGKGLEWLAHIWWDDDKRYNPALKSRLTISKDTSSNQVFLKIASVDTADTATYYCAQINPAWFAYWGQGTLVTVSSVEGGSGGSGGSGGSGGVDEVQLLESGGGLVQPGGSLRLSCAASGFTFRNHDMGWVRQAPGKGLEWVSAISGSGGSTYYANSVKGRFTISRDNSKNTLYLQMNSLRAEDTAVYYCAEPKPMDTEFDYRSPGTLVTVSSGGGGSGGGGSGGGGSDIQMTQSPSSLSASVGDRVTITCRASQYIYDYLNWYQQKPGKAPKLLIYAASYLQSGVPSRFSGSGSGTDFTLTISSLQPEDFATYYCQQYHHPSTFGQGTKVEIKRHHHHHH

SEQ ID NO: 352 - protein, 699 aa, synthetic construct
QVTLKESGPGILQPSQTLSLTCSFSGFSLRTSGMGVGWIRQPSGKGLEWLAHIWWDDDKRYNPALKSRLTISKDTSSNQVFLKIASVDTADTATYYCAQINPAWFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPPVAGPSVFLFPPKPKDTLMISRTPEVTCVVVGVSHEDPEVKFNWYVDGVEVHNAKTKPREEQYQSTYRVVSVLTVLHQDWLNGKEYKCKVSNKQLPSPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSDIQMTQSPSSLSASVGDRVTITCRASQYIYDYLNWYQQKPGKAPKLLIYAASYLQSGVPSRFSGSGSGTDFTLTISSLQPEDFATYYCQQYHHPSTFGQGTKVEIKRGGGGSGGGGSGGGGSEVQLLESGGGLVQPGGSLRLSCAASGFTFRNHDMGWVRQAPGKGLEWVSAISGSGGSTYYANSVKGRFTISRDNSKNTLYLQMNSLRAEDTAVYYCAEPKPMDTEFDYRSPGTLVTVSSHHHHHH

SEQ ID NO: 353 - protein, 699 aa, synthetic construct
QVTLKESGPGILQPSQTLSLTCSFSGFSLRTSGMGVGWIRQPSGKGLEWLAHIWWDDDKRYNPALKSRLTISKDTSSNQVFLKIASVDTADTATYYCAQINPAWFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTSPPSPAPPVAGPSVFLFPPKPKDTLMISRTPEVTCVVVGVSHEDPEVKFNWYVDGVEVHNAKTKPREEQYQSTYRVVSVLTVLHQDWLNGKEYKCKVSNKQLPSPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSDIQMTQSPSSLSASVGDRVTITCRASQYIYDYLNWYQQKPGKAPKLLIYAASYLQSGVPSRFSGSGSGTDFTLTISSLQPEDFATYYCQQYHHPSTFGQGTKVEIKRGGGGSGGGGSGGGGSEVQLLESGGGLVQPGGSLRLSCAASGFTFRNHDMGWVRQAPGKGLEWVSAISGSGGSTYYANSVKGRFTISRDNSKNTLYLQMNSLRAEDTAVYYCAEPKPMDTEFDYRSPGTLVTVSSHHHHHH

SEQ ID NO: 354 - protein, 592 aa, synthetic construct
QVTLKESGPGILQPSQTLSLTCSFSGFSLRTSGMGVGWIRQPSGKGLEWLAHIWWDDDKRYNPALKSRLTISKDTSSNQVFLKIASVDTADTATYYCAQINPAWFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPPVAGPSVFLFPPKPKDTLMISRTPEVTCVVVGVSHEDPEVKFNWYVDGVEVHNAKTKPREEQYQSTYRVVSVLTVLHQDWLNGKEYKCKVSNKQLPSPIEKTISKAKGGGGSDIQMTQSPSSLSASVGDRVTITCRASQYIYDYLNWYQQKPGKAPKLLIYAASYLQSGVPSRFSGSGSGTDFTLTISSLQPEDFATYYCQQYHHPSTFGQGTKVEIKRGGGGSGGGGSGGGGSEVQLLESGGGLVQPGGSLRLSCAASGFTFRNHDMGWVRQAPGKGLEWVSAISGSGGSTYYANSVKGRFTISRDNSKNTLYLQMNSLRAEDTAVYYCAEPKPMDTEFDYRSPGTLVTVSSHHHHHH

SEQ ID NO: 355 - protein, 592 aa, synthetic construct
QVTLKESGPGILQPSQTLSLTCSFSGFSLRTSGMGVGWIRQPSGKGLEWLAHIWWDDDKRYNPALKSRLTISKDTSSNQVFLKIASVDTADTATYYCAQINPAWFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTSPPSPAPPVAGPSVFLFPPKPKDTLMISRTPEVTCVVVGVSHEDPEVKFNWYVDGVEVHNAKTKPREEQYQSTYRVVSVLTVLHQDWLNGKEYKCKVSNKQLPSPIEKTISKAKGGGGSDIQMTQSPSSLSASVGDRVTITCRASQYIYDYLNWYQQKPGKAPKLLIYAASYLQSGVPSRFSGSGSGTDFTLTISSLQPEDFATYYCQQYHHPSTFGQGTKVEIKRGGGGSGGGGSGGGGSEVQLLESGGGLVQPGGSLRLSCAASGFTFRNHDMGWVRQAPGKGLEWVSAISGSGGSTYYANSVKGRFTISRDNSKNTLYLQMNSLRAEDTAVYYCAEPKPMDTEFDYRSPGTLVTVSSHHHHHH

SEQ ID NO: 356 - protein, 604 aa, synthetic construct
QVTLKESGPGILQPSQTLSLTCSFSGFSLRTSGMGVGWIRQPSGKGLEWLAHIWWDDDKRYNPALKSRLTISKDTSSNQVFLKIASVDTADTATYYCAQINPAWFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPPVAGPSVFLFPPKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSDIQMTQSPSSLSASVGDRVTITCRASQYIYDYLNWYQQKPGKAPKLLIYAASYLQSGVPSRFSGSGSGTDFTLTISSLQPEDFATYYCQQYHHPSTFGQGTKVEIKRGGGGSGGGGSGGGGSEVQLLESGGGLVQPGGSLRLSCAASGFTFRNHDMGWVRQAPGKGLEWVSAISGSGGSTYYANSVKGRFTISRDNSKNTLYLQMNSLRAEDTAVYYCAEPKPMDTEFDYRSPGTLVTVSSHHHHH

SEQ ID NO: 357 - protein, 605 aa, synthetic construct
QVTLKESGPGILQPSQTLSLTCSFSGFSLRTSGMGVGWIRQPSGKGLEWLAHIWWDDDKRYNPALKSRLTISKDTSSNQVFLKIASVDTADTATYYCAQINPAWFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTSPPSPAPPVAGPSVFLFPPKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSDIQMTQSPSSLSASVGDRVTITCRASQYIYDYLNWYQQKPGKAPKLLIYAASYLQSGVPSRFSGSGSGTDFTLTISSLQPEDFATYYCQQYHHPSTFGQGTKVEIKRGGGGSGGGGSGGGGSEVQLLESGGGLVQPGGSLRLSCAASGFTFRNHDMGWVRQAPGKGLEWVSAISGSGGSTYYANSVKGRFTISRDNSKNTLYLQMNSLRAEDTAVYYCAEPKPMDTEFDYRSPGTLVTVSSHHHHHH

SEQ ID NO: 358 - protein, 493 aa, synthetic construct
QVTLKESGPGILQPSQTLSLTCSFSGFSLRTSGMGVGWIRQPSGKGLEWLAHIWWDDDKRYNPALKSRLTISKDTSSNQVFLKIASVDTADTATYYCAQINPAWFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPPVAGPSVFLFPPKDIQMTQSPSSLSASVGDRVTITCRASQYIYDYLNWYQQKPGKAPKLLIYAASYLQSGVPSRFSGSGSGTDFTLTISSLQPEDFATYYCQQYHHPSTFGQGTKVEIKRGGGGSGGGGSGGGGSEVQLLESGGGLVQPGGSLRLSCAASGFTFRNHDMGWVRQAPGKGLEWVSAISGSGGSTYYANSVKGRFTISRDNSKNTLYLQMNSLRAEDTAVYYCAEPKPMDTEFDYRSPGTLVTVSSHHHHHH

SEQ ID NO: 359 - protein, 478 aa, synthetic construct
QVTLKESGPGILQPSQTLSLTCSFSGFSLRTSGMGVGWIRQPSGKGLEWLAHIWWDDDKRYNPALKSRLTISKDTSSNQVFLKIASVDTADTATYYCAQINPAWFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTGGGGSDIQMTQSPSSLSASVGDRVTITCRASQYIYDYLNWYQQKPGKAPKLLIYAASYLQSGVPSRFSGSGSGTDFTLTISSLQPEDFATYYCQQYHHPSTFGQGTKVEIKRGGGGSGGGGSGGGGSEVQLLESGGGLVQPGGSLRLSCAASGFTFRNHDMGWVRQAPGKGLEWVSAISGSGGSTYYANSVKGRFTISRDNSKNTLYLQMNSLRAEDTAVYYCAEPKPMDTEFDYRSPGTLVTVSSHHHHHH

SEQ ID NO: 360 - protein, 265 aa, synthetic construct
DIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSGHNYTTRNILPGLNITSHHHHHH

SEQ ID NO: 361 - protein, 27 aa, synthetic construct
GHNYTTRNILPGLNITSEPKSSDKTHT

SEQ ID NO: 362 - protein, 29 aa, synthetic construct
GHNYTTRNILPGLNITSGSEPKSSDKTHT

SEQ ID NO: 363 - protein, 32 aa, synthetic construct
GHNYTTRNILPGLNITSGGGGSEPKSSDKTHT

SEQ ID NO: 364 - protein, 37 aa, synthetic construct
GHNYTTRNILPGLNITSTFHKTSGSGKEPKSSDKTHT

SEQ ID NO: 365 - protein, 39 aa, synthetic construct
GHNYTTRNILPGLNITSTFHKTSGSGKGSEPKSSDKTHT

SEQ ID NO: 366 - protein, 42 aa, synthetic construct
GHNYTTRNILPGLNITSTFHKTSGSGKGGGGSEPKSSDKTHT

SEQ ID NO: 367 - protein, 29 aa, synthetic construct
GHNYTTRNILPGLNITSEPKSSDKTHTGS

SEQ ID NO: 368 - protein, 31 aa, synthetic construct
GHNYTTRNILPGLNITSGSEPKSSDKTHTGS

SEQ ID NO: 369 - protein, 34 aa, synthetic construct
GHNYTTRNILPGLNITSGGGGSEPKSSDKTHTGS

SEQ ID NO: 370 - protein, 39 aa, synthetic construct
GHNYTTRNILPGLNITSTFHKTSGSGKEPKSSDKTHTGS

SEQ ID NO: 371 - protein, 41 aa, synthetic construct
GHNYTTRNILPGLNITSTFHKTSGSGKGSEPKSSDKTHTGS

SEQ ID NO: 372 - protein, 44 aa, synthetic construct
GHNYTTRNILPGLNITSTFHKTSGSGKGGGGSEPKSSDKTHTGS

SEQ ID NO: 373 - protein, 32 aa, synthetic construct
GHNYTTRNILPGLNITSEPKSSDKTHTGGGGS

SEQ ID NO: 374 - protein, 34 aa, synthetic construct
GHNYTTRNILPGLNITSGSEPKSSDKTHTGGGGS

SEQ ID NO: 375 - protein, 37 aa, synthetic construct
GHNYTTRNILPGLNITSGGGGSEPKSSDKTHTGGGGS

SEQ ID NO: 376 - protein, 42 aa, synthetic construct
GHNYTTRNILPGLNITSTFHKTSGSGKEPKSSDKTHTGGGGS

SEQ ID NO: 377 - protein, 44 aa, synthetic construct
GHNYTTRNILPGLNITSTFHKTSGSGKGSEPKSSDKTHTGGGGS

SEQ ID NO: 378 - protein, 47 aa, synthetic construct
GHNYTTRNILPGLNITSTFHKTSGSGKGGGGSEPKSSDKTHTGGGGS

SEQ ID NO: 379 - protein, 30 aa, synthetic construct
DFPGHNYTTRNILPGLNITSEPKSSDKTHT

SEQ ID NO: 380 - protein, 32 aa, synthetic construct
DFPGHNYTTRNILPGLNITSGSEPKSSDKTHT

SEQ ID NO: 381 - protein, 35 aa, synthetic construct
DFPGHNYTTRNILPGLNITSGGGGSEPKSSDKTHT

SEQ ID NO: 382 - protein, 40 aa, synthetic construct
DFPGHNYTTRNILPGLNITSTFHKTSGSGKEPKSSDKTHT

SEQ ID NO: 383 - protein, 42 aa, synthetic construct
DFPGHNYTTRNILPGLNITSTFHKTSGSGKGSEPKSSDKTHT

SEQ ID NO: 384 - protein, 45 aa, synthetic construct
DFPGHNYTTRNILPGLNITSTFHKTSGSGKGGGGSEPKSSDKTHT

SEQ ID NO: 385 - protein, 32 aa, synthetic construct
DFPGHNYTTRNILPGLNITSEPKSSDKTHTGS

SEQ ID NO: 386 - protein, 34 aa, synthetic construct
DFPGHNYTTRNILPGLNITSGSEPKSSDKTHTGS

SEQ ID NO: 387 - protein, 37 aa, synthetic construct
DFPGHNYTTRNILPGLNITSGGGGSEPKSSDKTHTGS

SEQ ID NO: 388 - protein, 42 aa, synthetic construct
DFPGHNYTTRNILPGLNITSTFHKTSGSGKEPKSSDKTHTGS

SEQ ID NO: 389 - protein, 44 aa, synthetic construct
DFPGHNYTTRNILPGLNITSTFHKTSGSGKGSEPKSSDKTHTGS

SEQ ID NO: 390 - protein, 47 aa, synthetic construct
DFPGHNYTTRNILPGLNITSTFHKTSGSGKGGGGSEPKSSDKTHTGS

SEQ ID NO: 391 - protein, 35 aa, synthetic construct
DFPGHNYTTRNILPGLNITSEPKSSDKTHTGGGGS

SEQ ID NO: 392 - protein, 37 aa, synthetic construct
DFPGHNYTTRNILPGLNITSGSEPKSSDKTHTGGGGS

SEQ ID NO: 393 - protein, 40 aa, synthetic construct
DFPGHNYTTRNILPGLNITSGGGGSEPKSSDKTHTGGGGS

SEQ ID NO: 394 - protein, 45 aa, synthetic construct
DFPGHNYTTRNILPGLNITSTFHKTSGSGKEPKSSDKTHTGGGGS

SEQ ID NO: 395 - protein, 47 aa, synthetic construct
DFPGHNYTTRNILPGLNITSTFHKTSGSGKGSEPKSSDKTHTGGGGS

SEQ ID NO: 396 - protein, 50 aa, synthetic construct
DFPGHNYTTRNILPGLNITSTFHKTSGSGKGGGGSEPKSSDKTHTGGGGS